SEQUENCE LISTING (508 sequences)

SEQ ID NO: 1 - protein, 5 aa, synthetic construct
TYTMH

SEQ ID NO: 2 - protein, 5 aa, synthetic construct
TYPMH

SEQ ID NO: 3 - protein, 5 aa, synthetic construct
TYGMH

SEQ ID NO: 4 - protein, 10 aa, synthetic construct
GFTFRTYTMH

SEQ ID NO: 5 - protein, 10 aa, synthetic construct
GFTFRTYPMH

SEQ ID NO: 6 - protein, 10 aa, synthetic construct
GFTFRTYGMH

SEQ ID NO: 7 - protein, 8 aa, synthetic construct
GFTFRTYT

SEQ ID NO: 8 - protein, 8 aa, synthetic construct
GFTFRTYP

SEQ ID NO: 9 - protein, 8 aa, synthetic construct
GFTFRTYG

SEQ ID NO: 10 - protein, 17 aa, synthetic construct
VIWYDGSNKHYADSVKG

SEQ ID NO: 11 - protein, 6 aa, synthetic construct
WYDGSN

SEQ ID NO: 12 - protein, 8 aa, synthetic construct
IWYDGSNK

SEQ ID NO: 13 - protein, 13 aa, synthetic construct
APQWELVHEAFDI

SEQ ID NO: 14 - protein, 13 aa, synthetic construct
ALIWELFHEYFDI

SEQ ID NO: 15 - protein, 13 aa, synthetic construct
ALLWELFHEYFDI

SEQ ID NO: 16 - protein, 15 aa, synthetic construct
ARAPQWELVHEAFDI

SEQ ID NO: 17 - protein, 15 aa, synthetic construct
ARALIWELFHEYFDI

SEQ ID NO: 18 - protein, 15 aa, synthetic construct
ARALLWELFHEYFDI

SEQ ID NO: 19 - protein, 11 aa, synthetic construct
GGNNLGSKSVH

SEQ ID NO: 20 - protein, 11 aa, synthetic construct
GGNNLISKSVH

SEQ ID NO: 21 - protein, 11 aa, synthetic construct
GGNNLVSKSVH

SEQ ID NO: 22 - protein, 6 aa, synthetic construct
NLGSKS

SEQ ID NO: 23 - protein, 6 aa, synthetic construct
NLISKS

SEQ ID NO: 24 - protein, 6 aa, synthetic construct
NLVSKS

SEQ ID NO: 25 - protein, 7 aa, synthetic construct
DVSDRPS

SEQ ID NO: 26 - protein, 7 aa, synthetic construct
DDSDRPS

SEQ ID NO: 27 - protein, 11 aa, synthetic construct
QVWDSSSTFVV

SEQ ID NO: 28 - protein, 11 aa, synthetic construct
QVWDSSSTYVV

SEQ ID NO: 29 - protein, 11 aa, synthetic construct
QVWDSSSDHVV

SEQ ID NO: 30 - protein, 11 aa, synthetic construct
QVWDSSSTIVV

SEQ ID NO: 31 - protein, 11 aa, synthetic construct
QVWDKSSTYVV

SEQ ID NO: 32 - protein, 122 aa, synthetic construct
QMQLVESGGGVVQPGRSLRLSCAASGFTFRTYTMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 33 - protein, 122 aa, synthetic construct
QMQLVESGGGVVQPGRSLRLSCAASGFTFRTYPMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 34 - protein, 122 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 35 - protein, 122 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARALLWELFHEYFDIWGQGTMVTVSS

SEQ ID NO: 36 - protein, 122 aa, synthetic construct
QMQLVESGGGVVQPGRSLRLSCAASGFTFRTYPMHWVRQAPGKGLEWVAVIWYDGSNKHYADSVKGRFTITRDNSKNTLNLQMNSLRAEDTAVYYCARALLWELFHEYFDIWGQGTMVTVSS

SEQ ID NO: 37 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTFVVFGGGTKLTVL

SEQ ID NO: 38 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTYVVFGGGTKLTVL

SEQ ID NO: 39 - protein, 108 aa, synthetic construct
SYVLTQPPSVSVAPGQTARITCGGNNLISKSVHWYQQKPGQAPVLVVYDDSDRPSWIPERFSGSNSGNTATLTISRGEAGDEADYYCQVWDSSSTYVVFGGGTKLTVL

SEQ ID NO: 40 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTYVVFGGGTKLTVL

SEQ ID NO: 41 - protein, 108 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSTFVVFGGGTKLTVL

SEQ ID NO: 42 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSDHVVFGGGTKLTVL

SEQ ID NO: 43 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTIVVFGGGTKLTVL

SEQ ID NO: 44 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDKSSTYVVFGGGTKLTVL

SEQ ID NO: 45 - protein, 108 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSTYVVFGGGTKLTVL

SEQ ID NO: 46 - protein, 107 aa, Homo sapiens [sequence omitted]

SEQ ID NO: 47 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 49 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 50 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 51 - protein, 325 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 52 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 66 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 67 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 68 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 69 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 70 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 71 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 72 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 73 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 74 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 75 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 76 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 77 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 78 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 79 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 81 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 82 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 83 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 84 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 85 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 86 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 87 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 88 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 89 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPG

SEQ ID NO: 90 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPG

SEQ ID NO: 91 - protein, 326 aa, synthetic construct; identical to SEQ ID NO: 49

SEQ ID NO: 92 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 93 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 94 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 95 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 96 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 97 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 98 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 99 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 100 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 101 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 102 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 103 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 104 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 105 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 106 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 107 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 108 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 109 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 110 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 111 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 112 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 113 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 135 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 136 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 137 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 138 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 139 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 140 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 141 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 142 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 143 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 144 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 145 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 146 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 147 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 148 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 149 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 150 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 151 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 152 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHRDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPG

SEQ ID NO: 153 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 154 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 155 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 156 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 157 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 158 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 159 - protein, 330 aa, Homo sapiens [sequence omitted]

SEQ ID NO: 160 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 162 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 164 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 178 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 179 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 180 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 181 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 182 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 183 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 184 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 185 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 186 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 187 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 188 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 189 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 190 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 191 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 192 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 193 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 194 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 195 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 196 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 197 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 198 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 199 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 200 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 201 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 202 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPGK

SEQ ID NO: 203 - protein, 327 aa, synthetic construct; identical to SEQ ID NO: 161

SEQ ID NO: 204 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 205 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 206 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 207 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 208 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 209 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 210 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 211 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 212 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 213 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 214 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 215 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 216 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 217 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 218 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 219 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 220 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 221 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 222 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 223 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 224 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 225 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 247 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 248 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 249 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 250 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 251 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 252 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 253 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 254 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 255 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 256 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 257 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 258 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 259 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 260 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 261 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 262 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 263 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 264 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHRDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 265 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 266 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 267 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 268 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 269 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 270 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 271 - protein, 11 aa, synthetic construct
RTSQNIRYYLN

SEQ ID NO: 272 - protein, 11 aa, synthetic construct
GGDNIGSKSVH

SEQ ID NO: 273 - protein, 11 aa, synthetic construct
GGQNIGTKSVH

SEQ ID NO: 274 - protein, 11 aa, synthetic construct
GGNKIGRKSVH

SEQ ID NO: 275 - protein, 11 aa, synthetic construct
GGNNIGGKSVH

SEQ ID NO: 276 - protein, 11 aa, synthetic construct
GGNNIGSKSVH

SEQ ID NO: 277 - protein, 11 aa, synthetic construct
GGHNIGTKSVH

SEQ ID NO: 278 - protein, 11 aa, synthetic construct
GGDSIGSKYVH

SEQ ID NO: 279 - protein, 11 aa, synthetic construct
GGQNIGTRSVH

SEQ ID NO: 280 - protein, 11 aa, synthetic construct
GGNNIGSETVH

SEQ ID NO: 281 - protein, 11 aa, synthetic construct
RGDNIETYSVH

SEQ ID NO: 282 - protein, 11 aa, synthetic construct
GGNNIGRKSAH

SEQ ID NO: 283 - protein, 11 aa, synthetic construct
EGTYLGAKSVH

SEQ ID NO: 284 - protein, 11 aa, synthetic construct
GGNNIGTKSVH

SEQ ID NO: 285 - protein, 11 aa, synthetic construct
GGQSIGTKSVH

SEQ ID NO: 286 - protein, 11 aa, synthetic construct
GANSIEDKNVH

SEQ ID NO: 287 - protein, 11 aa, synthetic construct
GGDNIGSYSVH

SEQ ID NO: 288 - protein, 11 aa, synthetic construct
GGNNVEDYSVH

SEQ ID NO: 289 - protein, 6 aa, synthetic construct
QNIRYY

SEQ ID NO: 290 - protein, 6 aa, synthetic construct
NIGSKS

SEQ ID NO: 291 - protein, 6 aa, synthetic construct
NIGTKS

SEQ ID NO: 292 - protein, 6 aa, synthetic construct
KIGRKS

SEQ ID NO: 293 - protein, 6 aa, synthetic construct
NIGGKS

SEQ ID NO: 294 - protein, 6 aa, synthetic construct
SIGSKY

SEQ ID NO: 295 - protein, 6 aa, synthetic construct
NIGTRS

SEQ ID NO: 296 - protein, 6 aa, synthetic construct
NIGSET

SEQ ID NO: 297 - protein, 6 aa, synthetic construct
NIETYS

SEQ ID NO: 298 - protein, 6 aa, synthetic construct
NIGRKS

SEQ ID NO: 299 - protein, 6 aa, synthetic construct
SIGTKS

SEQ ID NO: 300 - protein, 6 aa, synthetic construct
SIEDKN

SEQ ID NO: 301 - protein, 6 aa, synthetic construct
NIGSYS

SEQ ID NO: 302 - protein, 6 aa, synthetic construct
NVEDYS

SEQ ID NO: 303 - protein, 7 aa, synthetic construct
DVSSLQS

SEQ ID NO: 304 - protein, 7 aa, synthetic construct
EDSDRPS

SEQ ID NO: 305 - protein, 7 aa, synthetic construct
DDTDRPS

SEQ ID NO: 306 - protein, 7 aa, synthetic construct
DDSDRPP

SEQ ID NO: 307 - protein, 7 aa, synthetic construct
DDKERPS

SEQ ID NO: 308 - protein, 7 aa, synthetic construct
DDSERPS

SEQ ID NO: 309 - protein, 7 aa, synthetic construct
DDLKRPS

SEQ ID NO: 310 - protein, 7 aa, synthetic construct
DDNDRPS

SEQ ID NO: 311 - protein, 9 aa, synthetic construct
QQGYSPPLT

SEQ ID NO: 312 - protein, 11 aa, synthetic construct
QVWDSDSDHVV

SEQ ID NO: 313 - protein, 11 aa, synthetic construct
QVWDSSSDQSV

SEQ ID NO: 314 - protein, 11 aa, synthetic construct
QVWDSSSDEGV

SEQ ID NO: 315 - protein, 11 aa, synthetic construct
QVWDGSSDQSV

SEQ ID NO: 316 - protein, 11 aa, synthetic construct
QVWDTTTDHVV

SEQ ID NO: 317 - protein, 11 aa, synthetic construct
QVWDSSSDPVV

SEQ ID NO: 318 - protein, 11 aa, synthetic construct
QVWDRSSDQSV

SEQ ID NO: 319 - protein, 11 aa, synthetic construct
QVWDSGSGQTV

SEQ ID NO: 320 - protein, 11 aa, synthetic construct
HVWDSSSDHVM

SEQ ID NO: 321 - protein, 11 aa, synthetic construct
QVWDSDSDHLE

SEQ ID NO: 322 - protein, 11 aa, synthetic construct
QVWDSYSNHVV

SEQ ID NO: 323 - protein, 11 aa, synthetic construct
QVWDSGGDHVV

SEQ ID NO: 324 - protein, 11 aa, synthetic construct
QVWDDISDHIV

SEQ ID NO: 325 - protein, 11 aa, synthetic construct
QVWDMASDRVV

SEQ ID NO: 326 - protein, 11 aa, synthetic construct
QVWDTSSDQGV

SEQ ID NO: 327 - protein, 11 aa, synthetic construct
QVWDSTSGVVF

SEQ ID NO: 328 - protein, 107 aa, synthetic construct
DIQLTQSPSSLSASVGDRVAITCRTSQNIRYYLNWYQRKPGKAPRLLIYDVSSLQSGVPSRFSGRGSGTDFTLTISSLQAEDFATYYCQQGYSPPLTFGGGTKVEIK

SEQ ID NO: 329 - protein, 108 aa, synthetic construct
NFMLTQPPSVSVAPGKTAKMTCGGDNIGSKSVHWYQQMPGQAPVLVIYEDSDRPSGTPERFSGSNSGHTATLTISRVEAGDEADYYCQVWDSDSDHVVFGGGTTLTVL

SEQ ID NO: 330 - protein, 108 aa, synthetic construct
QSVLTQPPSVSVAPGQTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 331 - protein, 108 aa, synthetic construct
SYELTQPPSVSVAPGQTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 332 - protein, 108 aa, synthetic construct
SYVLTQPPSVSVAPGQTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 333 - protein, 108 aa, synthetic construct
SYVLTQPPSVSVAPGQTARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 334 - protein, 108 aa, synthetic construct
SYELTQPPSVSVAPGQAARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 335 - protein, 108 aa, synthetic construct
QSVLTQPPSVSVAPGQTARITCGGQNIGTKSVHWYQQKSGQAPILVVYDDSDRPSWIPERFSGSNFGTTATLTISRVEAGDEADYFCQVWDGSSDQSVFGPGTRVTVL

SEQ ID NO: 336 - protein, 108 aa, synthetic construct
SYELTQPPSVSVAPGQTASITCGGNNIGGKSVHWYQLKPGQAPVLVVYDDTDRPSWIPERFSGSNSANTATLTISGVEAGDEADYQCQVWDTTTDHVVFGGGTKLTVL

SEQ ID NO: 337 - protein, 108 aa, synthetic construct
SYVLTQPPSVSVAPGQTARITCGGNNIGSKSVHWYQQKPGQAPLLVVYDDSDRPSWIPERFSGSNSGNAATLTVSRVEAGDEADYYCQVWDSSSDPVVFGGGTKLTVL

SEQ ID NO: 338 - protein, 108 aa, synthetic construct
FYVLTQPPSVSVAPGQTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 339 - protein, 108 aa, synthetic construct
QSVLTQPPSVSVAPGQTARITCGGQNIGTKSVHWYQQKSGQAPILVVYDDSDRPPWIPERFSGSNFGTTATLTISRVEAGDEADYFCQVWDSSSDQSVFGPGTRVTVL

SEQ ID NO: 340 - protein, 108 aa, synthetic construct
QAVLTQPPSVSVAPGQTARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 341 - protein, 108 aa, synthetic construct
SYELTQPPSVSVAPGQTARITCGGHNIGTKSVHWYQQKSGQAPILVVYDDSDRPSWIPERFSGSNFGTTATLTISRVEAGDEADYFCQVWDRSSDQSVFGPGTRVTVL

SEQ ID NO: 342 - protein, 108 aa, synthetic construct
SYVLTQPPSVSVAPGQTARVTCGGDSIGSKYVHWYQQKPGQAPVLVVYDDSDRPSWIPERFSGSNFGTTATLTISRVEAGDEADYFCQVWDRSSDQSVFGPGTRVTVL

SEQ ID NO: 343 - protein, 108 aa, synthetic construct
SYELTQPPSVSVAPGQTARITCGGQNIGTKSVHWYQQKSGQAPILVVFDDSDRPSWVTERFSGSNFGTTATLTINGVEAGDEADYFCQVWDSGSGQTVFGPGTRVTVS

SEQ ID NO: 344 - protein, 108 aa, synthetic construct
SYELTQPPSVSVAPGQTARITCGGQNIGTRSVHWYQQKSGQAPILVVYDDSDRPSWIPERFSGSNFGTTATLTISRVEAGDEADYFCQVWDSSSDQSVFGPGTRVTVL

SEQ ID NO: 345 - protein, 108 aa, synthetic construct
SYELTQPPSVSVAPGQTARITCGGNNIGSETVHWYRQKAGQAPVLVVYDDSDRPSGIPERFSGSNSGNTAALTISRVEVGDEADYYCHVWDSSSDHVMFGGGTKLTVL

SEQ ID NO: 346 - protein, 108 aa, synthetic construct
SYELTQPPSVSVGPGKTATITCRGDNIETYSVHWYQQKPGQAPVLVVYDDSDRPSGIPERFSGSNSGNTATLTINRVGAGDEADYYCQVWDSDSDHLEFGGGTKLTVL

SEQ ID NO: 347 - protein, 108 aa, synthetic construct
SYELTQPPSVSVAPGQTARITCGGNNIGRKSAHWYQQRPGQAPVLVVYDDSDRPSWIPERFSGSNSGNTATLTISRVEAGDEADYYCQVWDSYSNHVVFGGGTKLTVL

SEQ ID NO: 348 - protein, 108 aa, synthetic construct
LPVLTQPPSVSVAPGQTARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 349 - protein, 108 aa, synthetic construct
SYVLTQPPSVSVAPGQTARITCGGHNIGTKSVHWYQQKSGQAPILAVYDDSDRPSWIPERFSGSNFGTTATLTISRVEAGDEADYFCQVWDRSSDQSVFGPGTRVTVL

SEQ ID NO: 350 - protein, 108 aa, synthetic construct
SYELTQPPSVSVGPGKTATITCRGDNIETYSVHWYQQKPGQAPVLVVYDDSDRPSGIPERFSGSNSGNTATLTINRVEARDEADYFCQVWDSGGDHVVFGGGTKLTVL

SEQ ID NO: 351 - protein, 108 aa, synthetic construct
SYVLTQPPSVSVAPGQTARITCGGNNIGSKSVHWYQQKPGQAPVLVVYDDSDRPSWIPERFSGTNSGNTATLTISRVEAGDEADYYCQVWDSSSDHVVFGGGTKLTVL

SEQ ID NO: 352 - protein, 108 aa, synthetic construct
SYELTQPPSVSVAPGKRARITCEGTYLGAKSVHWYQQKPGQAPVLVIYDDKERPSGIPERFSASNSGNTATLTITRVEAGDEADYHCQVWDDISDHIVFGGGTKLSVL

SEQ ID NO: 353 - protein, 108 aa, synthetic construct
SYVLTQPPSVSVAPGQTARITCGGNNIGTKSVHWYQQKSGQAPILVVYDDSDRPSWIPERFSGSNFGTTATLTISRVEAGDEADYFCQVWDSSSDQSVFGPGTRVTVL

SEQ ID NO: 354 - protein, 108 aa, synthetic construct
SYVLTQPPSVSVAPGQTARITCGGQSIGTKSVHWYQQKSGQAPILVVYDDSDRPSWIPERFSGSNFGTTATLTISRVEAGDEADYFCQVWDSSSDQSVFGPGTRVTVL

SEQ ID NO: 355 - protein, 108 aa, synthetic construct
SYELTQPPSVSVAPGQAASLTCGANSIEDKNVHWYQQRPGQAPLLVIYDDSERPSGIPERFSGSNSGNTATLTITRVAAGDEADYYCQVWDMASDRVVFGPGTTVSVL

SEQ ID NO: 356 - protein, 108 aa, synthetic construct
SYELTQPPSVSVAPGQTARITCGGNKIGRKSVHWYQQRPGQAPVLVVFDDSDRPSWISERFSGSNSGNTATLTIGRVEAGDEADYYCQVWDSSSDEGVFGGGTKLAVL

SEQ ID NO: 357 - protein, 108 aa, synthetic construct
SYELTQPPSVSVAPGRTARITCGGQNIGTKSVHWYQQKSGQAPILVVYDDSDRPSWIPERFSGSNFGTTATLTISRVEAGDEADYFCQVWDSSSDQSVFGPGTRVTVL

SEQ ID NO: 358 - protein, 108 aa, synthetic construct
QAGLTQPPSVSVAPGQTARITCGGNKIGRKSVHWYQQRPGQAPVLVVFDDSDRPSWISERFSGSNSGNTATLTISRVEAGDEADYYCQVWDSSSDEGVFGGGTKLTVL

SEQ ID NO: 359 - protein, 108 aa, synthetic construct
QSVLTQPPSVSVAPGQTARITCGGQNIGTKSVHWYQQKSGQAPILVVYDDSDRPSWIPERFSGSNFGTTATLTISRVEAGDEADYFCQVWDSSSDQSVFGTGTKVTVL

SEQ ID NO: 360 - protein, 108 aa, synthetic construct
SYVLTQPPSVSVGPGKTATITCRGDNIETYSVHWYQRKPGQAPVLVVYDDSDRPSGIPERFSGSNSGNTATLTISRVEAGDEADYYCQVWDSSSDHVVFGGGTKLTVL

SEQ ID NO: 361 - protein, 108 aa, synthetic construct
SYELTQPPSVSVAPGQTARITCGGNKIGRKSVHWYQQRPGQAPVLVVFDDSDRPSWISERFSGSNSGNTATLTISRVDAGDEADYYCQVWDSSSDEGVFGGGTKLTVL

SEQ ID NO: 362 - protein, 108 aa, synthetic construct
SYELTQPPSVSVAPGQTARITCGGNKIGRKSVHWYQQKPGQAPVLVVFDDSDRPSWISERFSGSNSGNTATLTISRVEAGDEADYHCQVWDTSSDQGVFGGGTKLTVL

SEQ ID NO: 363 - protein, 107 aa, synthetic construct
SYVLTQPPSVAVAPGQTAKITCGGDNIGSYSVHWYQQKPGQAPVLVVYDDLKRPSWIPERFFGSNSGNTATLTISRVEAGDEAVYYCQVWDSTSGVVFGGGTTVTVL

SEQ ID NO: 364 - protein, 108 aa, synthetic construct
SYELTQPPSVSVAPGKTARITCGGNNVEDYSVHWYHQKAGQAPVVVMYDDNDRPSGVPERFSGSNSGNTATLTIGRVEAGDEADYYCQVWDSSSDHVVFGGGTKLTVL

SEQ ID NO: 365 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 366 - protein, 159 aa, Macaca fascicularis
MKSLGQSKKEEVSFRKFFIFQLVGLVLTYDFTNCDFQKIEADYLRTISKDLITYMSGTKSTDFNNTVSCSNRPHCLTEIQSLTFNPTPRCASLAKEMFARKTKATLALWCPGYSETQINATQAMKKRRKRKVTTNKCLEQVSQLLGLWRRFIRTLLKKQ

SEQ ID NO: 367 - protein, 11 aa, synthetic construct
QVWDSSSDFVV

SEQ ID NO: 368 - protein, 11 aa, synthetic construct
QVWDSSSTHVV

SEQ ID NO: 369 - protein, 11 aa, synthetic construct
QVWDSSSTFVV

SEQ ID NO: 370 - protein, 11 aa, synthetic construct
QVWDSSSTIVV

SEQ ID NO: 371 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSDHVVFGGGTKLTVL

SEQ ID NO: 372 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDFVVFGGGTKLTVL

SEQ ID NO: 373 - protein, 108 aa, synthetic construct; identical to SEQ ID NO: 42

SEQ ID NO: 374 - protein, 108 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSDFVVFGGGTKLTVL

SEQ ID NO: 375 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTHVVFGGGTKLTVL

SEQ ID NO: 376 - protein, 108 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSDFVVFGGGTKLTVL

SEQ ID NO: 377 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSTFVVFGGGTKLTVL

SEQ ID NO: 378 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTHVVFGGGTKLTVL

SEQ ID NO: 379 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSDFVVFGGGTKLTVL

SEQ ID NO: 380 - skipped

SEQ ID NO: 381 - protein, 108 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSTIVVFGGGTKLTVL

SEQ ID NO: 382 - protein, 108 aa, synthetic construct; identical to SEQ ID NO: 37

SEQ ID NO: 383 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTFVVFGGGTKLTVL

SEQ ID NO: 384 - protein, 108 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTIVVFGGGTKLTVL

SEQ ID NO: 385 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 386 - protein, 122 aa, synthetic construct
QMQLVESGGGVVQPGRSLRLSCAASGFTFRTYPMHWVRQAPGKGLEWVAVIWYDGSNKHYADSVKGRFTITRDNSKNTLNLQMNSLRAEDTAVYYCARALIWELFHEYFDIWGQGTMVTVSS

SEQ ID NO: 387 - protein, 6 aa, synthetic construct
YLGAKS

SEQ ID NO: 388 - protein, 216 aa, synthetic construct
QSALTQPPSASGSLGQSVTISCTGTSSDVGGYNYVSWYQQHAGKAPKVIIYEVNKRPSGVPDRFSGSKSGNTASLTVSGLQAEDEADYYCSSYEGSDNFVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 389-400 - skipped (12 entries)

SEQ ID NO: 401 - protein, 5 aa, unidentified
AKEMF

SEQ ID NO: 402 - protein, 17 aa, unidentified
YLSTISKDLITYMSGTK

SEQ ID NO: 403 - protein, 10 aa, unidentified
SLAKEMFAMK

SEQ ID NO: 404 - protein, 9 aa, unidentified
WRRFNRPLL

SEQ ID NO: 405 - protein, 8 aa, unidentified
ISKDLITY

SEQ ID NO: 406 - protein, 7 aa, unidentified
KEMFAMK

SEQ ID NO: 407 - protein, 9 aa, unidentified
RRFNRPLLK

SEQ ID NO: 408 - protein, 7 aa, unidentified
SKDLITY

SEQ ID NO: 409 - protein, 509 aa, unidentified
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 410 - protein, 474 aa, unidentified
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 411 - protein, 447 aa, synthetic construct [sequence omitted]

SEQ ID NO: 412 - protein, 446 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 413 - protein, 445 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 414 - protein, 444 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 415 - protein, 443 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 416 - protein, 442 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEE

SEQ ID NO: 417 - protein, 432 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 418 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 419 - DNA, 1446 bp, synthetic construct
  source 1..1446: mol_type = other DNA
atgggagtgctaaaattcaagcacatctttttcagaagctttgttaaatcaagtggagtatcccagatagttttcaccttccttctgattccatgttgcttgactctgaatttcagagcacctcctgttattccaaatgtgcctttcctctgggcctggaatgccccaagtgaattttgtcttggaaaatttgatgagccactagatatgagcctcttctctttcataggaagcccccgaataaacgccaccgggcaaggtgttacaatattttatgttgatagacttggctactatccttacatagattcaatcacaggagtaactgtgaatggaggaatcccccagaagatttccttacaagaccatctggacaaagctaagaaagacattacattttatatgccagtagacaatttgggaatggctgttattgactgggaagaatggagacccacttgggcaagaaactggaaacctaaagatgtttacaagaataggtctattgaattggttcagcaacaaaatgtacaacttagtctcacagaggccactgagaaagcaaaacaagaatttgaaaaggcagggaaggatttcctggtagagactataaaattgggaaaattacttcggccaaatcacttgtggggttattatctttttccggattgttacaaccatcactataagaaacccggttacaatggaagttgcttcaatgtagaaataaaaagaaatgatgatctcagctggttgtggaatgaaagcactgctctttacccatccatttatttgaacactcagcagtctcctgtagctgctacactctatgtgcgcaatcgagttcgggaagccatcagagtttccaaaatacctgatgcaaaaagtccacttccggtttttgcatatacccgcatagtttttactgatcaagttttgaaattcctttctcaagatgaacttgtgtatacatttggcgaaactgttgctctgggtgcttctggaattgtaatatggggaaccctcagtataatgcgaagtatgaaatcttgcttgctcctagacaattacatggagactatactgaatccttacataatcaacgtcacactagcagccaaaatgtgtagccaagtgctttgccaggagcaaggagtgtgtataaggaaaaactggaattcaagtgactatcttcacctcaacccagataattttgctattcaacttgagaaaggtggaaagttcacagtacgtggaaaaccgacacttgaagacctggagcaattttctgaaaaattttattgcagctgttatagcaccttgagttgtaaggagaaagctgatgtaaaagacactgatgctgttgatgtgtgtattgctgatggtgtctgtatagatgcttttctaaaacctcccatggagacagaagaacctcaaattttctac

SEQ ID NO: 420 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 421 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 422 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 423 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 424 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 425 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 426 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 427 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 428 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 429 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 430 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 431 - protein, 453 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 432 - protein, 430 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 433 - protein, 433 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 434 - protein, 436 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKP

SEQ ID NO: 435 - protein, 452 aa, synthetic construct
FRGPLLPNRPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 436 - protein, 452 aa, synthetic construct
FRGPLLPNRPFTTVWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 437 - protein, 433 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 438 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 439 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 440 - protein, 451 aa, synthetic construct
APPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 441 - protein, 449 aa, synthetic construct
PVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 442 - protein, 432 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 443 - protein, 429 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVC

SEQ ID NO: 444 - protein, 425 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGPTLEDLEQFSEKFYCSCYSTLSCKEKADVKDTDAVDVCIAD

SEQ ID NO: 445 - protein, 423 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVC

SEQ ID NO: 446 - protein, 420 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAV

SEQ ID NO: 447 - protein, 433 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 448 - protein, 435 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPP

SEQ ID NO: 449 - protein, 436 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPM

SEQ ID NO: 450 - protein, 437 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPME

SEQ ID NO: 451 - protein, 438 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMET

SEQ ID NO: 452 - protein, 439 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETE

SEQ ID NO: 453 - protein, 454 aa, synthetic construct
NFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 454 - protein, 452 aa, synthetic construct
RAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 455 - protein, 450 aa, synthetic construct
PPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 456 - protein, 428 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 457 - protein, 429 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCID

SEQ ID NO: 458 - protein, 430 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 459 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 460 - protein, 441 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 461 - protein, 443 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 462 - protein, 445 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 463 - protein, 447 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNA

SEQ ID NO: 464 - protein, 449 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASP

SEQ ID NO: 465 - protein, 451 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPST

SEQ ID NO: 466 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 467 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 468 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGCWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 469 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGDWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 470 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 471 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 472 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 473 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 474 - protein, 431 aa, synthetic construct
FRGPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 475 - protein, 431 aa, synthetic construct; identical to SEQ ID NO: 474

SEQ ID NO: 476 - protein, 448 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 477 - protein, 214 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 478 - protein, 122 aa, synthetic construct; identical to SEQ ID NO: 34

SEQ ID NO: 479 - protein, 108 aa, synthetic construct; identical to SEQ ID NO: 385

SEQ ID NO: 480 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 481 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 482 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 483 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 484 - protein, 452 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 485 - protein, 214 aa, synthetic construct
SYVLTQPPSVSVAPGQTARITCGGNNLVSKSVHWYQQKPGQAPVLVVYDDSDRPSWIPERFSGSNSGNTATLTISRGEAGDEADYYCQVWDSSSTFVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 486 - protein, 451 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 487 - protein, 452 aa, synthetic construct; identical to SEQ ID NO: 484

SEQ ID NO: 488 - protein, 214 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTIVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 489 - protein, 452 aa, synthetic construct; identical to SEQ ID NO: 484

SEQ ID NO: 490 - protein, 214 aa, synthetic construct
SYELTQPPSVSVAPGQTARITCGGHNIGTKSVHWYQQKSGQAPILVVYDDSDRPSWIPERFSGSNFGTTATLTISRVEAGDEADYFCQVWDRSSDQSVFGPGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 491 - protein, 452 aa, synthetic construct; identical to SEQ ID NO: 484

SEQ ID NO: 492 - protein, 214 aa, synthetic construct
SYELTQPPSVSVAPGQTARITCGGNNIGRKSAHWYQQRPGQAPVLVVYDDSDRPSWIPERFSGSNSGNTATLTISRVEAGDEADYYCQVWDSYSNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 493 - protein, 452 aa, synthetic construct; identical to SEQ ID NO: 484

SEQ ID NO: 494 - protein, 214 aa, synthetic construct
SYVLTQPPSVSVAPGQTARITCGGHNIGTKSVHWYQQKSGQAPILAVYDDSDRPSWIPERFSGSNFGTTATLTISRVEAGDEADYFCQVWDRSSDQSVFGPGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 495 - protein, 452 aa, synthetic construct; identical to SEQ ID NO: 484

SEQ ID NO: 496 - protein, 214 aa, synthetic construct
SYVLTQPPSVSVAPGQTARITCGGQSIGTKSVHWYQQKSGQAPILVVYDDSDRPSWIPERFSGSNFGTTATLTISRVEAGDEADYFCQVWDSSSDQSVFGPGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 497 - protein, 452 aa, synthetic construct; identical to SEQ ID NO: 484

SEQ ID NO: 498 - protein, 214 aa, synthetic construct
SYELTQPPSVSVAPGQTARITCGGNKIGRKSVHWYQQRPGQAPVLVVFDDSDRPSWISERFSGSNSGNTATLTIGRVEAGDEADYYCQVWDSSSDEGVFGGGTKLAVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 499 - protein, 452 aa, synthetic construct; identical to SEQ ID NO: 484

SEQ ID NO: 500 - protein, 214 aa, synthetic construct
SYELTQPPSVSVAPGRTARITCGGQNIGTKSVHWYQQKSGQAPILVVYDDSDRPSWIPERFSGSNFGTTATLTISRVEAGDEADYFCQVWDSSSDQSVFGPGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 501 - protein, 452 aa, synthetic construct; identical to SEQ ID NO: 484

SEQ ID NO: 502 - protein, 214 aa, synthetic construct
SYELTQPPSVSVAPGQTARITCGGNKIGRKSVHWYQQKPGQAPVLVVFDDSDRPSWISERFSGSNSGNTATLTISRVEAGDEADYHCQVWDTSSDQGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 503 - protein, 452 aa, synthetic construct; identical to SEQ ID NO: 484

SEQ ID NO: 504 - protein, 214 aa, synthetic construct
SYVLTQPPSVSVAPGQTARITCGGNNLGSKSVHWYQQKPGQAPVLVVYDVSDRPSWIPERFSGSNSGNTATLTISRGEAGDEADYYCQVWDSSSTHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 505 - protein, 452 aa, synthetic construct; identical to SEQ ID NO: 484

SEQ ID NO: 506 - protein, 214 aa, synthetic construct
SYVLTQPPSVSVAPGQTARITCGGNNLVSKSVHWYQQKPGQAPVLVVYDVSDRPSWIPERFSGSNSGNTATLTISRGEAGDEADYYCQVWDSSSTFVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 507 - protein, 5 aa, Homo sapiens
SRDEL

SEQ ID NO: 508 - protein, 5 aa, Homo sapiens
SREEM

(19) United States
(12) Patent Application Publication
(10) Pub. No.: US 2026/0199459 A1
(43) Pub. Date: Jul. 16, 2026

(54) ANTIBODIES THAT BIND TSLP IN COMBINATION WITH HYALURONIDASE OR A VARIANT THEREOF

(71) Applicant: Apogee Therapeutics, Inc., Waltham, MA (US)

(72) Inventors: Aaron NOYES, Apex, NC (US); Rebecca Lucille DABORA, Bethlehem, PA (US)

(21) Appl. No.: 19/361,095

(22) Filed: Oct. 17, 2025

Related U.S. Application Data
(60) Provisional application No. 63/708,880, filed on Oct. 18, 2024.
(60) Provisional application No. 63/827,123, filed on Jun. 20, 2025.

(51) Int. Cl. A61K 39/395; A61K 38/47; A61K 39/00

(52) U.S. Cl. CPC A61K 39/3955; A61K 38/47; C12Y 302/01035; A61K 2039/505; A61K 2039/54

(57) ABSTRACT

Described herein are compositions and combinations comprising an antibody that binds thymic stromal lymphopoietin (TSLP) and a hyaluronidase or variant thereof, as well as related methods of treating an inflammatory disorder or disease in a human patient.

### CROSS REFERENCE TO RELATED APPLICATIONS

This application claims the benefit of U.S. Provisional Application No. 63/708,880 (filed Oct. 18, 2024) and U.S. Provisional Application No. 63/827,123 (filed Jun. 20, 2025), both of which are incorporated by reference herein in their entirety.

### SEQUENCE LISTING

The instant application contains a Sequence Listing which has been submitted electronically in XML file format and is hereby incorporated by reference in its entirety. Said XML copy, created on Oct. 28, 2025, is named AOJ-011_SL.xml and is 551,017 bytes in size.

### BACKGROUND

Thymic stromal lymphopoietin (TSLP) is an epithelial cell-derived cytokine produced in response to pro-inflammatory stimuli. TSLP has been discovered to promote allergic inflammatory responses primarily through its activity on dendritic and mast cells. Human TSLP expression has been reported to be increased in asthmatic airways correlating to disease severity. In addition, TSLP protein levels are detectable in the concentrated bronchoalveolar lavage (BAL) fluid of asthma patients and other patients suffering from allergic disorders. In addition, TSLP has also been found to promote fibrosis.

TSLP binds to a heterodimeric receptor consisting of the TSLP receptor (TSLPR) and an IL-7 receptor a chain (IL-7Rα) in dendritic cells, thereby activating the dendritic cells. Upon activation, the dendritic cells express inflammatory chemokines such as thymus- and activation-regulated chemokine (TARC (CCL17)), macrophage-derived chemokine (MDC (CCL22)), and the like.

Activation of dendritic cells by TSLP through the TSLPR is associated with disease pathology, including allergic inflammatory diseases, such as asthma, and autoimmune disease, such as systemic sclerosis.

Accordingly, there is a need in the art for antagonists to TSLP for preventing and treating diseases in which human TSLP and human TSLPR are involved in the disease pathology, such as inflammatory and fibrotic disorders. There is also a need for delivering relatively large doses of these TSLP antagonists subcutaneously. Accordingly, it is an object of the present invention to provide improved methods for treating patients with inflammatory diseases and conditions by administering antagonists to TSLP alone or in combination with a hyaluronidase or a variant thereof.

### SUMMARY

The present disclosure provides compositions and combinations comprising an antibody, or antigen binding fragment thereof, to TSLP and a hyaluronidase or a variant thereof, and methods of treating diseases in which human TSLP is involved in disease pathology, such as an inflammatory disorder or disease, by administering to a subject in need thereof an anti-TSLP antibody and a hyaluronidase or variant thereof. The antibodies of the disclosure can include Fc modifications and exhibit longer half-lives than an otherwise identical antibody that does not include the Fc modification, for example, certain anti-TSLP antibodies known in the art.

In a first aspect, the invention provides a composition comprising an isolated antibody, or antigen binding fragment thereof, that binds TSLP and a hyaluronidase or a variant thereof, wherein the antibody, or antigen binding fragment thereof comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein: CDR-H1 comprises an amino acid sequence set forth in any one of SEQ ID NOs: 1-9; CDR-H2 comprises an amino acid sequence set forth in any one of SEQ ID NOs: 10-12; CDR-H3 comprises an amino acid sequence set forth in any one of SEQ ID NOs: 13-18; CDR-L1 comprises an amino acid sequence set forth in any one of SEQ ID NOs: 19-24, 271-302; and 387; CDR-L2 comprises an amino acid sequence set forth in any one of SEQ ID NOs: 25-26, 303-310, and amino acid sequences DDS, DVS, EDS, DDT, DDK, DDL, and DDN; and CDR-L3 comprises an amino acid sequence set forth in any one of SEQ ID NOs: 27-31, 311-327, and 367-370.

In a first aspect, the invention provides a combination comprising an isolated antibody, or antigen binding fragment thereof, that binds TSLP and a hyaluronidase or a variant thereof, wherein the antibody, or antigen binding fragment thereof comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein: CDR-H1 comprises an amino acid sequence set forth in any one of SEQ ID NOs: 1-9; CDR-H2 comprises an amino acid sequence set forth in any one of SEQ ID NOs: 10-12; CDR-H3 comprises an amino acid sequence set forth in any one of SEQ ID NOs: 13-18; CDR-L1 comprises an amino acid sequence set forth in any one of SEQ ID NOs: 19-24, 271-302; and 387; CDR-L2 comprises an amino acid sequence set forth in any one of SEQ ID NOs: 25-26, 303-310, and amino acid sequences DDS, DVS, EDS, DDT, DDK, DDL, and DDN; and CDR-L3 comprises an amino acid sequence set forth in any one of SEQ ID NOs: 27-31, 311-327, and 367-370.

In another aspect, the invention provides a method of treating an inflammatory disorder or disease in a human patient in need thereof comprising administering to the patient an isolated antibody, or antigen binding fragment thereof, that binds TSLP and a hyaluronidase or a variant thereof, wherein the isolated antibody, or antigen binding fragment thereof, that binds TSLP, comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein: CDR-H1 comprises an amino acid sequence set forth in any one of SEQ ID NOs: 1-9; CDR-H2 comprises an amino acid sequence set forth in any one of SEQ ID NOs: 10-12; CDR-H3 comprises an amino acid sequence set forth in any one of SEQ ID NOs: 13-18; CDR-L1 comprises an amino acid sequence set forth in any one of SEQ ID NOs: 19-24, 271-302; and 387; CDR-L2 comprises an amino acid sequence set forth in any one of SEQ ID NOs: 25-26, 303-310, and amino acid sequences DDS, DVS, EDS, DDT, DDK, DDL, and DDN; and CDR-L3 comprises an amino acid sequence set forth in any one of SEQ ID NOs: 27-31, 311-327, and 367-370.

In some embodiments, the antibody, or an antigen binding fragment thereof, comprises: CDR-H1 comprising an amino acid sequence set forth in any one of SEQ ID NOs: 1-3; CDR-H2 comprising an amino acid sequence set forth in SEQ ID NO: 10; CDR-H3 comprising an amino acid sequence set forth in any one of SEQ ID NOs: 13-15; CDR-L1 comprising an amino acid sequence set forth in any one of SEQ ID NOs: 19-21 and 271-288; CDR-L2 comprising an amino acid sequence set forth in any one of SEQ ID NOs: 25-26 and 303-310; and CDR-L3 comprising an amino acid sequence set forth in any one of SEQ ID NOs: 27-31 and 311-327.

In some embodiments, the antibody, or an antigen binding fragment thereof, comprises: CDR-H1 comprising an amino acid sequence set forth in any one of SEQ ID NOs: 4-6; CDR-H2 comprising an amino acid sequence set forth in SEQ ID NO: 11; CDR-H3 comprising an amino acid sequence set forth in any one of SEQ ID NOs: 13-15; CDR-L1 comprising an amino acid sequence set forth in any one of SEQ ID NOs: 19-21 and 271-288; CDR-L2 comprising an amino acid sequence set forth in any one of SEQ ID NOs: 25-26 and 303-310; and CDR-L3 comprising an amino acid sequence set forth in any one of SEQ ID NOs: 27-31 and 311-327.

In some embodiments, the antibody, or an antigen binding fragment thereof, comprises: CDR-H1 comprising an amino acid sequence set forth in any one of SEQ ID NOs: 7-9; CDR-H2 comprising an amino acid sequence set forth in SEQ ID NO: 12; CDR-H3 comprising an amino acid sequence set forth in any one of SEQ ID NOs: 16-18; CDR-L1 comprising an amino acid sequence set forth in any one of SEQ ID NOs: 22-24, 289-302, and 387; CDR-L2 comprising an amino acid sequence of any one of amino acid sequences DDS, DVS, EDS, DDT, DDK, DDL, and DDN; and CDR-L3 comprising an amino acid sequence set forth in any one of SEQ ID NOs: 27-31 and 311-327.

In some embodiments, the antibody, or an antigen binding fragment thereof, comprises a CDR-H1 comprising an amino acid sequence set forth in any one of SEQ ID NOs: 1, 4, and 7; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 19 and 22; a CDR-L2 comprising an amino acid sequence set forth in SEQ ID NO: 25 or the amino acid sequence DVS; and a CDR-L3 comprising an amino acid sequence set forth in SEQ ID NO: 27.

In some embodiments, the antibody, or an antigen binding fragment thereof, comprises a CDR-H1 comprising an amino acid sequence set forth in any one of SEQ ID NOs: 2, 5, and 8; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 19 and 22; a CDR-L2 comprising an amino acid sequence set forth in SEQ ID NO: 25 or the amino acid sequence DVS; and a CDR-L3 comprising an amino acid sequence set forth in SEQ ID NO: 28.

In some embodiments, the antibody, or an antigen binding fragment thereof, comprises a CDR-H1 comprising an amino acid sequence set forth in any one of SEQ ID NOs: 2, 5, and 8; a CDR-H2 comprising an amino acid sequence set forth in any one of SEQ ID NOs: 10, 11, and 12; a CDR-H3 comprising an amino acid sequence set forth in any one of SEQ ID NOs: 14 and 17; a CDR-L1 comprising an amino acid sequence set forth in any one of SEQ ID NOs: 20 and 23; a CDR-L2 comprising an amino acid sequence set forth in SEQ ID NO: 26 or the amino acid sequence DDS; and a CDR-L3 comprising an amino acid sequence set forth in SEQ ID NO: 28.

In some embodiments, the antibody, or an antigen binding fragment thereof, comprises a CDR-H1 comprising an amino acid sequence set forth in any one of SEQ ID NOs: 2, 5, and 8; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 21 and 24; a CDR-L2 comprising an amino acid sequence set forth in SEQ ID NO: 25 or the amino acid sequence DVS; and a CDR-L3 comprising an amino acid sequence set forth in SEQ ID NO: 28.

In some embodiments, the antibody, or an antigen binding fragment thereof, comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 21 and 24; a CDR-L2 comprising an amino acid sequence set forth in SEQ ID NO: 26 or the amino acid sequence DDS; and a CDR-L3 comprising an amino acid sequence set forth in SEQ ID NO: 27.

In some embodiments, the antibody, or an antigen binding fragment thereof, comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 15 and 18; a CDR-L1 comprising an amino acid sequence set forth in anyone of SEQ ID NOs: 21 and 24; a CDR-L2 comprising an amino acid sequence set forth in SEQ ID NO: 25 or the amino acid sequence DVS; and a CDR-L3 comprising an amino acid sequence set forth in SEQ ID NO: 29.

In some embodiments, the antibody, or an antigen binding fragment thereof, comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 19 and 22; a CDR-L2 comprising an amino acid sequence set forth in SEQ ID NO: 25 or the amino acid sequence DVS; and a CDR-L3 comprising an amino acid sequence set forth in SEQ ID NO: 30.

In some embodiments, the antibody, or an antigen binding fragment thereof, comprises a CDR-H1 comprising an amino acid sequence set forth in any one of SEQ ID NOs: 1, 4, and 7; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 19 and 22; a CDR-L2 comprising an amino acid sequence set forth in SEQ ID NO: 26 or the amino acid sequence DDS; and a CDR-L3 comprising an amino acid sequence set forth in SEQ ID NO: 31.

In some embodiments, the antibody, or an antigen binding fragment thereof, comprises a CDR-H1 comprising an amino acid sequence set forth in any one of SEQ ID NOs: 2, 5, and 8; a CDR-H2 comprising an amino acid sequence set forth in any one of SEQ ID NOs: 10, 11, and 12; a CDR-H3 comprising an amino acid sequence set forth in any one of SEQ ID NOs: 15 and 18; a CDR-L1 comprising an amino acid sequence set forth in any one of SEQ ID NOs: 21 and 24; a CDR-L2 comprising an amino acid sequence set forth in SEQ ID NO: 26 or the amino acid sequence DDS; and a CDR-L3 comprising an amino acid sequence set forth in SEQ ID NO: 28.

In some embodiments, the antibody, or an antigen binding fragment thereof, comprises a VH sequence selected from the sequences set forth in any one of SEQ ID NOs: 32-36 and 386.

In some embodiments, the antibody, or an antigen binding fragment thereof, comprises a VL sequence selected from the sequences set forth in any one of SEQ ID NOs: 37-45, 328-364, 371-379, and 381-385.

In some embodiments, the antibody, or an antigen binding fragment thereof, comprises a VH sequence selected from an amino acid sequence set forth in any one of SEQ ID NOs: 32-36 and 386; and a VL sequence selected from an amino acid sequence set forth in any one of SEQ ID NOs: 37-45, 328-364, 371-379, and 381-385.

In another aspect, provided herein are isolated antibodies, or antigen binding fragments thereof, that bind TSLP, wherein the antibody, or an antigen binding fragment thereof, comprises a VH sequence selected from an amino acid sequence set forth in any one of SEQ ID NOs: 32-36 and 386, and a VL sequence selected from an amino acid sequence set forth in any one of SEQ ID NOs: 37-45, 328-364, 371-379, and 381-385.

In some embodiments, the antibody, or an antigen binding fragment thereof, comprises a VH sequence set forth in SEQ ID NO: 32 and a VL sequence set forth in SEQ ID NO: 37.

In some embodiments, the antibody, or an antigen binding fragment thereof, comprises a VH sequence set forth in SEQ ID NO: 33 and a VL sequence set forth in SEQ ID NO: 38.

In some embodiments, the antibody, or an antigen binding fragment thereof, comprises a VH sequence set forth in SEQ ID NO: 386 and a VL sequence set forth in SEQ ID NO: 39.

In some embodiments, the antibody, or an antigen binding fragment thereof, comprises a VH sequence set forth in SEQ ID NO: 33 and a VL sequence set forth in SEQ ID NO: 40.

In some embodiments, the antibody, or an antigen binding fragment thereof, comprises a VH sequence set forth in SEQ ID NO: 34 and a VL sequence set forth in SEQ ID NO: 41.

In some embodiments, the antibody, or an antigen binding fragment thereof, comprises a VH sequence set forth in SEQ ID NO: 35 and a VL sequence set forth in SEQ ID NO: 42.

In some embodiments, the antibody, or an antigen binding fragment thereof, comprises a VH sequence set forth in SEQ ID NO: 34 and a VL sequence set forth in SEQ ID NO: 43.

In some embodiments, the antibody, or an antigen binding fragment thereof, comprises a VH sequence set forth in SEQ ID NO: 32 and a VL sequence set forth in SEQ ID NO: 44.

In some embodiments, the antibody, or an antigen binding fragment thereof, comprises a VH sequence set forth in SEQ ID NO: 36 and a VL sequence set forth in SEQ ID NO: 45.

In some embodiments, the antibody, or an antigen binding fragment thereof, is a humanized, human, or chimeric antibody.

In some embodiments, the antibody, or an antigen binding fragment thereof, is a humanized antibody.

In some embodiments, the antibody, or an antigen binding fragment thereof, comprises a heavy chain human constant region of a class selected from IgG, IgA, IgD, IgE, and IgM.

In some embodiments, the antibody, or an antigen binding fragment thereof, comprises a human Fc region comprising a human heavy chain constant region of the class IgG and a subclass selected from IgG1, IgG2, IgG3, and IgG4.

In some embodiments, the human Fc region comprises a human IgG1 Fc region.

In some embodiments, the human Fc region comprises a human IgG4 Fc region.

In some embodiments, the human Fc region comprises a human IgG2 Fc region.

In some embodiments, the antibody, or an antigen binding fragment thereof, comprises a heavy chain comprising a constant heavy chain sequence selected from the sequences set forth in any one of SEQ ID NOs: 47-270. In some embodiments, the antibody, or an antigen binding fragment thereof, comprises a constant heavy chain sequence set forth in SEQ ID NO: 61 or SEQ ID NO: 173.

In some embodiments, the antibody, or an antigen binding fragment thereof, comprises a light chain comprising a constant light chain sequence set forth in SEQ ID NO: 46.

In some embodiments, antibody comprises a light chain comprising a human lambda light chain constant region. In some embodiments, the human lambda light chain constant region comprises a sequence set forth in any one of SEQ ID NOs: 480-483. In some embodiments, the human lambda light chain constant region comprises a sequence set forth in SEQ ID NO: 480.

In some embodiments, the antibody, or antigen binding fragment thereof, comprises a heavy chain constant domain having a means for increasing the half-life of the antibody.

In some embodiments, the antibody, or an antigen binding fragment thereof, comprises an Fc region comprising one or more amino acid substitutions, wherein the one or more amino acid substitutions result in an increase in one or more of antibody half-life, ADCC activity, ADCP activity, or CDC activity compared with the Fc without the one or more substitutions.

In some embodiments, the antibody, or an antigen binding fragment thereof, comprises an Fc region comprising one or more amino acid substitutions, wherein the one or more amino acid substitutions result in a decrease in one or more of ADCC activity, ADCP activity or CDC activity compared to an antibody comprising a wild-type Fc region.

In some embodiments, the one or more amino acid substitutions is selected from the group consisting of S228P (SP), T250Q, M252Y, S254T, T256E, T256D, H285D, T307A, T307Q, T307R, T307W, L309D, Q411H, Q311V, A378V, E380A, M428L, N434A, N434S, N297A, D265A, L234A, L235A, and N434W (or e.g., L239A, L240A, M257Y, S259T, and T261E using direct numbering); optionally, wherein the one or more amino acid substitutions comprises a plurality of amino acid substitutions selected from the group consisting of M428L/N434S (LS), M252Y/S254T/T256E (YTE) using EU numbering or M257Y/S259T/T261E (YTE) using direct numbering, T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA) using EU numbering or L239A/L240A (LALA) using direct numbering, M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV. In some embodiments, the one or more amino acid substitutions is selected from the group consisting of LS, YTE or M257Y/S259T/T261E (YTE using direct numbering), T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.

In some embodiments, the one or more amino acid substitutions comprises L234A/L235A (LALA) or L239A/L240A (LALA) using direct numbering. In some embodiments, the one or more amino acid substitutions comprises LALAGA and N434A.

In some embodiments, the one or more amino acid substitutions comprises M252Y, S254T, and T256E (YTE) or M257Y/S259T/T261E (YTE) using direct numbering and/or M428L and N434S (LS).

In some embodiments, the one or more amino acid substitutions comprises L234A/L235A (LALA) and M252Y, S254T, and T256E (YTE) or L239A/L240A (LALA) and M257Y/S259T/T261E (YTE) using direct numbering.

In some embodiments, the Fc region binds to Neonatal Fc receptor (FcRn).

In some embodiments, the Fc region binds an FcRn with higher affinity at pH 6.0 compared to an antibody comprising a wild-type Fc region.

In some embodiments, the antibody, or an antigen binding fragment thereof, is a monoclonal antibody.

In some embodiments, the antibody, or an antigen binding fragment thereof, binds a TSLP sequence set forth in SEQ ID NO: 365 or 366.

In some embodiments, the antibody, or an antigen binding fragment thereof, is for use in the treatment of an inflammatory disorder or disease.

In some embodiments, the antibody, or an antigen binding fragment thereof, is for use in the treatment of atopic dermatitis (AD). In some embodiments, the treatment reduces disease severity in a patient and disease severity is assessed by an atopic dermatitis disease severity outcome measure.

In some embodiments, the antibody, or an antigen binding fragment thereof, is for use in the treatment of asthma.

In some embodiments, the antibody, or an antigen binding fragment thereof, is for use in the treatment of chronic sinusitis with nasal polyps.

In some embodiments, the antibody, or an antigen binding fragment thereof, is for use in the treatment of Chronic Rhinosinusitis without Nasal Polyps (CRSsNP).

In some embodiments, the antibody, or an antigen binding fragment thereof, is for use in the treatment of eosinophilic esophagitis (EoE).

In some embodiments, the antibody, or an antigen binding fragment thereof, is for use in the treatment of an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE).

In some embodiments, the antibody, or an antigen binding fragment thereof, is for use in the treatment of Churg-Strauss syndrome/Eosinophilic granulomatosis with polyangiitis (EGPA).

In some embodiments, the antibody, or an antigen binding fragment thereof, is for use in the treatment of Prurigo Nodularis (PN).

In some embodiments, the antibody, or an antigen binding fragment thereof, is for use in the treatment of Chronic Spontaneous Urticaria (CSU).

In some embodiments, the antibody, or an antigen binding fragment thereof, is for use in the treatment of Chronic Pruritis of Unknown Origin (CPUO).

In some embodiments, the antibody, or an antigen binding fragment thereof, is for use in the treatment of Bullous Pemphigoid (BP).

In some embodiments, the antibody, or an antigen binding fragment thereof, is for use in the treatment of Cold Inducible Urticaria (ColdU).

In some embodiments, the antibody, or an antigen binding fragment thereof, is for use in the treatment of Allergic Fungal Rhinosinusitis (AFRS).

In some embodiments, the antibody, or an antigen binding fragment thereof, is for use in the treatment of Allergic Bronchopulmonary Aspergillosis (ABPA).

In some embodiments, the antibody, or an antigen binding fragment thereof, is for use in the treatment of Chronic Obstructive Pulmonary Disease (COPD).

In some embodiments, the antibody, or an antigen binding fragment thereof, is for use in the treatment of Crohn's disease.

In some embodiments, the antibody, or an antigen binding fragment thereof, is for use in the treatment of lupus.

In some embodiments, the antibody, or an antigen binding fragment thereof, is for use in the treatment of rheumatoid arthritis (RA).

In some embodiments, the antibody, or an antigen binding fragment thereof, is for use in the treatment of psoriasis.

In some embodiments, the antibody, or an antigen binding fragment thereof, is for use in the treatment of ulcerative colitis.

In some embodiments, the antibody, or an antigen binding fragment thereof, is for use in the treatment of hidradenitis suppurativa.

In some embodiments, the antibody, or an antigen binding fragment thereof, is for use in the treatment of celiac disease.

In some embodiments, the antibody, or an antigen binding fragment thereof, is for use in the treatment of systemic sclerosis.

In some embodiments, the antibody, or an antigen binding fragment thereof, is for use in the treatment of idiopathic pulmonary fibrosis.

In some embodiments, the antibody, or an antigen binding fragment thereof, is for use in the treatment of alopecia areata.

In another aspect, provided herein is an isolated polynucleotide or set of polynucleotides encoding an antibody disclosed herein, a VH thereof, a VL thereof, a light chain thereof, a heavy chain thereof, or an antigen-binding portion thereof, and optionally, wherein the polynucleotide or set of polynucleotides comprises cDNA.

In another aspect, provided herein is a vector or set of vectors comprising the polynucleotide or set of polynucleotides disclosed herein.

In another aspect, provided herein is a host cell comprising the polynucleotide or set of polynucleotides disclosed herein or the vector or set of vectors disclosed herein.

In another aspect, provided herein is a method of producing an antibody, or an antigen binding fragment thereof, the method comprising expressing the antibody with a host cell disclosed herein and isolating the expressed antibody.

In another aspect, provided herein is a pharmaceutical composition comprising an antibody, or an antigen binding fragment thereof, disclosed herein and a pharmaceutically acceptable excipient. In some embodiments, the pharmaceutical composition further comprises a hyaluronidase or a variant thereof.

In another aspect, provided herein is a kit comprising an antibody, or an antigen binding fragment thereof, disclosed herein or a pharmaceutical composition disclosed herein and instructions for use. In some embodiments, the kit further comprises a hyaluronidase or a variant thereof.

In another aspect, provided herein is a method for treating an inflammatory disorder or disease in a mammalian subject in need thereof, the method comprising administering to the mammalian subject a therapeutically effective amount of an antibody, or an antigen binding fragment thereof, disclosed herein or a pharmaceutical composition disclosed herein.

In some embodiments, the mammalian subject is a human.

In some embodiments, the inflammatory disorder or disease is atopic dermatitis.

In some embodiments, the inflammatory disorder or disease is asthma.

In some embodiments, the inflammatory disorder or disease is Chronic Obstructive Pulmonary Disease (COPD).

In some embodiments, the inflammatory disorder or disease is chronic sinusitis with nasal polyps. In some embodiments, the inflammatory disorder or disease is Chronic Rhinosinusitis without Nasal Polyps (CRSsNP). In some embodiments, the inflammatory disorder or disease is eosinophilic esophagitis (EoE). In some embodiments, the inflammatory disorder or disease is an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE). In some embodiments, the inflammatory disorder or disease is Churg-Strauss syndrome/Eosinophilic granulomatosis with polyangiitis (EGPA). In some embodiments, the inflammatory disorder or disease is Prurigo Nodularis (PN).

In some embodiments, the inflammatory disorder or disease is Chronic Spontaneous Urticaria (CSU). In some embodiments, the inflammatory disorder or disease is Chronic Pruritis of Unknown Origin (CPUO). In some embodiments, the inflammatory disorder or disease is Bullous Pemphigoid (BP). In some embodiments, the inflammatory disorder or disease is Cold Inducible Urticaria (ColdU). In some embodiments, the inflammatory disorder or disease is Allergic Fungal Rhinosinusitis (AFRS). In some embodiments, the inflammatory disorder or disease is Allergic Bronchopulmonary Aspergillosis (ABPA). In some embodiments, the inflammatory disorder or disease is celiac disease. In some embodiments, the inflammatory disorder or disease is an inflammatory bowel disease, such as Crohn's disease or ulcerative colitis. In some embodiments, the inflammatory disorder or disease is lupus. In some embodiments, the inflammatory disorder or disease is rheumatoid arthritis (RA). In some embodiments, the inflammatory disorder or disease is psoriasis. In some embodiments, the inflammatory disorder or disease is hidradenitis suppurativa. In some embodiments, the inflammatory disorder or disease is systemic sclerosis. In some embodiments, the inflammatory disorder or disease is idiopathic pulmonary fibrosis. In some embodiments, the inflammatory disorder or disease is alopecia areata.

In another aspect, provided herein is a method for treating a pathology associated with elevated levels of TSLP in a mammalian subject in need thereof, the method comprising administering to the mammalian subject a therapeutically effective amount of an antibody, or an antigen binding fragment thereof, disclosed herein or a pharmaceutical composition disclosed herein.

In another aspect, provided herein is a method of reducing biological activity of TSLP in a mammalian subject in need thereof, the method comprising administering to the mammalian subject a therapeutically effective amount of an antibody, or an antigen binding fragment thereof, disclosed herein or a pharmaceutical composition disclosed herein.

In another aspect, provided herein is a method of preventing an inflammatory disorder or disease in a mammalian subject in need thereof, the method comprising administering to the mammalian subject a therapeutically effective amount of an antibody, or an antigen binding fragments thereof, disclosed herein or a pharmaceutical composition disclosed herein.

In another aspect, provided herein is a method of reducing levels of Thymus and Activation Regulated Chemokine (TARC)/CCL17 in a mammalian subject in need thereof, the method comprising administering to the mammalian subject a therapeutically effective amount of an antibody, or an antigen binding fragment thereof, disclosed herein or a pharmaceutical composition disclosed herein.

In another aspect, provided herein is a method of reducing levels of macrophage-derived chemokine (MDC (CCL22)) in a mammalian subject in need thereof, the method comprising administering to the mammalian subject a therapeutically effective amount of an antibody, or an antigen binding fragment thereof, disclosed herein or a pharmaceutical composition disclosed herein.

In some embodiments, the mammalian subject is a human.

In some embodiments of any of the foregoing methods, the method further comprises administering a hyaluronidase or a variant thereof. In some embodiments of any of the foregoing methods, the pharmaceutical composition comprises an antibody, or antigen binding fragment thereof, described herein and a hyaluronidase or a variant thereof.

In another aspect, provided herein is an antibody or an antigen-binding fragment thereof that binds an epitope of TSLP within amino acids 15-31 of SEQ ID NO: 365 (YLSTISKDLITYMSGTK; SEQ ID NO: 402) as measured by cross-linking mass spectrometry.

In another aspect, provided herein is an antibody or an antigen-binding fragment thereof that binds an epitope of TSLP within amino acids 20-26 of SEQ ID NO: 365 (SKDLITY; SEQ ID NO: 408) and amino acids 66-70 of SEQ ID NO: 365 (AKEMF; SEQ ID NO: 401) but not amino acids 120-128 of SEQ ID NO: 365 (WRRFNRPLL; SEQ ID NO: 404).

In certain aspects, described herein are compositions comprising any of the antibodies, or antigen binding fragments thereof, that bind thymic stromal lymphopoietin (TSLP) described herein and a hyaluronidase or variant thereof.

In certain aspects, described herein are a combination of any one of the antibodies, or antigen binding fragments thereof, that bind TSLP described herein and a hyaluronidase or variant thereof.

In certain aspects, described herein are methods of treating an inflammatory disorder or disease in a human patient comprising co-administering to the patient any one of the antibodies, or antigen binding fragments thereof, that bind TSLP described herein and a hyaluronidase or variant thereof.

In certain embodiments, the antibody, or antigen binding fragment thereof, is a humanized, human, or chimeric antibody. In certain embodiments, the antibody, or antigen binding fragment thereof, is a humanized antibody. In certain embodiments, the antibody, or antigen binding fragment thereof, is a monoclonal antibody. In certain embodiments, the antibody, or antigen binding fragment thereof, comprises a heavy chain human constant region of a class selected from IgG, IgA, IgD, IgE, and IgM. In certain embodiments, the antibody, or antigen binding fragment thereof, comprises a human Fc region comprising a human heavy chain constant region of the class IgG and a subclass selected from IgG1, IgG2, IgG3, and IgG4. In certain embodiments, the human Fc region comprises a human IgG1 Fc region.

Although the EU numbering system is typically used to identify the positions of the various Fc mutations described herein, direct numbering can also be used. For example, in certain embodiments, an antibody, or antigen binding fragment thereof, described herein comprises an Fc region with YTE mutations at positions 257/259/261, respectively. In certain embodiments, an antibody, or antigen binding fragment thereof, described herein comprises an Fc region with LALA mutations at positions 239/240, respectively. In certain embodiments, an antibody, or antigen binding fragment thereof, described herein comprises an Fc region with YTE mutations at positions 257/259/261 and with LALA mutations at positions 239/240. In certain embodiments, the TSLP antibody described herein comprises the VH and VL of Antibody 5 and an Fc region comprising YTE mutations at positions 257/259/261, respectively and with LALA mutations at positions 239/240, respectively.

In certain embodiments the human Fc region comprises a human IgG1 Fc region with LALA mutations. In certain embodiments the human Fc region comprises a human IgG1 Fc region with YTE mutations. In certain embodiments the human Fc region comprises a human IgG1 Fc region with LALA and YTE mutations. In certain embodiments, when direct numbering is used these “YTE” and “LALA” mutations can be located at different amino acid position numbers. For example, in one embodiment, the human Fc region comprises a human IgG1 Fc region with LALA mutations at L239A/L240A and/or YTE mutations at M257Y/S259T/T261E.

In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In certain embodiments, the antibody, or antigen binding fragment thereof, that binds TSLP comprises a VH sequence set forth in SEQ ID NO: 34 and a VL sequence set forth in SEQ ID NO: 41. In certain embodiments, the antibody that binds TSLP comprises a heavy chain constant region sequence set forth in SEQ ID NO: 61 or 173 and a light chain constant region sequence set forth in SEQ ID NO: 480. In certain embodiments, the antibody that binds TSLP comprises a heavy chain sequence set forth in SEQ ID NO: 484 and/or 486 and a light chain sequence set forth in SEQ ID NO: 485. In some embodiments, the antibody that binds TSLP comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486.

In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29. In certain embodiments, the antibody, or antigen binding fragment thereof, that binds TSLP comprises a VH sequence set forth in SEQ ID NO: 478 and a VL sequence set forth in SEQ ID NO: 479. In certain embodiments, the antibody that binds TSLP comprises a heavy chain sequence set forth in SEQ ID NO: 476 and a light chain sequence set forth in SEQ ID NO: 477.

In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29, and comprises a human IgG sequence selected from a sequence set forth in any one of SEQ ID NOs: 47-50, 52-162, and 164-270. In certain embodiments, the antibody, or antigen binding fragment thereof, that binds TSLP comprises a VH sequence set forth in SEQ ID NO: 478 and a VL sequence set forth in SEQ ID NO: 479. In certain embodiments, the antibody does not comprise a human IgG2 constant heavy chain sequence (e.g., SEQ ID NO: 51 or SEQ ID NO: 163).

In certain embodiments, the antibody, or antigen binding fragment thereof, binds a TSLP sequence set forth in SEQ ID NO: 365 or 366. In certain embodiments, the Fc region of the antibody binds to Neonatal Fc receptor (FcRn). In certain embodiments, the Fc region of the antibody binds an FcRn with higher affinity at pH 6.0 compared to an antibody comprising a wild-type Fc region.

Any suitable hyaluronidase or variant thereof can be used in the compositions, combinations, and methods described herein. In certain embodiments, the hyaluronidase is a soluble hyaluronidase. In certain embodiments, the hyaluronidase is a recombinant human hyaluronidase. In certain embodiments, the recombinant human hyaluronidase or variant thereof comprises the amino acid sequence set forth in any one of SEQ ID NOs: 409-418 and 420-475, or a mixture thereof. In certain embodiments, the recombinant human hyaluronidase is rHuPH20 (i.e., a composition comprising one or more of SEQ ID NOs: 411-416). In certain embodiments, the rHuPH20 formulation is ENHANZE®.

In one embodiment, the recombinant human hyaluronidase includes a sequence of amino acids in any one of SEQ ID NOs: 409-418 and 420-475, or has at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence of amino acids included in SEQ ID NO:409-418 and 420-475 and retains hyaluronidase activity. In one embodiment, the recombinant human hyaluronidase comprises a sequence having at least 95%, 96%, 97%, 98%, or 99% to the amino acid sequence of SEQ ID NO: 409. In one embodiment, the recombinant human hyaluronidase comprises a sequence having at least 95%, 96%, 97%, 98%, or 99% to the amino acid sequence of SEQ ID NO: 411. In another embodiment, the recombinant human hyaluronidase comprises the amino acid sequence set forth in SEQ ID NO:409. In another embodiment, the recombinant human hyaluronidase consists of the amino acid sequence set forth in SEQ ID NO:409. In another embodiment, the recombinant human hyaluronidase comprises the amino acid sequence set forth in SEQ ID NO:410. In another embodiment, the recombinant human hyaluronidase consists of the amino acid sequence set forth in SEQ ID NO:410. In another embodiment, the recombinant human hyaluronidase comprises the amino acid sequence set forth in SEQ ID NO: 411. In another embodiment, the recombinant human hyaluronidase consists of the amino acid sequence set forth in SEQ ID NO:411. In another embodiment, the recombinant human hyaluronidase comprises the amino acid sequence set forth in SEQ ID NO:412. In another embodiment, the recombinant human hyaluronidase consists of the amino acid sequence set forth in SEQ ID NO:412. In another embodiment, the recombinant human hyaluronidase comprises the amino acid sequence set forth in SEQ ID NO:413. In another embodiment, the recombinant human hyaluronidase consists of the amino acid sequence set forth in SEQ ID NO:413. In another embodiment, the recombinant human hyaluronidase comprises the amino acid sequence set forth in SEQ ID NO:414. In another embodiment, the recombinant human hyaluronidase consists of the amino acid sequence set forth in SEQ ID NO:414. In another embodiment, the recombinant human hyaluronidase comprises the amino acid sequence set forth in SEQ ID NO: 415. In another embodiment, the recombinant human hyaluronidase consists of the amino acid sequence set forth in SEQ ID NO:415. In another embodiment, the recombinant human hyaluronidase comprises the amino acid sequence set forth in SEQ ID NO:416. In another embodiment, the recombinant human hyaluronidase consists of the amino acid sequence set forth in SEQ ID NO:416. In another embodiment, the recombinant human hyaluronidase comprises the amino acid sequence set forth in SEQ ID NO:417. In another embodiment, the recombinant human hyaluronidase consists of the amino acid sequence set forth in SEQ ID NO:417. In another embodiment, the recombinant human hyaluronidase comprises the amino acid sequence set forth in SEQ ID NO:418. In another embodiment, the recombinant human hyaluronidase consists of the amino acid sequence set forth in SEQ ID NO:418. In another embodiment, the recombinant human hyaluronidase comprises the amino acid sequence set forth in SEQ ID NO: 420. In another embodiment, the recombinant human hyaluronidase consists of the amino acid sequence set forth in SEQ ID NO:420. In another embodiment, the recombinant human hyaluronidase comprises the amino acid sequence set forth in SEQ ID NO:421. In another embodiment, the recombinant human hyaluronidase consists of the amino acid sequence set forth in SEQ ID NO:421. In another embodiment, the recombinant human hyaluronidase comprises the amino acid sequence set forth in SEQ ID NO:422. In another embodiment, the recombinant human hyaluronidase consists of the amino acid sequence set forth in SEQ ID NO:422. In another embodiment, the recombinant human hyaluronidase comprises the amino acid sequence set forth in SEQ ID NO:423. In another embodiment, the recombinant human hyaluronidase consists of the amino acid sequence set forth in SEQ ID NO:423. In another embodiment, the recombinant human hyaluronidase comprises the amino acid sequence set forth in SEQ ID NO: 424. In another embodiment, the recombinant human hyaluronidase consists of the amino acid sequence set forth in SEQ ID NO:424. In another embodiment, the recombinant human hyaluronidase comprises the amino acid sequence set forth in SEQ ID NO:425. In another embodiment, the recombinant human hyaluronidase consists of the amino acid sequence set forth in SEQ ID NO:425. In another embodiment, the recombinant human hyaluronidase comprises the amino acid sequence set forth in SEQ ID NO:426. In another embodiment, the recombinant human hyaluronidase consists of the amino acid sequence set forth in SEQ ID NO:426. In another embodiment, the recombinant human hyaluronidase comprises the amino acid sequence set forth in SEQ ID NO:427. In another embodiment, the recombinant human hyaluronidase consists of the amino acid sequence set forth in SEQ ID NO:427. In another embodiment, the recombinant human hyaluronidase comprises the amino acid sequence set forth in SEQ ID NO: 428. In another embodiment, the recombinant human hyaluronidase consists of the amino acid sequence set forth in SEQ ID NO:428. In another embodiment, the recombinant human hyaluronidase comprises the amino acid sequence set forth in SEQ ID NO:429. In another embodiment, the recombinant human hyaluronidase consists of the amino acid sequence set forth in SEQ ID NO:429. In another embodiment, the recombinant human hyaluronidase comprises the amino acid sequence set forth in SEQ ID NO:430. In another embodiment, the recombinant human hyaluronidase consists of the amino acid sequence set forth in SEQ ID NO:430. In another embodiment, the recombinant human hyaluronidase comprises the amino acid sequence set forth in SEQ ID NO:431. In another embodiment, the recombinant human hyaluronidase consists of the amino acid sequence set forth in SEQ ID NO:431. In another embodiment, the recombinant human hyaluronidase comprises the amino acid sequence set forth in SEQ ID NO: 432. In another embodiment, the recombinant human hyaluronidase consists of the amino acid sequence set forth in SEQ ID NO:432. In another embodiment, the recombinant human hyaluronidase comprises the amino acid sequence set forth in SEQ ID NO:433. In another embodiment, the recombinant human hyaluronidase consists of the amino acid sequence set forth in SEQ ID NO:433. In another embodiment, the recombinant human hyaluronidase comprises the amino acid sequence set forth in SEQ ID NO:434. In another embodiment, the recombinant human hyaluronidase consists of the amino acid sequence set forth in SEQ ID NO:434. In another embodiment, the recombinant human hyaluronidase comprises the amino acid sequence set forth in SEQ ID NO:435. In another embodiment, the recombinant human hyaluronidase consists of the amino acid sequence set forth in SEQ ID NO:435. In another embodiment, the recombinant human hyaluronidase comprises the amino acid sequence set forth in SEQ ID NO: 436. In another embodiment, the recombinant human hyaluronidase consists of the amino acid sequence set forth in SEQ ID NO:436. In another embodiment, the recombinant human hyaluronidase comprises the amino acid sequence set forth in SEQ ID NO:437. In another embodiment, the recombinant human hyaluronidase consists of the amino acid sequence set forth in SEQ ID NO:437. In another embodiment, the recombinant human hyaluronidase comprises the amino acid sequence set forth in SEQ ID NO:438. In another embodiment, the recombinant human hyaluronidase consists of the amino acid sequence set forth in SEQ ID NO:438. In another embodiment, the recombinant human hyaluronidase comprises the amino acid sequence set forth in SEQ ID NO:439. In another embodiment, the recombinant human hyaluronidase consists of the amino acid sequence set forth in SEQ ID NO:439. In another embodiment, the recombinant human hyaluronidase comprises the amino acid sequence set forth in SEQ ID NO: 440. In another embodiment, the recombinant human hyaluronidase consists of the amino acid sequence set forth in SEQ ID NO:440. In another embodiment, the recombinant human hyaluronidase comprises the amino acid sequence set forth in SEQ ID NO:441. In another embodiment, the recombinant human hyaluronidase consists of the amino acid sequence set forth in SEQ ID NO:441. In another embodiment, the recombinant human hyaluronidase comprises the amino acid sequence set forth in SEQ ID NO:442. In another embodiment, the recombinant human hyaluronidase consists of the amino acid sequence set forth in SEQ ID NO:442. In another embodiment, the recombinant human hyaluronidase comprises the amino acid sequence set forth in SEQ ID NO:443. In another embodiment, the recombinant human hyaluronidase consists of the amino acid sequence set forth in SEQ ID NO:443. In another embodiment, the recombinant human hyaluronidase comprises the amino acid sequence set forth in SEQ ID NO: 444. In another embodiment, the recombinant human hyaluronidase consists of the amino acid sequence set forth in SEQ ID NO:444. In another embodiment, the recombinant human hyaluronidase comprises the amino acid sequence set forth in SEQ ID NO:445. In another embodiment, the recombinant human hyaluronidase consists of the amino acid sequence set forth in SEQ ID NO:445. In another embodiment, the recombinant human hyaluronidase comprises the amino acid sequence set forth in SEQ ID NO:446. In another embodiment, the recombinant human hyaluronidase consists of the amino acid sequence set forth in SEQ ID NO:446. In another embodiment, the recombinant human hyaluronidase comprises the amino acid sequence set forth in SEQ ID NO:447. In another embodiment, the recombinant human hyaluronidase consists of the amino acid sequence set forth in SEQ ID NO:447. In another embodiment, the recombinant human hyaluronidase comprises the amino acid sequence set forth in SEQ ID NO: 448. In another embodiment, the recombinant human hyaluronidase consists of the amino acid sequence set forth in SEQ ID NO:448. In another embodiment, the recombinant human hyaluronidase comprises the amino acid sequence set forth in SEQ ID NO:449. In another embodiment, the recombinant human hyaluronidase consists of the amino acid sequence set forth in SEQ ID NO:449. In another embodiment, the recombinant human hyaluronidase comprises the amino acid sequence set forth in SEQ ID NO:450. In another embodiment, the recombinant human hyaluronidase consists of the amino acid sequence set forth in SEQ ID NO:450. In another embodiment, the recombinant human hyaluronidase comprises the amino acid sequence set forth in SEQ ID NO:451. In another embodiment, the recombinant human hyaluronidase consists of the amino acid sequence set forth in SEQ ID NO:451. In another embodiment, the recombinant human hyaluronidase comprises the amino acid sequence set forth in SEQ ID NO: 452. In another embodiment, the recombinant human hyaluronidase consists of the amino acid sequence set forth in SEQ ID NO:452. In another embodiment, the recombinant human hyaluronidase comprises the amino acid sequence set forth in SEQ ID NO:453. In another embodiment, the recombinant human hyaluronidase consists of the amino acid sequence set forth in SEQ ID NO:453. In another embodiment, the recombinant human hyaluronidase comprises the amino acid sequence set forth in SEQ ID NO:454. In another embodiment, the recombinant human hyaluronidase consists of the amino acid sequence set forth in SEQ ID NO:454. In another embodiment, the recombinant human hyaluronidase comprises the amino acid sequence set forth in SEQ ID NO:455. In another embodiment, the recombinant human hyaluronidase consists of the amino acid sequence set forth in SEQ ID NO:455. In another embodiment, the recombinant human hyaluronidase comprises the amino acid sequence set forth in SEQ ID NO: 456. In another embodiment, the recombinant human hyaluronidase consists of the amino acid sequence set forth in SEQ ID NO:456. In another embodiment, the recombinant human hyaluronidase comprises the amino acid sequence set forth in SEQ ID NO:457. In another embodiment, the recombinant human hyaluronidase consists of the amino acid sequence set forth in SEQ ID NO:457. In another embodiment, the recombinant human hyaluronidase comprises the amino acid sequence set forth in SEQ ID NO:458. In another embodiment, the recombinant human hyaluronidase consists of the amino acid sequence set forth in SEQ ID NO:458. In another embodiment, the recombinant human hyaluronidase comprises the amino acid sequence set forth in SEQ ID NO:459. In another embodiment, the recombinant human hyaluronidase consists of the amino acid sequence set forth in SEQ ID NO:459. In another embodiment, the recombinant human hyaluronidase comprises the amino acid sequence set forth in SEQ ID NO: 460. In another embodiment, the recombinant human hyaluronidase consists of the amino acid sequence set forth in SEQ ID NO:460. In another embodiment, the recombinant human hyaluronidase comprises the amino acid sequence set forth in SEQ ID NO:461. In another embodiment, the recombinant human hyaluronidase consists of the amino acid sequence set forth in SEQ ID NO:461. In another embodiment, the recombinant human hyaluronidase comprises the amino acid sequence set forth in SEQ ID NO:462. In another embodiment, the recombinant human hyaluronidase consists of the amino acid sequence set forth in SEQ ID NO:462. In another embodiment, the recombinant human hyaluronidase comprises the amino acid sequence set forth in SEQ ID NO:463. In another embodiment, the recombinant human hyaluronidase consists of the amino acid sequence set forth in SEQ ID NO:463. In another embodiment, the recombinant human hyaluronidase comprises the amino acid sequence set forth in SEQ ID NO: 464. In another embodiment, the recombinant human hyaluronidase consists of the amino acid sequence set forth in SEQ ID NO:464. In another embodiment, the recombinant human hyaluronidase comprises the amino acid sequence set forth in SEQ ID NO:465. In another embodiment, the recombinant human hyaluronidase consists of the amino acid sequence set forth in SEQ ID NO:465. In another embodiment, the recombinant human hyaluronidase comprises the amino acid sequence set forth in SEQ ID NO:466. In another embodiment, the recombinant human hyaluronidase consists of the amino acid sequence set forth in SEQ ID NO:466. In another embodiment, the recombinant human hyaluronidase comprises the amino acid sequence set forth in SEQ ID NO:467. In another embodiment, the recombinant human hyaluronidase consists of the amino acid sequence set forth in SEQ ID NO:467. In another embodiment, the recombinant human hyaluronidase comprises the amino acid sequence set forth in SEQ ID NO: 468. In another embodiment, the recombinant human hyaluronidase consists of the amino acid sequence set forth in SEQ ID NO:468. In another embodiment, the recombinant human hyaluronidase comprises the amino acid sequence set forth in SEQ ID NO:469. In another embodiment, the recombinant human hyaluronidase consists of the amino acid sequence set forth in SEQ ID NO:469. In another embodiment, the recombinant human hyaluronidase comprises the amino acid sequence set forth in SEQ ID NO:470. In another embodiment, the recombinant human hyaluronidase consists of the amino acid sequence set forth in SEQ ID NO:470. In another embodiment, the recombinant human hyaluronidase comprises the amino acid sequence set forth in SEQ ID NO:471. In another embodiment, the recombinant human hyaluronidase consists of the amino acid sequence set forth in SEQ ID NO:471. In another embodiment, the recombinant human hyaluronidase comprises the amino acid sequence set forth in SEQ ID NO: 472. In another embodiment, the recombinant human hyaluronidase consists of the amino acid sequence set forth in SEQ ID NO:472. In another embodiment, the recombinant human hyaluronidase comprises the amino acid sequence set forth in SEQ ID NO:473. In another embodiment, the recombinant human hyaluronidase consists of the amino acid sequence set forth in SEQ ID NO:473. In another embodiment, the recombinant human hyaluronidase comprises the amino acid sequence set forth in SEQ ID NO:474. In another embodiment, the recombinant human hyaluronidase consists of the amino acid sequence set forth in SEQ ID NO:474. In another embodiment, the recombinant human hyaluronidase comprises the amino acid sequence set forth in SEQ ID NO:475. In another embodiment, the recombinant human hyaluronidase consists of the amino acid sequence set forth in SEQ ID NO:475.

In certain embodiments, the hyaluronidase or variant thereof and the anti-TSLP antibody, or antigen binding fragment thereof, are administered in separate formulations. In certain embodiments, the hyaluronidase or variant thereof and the anti-TSLP antibody, or antigen binding fragment thereof, are administered simultaneously in separate formulations. In certain embodiments, the hyaluronidase or variant thereof is administered to the patient prior to administration of the anti-TSLP antibody, or antigen binding fragment thereof. In certain embodiments, the hyaluronidase or variant thereof is administered to the patient after administration of the anti-TSLP antibody, or antigen binding fragment thereof. In certain embodiments, the hyaluronidase or variant thereof and the anti-TSLP antibody, or antigen binding fragment thereof, are mixed and administered in a single formulation. In certain embodiments, the hyaluronidase or variant thereof and the anti-TSLP antibody, or antigen binding fragment thereof, are administered by subcutaneous injection. In certain embodiments, the hyaluronidase or variant thereof and the anti-TSLP antibody, or antigen binding fragment thereof, are administered by intravenous injection.

In certain embodiments, the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

In certain embodiments, methods of treating an inflammatory disorder or disease in a human patient are provided, wherein the method comprises co-administering to the patient any one of the antibodies, or antigen binding fragments thereof, that bind TSLP described herein and a hyaluronidase or variant thereof. In certain embodiments, the inflammatory disorder or disease is atopic dermatitis. In certain embodiments, the treatment reduces disease severity in the patient and wherein disease severity is assessed by an atopic dermatitis disease severity outcome measure. In certain embodiments, the inflammatory disorder or disease is asthma, idiopathic pulmonary fibrosis, alopecia areata, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), an inflammatory bowel disease, such as Crohn's disease or ulcerative colitis, lupus, rheumatoid arthritis (RA), psoriasis, hidradenitis suppurativa, celiac disease, or systemic sclerosis. In certain embodiments, the inflammatory disorder or disease is an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE).

In certain aspects, described herein are compositions comprising an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and a hyaluronidase or variant thereof. In other aspects, described herein are compositions comprising an antibody, or antigen binding fragment thereof, that binds TSLP, wherein the antibody, or antigen binding fragment thereof, comprises a VH sequence set forth in SEQ ID NO: 34 and a VL sequence set forth in SEQ ID NO: 41, and a hyaluronidase or variant thereof. In certain embodiments, the antibody, or antigen binding fragment thereof, that binds TSLP, comprises a heavy chain constant region sequence set forth in SEQ ID NO: 61 or 173 and a light chain constant region sequence set forth in SEQ ID NO: 480. In other aspects, described herein are compositions comprising an antibody that binds TSLP, wherein the antibody comprises a heavy chain sequence set forth in SEQ ID NO: 484 and/or 486 and a light chain sequence set forth in SEQ ID NO: 485, and a hyaluronidase or variant thereof. In some embodiments, the antibody that binds TSLP comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486.

In certain aspects, described herein are compositions comprising an antibody, or antigen binding fragment thereof, that binds TSLP and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29. In other aspects, described herein are compositions comprising an antibody, or antigen binding fragment thereof, that binds TSLP and a hyaluronidase or variant thereof, wherein the antibody, or antigen binding fragment thereof, comprises a VH sequence set forth in SEQ ID NO: 478 and a VL sequence set forth in SEQ ID NO: 479. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In other aspects, described herein are compositions comprising an antibody that binds TSLP and a hyaluronidase or variant thereof, wherein the antibody comprises a heavy chain sequence set forth in SEQ ID NO: 476 and a light chain sequence set forth in SEQ ID NO: 477.

In certain aspects, described herein are a combination of an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and a hyaluronidase or variant thereof. In other aspects, described herein are a combination of an antibody, or antigen binding fragment thereof, that binds TSLP, wherein the antibody, or antigen binding fragment thereof, comprises a VH sequence set forth in SEQ ID NO: 34 and a VL sequence set forth in SEQ ID NO: 41, and a hyaluronidase or variant thereof. In certain embodiments, the antibody, or antigen binding fragment thereof, that binds TSLP comprises a heavy chain constant region set forth in SEQ ID NO: 61 or 173 and a light chain constant region set forth in SEQ ID NO: 480. In other aspects, described herein are a combination of an antibody that binds TSLP, wherein the antibody comprises a heavy chain sequence set forth in SEQ ID NO: 484 and/or 486 and a light chain sequence set forth in SEQ ID NO: 485, and a hyaluronidase or variant thereof. In some embodiments, the antibody that binds TSLP comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486.

In certain aspects, described herein are a combination of an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29, and a hyaluronidase or variant thereof. In other aspects, described herein are a combination of an antibody, or antigen binding fragment thereof, that binds TSLP, wherein the antibody, or antigen binding fragment thereof, comprises a VH sequence set forth in SEQ ID NO: 478 and a VL sequence set forth in SEQ ID NO: 479, and a hyaluronidase or variant thereof. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In other aspects, described herein are a combination of an antibody that binds TSLP, wherein the antibody comprises a heavy chain sequence set forth in SEQ ID NO: 476 and a light chain sequence set forth in SEQ ID NO: 477, and a hyaluronidase or variant thereof.

In certain aspects, described herein are methods of treating an inflammatory disorder or disease in a human patient comprising co-administering to the patient an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and a hyaluronidase or variant thereof. In other aspects, described herein are methods of treating an inflammatory disorder or disease in a human patient comprising co-administering to the patient an antibody, or antigen binding fragment thereof, that binds TSLP, wherein the antibody, or antigen binding fragment thereof, comprises a VH sequence set forth in SEQ ID NO: 34 and a VL sequence set forth in SEQ ID NO: 41, and a hyaluronidase or variant thereof. In certain embodiments, the antibody, or antigen binding fragment thereof, comprises a heavy chain comprising a constant heavy chain sequence set forth in SEQ ID NO: 61 or 173. In certain embodiments, the antibody, or antigen binding fragment thereof, comprises a light chain comprising a constant light chain sequence set forth in SEQ ID NO: 480. In certain embodiments, the antibody that binds TSLP comprises a heavy chain sequence set forth in SEQ ID NO: 484 and/or 486 and a light chain sequence set forth in SEQ ID NO: 485. In some embodiments, the antibody that binds TSLP comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486.

In certain aspects, described herein are methods of treating an inflammatory disorder or disease in a human patient comprising co-administering to the patient an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29, and a hyaluronidase or variant thereof. In other aspects, described herein are methods of treating an inflammatory disorder or disease in a human patient comprising co-administering to the patient an antibody, or antigen binding fragment thereof, that binds TSLP, wherein the antibody, or antigen binding fragment thereof, comprises a VH sequence set forth in SEQ ID NO: 478 and a VL sequence set forth in SEQ ID NO: 479, and a hyaluronidase or variant thereof. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In other aspects, described herein are methods of treating an inflammatory disorder or disease in a human patient comprising co-administering to the patient an antibody that binds TSLP, wherein the antibody comprises a heavy chain sequence set forth in SEQ ID NO: 476 and a light chain sequence set forth in SEQ ID NO: 477, and a hyaluronidase or variant thereof.

In certain aspects, described herein are kits for treating an inflammatory disorder or disease in a human patient, the kit comprising: (a) a dose of any one of the antibodies, or antigen binding fragments thereof, that binds TSLP described herein; (b) a dose of a hyaluronidase or variant thereof; and (c) instructions. In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In certain embodiments, the antibody, or antigen binding fragment thereof, comprises a VH sequence set forth in SEQ ID NO: 34 and a VL sequence set forth in SEQ ID NO: 41. In certain embodiments, the antibody, or antigen binding fragment thereof, comprises a heavy chain constant region set forth in SEQ ID NO: 61 or 173 and a light chain constant region set forth in SEQ ID NO: 480. In certain embodiments, the antibody comprises a heavy chain sequence set forth in SEQ ID NO: 484 and/or 486 and a light chain sequence set forth in SEQ ID NO: 485. In some embodiments, the antibody that binds TSLP comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486. In other embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29. In other embodiments, the antibody, or antigen binding fragment thereof, comprises a VH sequence set forth in SEQ ID NO: 478 and a VL sequence set forth in SEQ ID NO: 479. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In other embodiments, the antibody comprises a heavy chain sequence set forth in SEQ ID NO: 476 and a light chain sequence set forth in SEQ ID NO: 477.

In certain embodiments, the hyaluronidase is a soluble hyaluronidase. In certain embodiments, the hyaluronidase is a recombinant human hyaluronidase or variant thereof. In certain embodiments, the hyaluronidase is a recombinant human hyaluronidase. In certain embodiments, the recombinant human hyaluronidase comprises the amino acid sequence set forth in any one of SEQ ID NOs: 409-418 and 420-475, or a mixture thereof. In certain embodiments, the recombinant human hyaluronidase is rHuPH20 (i.e., a composition comprising one or more of SEQ ID NOs: 411-416). In certain embodiments, the rHuPH20 formulation is ENHANZE®.

In certain embodiments, the kit is for use in treating an inflammatory disorder or disease. In certain embodiments, the inflammatory disorder or disease is atopic dermatitis. In certain embodiments, the inflammatory disorder or disease is asthma, idiopathic pulmonary fibrosis, alopecia areata, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), an inflammatory bowel disease, such as Crohn's disease or ulcerative colitis, lupus, rheumatoid arthritis (RA), psoriasis, hidradenitis suppurativa, celiac disease, or systemic sclerosis. In certain embodiments, the inflammatory disorder or disease is an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE).

### DETAILED DESCRIPTION

Described herein are compositions and combinations containing anti-TSLP antibodies, or antigen binding fragments thereof, and methods of use thereof. The compositions and combinations containing an anti-TSLP antibody, or antigen binding fragment thereof, described herein further comprise a hyaluronidase or a variant thereof. Also described herein are methods of treating a subject having an inflammatory disorder or disease, such as atopic dermatitis, asthma, or COPD, by administering an anti-TSLP antibody, or antigen binding fragment thereof, described herein and a hyaluronidase or a variant thereof. The anti-TSLP antibody, or antigen binding fragment thereof, and the hyaluronidase or a variant thereof may be administered separately or may be co-formulated and administered in a single composition.

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably herein and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

As used herein, the term “eukaryote” refers to organisms belonging to the phylogenetic domain Eukarya, such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

An “effective amount” or “therapeutically effective amount” as used herein refers to an amount of therapeutic compound, such as an anti-TSLP antibody, or an antigen binding fragment thereof, administered to an individual, either as a single dose or as part of a series of doses, which is effective to produce or contribute to a desired therapeutic effect, either alone or in combination with another therapeutic modality. Examples of a desired therapeutic effect are reducing an aberrant immune response, slowing or delaying disease development, stabilization of disease, and amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

As used herein, the terms “subject,” “patient,” and “individual” mean a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an antibody, or an antigen binding fragment thereof, provided herein. In some aspects, the disease or condition is atopic dermatitis. In some aspects, the disease or condition is asthma.

The term “in vitro” refers to processes that occur in a living cell growing separate from a living organism, e.g., growing in tissue culture.

The term “in vivo” refers to processes that occur in a living organism.

The term “package insert” is used to refer to instructions customarily included in commercial packages of therapeutic or diagnostic products (e.g., kits) that contain information about the indications, usage, dosage, administration, combination therapy, contraindications, and/or warnings concerning the use of such therapeutic or diagnostic products.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

As used herein, the term “pharmaceutically acceptable” refers to those compounds, materials, compositions, and/or dosage forms, which are suitable for contact with the tissues of a subject, such as a mammal (e.g., a human) without excessive toxicity, irritation, allergic response, and other problem complications commensurate with a reasonable benefit/risk ratio. Preferably, the term “pharmaceutically acceptable” means approved by a regulatory agency of the Federal or a state government or listed in the U.S. Pharmacopeia or other generally recognized pharmacopeia for use in mammals and more particularly in humans.

The terms “co-administration,” “co-administer,” and “in combination with” include the administration of two or more therapeutic agents either simultaneously, concurrently, or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value+10%, +5%, or +1%. In certain embodiments, where applicable, the term “about” indicates the designated value(s) ±one standard deviation of that value(s).

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

For any of the structural and functional characteristics described herein, methods of determining these characteristics are known in the art.

The term “optionally” is meant, when used sequentially, to include from one to all of the enumerated combinations and contemplates all sub-combinations.

The term “amino acid” refers to, for example, the twenty common naturally occurring amino acids. Naturally occurring amino acids include alanine (Ala; A), arginine (Arg; R), asparagine (Asn; N), aspartic acid (Asp; D), cysteine (Cys; C), glutamic acid (Glu; E), glutamine (Gln; Q), Glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope).

The term “kd” (sec−1), as used herein, refers to the dissociation rate constant of a particular antibody-antigen interaction. This value is also referred to as the koff value.

The term “ka” (M−1×sec−1), as used herein, refers to the association rate constant of a particular antibody-antigen interaction. This value is also referred to as the kon value.

The term “KD” (M), as used herein, refers to the dissociation equilibrium constant of a particular antibody-antigen interaction. KD=kd/ka. In some embodiments, the affinity of an antibody is described in terms of the KD for an interaction between such antibody and its antigen. For clarity, as known in the art, a smaller KD value indicates a higher affinity interaction, while a larger KD value indicates a lower affinity interaction.

The term “KA” (M−1), as used herein, refers to the association equilibrium constant of a particular antibody-antigen interaction. KA=ka/kd.

As used herein, “administration” refers to providing or giving a subject a therapeutic agent (e.g., an anti-TSLP antibody, or an antigen binding fragment thereof, described herein) by any effective route. Exemplary routes of administration are described herein below.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the terms “nucleic acid” and “polynucleotide,” used interchangeably herein, refer to a polymeric form of nucleosides in any length. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically- or biologically-modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in TABLE 1 below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in cubic angstroms: 50-100 is small, 100-150 is intermediate, 150-200 isbased on volume in cubic angstroms: 50-100 is small, 100-150 is intermediate, 150-200 is large, and >200 is bulky
From TABLE 1 it is appreciated that the conservative amino acid families include (i) G, A, V, L, and I; (ii) D and E; (iii) C, S, and T; (iv) H, K, and R; (v) N and Q; and (vi) F, Y, and W.
A conservative mutation or substitution is therefore one that substitutes one amino acid for a member of the same amino acid family (e.g., a substitution of Ser for Thr or Lys for Arg).

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

An “anti-TSLP antibody,” “TSLP antibody,” or “TSLP specific antibody” is an antibody, as provided herein, which specifically binds to the antigen TSLP.

The term “epitope” means a portion of an antigen that specifically binds to an antibody.

The term “hypervariable region” or “HVR,” as used herein, refers to each of the regions of an antibody variable domain which are hypervariable in sequence and/or form structurally defined loops (“hypervariable loops”).

The term “antigen-binding domain” means the portion of an antibody that is capable of specifically binding to an antigen or epitope.

The term “chimeric antibody” refers to an antibody in which a portion of the heavy and/or light chain is derived from a particular source or species, while the remainder of the heavy and/or light chain is derived from a different source or species.

The term “human antibody” or “fully human antibody” refers to an antibody which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “humanized antibody” refers to a protein having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

The term “multispecific antibody” refers to an antibody that comprises two or more different antigen-binding domains that collectively specifically bind two or more different epitopes.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately, the scFv comprises a polypeptide chain wherein the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains, respectively. The variable domains comprise the complementarity determining loops (CDR, also referred to as hypervariable region (HVR)) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv, see Pluckthun in The Pharmacology of Monoclonal Antibodies, vol. 113, Rosenburg and Moore eds., Springer-Verlag, New York, pp. 269-315 (1994).

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., (1998) FEBS Letters 414:521-526 and Muyldermans et al. (2001) Trends in Biochem. Sci. 26:230-245, each of which is incorporated by reference in its entirety. Single domain antibodies are also known as sdAbs or nanobodies. SdAbs are fairly stable and easy to express as fusion partner with the Fc chain of an antibody (Harmsen M M, De Haard H J (2007) “Properties, production, and applications of camelid single-domain antibody fragments” Appl. Microbiol Biotechnol. 77 (1): 13-22).

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “antibody fragment” refers to an antibody that comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

The term “Fc domain” or “Fc region” herein is used to define a C-terminal region of an immunoglobulin heavy chain that contains at least a portion of the constant region. The term includes native sequence Fc regions and variant Fc regions.

The term “substantially purified” refers to a construct described herein, or variant thereof that may be substantially or essentially free of components that normally accompany or interact with the protein as found in its naturally occurring environment, i.e. a native cell, or host cell in the case of recombinantly produced antibody that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, (1981) Adv. Appl. Math. 2:482, by the homology alignment algorithm of Needleman & Wunsch, (1970) J. Mol. Biol. 48:443, by the search for similarity method of Pearson & Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of an algorithm that is suitable for determining percent sequence identity and sequence similarity is the BLAST algorithm, which is described in Altschul et al. (1990) J. Mol. Biol. 215:403-410. Software for performing BLAST analyses is publicly available through the National Center for Biotechnology Information (www.ncbi.nlm.nih.gov/).

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

The term “atopic dermatitis disease severity outcome measure” refers to a determination of certain signs, symptoms, features or parameters that have been associated with atopic dermatitis and that can be quantitatively or qualitatively assessed. Exemplary atopic dermatitis disease severity outcome measures include “Eczema Area and Severity Index” (EASI), “Severity Scoring of Atopic Dermatitis” (SCORAD), “Validated Investigator Global Assessment-Atopic Dermatitis” (vIGA-AD), “Investigator Global Assessment of Signs” (IGSA), Rajka/Langeland Atopic Dermatitis Severity Score, “Body Surface Area” (BSA), and patient-reported outcomes including Pruritus Visual Analog Scale (an aspect of disease severity assessed as part of SCORAD), Sleep Loss Visual Analog Scale (an aspect of disease severity assessed as part of SCORAD), Atopic Dermatitis Symptom Diary (ADSD), Atopic Dermatitis Impact Questionnaire (ADIQ), Dermatology Life Quality Index (DLQI) (Finlay and Khan, Clin Exper Dermatol 1994; 19:210), 5-D Itch Scale (Elman et al., Br J Dermatol 2010; 162 (3): 587-593), Itch Numeric Rating Scale (I-NRS) (see Naegeli et al., International Journal of Dermatology. 2015; 54 (6): 715-722; and Newton L, et al., J. Patient Rep. Outcomes. 2019; 3 (1): 42), and the Patient-Oriented Eczema Measure (POEM) (www.nottingham.ac.uk/research/groups/cebd/resources/poem.aspx).

### Hyaluronidases

As used herein, “hyaluronidase” refers to an enzyme that degrades hyaluronic acid, which constitutes an essential part of the extracellular matrix. Included in this definition are naturally occurring hyaluronidases, recombinant hyaluronidases, both human and from other sources, as well as variants thereof. Such hyaluronidases include, but are not limited to, nonhuman hyaluronidases, including bacterial hyaluronidases, bovine hyaluronidases, ovine hyaluronidases, and variants thereof. Reference to hyaluronidase refers to all forms, including variants.

Hyaluronidases were initially discovered in bacteria, and are now known to be widely distributed in nature and have been found in many different species, including insects, snakes, and mammals. Human hyaluronidase is present both in organs (e.g., testis, spleen, skin, eyes, liver, kidneys, uterus, and placenta) and in body fluids (e.g., tears, blood, and semen). In the human, six different hyaluronidases, HYAL1-4, HYAL-P1 and PH-20, have been identified. PH-20 exerts the strongest biologic activity, is found in high concentrations in the testicles, and can be localized on the head and the acrosome of human spermatozoa (see, e.g., Weber G C, et al., Adv. Exp. Med. Biol. 2019; 1148:255-277).

Hyaluronidases are classified into three categories according to their mechanism of action (see, e.g., Jung H., Arch. Plast. Surg. 2020 July; 47(4): 297-300). First, mammalian hyaluronidases are endo-β-N-acetylhexosaminidases that break down β-1,4 glycosidic linkages to form tetrasaccharides. Second, leech/hookworm hyaluronidases are endo-β-D-glucuronidases that break down β-1,3 glycosidic bonds to form pentasaccharides and hexasaccharides. Finally, microbial hyaluronidases are classified as hyaluronate lyases. Unlike other hyaluronidases, they do not catalyze hydrolysis reactions. Instead, they produce unsaturated disaccharides through a β-elimination reaction at β-1,4 glycosidic linkage.

Hyaluronidases can also be classified into two types according to the pH at which they are most active (see, e.g., Jung H., Arch. Plast. Surg. 2020 July; 47(4): 297-300). Acid-active hyaluronidases are activated at a pH of 3 to 4. Neutral-active hyaluronidases, which include the hyaluronidase enzymes found in snake and bee venom, are activated at a pH of 5 to 8 (see, e.g., Cavallini M, et al., Aesthet. Surg. J. 2013; 33:1167-74).

Hyaluronidase use has become more diverse and widespread in clinical practice. Today, animal-derived bovine or ovine testicular hyaluronidases, as well as synthetic hyaluronidases, are clinically applied as adjuncts to increase the bioavailability of drugs, for the therapy of extravasations, or for the management of complications associated with the aesthetic injection of hyaluronic acid-based fillers.

Exemplary of hyaluronan degrading enzymes are hyaluronidases, and particular chondroitinases and lyases that have the ability to depolymerize hyaluronan. Exemplary chondroitinases that are hyaluronan degrading enzymes include, but are not limited to, chondroitin ABC lyase (also known as chondroitinase ABC), chondroitin AC lyase (also known as chondroitin sulfate lyase or chondroitin sulfate eliminase) and chondroitin C lyase. Chondroitin ABC lyase comprises two enzymes, chondroitin-sulfate-ABC endolyase (EC 4.2.2.20) and chondroitin-sulfate-ABC exolyase (EC 4.2.2.21). Exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase (e.g., see Sato et al. (1994) Appl. Microbiol. Biotechnol. 41 (1): 39-46)). Exemplary chondroitinase AC enzymes from the bacteria include, but are not limited to, those from Flavobacterium heparinum, Victivallis vadensis, and Arthrobacter aurescens (see, e.g., Tkalec et al. (2000) Applied and Environmental Microbiology 66 (1): 29-35; Ernst et al. (1995) Critical Reviews in Biochemistry and Molecular Biology 30 (5): 387-444). Exemplary chondroitinase C enzymes from the bacteria include, but are not limited to, those from Streptococcus and Flavobacterium (Hibi et al. (1989) FEMS-Microbiol-Lett. 48 (2): 121-4; Michelacci et al. (1976) J. Biol. Chem. 251:1154-8; Tsuda et al. (1999) Eur. J. Biochem. 262:127-133).

Hyaluronidases include bacterial hyaluronidases (EC 4.2.2.1 or EC 4.2.99.1), hyaluronidases from leeches, other parasites, and crustaceans (EC 3.2.1.36), and mammalian-type hyaluronidases (EC 3.2.1.35). Hyaluronidases include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include hyaluronidases from cows (SEQ ID NOs: 10, 11, 64 of U.S. Pat. No. 8,568,713) and BH55 (U.S. Pat. Nos. 5,747,027 and 5,827,721), yellow jacket wasp (SEQ ID NOs: 12 and 13 of U.S. Pat. No. 8,568,713), honey bee (SEQ ID NO:14 of U.S. Pat. No. 8,568,713), white-face hornet (SEQ ID NO:15 of U.S. Pat. No. 8,568,713), paper wasp (SEQ ID NO: 16 of U.S. Pat. No. 8,568,713), mouse (SEQ ID NOs: 17-19, and 32 of U.S. Pat. No. 8,568,713), pig (SEQ ID NOs: 20-21 of U.S. Pat. No. 8,568,713), rat (SEQ ID NOs: 22-24, and 31 of U.S. Pat. No. 8,568,713), rabbit (SEQ ID NO:25 of U.S. Pat. No. 8,568,713), sheep (SEQ ID NOs: 26, 27, 63 and 65 of U.S. Pat. No. 8,568,713), orangutan (SEQ ID NO:28 of U.S. Pat. No. 8,568,713), cynomolgus monkey (SEQ ID NO:29 of U.S. Pat. No. 8,568,713), guinea pig (SEQ ID NO:30 of U.S. Pat. No. 8,568,713), Arthrobacter sp. (strain FB24) (SEQ ID NO: 67 of U.S. Pat. No. 8,568,713), Bdellovibrio bacteriovorus (SEQ ID NO:68 of U.S. Pat. No. 8,568,713), Propionibacterium acnes (SEQ ID NO:69 of U.S. Pat. No. 8,568,713), Streptococcus agalactiae ((SEQ ID NO:70 of U.S. Pat. No. 8,568,713); 18RS21 (SEQ ID NO: 71 of U.S. Pat. No. 8,568,713); serotype Ia (SEQ ID NO:72 of U.S. Pat. No. 8,568,713); serotype III (SEQ ID NO:73 of U.S. Pat. No. 8,568,713), Staphylococcus aureus (strain COL) (SEQ ID NO:74 of U.S. Pat. No. 8,568,713); strain MRSA252 (SEQ ID NOs: 75 and 76 of U.S. Pat. No. 8,568,713); strain MSSA476 (SEQ ID NO:77 of U.S. Pat. No. 8,568,713); strain NCTC 8325 (SEQ ID NO:78 of U.S. Pat. No. 8,568,713); strain bovine RF122 (SEQ ID NOs: 79 and 80 of U.S. Pat. No. 8,568,713); strain USA300 (SEQ ID NO: 81 of U.S. Pat. No. 8,568,713), Streptococcus pneumoniae (SEQ ID NO:82 of U.S. Pat. No. 8,568,713); strain ATCC BAA-255/R6 (SEQ ID NO:83 of U.S. Pat. No. 8,568,713); serotype 2, strain D39/NCTC 7466 (SEQ ID NO:84 of U.S. Pat. No. 8,568,713), Streptococcus pyogenes (serotype (SEQ ID NO:85 of U.S. Pat. No. 8,568,713); serotype M2, strain MGAS10270 (SEQ ID NO:86 of U.S. Pat. No. 8,568,713); serotype M4, strain MGAS10750 (SEQ ID NO:87 of U.S. Pat. No. 8,568,713); serotype M6 (SEQ ID NO:88 of U.S. Pat. No. 8,568,713); serotype M12, strain MGAS2096 (SEQ ID NOs: 89 and 90 of U.S. Pat. No. 8,568,713); serotype M12, strain MGAS9429 (SEQ ID NO:91 of U.S. Pat. No. 8,568,713); serotype M28 (SEQ ID NO:92 of U.S. Pat. No. 8,568,713); Streptococcus suis (SEQ ID NOs: 93-95 of U.S. Pat. No. 8,568,713); Vibrio fischeri (strain ATCC 700601/ES114 (SEQ ID NO: 96 of U.S. Pat. No. 8,568,713), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607). Hyaluronidases also include those of human origin. Exemplary human hyaluronidases include PH20 (SEQ ID NO:1 of U.S. Pat. No. 8,568,713), HYAL1 (SEQ ID NO:36 of U.S. Pat. No. 8,568,713), HYAL2 (SEQ ID NO:37 of U.S. Pat. No. 8,568,713), HYAL3 (SEQ ID NO:38 of U.S. Pat. No. 8,568,713), and HYAL4 (SEQ ID NO:36 of U.S. Pat. No. 8,568,713). The sequences and contents of U.S. Pat. No. 8,568,713 are expressly incorporated herein by reference. Also included amongst hyaluronidases are soluble hyaluronidases, including, ovine and bovine PH20, soluble human PH20 and soluble rHuPH20. Examples of commercially available bovine or ovine soluble hyaluronidases are Vitrase® (ovine hyaluronidase) and Amphadase® (bovine hyaluronidase).

Hyaluronidases as described herein include precursor hyaluronan degrading enzyme polypeptides and mature hyaluronan degrading enzyme polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 60%, 65%, 70%, 75%, 80%, 85%, 90%, 95%, 96%, 97%, 98%, 99% or more sequence identity to the polypeptides set forth in SEQ ID NOs: 409-418 and 420-475. Hyaluronidases also include those that contain chemical or posttranslational modifications and those that do not contain chemical or posttranslational modifications. Such modifications include, but are not limited to, pegylation, albumination, glycosylation, farnesylation, carboxylation, hydroxylation, phosphorylation, and other polypeptide modifications known in the art.

As used herein, a soluble hyaluronidase refers to a polypeptide characterized by its solubility under physiologic conditions. Soluble hyaluronidases can be distinguished, for example, by its partitioning into the aqueous phase of a Triton X-114 solution warmed to 37° C. (Bordier et al., (1981) J. Biol. Chem., 256:1604-7). Membrane-anchored, such as lipid anchored hyaluronidases, will partition into the detergent rich phase, but will partition into the detergent-poor or aqueous phase following treatment with Phospholipase-C. Included among soluble hyaluronidases are membrane anchored hyaluronidases in which one or more regions associated with anchoring of the hyaluronidase to the membrane has been removed or modified, where the soluble form retains hyaluronidase activity. Soluble hyaluronidases include recombinant soluble hyaluronidases and those contained in or purified from natural sources.

As used herein, activity refers to a functional activity or activities of a polypeptide or portion thereof associated with a full-length (complete) protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, hyaluronidase activity refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, Md.). A Reference Standard solution can be used in an assay to ascertain the relative activity, in units, of any hyaluronidase. In vitro assays to determine the hyaluronidase activity of hyaluronidases, such as soluble rHuPH20, are known in the art. Exemplary assays include the microturbidity assay described below (see, e.g., Example 3 of U.S. Pat. No. 8,568,713) that measures cleavage of hyaluronic acid by hyaluronidase indirectly by detecting the insoluble precipitate formed when the uncleaved hyaluronic acid binds with serum albumin. Reference Standards can be used, for example, to generate a standard curve to determine the activity in Units of the hyaluronidase being tested.

As used herein, “functionally equivalent amount” or grammatical variations thereof, with reference to a hyaluronan degrading enzyme, refers to the amount of hyaluronan degrading enzyme that achieves the same effect as an amount (such as a known number of Units of hyaluronidase activity) of a reference enzyme, such as a hyaluronidase. For example, the activity of any hyaluronan degrading enzyme can be compared to the activity of rHuPH20 to determine the functionally equivalent amount of a hyaluronan degrading enzyme that would achieve the same effect as a known amount of rHuPH20. For example, the ability of a hyaluronan degrading enzyme to act as a spreading or diffusing agent can be assessed by injecting it into the lateral skin of mice with trypan blue (see, e.g., U.S. Pat. Publication No. 20050260186), and the amount of hyaluronan degrading enzyme required to achieve the same amount of diffusion as, for example, 100 units of a Hyaluronidase Reference Standard, can be determined. The amount of hyaluronan degrading enzyme required is, therefore, functionally equivalent to 100 units.

Exemplary hyaluronan degrading enzymes are hyaluronidases, particularly soluble hyaluronidases, such as a PH20, or a truncated or variant form thereof. The PH20 can be, for example, an ovine, bovine or truncated human PH20. The human PH20 mRNA transcript is normally translated to generate a 509 amino acid precursor polypeptide (SEQ ID NO:409; and replicated below) containing a 35 amino acid signal sequence at the N-terminus (amino acid residue positions 1-35) and a 19 amino acid glycosylphosphatidylinositol (GPI) anchor attachment signal sequence at the C-terminus (amino acid residue positions 491-509). The mature PH20 is, therefore, a 474 amino acid polypeptide set forth in SEQ ID NO:410. Following transport of the precursor polypeptide to the ER and removal of the signal peptide, the C-terminal GPI-attachment signal peptide is cleaved to facilitate covalent attachment of a GPI anchor to the newly-formed C-terminal amino acid at the amino acid position corresponding to position 490 of the precursor polypeptide set forth in SEQ ID NO:409. Thus, a 474 amino acid GPI-anchored mature polypeptide with an amino acid sequence set forth in SEQ ID NO:410 is produced.

The amino acid sequence of the human PH20 precursor polypeptide (SEQ ID NO: 409; 509 amino acids) is as follows:

MGVLKFKHIFFRSFVKSSGVSQIVFTFLLIPCCLTLNFRAPPVIPNVPF
LWAWNAPSEFCLGKFDEPLDMSLFSFIGSPRINATGQGVTIFYVDRLGY
YPYIDSITGVTVNGGIPQKISLQDHLDKAKKDITFYMPVDNLGMAVIDW
EEWRPTWARNWKPKDVYKNRSIELVQQQNVQLSLTEATEKAKQEFEKAG
KDFLVETIKLGKLLRPNHLWGYYLFPDCYNHHYKKPGYNGSCFNVEIKR
NDDLSWLWNESTALYPSIYLNTQQSPVAATLYVRNRVREAIRVSKIPDA
KSPLPVFAYTRIVFTDQVLKFLSQDELVYTFGETVALGASGIVIWGTLS
IMRSMKSCLLLDNYMETILNPYIINVTLAAKMCSQVLCQEQGVCIRKNW
NSSDYLHLNPDNFAIQLEKGGKFTVRGKPTLEDLEQFSEKFYCSCYSTL
SCKEKADVKDTDAVDVCIADGVCIDAFLKPPMETEEPQIFYNASPSTLS
ATMFIVSILFLIISSVASL.

The amino acid sequence of the mature PH20 polypeptide (SEQ ID NO: 410; 474 amino acids) is as follows:

LNFRAPPVIPNVPFLWAWNAPSEFCLGKFDEPLDMSLFSFIGSPRINAT
GQGVTIFYVDRLGYYPYIDSITGVTVNGGIPQKISLQDHLDKAKKDITF
YMPVDNLGMAVIDWEEWRPTWARNWKPKDVYKNRSIELVQQQNVQLSLT
EATEKAKQEFEKAGKDFLVETIKLGKLLRPNHLWGYYLFPDCYNHHYKK
PGYNGSCFNVEIKRNDDLSWLWNESTALYPSIYLNTQQSPVAATLYVRN
RVREAIRVSKIPDAKSPLPVFAYTRIVFTDQVLKFLSQDELVYTFGETV
ALGASGIVIWGTLSIMRSMKSCLLLDNYMETILNPYIINVTLAAKMCSQ
VLCQEQGVCIRKNWNSSDYLHLNPDNFAIQLEKGGKFTVRGKPTLEDLE
QFSEKFYCSCYSTLSCKEKADVKDTDAVDVCIADGVCIDAFLKPPMETE
EPQIFYNASPSTLSATMFIVSILFLIISSVASL.

Human PH20 exhibits hyaluronidase activity at both neutral and acid pH. In one aspect, human PH20 is the prototypical neutral-active hyaluronidase that is generally locked to the plasma membrane via a GPI anchor. In another aspect, PH20 is expressed on the inner acrosomal membrane where it has hyaluronidase activity at both neutral and acid pH. It appears that PH20 contains two catalytic sites at distinct regions of the polypeptide: the Peptide 1 and Peptide 3 regions (Cherr et al., (2001) Matrix Biology 20:515-525). Evidence suggests that the Peptide 1 region of PH20, which corresponds to amino acid positions 107-137 of the mature polypeptide set forth in SEQ ID NO:410 and positions 142-172 of the precursor polypeptide set forth in SEQ ID NO:409, is required for enzyme activity at neutral pH. Amino acids at positions 111 and 113 (corresponding to the mature PH20 polypeptide set forth in SEQ ID NO:410) within this region appear to be important for activity, as mutagenesis by amino acid replacement results in PH20 polypeptides with 3% hyaluronidase activity or undetectable hyaluronidase activity, respectively, compared to the wild-type PH20 (Arming et al., (1997) Eur. J. Biochem. 247:810-814).

The Peptide 3 region, which corresponds to amino acid positions 242-262 of the mature polypeptide set forth in SEQ ID NO:410, and positions 277-297 of the precursor polypeptide set forth in SEQ ID NO:409, appears to be important for enzyme activity at acidic pH. Within this region, amino acids at positions 249 and 252 of the mature PH20 polypeptide appear to be essential for activity, and mutagenesis of either one results in a polypeptide essentially devoid of activity (Arming et al., (1997) Eur. J. Biochem. 247:810-814).

In addition to the catalytic sites, PH20 also contains a hyaluronan-binding site. Experimental evidence suggest that this site is located in the Peptide 2 region, which corresponds to amino acid positions 205-235 of the precursor polypeptide set forth in SEQ ID NO: 409 and positions 170-200 of the mature polypeptide set forth in SEQ ID NO:410. This region is highly conserved among hyaluronidases and is similar to the heparin binding motif. Mutation of the arginine residue at position 176 (corresponding to the mature PH20 polypeptide set forth in SEQ ID NO: 410) to a glycine results in a polypeptide with only about 1% of the hyaluronidase activity of the wild type polypeptide (Arming et al., (1997) Eur. J. Biochem. 247:810-814).

There are seven potential N-linked glycosylation sites in human PH20 at N82, N166, N235, N254, N368, N393, N490 of the polypeptide exemplified in SEQ ID NO:409. Because amino acids 36 to 464 of SEQ ID NO:409 appear to contain the minimally active human PH20 hyaluronidase domain, the N-linked glycosylation site N-490 is not required for proper hyaluronidase activity. There are six disulfide bonds in human PH20. Two disulfide bonds between the cysteine residues C60 and C351 and between C224 and C238 of the polypeptide exemplified in SEQ ID NO:409 (corresponding to residues C25 and C316, and C189 and C203 of the mature polypeptide set forth in SEQ ID NO:410, respectively). A further four disulfide bonds are formed between the cysteine residues C376 and C387; between C381 and C435; between C437 and C443; and between C458 and C464 of the polypeptide exemplified in SEQ ID NO: 409 (corresponding to residues C341 and C352; between C346 and C400; between C402 and C408; and between C423 and C429 of the mature polypeptide set forth in SEQ ID NO:410, respectively).

As used herein, soluble recombinant human PH20 (rHuPH20) refers to a soluble form of human PH20 that is recombinantly expressed in Chinese Hamster Ovary (CHO) cells. Soluble human PH20 or sHuPH20 includes mature polypeptides lacking all or a portion of the glycosylphospatidylinositol (GPI) attachment site at the C-terminus such that upon expression, the polypeptides are soluble.

Soluble rHuPH20 is encoded by a nucleic acid that includes the signal sequence and is set forth in SEQ ID NO:419. Also included are DNA molecules that are allelic variants thereof and other soluble variants. The nucleic acid encoding soluble rHuPH20 is expressed in CHO cells which secrete the mature polypeptide.

SEQ ID NO:419 encodes amino acids 1-482 of the human PH20 precursor polypeptide (amino acids 1-482 of SEQ ID NO: 409). Post translational processing removes the 35 amino acid signal sequence, leaving a 447 amino acid soluble recombinant human PH20 (SEQ ID NO: 411). As produced in the culture medium, there is heterogeneity at the C-terminus such that the product, designated rHuPH20, includes a mixture of species that can include any one or more of SEQ ID NOs: 411-416 in various abundance. Typically, rHuPH20 is produced in cells that facilitate correct N-glycosylation to retain activity, such as CHO cells (e.g., DG44 CHO cells). rHuPH20 may be produced by expressing a polynucleotide encoding amino acids 36-482 of SEQ ID NO: 409 in a cell, such as a CHO cell, and the polynucleotide encoding amino acids 36-482 of SEQ ID NO: 409 may be linked to the native signal sequence (amino acids 1-35 of SEQ ID NO: 409, as in SEQ ID NO: 419) or a heterologous signal sequence. The production of rHuPH20 is described in U.S. Pat. Nos. 8,568,713 and 8,343,487, each of which is incorporated herein by reference.

Exemplary sHuPH20 polypeptides include mature polypeptides having an amino acid sequence set forth in any one of SEQ ID NOs: 411-418 and 420-475. Other variants can have 60%, 70%, 80%, 85%, 90%, 91%, 92%, 93%, 94%, 95%, 96%, 97%, 98%, 99% or more sequence identity to any of SEQ ID NOs: 411-418 and 420-475, as long they retain a hyaluronidase activity and are soluble. Corresponding allelic variants and other variants also are included, including those corresponding to the precursor human PH20 polypeptide set forth in SEQ ID NO:409 and the mature human PH20 polypeptide set forth in SEQ ID NO: 410.

rHuPH20 was approved by the US Food and Drug Administration in 2005 as an adjuvant to increase the dispersion and absorption of other injected drugs. Recombinant human hyaluronidase PH20 is a transiently and locally-acting permeation enhancer that increases the dispersion and absorption of other injected agents. Recombinant human hyaluronidase PH20 depolymerizes hyaluronic acid (HA) at the injection site causing rapid decrease in the viscosity of the extracellular matrix, allowing bulk fluid flow and facilitating dispersion and absorption of co-administered agents (rHuPH20 Investigator's Brochure, 2018).

rHuPH20 is a glycosylated single chain protein with up to 447 amino acids, synthesized in CHO cells. As described above, rHuPH20 may be produced from a polynucleotide encoding amino acids 1-482 of SEQ ID NO: 409 (set forth in SEQ ID NO: 419), and post translational processing removes the 35 amino acid signal sequence, resulting in a polypeptide containing amino acids 36-482 of SEQ ID NO: 409 (set forth in SEQ ID NO: 411). As produced in the culture medium there is heterogeneity at the C-terminus such that the resulting rHuPH20 includes a mixture of species that can include any one or more of SEQ ID NOs: 411-416 in various abundance. Recombinant human hyaluronidase PH20 degrades HA under physiologic conditions and acts as a spreading factor in vivo. Therefore, when combined (co-mixed) or co-formulated with certain injectable drugs, rHUPH20 facilitates the absorption and dispersion of these drugs by temporarily reducing resistance to bulk fluid flow in the subcutaneous space. The permeability barrier in these tissues is restored to pre-injection levels within 24 to 48 hours after injection of rHuPH20.

Any suitable hyaluronidase (e.g., a recombinant human hyaluronidase) can be used in the methods, compositions, and combinations described herein, including, but not limited to, those described in U.S. Pat. No. 7,767,429 (e.g., SEQ ID NO:1), U.S. Pat. No. 7,846,431 (e.g., SEQ ID NO:1), U.S. Pat. No. 7,871,607 (e.g., SEQ ID NO:1), U.S. Pat. No. 8,105,586 (e.g., SEQ ID NO:1), U.S. Pat. No. 8,202,517 (e.g., SEQ ID NO:1), U.S. Pat. No. 8,257,699 (e.g., SEQ ID NO:1), U.S. Pat. No. 8,450,470 (e.g., SEQ ID NO:1), U.S. Pat. No. 8,431,124 (e.g., SEQ ID NO:1), U.S. Pat. No. 8,431,380 (e.g., SEQ ID NO:1), U.S. Pat. No. 8,580,252 (e.g., SEQ ID NO:1), U.S. Pat. No. 8,765,685 (e.g., SEQ ID NO:1), U.S. Pat. No. 8,772,246 (e.g., SEQ ID NO:1), U.S. Pat. No. 9,211,315 (e.g., SEQ ID NO:1), U.S. Pat. No. 9,562,223 (e.g., SEQ ID NO:1), U.S. Pat. No. 9,677,061 (e.g., SEQ ID NO:1), US Patent No.: U.S. Pat. No. 9,677,062 (e.g., SEQ ID NO:1), U.S. Pat. No. 5,721,348 (e.g., SEQ ID NO: 6), US20210155913, US20210363270, and US20220289864, the contents of each of which are expressly incorporated herein by reference. The generation of such recombinant human hyaluronidases is described in U.S. Pat. Nos. 7,767,429; 7,871,607; and US20060104968, the contents of each of which are expressly incorporated herein by reference.

An exemplary recombinant human hyaluronidase is a soluble hyaluronidase provided as a composition designated rHuPH20, i.e., the active ingredient in the commercial product Hylenex® recombinant (hyaluronidase human injection), which is supplied as ENHANZE® drug product. ENHANZE® is a formulation containing rHuPH20 that can be admixed with an antibody prior to subcutaneous injection.

Hyaluronidases that have altered properties, such as increased stability and/or activity, have been produced and can also be used, including, but not limited to those described in U.S. Pat. Nos. 9,447,401, 10,865,400, and 11,041,149, the contents of each of which are expressly incorporated herein by reference. These patents provide about 7000 examples in which the effects of replacing each amino acid with 15 other amino acids on activity and stability were identified and described.

Other variants are known to those of skill in the art including those described, for example, in WO2020/022791 and WO2020197230A, the contents of each of which are expressly incorporated herein by reference. These variant polypeptides include replacements, insertions, and deletions, including one or more amino acid residues S343E, M345T, K349E, L353A, L354I, N356E, and 136 IT. Variants that contain such modifications and others are set forth in SEQ ID NOs: 60-115 from WO2020/022791. WO2021/150079 also provides variant polypeptides described as having increased stability.

In one embodiment, the recombinant human hyaluronidase includes a sequence of amino acids in any one of SEQ ID NOs: 409-418 and 420-475, or has at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence of amino acids included in any one of SEQ ID NOs: 409-418 and 420-475 and retains hyaluronidase activity. In one embodiment, the recombinant human hyaluronidase comprises a sequence having at least 95%, 96%, 97%, 98%, or 99% to the amino acid sequence of SEQ ID NO: 409. In one embodiment, the recombinant human hyaluronidase comprises a sequence having at least 95%, 96%, 97%, 98%, or 99% to the amino acid sequence of SEQ ID NO: 411.

In another embodiment, the recombinant human hyaluronidase comprises the amino acid sequence set forth in SEQ ID NO:409. In another embodiment, the recombinant human hyaluronidase consists of the amino acid sequence set forth in SEQ ID NO:409. In another embodiment, the recombinant human hyaluronidase comprises the amino acid sequence set forth in SEQ ID NO:410. In another embodiment, the recombinant human hyaluronidase consists of the amino acid sequence set forth in SEQ ID NO:410. In another embodiment, the recombinant human hyaluronidase comprises the amino acid sequence set forth in SEQ ID NO:411. In another embodiment, the recombinant human hyaluronidase consists of the amino acid sequence set forth in SEQ ID NO:411. In another embodiment, the recombinant human hyaluronidase comprises the amino acid sequence set forth in SEQ ID NO:412. In another embodiment, the recombinant human hyaluronidase consists of the amino acid sequence set forth in SEQ ID NO: 412. In another embodiment, the recombinant human hyaluronidase comprises the amino acid sequence set forth in SEQ ID NO:413. In another embodiment, the recombinant human hyaluronidase consists of the amino acid sequence set forth in SEQ ID NO:413. In another embodiment, the recombinant human hyaluronidase comprises the amino acid sequence set forth in SEQ ID NO:414. In another embodiment, the recombinant human hyaluronidase consists of the amino acid sequence set forth in SEQ ID NO:414. In another embodiment, the recombinant human hyaluronidase comprises the amino acid sequence set forth in SEQ ID NO:415. In another embodiment, the recombinant human hyaluronidase consists of the amino acid sequence set forth in SEQ ID NO:415. In another embodiment, the recombinant human hyaluronidase comprises the amino acid sequence set forth in SEQ ID NO:416. In another embodiment, the recombinant human hyaluronidase consists of the amino acid sequence set forth in SEQ ID NO: 416. In another embodiment, the recombinant human hyaluronidase comprises the amino acid sequence set forth in SEQ ID NO:417. In another embodiment, the recombinant human hyaluronidase consists of the amino acid sequence set forth in SEQ ID NO:417. In another embodiment, the recombinant human hyaluronidase comprises the amino acid sequence set forth in SEQ ID NO:418. In another embodiment, the recombinant human hyaluronidase consists of the amino acid sequence set forth in SEQ ID NO:418. In another embodiment, the recombinant human hyaluronidase comprises the amino acid sequence set forth in SEQ ID NO:420. In another embodiment, the recombinant human hyaluronidase consists of the amino acid sequence set forth in SEQ ID NO:420. In another embodiment, the recombinant human hyaluronidase comprises the amino acid sequence set forth in SEQ ID NO:421. In another embodiment, the recombinant human hyaluronidase consists of the amino acid sequence set forth in SEQ ID NO: 421. In another embodiment, the recombinant human hyaluronidase comprises the amino acid sequence set forth in SEQ ID NO:422. In another embodiment, the recombinant human hyaluronidase consists of the amino acid sequence set forth in SEQ ID NO:422. In another embodiment, the recombinant human hyaluronidase comprises the amino acid sequence set forth in SEQ ID NO:423. In another embodiment, the recombinant human hyaluronidase consists of the amino acid sequence set forth in SEQ ID NO:423. In another embodiment, the recombinant human hyaluronidase comprises the amino acid sequence set forth in SEQ ID NO:424. In another embodiment, the recombinant human hyaluronidase consists of the amino acid sequence set forth in SEQ ID NO:424. In another embodiment, the recombinant human hyaluronidase comprises the amino acid sequence set forth in SEQ ID NO:425. In another embodiment, the recombinant human hyaluronidase consists of the amino acid sequence set forth in SEQ ID NO: 425. In another embodiment, the recombinant human hyaluronidase comprises the amino acid sequence set forth in SEQ ID NO:426. In another embodiment, the recombinant human hyaluronidase consists of the amino acid sequence set forth in SEQ ID NO:426. In another embodiment, the recombinant human hyaluronidase comprises the amino acid sequence set forth in SEQ ID NO:427. In another embodiment, the recombinant human hyaluronidase consists of the amino acid sequence set forth in SEQ ID NO:427. In another embodiment, the recombinant human hyaluronidase comprises the amino acid sequence set forth in SEQ ID NO:428. In another embodiment, the recombinant human hyaluronidase consists of the amino acid sequence set forth in SEQ ID NO:428. In another embodiment, the recombinant human hyaluronidase comprises the amino acid sequence set forth in SEQ ID NO:429. In another embodiment, the recombinant human hyaluronidase consists of the amino acid sequence set forth in SEQ ID NO: 429. In another embodiment, the recombinant human hyaluronidase comprises the amino acid sequence set forth in SEQ ID NO:430. In another embodiment, the recombinant human hyaluronidase consists of the amino acid sequence set forth in SEQ ID NO:430. In another embodiment, the recombinant human hyaluronidase comprises the amino acid sequence set forth in SEQ ID NO:431. In another embodiment, the recombinant human hyaluronidase consists of the amino acid sequence set forth in SEQ ID NO:431. In another embodiment, the recombinant human hyaluronidase comprises the amino acid sequence set forth in SEQ ID NO:432. In another embodiment, the recombinant human hyaluronidase consists of the amino acid sequence set forth in SEQ ID NO:432. In another embodiment, the recombinant human hyaluronidase comprises the amino acid sequence set forth in SEQ ID NO:433. In another embodiment, the recombinant human hyaluronidase consists of the amino acid sequence set forth in SEQ ID NO: 433. In another embodiment, the recombinant human hyaluronidase comprises the amino acid sequence set forth in SEQ ID NO:434. In another embodiment, the recombinant human hyaluronidase consists of the amino acid sequence set forth in SEQ ID NO:434. In another embodiment, the recombinant human hyaluronidase comprises the amino acid sequence set forth in SEQ ID NO:435. In another embodiment, the recombinant human hyaluronidase consists of the amino acid sequence set forth in SEQ ID NO:435. In another embodiment, the recombinant human hyaluronidase comprises the amino acid sequence set forth in SEQ ID NO:436. In another embodiment, the recombinant human hyaluronidase consists of the amino acid sequence set forth in SEQ ID NO:436. In another embodiment, the recombinant human hyaluronidase comprises the amino acid sequence set forth in SEQ ID NO:437. In another embodiment, the recombinant human hyaluronidase consists of the amino acid sequence set forth in SEQ ID NO: 437. In another embodiment, the recombinant human hyaluronidase comprises the amino acid sequence set forth in SEQ ID NO:438. In another embodiment, the recombinant human hyaluronidase consists of the amino acid sequence set forth in SEQ ID NO:438. In another embodiment, the recombinant human hyaluronidase comprises the amino acid sequence set forth in SEQ ID NO:439. In another embodiment, the recombinant human hyaluronidase consists of the amino acid sequence set forth in SEQ ID NO:439. In another embodiment, the recombinant human hyaluronidase comprises the amino acid sequence set forth in SEQ ID NO:440. In another embodiment, the recombinant human hyaluronidase consists of the amino acid sequence set forth in SEQ ID NO:440. In another embodiment, the recombinant human hyaluronidase comprises the amino acid sequence set forth in SEQ ID NO:441. In another embodiment, the recombinant human hyaluronidase consists of the amino acid sequence set forth in SEQ ID NO: 441. In another embodiment, the recombinant human hyaluronidase comprises the amino acid sequence set forth in SEQ ID NO:442. In another embodiment, the recombinant human hyaluronidase consists of the amino acid sequence set forth in SEQ ID NO:442. In another embodiment, the recombinant human hyaluronidase comprises the amino acid sequence set forth in SEQ ID NO:443. In another embodiment, the recombinant human hyaluronidase consists of the amino acid sequence set forth in SEQ ID NO:443. In another embodiment, the recombinant human hyaluronidase comprises the amino acid sequence set forth in SEQ ID NO:444. In another embodiment, the recombinant human hyaluronidase consists of the amino acid sequence set forth in SEQ ID NO:444. In another embodiment, the recombinant human hyaluronidase comprises the amino acid sequence set forth in SEQ ID NO:445. In another embodiment, the recombinant human hyaluronidase consists of the amino acid sequence set forth in SEQ ID NO: 445. In another embodiment, the recombinant human hyaluronidase comprises the amino acid sequence set forth in SEQ ID NO:446. In another embodiment, the recombinant human hyaluronidase consists of the amino acid sequence set forth in SEQ ID NO:446. In another embodiment, the recombinant human hyaluronidase comprises the amino acid sequence set forth in SEQ ID NO:447. In another embodiment, the recombinant human hyaluronidase consists of the amino acid sequence set forth in SEQ ID NO:447. In another embodiment, the recombinant human hyaluronidase comprises the amino acid sequence set forth in SEQ ID NO:448. In another embodiment, the recombinant human hyaluronidase consists of the amino acid sequence set forth in SEQ ID NO:448. In another embodiment, the recombinant human hyaluronidase comprises the amino acid sequence set forth in SEQ ID NO:449. In another embodiment, the recombinant human hyaluronidase consists of the amino acid sequence set forth in SEQ ID NO: 449. In another embodiment, the recombinant human hyaluronidase comprises the amino acid sequence set forth in SEQ ID NO:450. In another embodiment, the recombinant human hyaluronidase consists of the amino acid sequence set forth in SEQ ID NO:450. In another embodiment, the recombinant human hyaluronidase comprises the amino acid sequence set forth in SEQ ID NO:451. In another embodiment, the recombinant human hyaluronidase consists of the amino acid sequence set forth in SEQ ID NO:451. In another embodiment, the recombinant human hyaluronidase comprises the amino acid sequence set forth in SEQ ID NO:452. In another embodiment, the recombinant human hyaluronidase consists of the amino acid sequence set forth in SEQ ID NO:452. In another embodiment, the recombinant human hyaluronidase comprises the amino acid sequence set forth in SEQ ID NO:453. In another embodiment, the recombinant human hyaluronidase consists of the amino acid sequence set forth in SEQ ID NO: 453. In another embodiment, the recombinant human hyaluronidase comprises the amino acid sequence set forth in SEQ ID NO:454. In another embodiment, the recombinant human hyaluronidase consists of the amino acid sequence set forth in SEQ ID NO:454. In another embodiment, the recombinant human hyaluronidase comprises the amino acid sequence set forth in SEQ ID NO:455. In another embodiment, the recombinant human hyaluronidase consists of the amino acid sequence set forth in SEQ ID NO:455. In another embodiment, the recombinant human hyaluronidase comprises the amino acid sequence set forth in SEQ ID NO:456. In another embodiment, the recombinant human hyaluronidase consists of the amino acid sequence set forth in SEQ ID NO:456. In another embodiment, the recombinant human hyaluronidase comprises the amino acid sequence set forth in SEQ ID NO:457. In another embodiment, the recombinant human hyaluronidase consists of the amino acid sequence set forth in SEQ ID NO: 457. In another embodiment, the recombinant human hyaluronidase comprises the amino acid sequence set forth in SEQ ID NO:458. In another embodiment, the recombinant human hyaluronidase consists of the amino acid sequence set forth in SEQ ID NO:458. In another embodiment, the recombinant human hyaluronidase comprises the amino acid sequence set forth in SEQ ID NO:459. In another embodiment, the recombinant human hyaluronidase consists of the amino acid sequence set forth in SEQ ID NO:459. In another embodiment, the recombinant human hyaluronidase comprises the amino acid sequence set forth in SEQ ID NO:460. In another embodiment, the recombinant human hyaluronidase consists of the amino acid sequence set forth in SEQ ID NO:460. In another embodiment, the recombinant human hyaluronidase comprises the amino acid sequence set forth in SEQ ID NO:461. In another embodiment, the recombinant human hyaluronidase consists of the amino acid sequence set forth in SEQ ID NO: 461. In another embodiment, the recombinant human hyaluronidase comprises the amino acid sequence set forth in SEQ ID NO:462. In another embodiment, the recombinant human hyaluronidase consists of the amino acid sequence set forth in SEQ ID NO:462. In another embodiment, the recombinant human hyaluronidase comprises the amino acid sequence set forth in SEQ ID NO:463. In another embodiment, the recombinant human hyaluronidase consists of the amino acid sequence set forth in SEQ ID NO:463. In another embodiment, the recombinant human hyaluronidase comprises the amino acid sequence set forth in SEQ ID NO:464. In another embodiment, the recombinant human hyaluronidase consists of the amino acid sequence set forth in SEQ ID NO:464. In another embodiment, the recombinant human hyaluronidase comprises the amino acid sequence set forth in SEQ ID NO:465. In another embodiment, the recombinant human hyaluronidase consists of the amino acid sequence set forth in SEQ ID NO: 465. In another embodiment, the recombinant human hyaluronidase comprises the amino acid sequence set forth in SEQ ID NO:466. In another embodiment, the recombinant human hyaluronidase consists of the amino acid sequence set forth in SEQ ID NO:466. In another embodiment, the recombinant human hyaluronidase comprises the amino acid sequence set forth in SEQ ID NO:467. In another embodiment, the recombinant human hyaluronidase consists of the amino acid sequence set forth in SEQ ID NO:467. In another embodiment, the recombinant human hyaluronidase comprises the amino acid sequence set forth in SEQ ID NO:468. In another embodiment, the recombinant human hyaluronidase consists of the amino acid sequence set forth in SEQ ID NO:468. In another embodiment, the recombinant human hyaluronidase comprises the amino acid sequence set forth in SEQ ID NO:469. In another embodiment, the recombinant human hyaluronidase consists of the amino acid sequence set forth in SEQ ID NO: 469. In another embodiment, the recombinant human hyaluronidase comprises the amino acid sequence set forth in SEQ ID NO:470. In another embodiment, the recombinant human hyaluronidase consists of the amino acid sequence set forth in SEQ ID NO:470. In another embodiment, the recombinant human hyaluronidase comprises the amino acid sequence set forth in SEQ ID NO:471. In another embodiment, the recombinant human hyaluronidase consists of the amino acid sequence set forth in SEQ ID NO:471. In another embodiment, the recombinant human hyaluronidase comprises the amino acid sequence set forth in SEQ ID NO:472. In another embodiment, the recombinant human hyaluronidase consists of the amino acid sequence set forth in SEQ ID NO:472. In another embodiment, the recombinant human hyaluronidase comprises the amino acid sequence set forth in SEQ ID NO:473. In another embodiment, the recombinant human hyaluronidase consists of the amino acid sequence set forth in SEQ ID NO: 473. In another embodiment, the recombinant human hyaluronidase comprises the amino acid sequence set forth in SEQ ID NO:474. In another embodiment, the recombinant human hyaluronidase consists of the amino acid sequence set forth in SEQ ID NO:474. In another embodiment, the recombinant human hyaluronidase comprises the amino acid sequence set forth in SEQ ID NO:475. In another embodiment, the recombinant human hyaluronidase consists of the amino acid sequence set forth in SEQ ID NO:475.

### TSLP Binding Proteins

### Antibody Structure

Provided herein are TSLP binding proteins that bind to TSLP. In certain embodiments, the TSLP binding protein is an antibody, or an antigen binding fragment thereof. The present application also provides compositions and combinations comprising an antibody, or antigen binding fragment thereof, that binds TSLP as well as methods of use thereof. In certain embodiments, the TSLP binding protein is an antibody, or an antigen binding fragment thereof, comprising a modified Fc region. Exemplary TSLP antibodies are described in PCT Application No. PCT/US2025/034623, which is incorporated herein by reference in its entirety. In some embodiments, a TSLP antibody, or an antigen binding fragment thereof, described herein is selected from a TSLP antibody described in PCT Application No. PCT/US2025/034623.

The recognized immunoglobulin genes include the kappa, lambda, alpha, gamma, delta, epsilon and mu constant region genes, as well as the myriad immunoglobulin variable region genes. Light chains are classified as either kappa or lambda. There are four subtypes of human lambda light chain constant region (21, 22, 23, 27), which are encoded by the genes IGLC1, IGCL2, IGLC3, and IGLC7. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains, respectively. The IgG1 heavy chain comprises the VH, CH1, CH2, and CH3 domains, respectively from the N- to C-terminus. The light chain comprises the VL and CL domains from N- to C-terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antibody provided herein, is from or is derived from an immunoglobulin-based construct such as a diabody or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody, or any chimeric antibody.

In some embodiments, the antibodies provided herein comprise a heavy chain. In one embodiment, the heavy chain is an IgA. In one embodiment, the heavy chain is an IgD. In one embodiment, the heavy chain is an IgE. In one embodiment, the heavy chain is an IgG. In one embodiment, the heavy chain is an IgM. In one embodiment, the heavy chain is an IgG1. In one embodiment, the heavy chain is an IgG2. In one embodiment, the heavy chain is an IgG3. In one embodiment, the heavy chain is an IgG4. In one embodiment, the heavy chain is an IgA1. In one embodiment, the heavy chain is an IgA2.

In some embodiments, an antibody is an IgG1 antibody. In some embodiments, an antibody is an IgG3 antibody. In some embodiments, an antibody is an IgG2 antibody. In some embodiments, an antibody is an IgG4 antibody.

Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al. (1987) J Mol Biol 196:901-917, where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al. (1997) J. Mol. Biol., 273:927-948 (“Chothia” numbering scheme); MacCallum et al. (1996) J. Mol. Biol. 262:732-745 (“Contact” numbering scheme); Lefranc et al. (2003) Dev. Comp. Immunol. 27:55-77 (“IMGT” numbering scheme); and Honegge and Plückthun (2001) J. Mol. Biol. 309:657-70 (“AHo” numbering scheme); each of which is incorporated by reference in its entirety.

TABLE 2 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 2
Residues in CDRs according to Kabat and Chothia numbering schemes

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to TSLP or TSLP variants with different point-mutations, or to chimeric TSLP variants.

To screen for antibodies which bind to an epitope on a target antigen bound by an antibody of interest (e.g., TSLP and/or TSLP receptor), a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

Chimeric antibodies are antibodies in which a portion of the heavy and/or light chain is derived from a particular source or species, while the remainder of the heavy and/or light chain is derived from a different source or species.

Human, or fully human, antibodies are antibodies which possess an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297; 5,886,152; and 5,877,293. For further details, see Jones et al. (1986) Nature 321:522-525; Riechmann et al. (1988) Nature 332:323-329; and Presta (1992) Curr. Op. Struct. Biol. 2:593-596, each of which is incorporated by reference in its entirety.

Anti-TSLP antibodies can include those described herein such as the clones set forth in the drawings and/or tables. In some embodiments, the antibody comprises an alternative scaffold. In some embodiments, the antibody consists of an alternative scaffold. In some embodiments, the antibody consists essentially of an alternative scaffold. In some embodiments, the antibody comprises an antibody fragment. In some embodiments, the antibody consists of an antibody fragment. In some embodiments, the antibody consists essentially of an antibody fragment.

In some embodiments, the antibodies are monoclonal antibodies.

In some embodiments, the antibodies are polyclonal antibodies.

In some embodiments, the antibodies are produced by hybridomas. In other embodiments, the antibodies are produced by recombinant cells engineered to express the desired variable and constant domains.

In some embodiments, the antibodies may be single chain antibodies or other antibody derivatives retaining the antigen specificity and the lower hinge region or a variant thereof.

In some embodiments, the antibodies may be polyfunctional antibodies, recombinant antibodies, fully human antibodies, humanized antibodies, or fragments or variants thereof. In particular embodiments, the antibody fragment or a derivative thereof is selected from a Fab fragment, a Fab′2 fragment, a CDR and scFv.

TABLE 3
Sequences of anti-TSLP antibody constructs-CDRs

| | | | | | | | | | | | | |
|---|---|---|---|---|---|---|---|---|---|---|---|---|
| | | SEQ | | SEQ | | SEQ | | SEQ | | SEQ | | SEQ |
| | | ID | | ID | | ID | | ID | | ID | | ID |
| Antibody | CDRH1 | NO: | CDRH2 | NO: | CDRH3 | NO: | CDRL1 | NO: | CDRL2 | NO: | CDRL3 | NO: |

Kabat Numbering

| | | | | | | | | | | | | |
|---|---|---|---|---|---|---|---|---|---|---|---|---|
| Antibody | TYTMH | 1 | VIWYDG | 10 | APQWEL | 13 | GGNNLG | 19 | DVSD | 25 | QVWDSS | 27 |
| 1 | | | SNKHYA | | VHEAFDI | | SKSVH | | RPS | | STFVV | |
| | | | DSVKG | | | | | | | | | |
| Antibody | TYPMH | 2 | VIWYDG | 10 | APQWEL | 13 | GGNNLG | 19 | DVSD | 25 | QVWDSS | 28 |
| 2 | | | SNKHYA | | VHEAFDI | | SKSVH | | RPS | | STYVV | |
| | | | DSVKG | | | | | | | | | |
| Antibody | TYPMH | 2 | VIWYDG | 10 | ALIWELF | 14 | GGNNLI | 20 | DDSD | 26 | QVWDSS | 28 |
| 3 | | | SNKHYA | | HEYFDI | | SKSVH | | RPS | | STYVV | |
| | | | DSVKG | | | | | | | | | |
| Antibody | TYPMH | 2 | VIWYDG | 10 | APQWEL | 13 | GGNNLV | 21 | DVSD | 25 | QVWDSS | 28 |
| 4 | | | SNKHYA | | VHEAFDI | | SKSVH | | RPS | | STYVV | |
| | | | DSVKG | | | | | | | | | |
| Antibody | TYGMH | 3 | VIWYDG | 10 | APQWEL | 13 | GGNNLV | 21 | DDSD | 26 | QVWDSS | 27 |
| 5 | | | SNKHYA | | VHEAFDI | | SKSVH | | RPS | | STFVV | |
| | | | DSVKG | | | | | | | | | |
| Antibody | TYGMH | 3 | VIWYDG | 10 | ALLWEL | 15 | GGNNLV | 21 | DVSD | 25 | QVWDSS | 29 |
| 6 | | | SNKHYA | | FHEYFDI | | SKSVH | | RPS | | SDHVV | |
| | | | DSVKG | | | | | | | | | |
| Antibody | TYGMH | 3 | VIWYDG | 10 | APQWEL | 13 | GGNNLG | 19 | DVSD | 25 | QVWDSS | 30 |
| 7 | | | SNKHYA | | VHEAFDI | | SKSVH | | RPS | | STIVV | |
| | | | DSVKG | | | | | | | | | |
| Antibody | TYTMH | 1 | VIWYDG | 10 | APQWEL | 13 | GGNNLG | 19 | DDSD | 26 | QVWDK | 31 |
| 8 | | | SNKHYA | | VHEAFDI | | SKSVH | | RPS | | SSTYVV | |
| | | | DSVKG | | | | | | | | | |
| Antibody | TYPMH | 2 | VIWYDG | 10 | ALLWEL | 15 | GGNNLV | 21 | DDSD | 26 | QVWDSS | 28 |
| 9 | | | SNKHYA | | FHEYFDI | | SKSVH | | RPS | | STYVV | |
| | | | DSVKG | | | | | | | | | |
| Antibody | TYGMH | 3 | VIWYDG | 10 | APQWEL | 13 | RTSQNI | 271 | DVSSL | 303 | QQGYSP | 311 |
| 10 | | | SNKHYA | | VHEAFDI | | RYYLN | | QS | | PLT | |
| | | | DSVKG | | | | | | | | | |
| Antibody | TYGMH | 3 | VIWYDG | 10 | APQWEL | 13 | GGDNIG | 272 | EDSD | 304 | QVWDS | 312 |
| 11 | | | SNKHYA | | VHEAFDI | | SKSVH | | RPS | | DSDHVV | |
| | | | DSVKG | | | | | | | | | |
| Antibody | TYGMH | 3 | VIWYDG | 10 | APQWEL | 13 | GGQNIG | 273 | DDSD | 26 | QVWDSS | 313 |
| 12 | | | SNKHYA | | VHEAFDI | | TKSVH | | RPS | | SDQSV | |
| | | | DSVKG | | | | | | | | | |
| Antibody | TYGMH | 3 | VIWYDG | 10 | APQWEL | 13 | GGQNIG | 273 | DDSD | 26 | QVWDSS | 313 |
| 13 | | | SNKHYA | | VHEAFDI | | TKSVH | | RPS | | SDQSV | |
| | | | DSVKG | | | | | | | | | |
| Antibody | TYGMH | 3 | VIWYDG | 10 | APQWEL | 13 | GGQNIG | 273 | DDSD | 26 | QVWDSS | 313 |
| 14 | | | SNKHYA | | VHEAFDI | | TKSVH | | RPS | | SDQSV | |
| | | | DSVKG | | | | | | | | | |
| Antibody | TYGMH | 3 | VIWYDG | 10 | APQWEL | 13 | GGNKIG | 274 | DDSD | 26 | QVWDSS | 314 |
| 15 | | | SNKHYA | | VHEAFDI | | RKSVH | | RPS | | SDEGV | |
| | | | DSVKG | | | | | | | | | |
| Antibody | TYGMH | 3 | VIWYDG | 10 | APQWEL | 13 | GGNKIG | 274 | DDSD | 26 | QVWDSS | 314 |
| 16 | | | SNKHYA | | VHEAFDI | | RKSVH | | RPS | | SDEGV | |
| | | | DSVKG | | | | | | | | | |
| Antibody | TYGMH | 3 | VIWYDG | 10 | APQWEL | 13 | GGQNIG | 273 | DDSD | 26 | QVWDG | 315 |
| 17 | | | SNKHYA | | VHEAFDI | | TKSVH | | RPS | | SSDQSV | |
| | | | DSVKG | | | | | | | | | |
| Antibody | TYGMH | 3 | VIWYDG | 10 | APQWEL | 13 | GGNNIG | 275 | DDTD | 305 | QVWDT | 316 |
| 18 | | | SNKHYA | | VHEAFDI | | GKSVH | | RPS | | TTDHVV | |
| | | | DSVKG | | | | | | | | | |
| Antibody | TYGMH | 3 | VIWYDG | 10 | APQWEL | 13 | GGNNIG | 276 | DDSD | 26 | QVWDSS | 317 |
| 19 | | | SNKHYA | | VHEAFDI | | SKSVH | | RPS | | SDPVV | |
| | | | DSVKG | | | | | | | | | |
| Antibody | TYGMH | 3 | VIWYDG | 10 | APQWEL | 13 | GGQNIG | 273 | DDSD | 26 | QVWDSS | 313 |
| 20 | | | SNKHYA | | VHEAFDI | | TKSVH | | RPS | | SDQSV | |
| | | | DSVKG | | | | | | | | | |
| Antibody | TYGMH | 3 | VIWYDG | 10 | APQWEL | 13 | GGQNIG | 273 | DDSD | 306 | QVWDSS | 313 |
| 21 | | | SNKHYA | | VHEAFDI | | TKSVH | | RPP | | SDQSV | |
| | | | DSVKG | | | | | | | | | |
| Antibody | TYGMH | 3 | VIWYDG | 10 | APQWEL | 13 | GGNKIG | 274 | DDSD | 26 | QVWDSS | 314 |
| 22 | | | SNKHYA | | VHEAFDI | | RKSVH | | RPS | | SDEGV | |
| | | | DSVKG | | | | | | | | | |
| Antibody | TYGMH | 3 | VIWYDG | 10 | APQWEL | 13 | GGHNIG | 277 | DDSD | 26 | QVWDR | 318 |
| 23 | | | SNKHYA | | VHEAFDI | | TKSVH | | RPS | | SSDQSV | |
| | | | DSVKG | | | | | | | | | |
| Antibody | TYGMH | 3 | VIWYDG | 10 | APQWEL | 13 | GGDSIG | 278 | DDSD | 26 | QVWDR | 318 |
| 24 | | | SNKHYA | | VHEAFDI | | SKYVH | | RPS | | SSDQSV | |
| | | | DSVKG | | | | | | | | | |
| Antibody | TYGMH | 3 | VIWYDG | 10 | APQWEL | 13 | GGQNIG | 273 | DDSD | 26 | QVWDS | 319 |
| 25 | | | SNKHYA | | VHEAFDI | | TKSVH | | RPS | | GSGQTV | |
| | | | DSVKG | | | | | | | | | |
| Antibody | TYGMH | 3 | VIWYDG | 10 | APQWEL | 13 | GGQNIG | 279 | DDSD | 26 | QVWDSS | 313 |
| 26 | | | SNKHYA | | VHEAFDI | | TRSVH | | RPS | | SDQSV | |
| | | | DSVKG | | | | | | | | | |
| Antibody | TYGMH | 3 | VIWYDG | 10 | APQWEL | 13 | GGNNIG | 280 | DDSD | 26 | HVWDSS | 320 |
| 27 | | | SNKHYA | | VHEAFDI | | SETVH | | RPS | | SDHVM | |
| | | | DSVKG | | | | | | | | | |
| Antibody | TYGMH | 3 | VIWYDG | 10 | APQWEL | 13 | RGDNIE | 281 | DDSD | 26 | QVWDS | 321 |
| 28 | | | SNKHYA | | VHEAFDI | | TYSVH | | RPS | | DSDHLE | |
| | | | DSVKG | | | | | | | | | |
| Antibody | TYGMH | 3 | VIWYDG | 10 | APQWEL | 13 | GGNNIG | 282 | DDSD | 26 | QVWDS | 322 |
| 29 | | | SNKHYA | | VHEAFDI | | RKSAH | | RPS | | YSNHVV | |
| | | | DSVKG | | | | | | | | | |
| Antibody | TYGMH | 3 | VIWYDG | 10 | APQWEL | 13 | GGNKIG | 274 | DDSD | 26 | QVWDSS | 314 |
| 30 | | | SNKHYA | | VHEAFDI | | RKSVH | | RPS | | SDEGV | |
| | | | DSVKG | | | | | | | | | |
| Antibody | TYGMH | 3 | VIWYDG | 10 | APQWEL | 13 | GGHNIG | 277 | DDSD | 26 | QVWDR | 318 |
| 31 | | | SNKHYA | | VHEAFDI | | TKSVH | | RPS | | SSDQSV | |
| | | | DSVKG | | | | | | | | | |
| Antibody | TYGMH | 3 | VIWYDG | 10 | APQWEL | 13 | RGDNIE | 281 | DDSD | 26 | QVWDS | 323 |
| 32 | | | SNKHYA | | VHEAFDI | | TYSVH | | RPS | | GGDHV | |
| | | | DSVKG | | | | | | | | V | |
| Antibody | TYGMH | 3 | VIWYDG | 10 | APQWEL | 13 | GGNNIG | 276 | DDSD | 26 | QVWDSS | 29 |
| 33 | | | SNKHYA | | VHEAFDI | | SKSVH | | RPS | | SDHVV | |
| | | | DSVKG | | | | | | | | | |
| Antibody | TYGMH | 3 | VIWYDG | 10 | APQWEL | 13 | EGTYLG | 283 | DDKE | 307 | QVWDDI | 324 |
| 34 | | | SNKHYA | | VHEAFDI | | AKSVH | | RPS | | SDHIV | |
| | | | DSVKG | | | | | | | | | |
| Antibody | TYGMH | 3 | VIWYDG | 10 | APQWEL | 13 | GGNNIG | 284 | DDSD | 26 | QVWDSS | 313 |
| 35 | | | SNKHYA | | VHEAFDI | | TKSVH | | RPS | | SDQSV | |
| | | | DSVKG | | | | | | | | | |
| Antibody | TYGMH | 3 | VIWYDG | 10 | APQWEL | 13 | GGQSIG | 285 | DDSD | 26 | QVWDSS | 313 |
| 36 | | | SNKHYA | | VHEAFDI | | TKSVH | | RPS | | SDQSV | |
| | | | DSVKG | | | | | | | | | |
| Antibody | TYGMH | 3 | VIWYDG | 10 | APQWEL | 13 | GANSIE | 286 | DDSE | 308 | QVWDM | 325 |
| 37 | | | SNKHYA | | VHEAFDI | | DKNVH | | RPS | | ASDRVV | |
| | | | DSVKG | | | | | | | | | |
| Antibody | TYGMH | 3 | VIWYDG | 10 | APQWEL | 13 | GGNKIG | 274 | DDSD | 26 | QVWDSS | 314 |
| 38 | | | SNKHYA | | VHEAFDI | | RKSVH | | RPS | | SDEGV | |
| | | | DSVKG | | | | | | | | | |
| Antibody | TYGMH | 3 | VIWYDG | 10 | APQWEL | 13 | GGQNIG | 273 | DDSD | 26 | QVWDSS | 313 |
| 39 | | | SNKHYA | | VHEAFDI | | TKSVH | | RPS | | SDQSV | |
| | | | DSVKG | | | | | | | | | |
| Antibody | TYGMH | 3 | VIWYDG | 10 | APQWEL | 13 | GGNKIG | 274 | DDSD | 26 | QVWDSS | 314 |
| 40 | | | SNKHYA | | VHEAFDI | | RKSVH | | RPS | | SDEGV | |
| | | | DSVKG | | | | | | | | | |
| Antibody | TYGMH | 3 | VIWYDG | 10 | APQWEL | 13 | GGQNIG | 273 | DDSD | 26 | QVWDSS | 313 |
| 41 | | | SNKHYA | | VHEAFDI | | TKSVH | | RPS | | SDQSV | |
| | | | DSVKG | | | | | | | | | |
| Antibody | TYGMH | 3 | VIWYDG | 10 | APQWEL | 13 | RGDNIE | 281 | DDSD | 26 | QVWDSS | 29 |
| 42 | | | SNKHYA | | VHEAFDI | | TYSVH | | RPS | | SDHVV | |
| | | | DSVKG | | | | | | | | | |
| Antibody | TYGMH | 3 | VIWYDG | 10 | APQWEL | 13 | GGNKIG | 274 | DDSD | 26 | QVWDSS | 314 |
| 43 | | | SNKHYA | | VHEAFDI | | RKSVH | | RPS | | SDEGV | |
| | | | DSVKG | | | | | | | | | |
| Antibody | TYGMH | 3 | VIWYDG | 10 | APQWEL | 13 | GGNKIG | 274 | DDSD | 26 | QVWDTS | 326 |
| 44 | | | SNKHYA | | VHEAFDI | | RKSVH | | RPS | | SDQGV | |
| | | | DSVKG | | | | | | | | | |
| Antibody | TYGMH | 3 | VIWYDG | 10 | APQWEL | 13 | GGDNIG | 287 | DDLK | 309 | QVWDST | 327 |
| 45 | | | SNKHYA | | VHEAFDI | | SYSVH | | RPS | | SGVVF | |
| | | | DSVKG | | | | | | | | | |
| Antibody | TYGMH | 3 | VIWYDG | 10 | APQWEL | 13 | GGNNVE | 288 | DDND | 310 | QVWDSS | 29 |
| 46 | | | SNKHYA | | VHEAFDI | | DYSVH | | RPS | | SDHVV | |
| | | | DSVKG | | | | | | | | | |

Chothia Numbering

| | | | | | | | | | | | | |
|---|---|---|---|---|---|---|---|---|---|---|---|---|
| Antibody | GFTFRT | 4 | WYDGS | 11 | APQWEL | 13 | GGNNLG | 19 | DVSD | 25 | QVWDSS | 27 |
| 1 | YTMH | | N | | VHEAFDI | | SKSVH | | RPS | | STFVV | |
| Antibody | GFTFRT | 5 | WYDGS | 11 | APQWEL | 13 | GGNNLG | 19 | DVSD | 25 | QVWDSS | 28 |
| 2 | YPMH | | N | | VHEAFDI | | SKSVH | | RPS | | STYVV | |
| Antibody | GFTFRT | 5 | WYDGS | 11 | ALIWELF | 14 | GGNNLI | 20 | DDSD | 26 | QVWDSS | 28 |
| 3 | YPMH | | N | | HEYFDI | | SKSVH | | RPS | | STYVV | |
| Antibody | GFTFRT | 5 | WYDGS | 11 | APQWEL | 13 | GGNNLV | 21 | DVSD | 25 | QVWDSS | 28 |
| 4 | YPMH | | N | | VHEAFDI | | SKSVH | | RPS | | STYVV | |
| Antibody | GFTFRT | 6 | WYDGS | 11 | APQWEL | 13 | GGNNLV | 21 | DDSD | 26 | QVWDSS | 27 |
| 5 | YGMH | | N | | VHEAFDI | | SKSVH | | RPS | | STFVV | |
| Antibody | GFTFRT | 6 | WYDGS | 11 | ALLWEL | 15 | GGNNLV | 21 | DVSD | 25 | QVWDSS | 29 |
| 6 | YGMH | | N | | FHEYFDI | | SKSVH | | RPS | | SDHVV | |
| Antibody | GFTFRT | 6 | WYDGS | 11 | APQWEL | 13 | GGNNLG | 19 | DVSD | 25 | QVWDSS | 30 |
| 7 | YGMH | | N | | VHEAFDI | | SKSVH | | RPS | | STIVV | |
| Antibody | GFTFRT | 4 | WYDGS | 11 | APQWEL | 13 | GGNNLG | 19 | DDSD | 26 | QVWDK | 31 |
| 8 | YTMH | | N | | VHEAFDI | | SKSVH | | RPS | | SSTYVV | |
| Antibody | GFTFRT | 5 | WYDGS | 11 | ALLWEL | 15 | GGNNLV | 21 | DDSD | 26 | QVWDSS | 28 |
| 9 | YPMH | | N | | FHEYFDI | | SKSVH | | RPS | | STYVV | |
| Antibody | GFTFRT | 6 | WYDGS | 11 | APQWEL | 13 | RTSQNI | 271 | DVSSL | 303 | QQGYSP | 311 |
| 10 | YGMH | | N | | VHEAFDI | | RYYLN | | QS | | PLT | |
| Antibody | GFTFRT | 6 | WYDGS | 11 | APQWEL | 13 | GGDNIG | 272 | EDSD | 304 | QVWDS | 312 |
| 11 | YGMH | | N | | VHEAFDI | | SKSVH | | RPS | | DSDHVV | |
| Antibody | GFTFRT | 6 | WYDGS | 11 | APQWEL | 13 | GGQNIG | 273 | DDSD | 26 | QVWDSS | 313 |
| 12 | YGMH | | N | | VHEAFDI | | TKSVH | | RPS | | SDQSV | |
| Antibody | GFTFRT | 6 | WYDGS | 11 | APQWEL | 13 | GGQNIG | 273 | DDSD | 26 | QVWDSS | 313 |
| 13 | YGMH | | N | | VHEAFDI | | TKSVH | | RPS | | SDQSV | |
| Antibody | GFTFRT | 6 | WYDGS | 11 | APQWEL | 13 | GGQNIG | 273 | DDSD | 26 | QVWDSS | 313 |
| 14 | YGMH | | N | | VHEAFDI | | TKSVH | | RPS | | SDQSV | |
| Antibody | GFTFRT | 6 | WYDGS | 11 | APQWEL | 13 | GGNKIG | 274 | DDSD | 26 | QVWDSS | 314 |
| 15 | YGMH | | N | | VHEAFDI | | RKSVH | | RPS | | SDEGV | |
| Antibody | GFTFRT | 6 | WYDGS | 11 | APQWEL | 13 | GGNKIG | 274 | DDSD | 26 | QVWDSS | 314 |
| 16 | YGMH | | N | | VHEAFDI | | RKSVH | | RPS | | SDEGV | |
| Antibody | GFTFRT | 6 | WYDGS | 11 | APQWEL | 13 | GGQNIG | 273 | DDSD | 26 | QVWDG | 315 |
| 17 | YGMH | | N | | VHEAFDI | | TKSVH | | RPS | | SSDQSV | |
| Antibody | GFTFRT | 6 | WYDGS | 11 | APQWEL | 13 | GGNNIG | 275 | DDTD | 305 | QVWDT | 316 |
| 18 | YGMH | | N | | VHEAFDI | | GKSVH | | RPS | | TTDHVV | |
| Antibody | GFTFRT | 6 | WYDGS | 11 | APQWEL | 13 | GGNNIG | 276 | DDSD | 26 | QVWDSS | 317 |
| 19 | YGMH | | N | | VHEAFDI | | SKSVH | | RPS | | SDPVV | |
| Antibody | GFTFRT | 6 | WYDGS | 11 | APQWEL | 13 | GGQNIG | 273 | DDSD | 26 | QVWDSS | 313 |
| 20 | YGMH | | N | | VHEAFDI | | TKSVH | | RPS | | SDQSV | |
| Antibody | GFTFRT | 6 | WYDGS | 11 | APQWEL | 13 | GGQNIG | 273 | DDSD | 306 | QVWDSS | 313 |
| 21 | YGMH | | N | | VHEAFDI | | TKSVH | | RPP | | SDQSV | |
| Antibody | GFTFRT | 6 | WYDGS | 11 | APQWEL | 13 | GGNKIG | 274 | DDSD | 26 | QVWDSS | 314 |
| 22 | YGMH | | N | | VHEAFDI | | RKSVH | | RPS | | SDEGV | |
| Antibody | GFTFRT | 6 | WYDGS | 11 | APQWEL | 13 | GGHNIG | 277 | DDSD | 26 | QVWDR | 318 |
| 23 | YGMH | | N | | VHEAFDI | | TKSVH | | RPS | | SSDQSV | |
| Antibody | GFTFRT | 6 | WYDGS | 11 | APQWEL | 13 | GGDSIG | 278 | DDSD | 26 | QVWDR | 318 |
| 24 | YGMH | | N | | VHEAFDI | | SKYVH | | RPS | | SSDQSV | |
| Antibody | GFTFRT | 6 | WYDGS | 11 | APQWEL | 13 | GGQNIG | 273 | DDSD | 26 | QVWDS | 319 |
| 25 | YGMH | | N | | VHEAFDI | | TKSVH | | RPS | | GSGQTV | |
| Antibody | GFTFRT | 6 | WYDGS | 11 | APQWEL | 13 | GGQNIG | 279 | DDSD | 26 | QVWDSS | 313 |
| 26 | YGMH | | N | | VHEAFDI | | TRSVH | | RPS | | SDQSV | |
| Antibody | GFTFRT | 6 | WYDGS | 11 | APQWEL | 13 | GGNNIG | 280 | DDSD | 26 | HVWDSS | 320 |
| 27 | YGMH | | N | | VHEAFDI | | SETVH | | RPS | | SDHVM | |
| Antibody | GFTFRT | 6 | WYDGS | 11 | APQWEL | 13 | RGDNIE | 281 | DDSD | 26 | QVWDS | 321 |
| 28 | YGMH | | N | | VHEAFDI | | TYSVH | | RPS | | DSDHLE | |
| Antibody | GFTFRT | 6 | WYDGS | 11 | APQWEL | 13 | GGNNIG | 282 | DDSD | 26 | QVWDS | 322 |
| 29 | YGMH | | N | | VHEAFDI | | RKSAH | | RPS | | YSNHVV | |
| Antibody | GFTFRT | 6 | WYDGS | 11 | APQWEL | 13 | GGNKIG | 274 | DDSD | 26 | QVWDSS | 314 |
| 30 | YGMH | | N | | VHEAFDI | | RKSVH | | RPS | | SDEGV | |
| Antibody | GFTFRT | 6 | WYDGS | 11 | APQWEL | 13 | GGHNIG | 277 | DDSD | 26 | QVWDR | 318 |
| 31 | YGMH | | N | | VHEAFDI | | TKSVH | | RPS | | SSDQSV | |
| Antibody | GFTFRT | 6 | WYDGS | 11 | APQWEL | 13 | RGDNIE | 281 | DDSD | 26 | QVWDS | 323 |
| 32 | YGMH | | N | | VHEAFDI | | TYSVH | | RPS | | GGDHV | |
| | | | | | | | | | | | V | |
| Antibody | GFTFRT | 6 | WYDGS | 11 | APQWEL | 13 | GGNNIG | 276 | DDSD | 26 | QVWDSS | 29 |
| 33 | YGMH | | N | | VHEAFDI | | SKSVH | | RPS | | SDHVV | |
| Antibody | GFTFRT | 6 | WYDGS | 11 | APQWEL | 13 | EGTYLG | 283 | DDKE | 307 | QVWDDI | 324 |
| 34 | YGMH | | N | | VHEAFDI | | AKSVH | | RPS | | SDHIV | |
| Antibody | GFTFRT | 6 | WYDGS | 11 | APQWEL | 13 | GGNNIG | 284 | DDSD | 26 | QVWDSS | 313 |
| 35 | YGMH | | N | | VHEAFDI | | TKSVH | | RPS | | SDQSV | |
| Antibody | GFTFRT | 6 | WYDGS | 11 | APQWEL | 13 | GGQSIG | 285 | DDSD | 26 | QVWDSS | 313 |
| 36 | YGMH | | N | | VHEAFDI | | TKSVH | | RPS | | SDQSV | |
| Antibody | GFTFRT | 6 | WYDGS | 11 | APQWEL | 13 | GANSIE | 286 | DDSE | 308 | QVWDM | 325 |
| 37 | YGMH | | N | | VHEAFDI | | DKNVH | | RPS | | ASDRVV | |
| Antibody | GFTFRT | 6 | WYDGS | 11 | APQWEL | 13 | GGNKIG | 274 | DDSD | 26 | QVWDSS | 314 |
| 38 | YGMH | | N | | VHEAFDI | | RKSVH | | RPS | | SDEGV | |
| Antibody | GFTFRT | 6 | WYDGS | 11 | APQWEL | 13 | GGQNIG | 273 | DDSD | 26 | QVWDSS | 313 |
| 39 | YGMH | | N | | VHEAFDI | | TKSVH | | RPS | | SDQSV | |
| Antibody | GFTFRT | 6 | WYDGS | 11 | APQWEL | 13 | GGNKIG | 274 | DDSD | 26 | QVWDSS | 314 |
| 40 | YGMH | | N | | VHEAFDI | | RKSVH | | RPS | | SDEGV | |
| Antibody | GFTFRT | 6 | WYDGS | 11 | APQWEL | 13 | GGQNIG | 273 | DDSD | 26 | QVWDSS | 313 |
| 41 | YGMH | | N | | VHEAFDI | | TKSVH | | RPS | | SDQSV | |
| Antibody | GFTFRT | 6 | WYDGS | 11 | APQWEL | 13 | RGDNIE | 281 | DDSD | 26 | QVWDSS | 29 |
| 42 | YGMH | | N | | VHEAFDI | | TYSVH | | RPS | | SDHVV | |
| Antibody | GFTFRT | 6 | WYDGS | 11 | APQWEL | 13 | GGNKIG | 274 | DDSD | 26 | QVWDSS | 314 |
| 43 | YGMH | | N | | VHEAFDI | | RKSVH | | RPS | | SDEGV | |
| Antibody | GFTFRT | 6 | WYDGS | 11 | APQWEL | 13 | GGNKIG | 274 | DDSD | 26 | QVWDTS | 326 |
| 44 | YGMH | | N | | VHEAFDI | | RKSVH | | RPS | | SDQGV | |
| Antibody | GFTFRT | 6 | WYDGS | 11 | APQWEL | 13 | GGDNIG | 287 | DDLK | 309 | QVWDST | 327 |
| 45 | YGMH | | N | | VHEAFDI | | SYSVH | | RPS | | SGVVF | |
| Antibody | GFTFRT | 6 | WYDGS | 11 | APQWEL | 13 | GGNNVE | 288 | DDND | 310 | QVWDSS | 29 |
| 46 | YGMH | | N | | VHEAFDI | | DYSVH | | RPS | | SDHVV | |

IMGT Numbering

| | | | | | | | | | | | | |
|---|---|---|---|---|---|---|---|---|---|---|---|---|
| Antibody | GFTFRT | 7 | IWYDGS | 12 | ARAPQW | 16 | NLGSKS | 22 | DVS | — | QVWDSS | 27 |
| 1 | YT | | NK | | ELVHEAF | | | | | | STFVV | |
| | | | | | DI | | | | | | | |
| Antibody | GFTFRT | 8 | IWYDGS | 12 | ARAPQW | 16 | NLGSKS | 22 | DVS | — | QVWDSS | 28 |
| 2 | YP | | NK | | ELVHEAF | | | | | | STYVV | |
| | | | | | DI | | | | | | | |
| Antibody | GFTFRT | 8 | IWYDGS | 12 | ARALIW | 17 | NLISKS | 23 | DDS | — | QVWDSS | 28 |
| 3 | YP | | NK | | ELFHEYF | | | | | | STYVV | |
| | | | | | DI | | | | | | | |
| Antibody | GFTFRT | 8 | IWYDGS | 12 | ARAPQW | 16 | NLVSKS | 24 | DVS | — | QVWDSS | 28 |
| 4 | YP | | NK | | ELVHEAF | | | | | | STYVV | |
| | | | | | DI | | | | | | | |
| Antibody | GFTFRT | 9 | IWYDGS | 12 | ARAPQW | 16 | NLVSKS | 24 | DDS | — | QVWDSS | 27 |
| 5 | YG | | NK | | ELVHEAF | | | | | | STFVV | |
| | | | | | DI | | | | | | | |
| Antibody | GFTFRT | 9 | IWYDGS | 12 | ARALLW | 18 | NLVSKS | 24 | DVS | — | QVWDSS | 29 |
| 6 | YG | | NK | | ELFHEYF | | | | | | SDHVV | |
| | | | | | DI | | | | | | | |
| Antibody | GFTFRT | 9 | IWYDGS | 12 | ARAPQW | 16 | NLGSKS | 22 | DVS | — | QVWDSS | 30 |
| 7 | YG | | NK | | ELVHEAF | | | | | | STIVV | |
| | | | | | DI | | | | | | | |
| Antibody | GFTFRT | 7 | IWYDGS | 12 | ARAPQW | 16 | NLGSKS | 22 | DDS | — | QVWDK | 31 |
| 8 | YT | | NK | | ELVHEAF | | | | | | SSTYVV | |
| | | | | | DI | | | | | | | |
| Antibody | GFTFRT | 8 | IWYDGS | 12 | ARALLW | 18 | NLVSKS | 24 | DDS | — | QVWDSS | 28 |
| 9 | YP | | NK | | ELFHEYF | | | | | | STYVV | |
| | | | | | DI | | | | | | | |
| Antibody | GFTFRT | 9 | IWYDGS | 12 | ARAPQW | 16 | QNIRYY | 289 | DVS | — | QQGYSP | 311 |
| 10 | YG | | NK | | ELVHEAF | | | | | | PLT | |
| | | | | | DI | | | | | | | |
| Antibody | GFTFRT | 9 | IWYDGS | 12 | ARAPQW | 16 | NIGSKS | 290 | EDS | — | QVWDS | 312 |
| 11 | YG | | NK | | ELVHEAF | | | | | | DSDHVV | |
| | | | | | DI | | | | | | | |
| Antibody | GFTFRT | 9 | IWYDGS | 12 | ARAPQW | 16 | NIGTKS | 291 | DDS | — | QVWDSS | 313 |
| 12 | YG | | NK | | ELVHEAF | | | | | | SDQSV | |
| | | | | | DI | | | | | | | |
| Antibody | GFTFRT | 9 | IWYDGS | 12 | ARAPQW | 16 | NIGTKS | 291 | DDS | — | QVWDSS | 313 |
| 13 | YG | | NK | | ELVHEAF | | | | | | SDQSV | |
| | | | | | DI | | | | | | | |
| Antibody | GFTFRT | 9 | IWYDGS | 12 | ARAPQW | 16 | NIGTKS | 291 | DDS | — | QVWDSS | 313 |
| 14 | YG | | NK | | ELVHEAF | | | | | | SDQSV | |
| | | | | | DI | | | | | | | |
| Antibody | GFTFRT | 9 | IWYDGS | 12 | ARAPQW | 16 | KIGRKS | 292 | DDS | — | QVWDSS | 314 |
| 15 | YG | | NK | | ELVHEAF | | | | | | SDEGV | |
| | | | | | DI | | | | | | | |
| Antibody | GFTFRT | 9 | IWYDGS | 12 | ARAPQW | 16 | KIGRKS | 292 | DDS | — | QVWDSS | 314 |
| 16 | YG | | NK | | ELVHEAF | | | | | | SDEGV | |
| | | | | | DI | | | | | | | |
| Antibody | GFTFRT | 9 | IWYDGS | 12 | ARAPQW | 16 | NIGTKS | 291 | DDS | — | QVWDG | 315 |
| 17 | YG | | NK | | ELVHEAF | | | | | | SSDQSV | |
| | | | | | DI | | | | | | | |
| Antibody | GFTFRT | 9 | IWYDGS | 12 | ARAPQW | 16 | NIGGKS | 293 | DDT | — | QVWDT | 316 |
| 18 | YG | | NK | | ELVHEAF | | | | | | TTDHVV | |
| | | | | | DI | | | | | | | |
| Antibody | GFTFRT | 9 | IWYDGS | 12 | ARAPQW | 16 | NIGSKS | 290 | DDS | — | QVWDSS | 317 |
| 19 | YG | | NK | | ELVHEAF | | | | | | SDPVV | |
| | | | | | DI | | | | | | | |
| Antibody | GFTFRT | 9 | IWYDGS | 12 | ARAPQW | 16 | NIGTKS | 291 | DDS | — | QVWDSS | 313 |
| 20 | YG | | NK | | ELVHEAF | | | | | | SDQSV | |
| | | | | | DI | | | | | | | |
| Antibody | GFTFRT | 9 | IWYDGS | 12 | ARAPQW | 16 | NIGTKS | 291 | DDS | — | QVWDSS | 313 |
| 21 | YG | | NK | | ELVHEAF | | | | | | SDQSV | |
| | | | | | DI | | | | | | | |
| Antibody | GFTFRT | 9 | IWYDGS | 12 | ARAPQW | 16 | KIGRKS | 292 | DDS | — | QVWDSS | 314 |
| 22 | YG | | NK | | ELVHEAF | | | | | | SDEGV | |
| | | | | | DI | | | | | | | |
| Antibody | GFTFRT | 9 | IWYDGS | 12 | ARAPQW | 16 | NIGTKS | 291 | DDS | — | QVWDR | 318 |
| 23 | YG | | NK | | ELVHEAF | | | | | | SSDQSV | |
| | | | | | DI | | | | | | | |
| Antibody | GFTFRT | 9 | IWYDGS | 12 | ARAPQW | 16 | SIGSKY | 294 | DDS | — | QVWDR | 318 |
| 24 | YG | | NK | | ELVHEAF | | | | | | SSDQSV | |
| | | | | | DI | | | | | | | |
| Antibody | GFTFRT | 9 | IWYDGS | 12 | ARAPQW | 16 | NIGTKS | 291 | DDS | — | QVWDS | 319 |
| 25 | YG | | NK | | ELVHEAF | | | | | | GSGQTV | |
| | | | | | DI | | | | | | | |
| Antibody | GFTFRT | 9 | IWYDGS | 12 | ARAPQW | 16 | NIGTRS | 295 | DDS | — | QVWDSS | 313 |
| 26 | YG | | NK | | ELVHEAF | | | | | | SDQSV | |
| | | | | | DI | | | | | | | |
| Antibody | GFTFRT | 9 | IWYDGS | 12 | ARAPQW | 16 | NIGSET | 296 | DDS | — | HVWDSS | 320 |
| 27 | YG | | NK | | ELVHEAF | | | | | | SDHVM | |
| | | | | | DI | | | | | | | |
| Antibody | GFTFRT | 9 | IWYDGS | 12 | ARAPQW | 16 | NIETYS | 297 | DDS | — | QVWDS | 321 |
| 28 | YG | | NK | | ELVHEAF | | | | | | DSDHLE | |
| | | | | | DI | | | | | | | |
| Antibody | GFTFRT | 9 | IWYDGS | 12 | ARAPQW | 16 | NIGRKS | 298 | DDS | — | QVWDS | 322 |
| 29 | YG | | NK | | ELVHEAF | | | | | | YSNHVV | |
| | | | | | DI | | | | | | | |
| Antibody | GFTFRT | 9 | IWYDGS | 12 | ARAPQW | 16 | KIGRKS | 292 | DDS | — | QVWDSS | 314 |
| 30 | YG | | NK | | ELVHEAF | | | | | | SDEGV | |
| | | | | | DI | | | | | | | |
| Antibody | GFTFRT | 9 | IWYDGS | 12 | ARAPQW | 16 | NIGTKS | 291 | DDS | — | QVWDR | 318 |
| 31 | YG | | NK | | ELVHEAF | | | | | | SSDQSV | |
| | | | | | DI | | | | | | | |
| Antibody | GFTFRT | 9 | IWYDGS | 12 | ARAPQW | 16 | NIETYS | 297 | DDS | — | QVWDS | 323 |
| 32 | YG | | NK | | ELVHEAF | | | | | | GGDHV | |
| | | | | | DI | | | | | | V | |
| Antibody | GFTFRT | 9 | IWYDGS | 12 | ARAPQW | 16 | NIGSKS | 290 | DDS | — | QVWDSS | 29 |
| 33 | YG | | NK | | ELVHEAF | | | | | | SDHVV | |
| | | | | | DI | | | | | | | |
| Antibody | GFTFRT | 9 | IWYDGS | 12 | ARAPQW | 16 | YLGAKS | 387 | DDK | — | QVWDDI | 324 |
| 34 | YG | | NK | | ELVHEAF | | | | | | SDHIV | |
| | | | | | DI | | | | | | | |
| Antibody | GFTFRT | 9 | IWYDGS | 12 | ARAPQW | 16 | NIGTKS | 291 | DDS | — | QVWDSS | 313 |
| 35 | YG | | NK | | ELVHEAF | | | | | | SDQSV | |
| | | | | | DI | | | | | | | |
| Antibody | GFTFRT | 9 | IWYDGS | 12 | ARAPQW | 16 | SIGTKS | 299 | DDS | — | QVWDSS | 313 |
| 36 | YG | | NK | | ELVHEAF | | | | | | SDQSV | |
| | | | | | DI | | | | | | | |
| Antibody | GFTFRT | 9 | IWYDGS | 12 | ARAPQW | 16 | SIEDKN | 300 | DDS | — | QVWDM | 325 |
| 37 | YG | | NK | | ELVHEAF | | | | | | ASDRVV | |
| | | | | | DI | | | | | | | |
| Antibody | GFTFRT | 9 | IWYDGS | 12 | ARAPQW | 16 | KIGRKS | 292 | DDS | — | QVWDSS | 314 |
| 38 | YG | | NK | | ELVHEAF | | | | | | SDEGV | |
| | | | | | DI | | | | | | | |
| Antibody | GFTFRT | 9 | IWYDGS | 12 | ARAPQW | 16 | NIGTKS | 291 | DDS | — | QVWDSS | 313 |
| 39 | YG | | NK | | ELVHEAF | | | | | | SDQSV | |
| | | | | | DI | | | | | | | |
| Antibody | GFTFRT | 9 | IWYDGS | 12 | ARAPQW | 16 | KIGRKS | 292 | DDS | — | QVWDSS | 314 |
| 40 | YG | | NK | | ELVHEAF | | | | | | SDEGV | |
| | | | | | DI | | | | | | | |
| Antibody | GFTFRT | 9 | IWYDGS | 12 | ARAPQW | 16 | NIGTKS | 291 | DDS | — | QVWDSS | 313 |
| 41 | YG | | NK | | ELVHEAF | | | | | | SDQSV | |
| | | | | | DI | | | | | | | |
| Antibody | GFTFRT | 9 | IWYDGS | 12 | ARAPQW | 16 | NIETYS | 297 | DDS | — | QVWDSS | 29 |
| 42 | YG | | NK | | ELVHEAF | | | | | | SDHVV | |
| | | | | | DI | | | | | | | |
| Antibody | GFTFRT | 9 | IWYDGS | 12 | ARAPQW | 16 | KIGRKS | 292 | DDS | — | QVWDSS | 314 |
| 43 | YG | | NK | | ELVHEAF | | | | | | SDEGV | |
| | | | | | DI | | | | | | | |
| Antibody | GFTFRT | 9 | IWYDGS | 12 | ARAPQW | 16 | KIGRKS | 292 | DDS | — | QVWDTS | 326 |
| 44 | YG | | NK | | ELVHEAF | | | | | | SDQGV | |
| | | | | | DI | | | | | | | |
| Antibody | GFTFRT | 9 | IWYDGS | 12 | ARAPQW | 16 | NIGSYS | 301 | DDL | — | QVWDST | 327 |
| 45 | YG | | NK | | ELVHEAF | | | | | | SGVVF | |
| | | | | | DI | | | | | | | |
| Antibody | GFTFRT | 9 | IWYDGS | 12 | ARAPQW | 16 | NVEDYS | 302 | DDN | — | QVWDSS | 29 |
| 46 | YG | | NK | | ELVHEAF | | | | | | SDHVV | |
| | | | | | DI | | | | | | | |

TABLE 4
Sequences of anti-TSLP antibody constructs-VH and VL

| Antibody | SEQ ID NO | VH | SEQ ID NO | VL |
|---|---|---|---|---|
| Antibody | 32 | QMQLVESGGGVVQPGRSLRLSCA | 37 | SYVLTQPPSVSVAPGQTAR |
| 1 | | ASGFTFRTYTMHWVRQAPGKGL | | ITCGGNNLGSKSVHWYQQ |
| | | EWVAVIWYDGSNKHYADSVKGR | | KPGQAPVLVVYDVSDRPS |
| | | FTITRDNSKNTLNLQMNSLRAED | | WIPERFSGSNSGNTATLTIS |
| | | TAVYYCARAPQWELVHEAFDIW | | RGEAGDEADYYCQVWDS |
| | | GQGTMVTVSS | | SSTFVVFGGGTKLTVL |
| Antibody | 33 | QMQLVESGGGVVQPGRSLRLSCA | 38 | SYVLTQPPSVSVAPGQTAR |
| 2 | | ASGFTFRTYPMHWVRQAPGKGL | | ITCGGNNLGSKSVHWYQQ |
| | | EWVAVIWYDGSNKHYADSVKGR | | KPGQAPVLVVYDVSDRPS |
| | | FTITRDNSKNTLNLQMNSLRAED | | WIPERFSGSNSGNTATLTIS |
| | | TAVYYCARAPQWELVHEAFDIW | | RGEAGDEADYYCQVWDS |
| | | GQGTMVTVSS | | SSTYVVFGGGTKLTVL |
| Antibody | 386 | QMQLVESGGGVVQPGRSLRLSCA | 39 | SYVLTQPPSVSVAPGQTAR |
| 3 | | ASGFTFRTYPMHWVRQAPGKGL | | ITCGGNNLISKSVHWYQQ |
| | | EWVAVIWYDGSNKHYADSVKGR | | KPGQAPVLVVYDDSDRPS |
| | | FTITRDNSKNTLNLQMNSLRAED | | WIPERFSGSNSGNTATLTIS |
| | | TAVYYCARALIWELFHEYFDIWG | | RGEAGDEADYYCQVWDS |
| | | QGTMVTVSS | | SSTYVVFGGGTKLTVL |
| Antibody | 33 | QMQLVESGGGVVQPGRSLRLSCA | 40 | SYVLTQPPSVSVAPGQTAR |
| 4 | | ASGFTFRTYPMHWVRQAPGKGL | | ITCGGNNLVSKSVHWYQQ |
| | | EWVAVIWYDGSNKHYADSVKGR | | KPGQAPVLVVYDVSDRPS |
| | | FTITRDNSKNTLNLQMNSLRAED | | WIPERFSGSNSGNTATLTIS |
| | | TAVYYCARAPQWELVHEAFDIW | | RGEAGDEADYYCQVWDS |
| | | GQGTMVTVSS | | SSTYVVFGGGTKLTVL |
| Antibody | 34 | QMQLVESGGGVVQPGRSLRLSCA | 41 | SYVLTQPPSVSVAPGQTAR |
| 5 | | ASGFTFRTYGMHWVRQAPGKGL | | ITCGGNNLVSKSVHWYQQ |
| | | EWVAVIWYDGSNKHYADSVKGR | | KPGQAPVLVVYDDSDRPS |
| | | FTITRDNSKNTLNLQMNSLRAED | | WIPERFSGSNSGNTATLTIS |
| | | TAVYYCARAPQWELVHEAFDIW | | RGEAGDEADYYCQVWDS |
| | | GQGTMVTVSS | | SSTFVVFGGGTKLTVL |
| Antibody | 35 | QMQLVESGGGVVQPGRSLRLSCA | 42 | SYVLTQPPSVSVAPGQTAR |
| 6 | | ASGFTFRTYGMHWVRQAPGKGL | | ITCGGNNLVSKSVHWYQQ |
| | | EWVAVIWYDGSNKHYADSVKGR | | KPGQAPVLVVYDVSDRPS |
| | | FTITRDNSKNTLNLQMNSLRAED | | WIPERFSGSNSGNTATLTIS |
| | | TAVYYCARALLWELFHEYFDIWG | | RGEAGDEADYYCQVWDS |
| | | QGTMVTVSS | | SSDHVVFGGGTKLTVL |
| Antibody | 34 | QMQLVESGGGVVQPGRSLRLSCA | 43 | SYVLTQPPSVSVAPGQTAR |
| 7 | | ASGFTFRTYGMHWVRQAPGKGL | | ITCGGNNLGSKSVHWYQQ |
| | | EWVAVIWYDGSNKHYADSVKGR | | KPGQAPVLVVYDVSDRPS |
| | | FTITRDNSKNTLNLQMNSLRAED | | WIPERFSGSNSGNTATLTIS |
| | | TAVYYCARAPQWELVHEAFDIW | | RGEAGDEADYYCQVWDS |
| | | GQGTMVTVSS | | SSTIVVFGGGTKLTVL |
| Antibody | 32 | QMQLVESGGGVVQPGRSLRLSCA | 44 | SYVLTQPPSVSVAPGQTAR |
| 8 | | ASGFTFRTYTMHWVRQAPGKGL | | ITCGGNNLGSKSVHWYQQ |
| | | EWVAVIWYDGSNKHYADSVKGR | | KPGQAPVLVVYDDSDRPS |
| | | FTITRDNSKNTLNLQMNSLRAED | | WIPERFSGSNSGNTATLTIS |
| | | TAVYYCARAPQWELVHEAFDIW | | RGEAGDEADYYCQVWDK |
| | | GQGTMVTVSS | | SSTYVVFGGGTKLTVL |
| Antibody | 36 | QMQLVESGGGVVQPGRSLRLSCA | 45 | SYVLTQPPSVSVAPGQTAR |
| 9 | | ASGFTFRTYPMHWVRQAPGKGL | | ITCGGNNLVSKSVHWYQQ |
| | | EWVAVIWYDGSNKHYADSVKGR | | KPGQAPVLVVYDDSDRPS |
| | | FTITRDNSKNTLNLQMNSLRAED | | WIPERFSGSNSGNTATLTIS |
| | | TAVYYCARALLWELFHEYFDIWG | | RGEAGDEADYYCQVWDS |
| | | QGTMVTVSS | | SSTYVVFGGGTKLTVL |
| Antibody | 34 | QMQLVESGGGVVQPGRSLRLSCA | 328 | DIQLTQSPSSLSASVGDRV |
| 10 | | ASGFTFRTYGMHWVRQAPGKGL | | AITCRTSQNIRYYLNWYQR |
| | | EWVAVIWYDGSNKHYADSVKGR | | KPGKAPRLLIYDVSSLQSG |
| | | FTITRDNSKNTLNLQMNSLRAED | | VPSRFSGRGSGTDFTLTISS |
| | | TAVYYCARAPQWELVHEAFDIW | | LQAEDFATYYCQQGYSPP |
| | | GQGTMVTVSS | | LTFGGGTKVEIK |
| Antibody | 34 | QMQLVESGGGVVQPGRSLRLSCA | 329 | NFMLTQPPSVSVAPGKTA |
| 11 | | ASGFTFRTYGMHWVRQAPGKGL | | KMTCGGDNIGSKSVHWYQ |
| | | EWVAVIWYDGSNKHYADSVKGR | | QMPGQAPVLVIYEDSDRPS |
| | | FTITRDNSKNTLNLQMNSLRAED | | GTPERFSGSNSGHTATLTIS |
| | | TAVYYCARAPQWELVHEAFDIW | | RVEAGDEADYYCQVWDS |
| | | GQGTMVTVSS | | DSDHVVFGGGTTLTVL |
| Antibody | 34 | QMQLVESGGGVVQPGRSLRLSCA | 330 | QSVLTQPPSVSVAPGQTAR |
| 12 | | ASGFTFRTYGMHWVRQAPGKGL | | ITCGGQNIGTKSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDSS |
| | | GQGTMVTVSS | | SDQSVFGPGTRVTVL |
| Antibody | 34 | QMQLVESGGGVVQPGRSLRLSCA | 331 | SYELTQPPSVSVAPGQTAR |
| 13 | | ASGFTFRTYGMHWVRQAPGKGL | | ITCGGQNIGTKSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDSS |
| | | GQGTMVTVSS | | SDQSVFGPGTRVTVL |
| Antibody | 34 | QMQLVESGGGVVQPGRSLRLSCA | 332 | SYVLTQPPSVSVAPGQTAR |
| 14 | | ASGFTFRTYGMHWVRQAPGKGL | | ITCGGQNIGTKSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDSS |
| | | GQGTMVTVSS | | SDQSVFGPGTRVTVL |
| Antibody | 34 | QMQLVESGGGVVQPGRSLRLSCA | 333 | SYVLTQPPSVSVAPGQTAR |
| 15 | | ASGFTFRTYGMHWVRQAPGKGL | | ITCGGNKIGRKSVHWYQQ |
| | | EWVAVIWYDGSNKHYADSVKGR | | RPGQAPVLVVFDDSDRPS |
| | | FTITRDNSKNTLNLQMNSLRAED | | WISERFSGSNSGNTATLTIS |
| | | TAVYYCARAPQWELVHEAFDIW | | RVEAGDEADYYCQVWDS |
| | | GQGTMVTVSS | | SSDEGVFGGGTKLTVL |
| Antibody | 34 | QMQLVESGGGVVQPGRSLRLSCA | 334 | SYELTQPPSVSVAPGQAAR |
| 16 | | ASGFTFRTYGMHWVRQAPGKGL | | ITCGGNKIGRKSVHWYQQ |
| | | EWVAVIWYDGSNKHYADSVKGR | | RPGQAPVLVVFDDSDRPS |
| | | FTITRDNSKNTLNLQMNSLRAED | | WISERFSGSNSGNTATLTIS |
| | | TAVYYCARAPQWELVHEAFDIW | | RVEAGDEADYYCQVWDS |
| | | GQGTMVTVSS | | SSDEGVFGGGTKLTVL |
| Antibody | 34 | QMQLVESGGGVVQPGRSLRLSCA | 335 | QSVLTQPPSVSVAPGQTAR |
| 17 | | ASGFTFRTYGMHWVRQAPGKGL | | ITCGGQNIGTKSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDG |
| | | GQGTMVTVSS | | SSDQSVFGPGTRVTVL |
| Antibody | 34 | QMQLVESGGGVVQPGRSLRLSCA | 336 | SYELTQPPSVSVAPGQTASI |
| 18 | | ASGFTFRTYGMHWVRQAPGKGL | | TCGGNNIGGKSVHWYQLK |
| | | EWVAVIWYDGSNKHYADSVKGR | | PGQAPVLVVYDDTDRPSW |
| | | FTITRDNSKNTLNLQMNSLRAED | | IPERFSGSNSANTATLTISG |
| | | TAVYYCARAPQWELVHEAFDIW | | VEAGDEADYQCQVWDTT |
| | | GQGTMVTVSS | | TDHVVFGGGTKLTVL |
| Antibody | 34 | QMQLVESGGGVVQPGRSLRLSCA | 337 | SYVLTQPPSVSVAPGQTAR |
| 19 | | ASGFTFRTYGMHWVRQAPGKGL | | ITCGGNNIGSKSVHWYQQ |
| | | EWVAVIWYDGSNKHYADSVKGR | | KPGQAPLLVVYDDSDRPS |
| | | FTITRDNSKNTLNLQMNSLRAED | | WIPERFSGSNSGNAATLTV |
| | | TAVYYCARAPQWELVHEAFDIW | | SRVEAGDEADYYCQVWD |
| | | GQGTMVTVSS | | SSSDPVVFGGGTKLTVL |
| Antibody | 34 | QMQLVESGGGVVQPGRSLRLSCA | 338 | FYVLTQPPSVSVAPGQTAR |
| 20 | | ASGFTFRTYGMHWVRQAPGKGL | | ITCGGQNIGTKSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDSS |
| | | GQGTMVTVSS | | SDQSVFGPGTRVTVL |
| Antibody | 34 | QMQLVESGGGVVQPGRSLRLSCA | 339 | QSVLTQPPSVSVAPGQTAR |
| 21 | | ASGFTFRTYGMHWVRQAPGKGL | | ITCGGQNIGTKSVHWYQQ |
| | | EWVAVIWYDGSNKHYADSVKGR | | KSGQAPILVVYDDSDRPP |
| | | FTITRDNSKNTLNLQMNSLRAED | | WIPERFSGSNFGTTATLTIS |
| | | TAVYYCARAPQWELVHEAFDIW | | RVEAGDEADYFCQVWDSS |
| | | GQGTMVTVSS | | SDQSVFGPGTRVTVL |
| Antibody | 34 | QMQLVESGGGVVQPGRSLRLSCA | 340 | QAVLTQPPSVSVAPGQTAR |
| 22 | | ASGFTFRTYGMHWVRQAPGKGL | | ITCGGNKIGRKSVHWYQQ |
| | | EWVAVIWYDGSNKHYADSVKGR | | RPGQAPVLVVFDDSDRPS |
| | | FTITRDNSKNTLNLQMNSLRAED | | WISERFSGSNSGNTATLTIS |
| | | TAVYYCARAPQWELVHEAFDIW | | RVEAGDEADYYCQVWDS |
| | | GQGTMVTVSS | | SSDEGVFGGGTKLTVL |
| Antibody | 34 | QMQLVESGGGVVQPGRSLRLSCA | 341 | SYELTQPPSVSVAPGQTAR |
| 23 | | ASGFTFRTYGMHWVRQAPGKGL | | ITCGGHNIGTKSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDRS |
| | | GQGTMVTVSS | | SDQSVFGPGTRVTVL |
| Antibody | 34 | QMQLVESGGGVVQPGRSLRLSCA | 342 | SYVLTQPPSVSVAPGQTAR |
| 24 | | ASGFTFRTYGMHWVRQAPGKGL | | VTCGGDSIGSKYVHWYQQ |
| | | EWVAVIWYDGSNKHYADSVKGR | | KPGQAPVLVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDRS |
| | | GQGTMVTVSS | | SDQSVFGPGTRVTVL |
| Antibody | 34 | QMQLVESGGGVVQPGRSLRLSCA | 343 | SYELTQPPSVSVAPGQTAR |
| 25 | | ASGFTFRTYGMHWVRQAPGKGL | | ITCGGQNIGTKSVHWYQQ |
| | | EWVAVIWYDGSNKHYADSVKGR | | KSGQAPILVVFDDSDRPSW |
| | | FTITRDNSKNTLNLQMNSLRAED | | VTERFSGSNFGTTATLTIN |
| | | TAVYYCARAPQWELVHEAFDIW | | GVEAGDEADYFCQVWDS |
| | | GQGTMVTVSS | | GSGQTVFGPGTRVTVS |
| Antibody | 34 | QMQLVESGGGVVQPGRSLRLSCA | 344 | SYELTQPPSVSVAPGQTAR |
| 26 | | ASGFTFRTYGMHWVRQAPGKGL | | ITCGGQNIGTRSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDSS |
| | | GQGTMVTVSS | | SDQSVFGPGTRVTVL |
| Antibody | 34 | QMQLVESGGGVVQPGRSLRLSCA | 345 | SYELTQPPSVSVAPGQTAR |
| 27 | | ASGFTFRTYGMHWVRQAPGKGL | | ITCGGNNIGSETVHWYRQ |
| | | EWVAVIWYDGSNKHYADSVKGR | | KAGQAPVLVVYDDSDRPS |
| | | FTITRDNSKNTLNLQMNSLRAED | | GIPERFSGSNSGNTAALTIS |
| | | TAVYYCARAPQWELVHEAFDIW | | RVEVGDEADYYCHVWDS |
| | | GQGTMVTVSS | | SSDHVMFGGGTKLTVL |
| Antibody | 34 | QMQLVESGGGVVQPGRSLRLSCA | 346 | SYELTQPPSVSVGPGKTATI |
| 28 | | ASGFTFRTYGMHWVRQAPGKGL | | TCRGDNIETYSVHWYQQK |
| | | EWVAVIWYDGSNKHYADSVKGR | | PGQAPVLVVYDDSDRPSGI |
| | | FTITRDNSKNTLNLQMNSLRAED | | PERFSGSNSGNTATLTINR |
| | | TAVYYCARAPQWELVHEAFDIW | | VGAGDEADYYCQVWDSD |
| | | GQGTMVTVSS | | SDHLEFGGGTKLTVL |
| Antibody | 34 | QMQLVESGGGVVQPGRSLRLSCA | 347 | SYELTQPPSVSVAPGQTAR |
| 29 | | ASGFTFRTYGMHWVRQAPGKGL | | ITCGGNNIGRKSAHWYQQ |
| | | EWVAVIWYDGSNKHYADSVKGR | | RPGQAPVLVVYDDSDRPS |
| | | FTITRDNSKNTLNLQMNSLRAED | | WIPERFSGSNSGNTATLTIS |
| | | TAVYYCARAPQWELVHEAFDIW | | RVEAGDEADYYCQVWDS |
| | | GQGTMVTVSS | | YSNHVVFGGGTKLTVL |
| Antibody | 34 | QMQLVESGGGVVQPGRSLRLSCA | 348 | LPVLTQPPSVSVAPGQTAR |
| 30 | | ASGFTFRTYGMHWVRQAPGKGL | | ITCGGNKIGRKSVHWYQQ |
| | | EWVAVIWYDGSNKHYADSVKGR | | RPGQAPVLVVFDDSDRPS |
| | | FTITRDNSKNTLNLQMNSLRAED | | WISERFSGSNSGNTATLTIS |
| | | TAVYYCARAPQWELVHEAFDIW | | RVEAGDEADYYCQVWDS |
| | | GQGTMVTVSS | | SSDEGVFGGGTKLTVL |
| Antibody | 34 | QMQLVESGGGVVQPGRSLRLSCA | 349 | SYVLTQPPSVSVAPGQTAR |
| 31 | | ASGFTFRTYGMHWVRQAPGKGL | | ITCGGHNIGTKSVHWYQQ |
| | | EWVAVIWYDGSNKHYADSVKGR | | KSGQAPILAVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDRS |
| | | GQGTMVTVSS | | SDQSVFGPGTRVTVL |
| Antibody | 34 | QMQLVESGGGVVQPGRSLRLSCA | 350 | SYELTQPPSVSVGPGKTATI |
| 32 | | ASGFTFRTYGMHWVRQAPGKGL | | TCRGDNIETYSVHWYQQK |
| | | EWVAVIWYDGSNKHYADSVKGR | | PGQAPVLVVYDDSDRPSGI |
| | | FTITRDNSKNTLNLQMNSLRAED | | PERFSGSNSGNTATLTINR |
| | | TAVYYCARAPQWELVHEAFDIW | | VEARDEADYFCQVWDSG |
| | | GQGTMVTVSS | | GDHVVFGGGTKLTVL |
| Antibody | 34 | QMQLVESGGGVVQPGRSLRLSCA | 351 | SYVLTQPPSVSVAPGQTAR |
| 33 | | ASGFTFRTYGMHWVRQAPGKGL | | ITCGGNNIGSKSVHWYQQ |
| | | EWVAVIWYDGSNKHYADSVKGR | | KPGQAPVLVVYDDSDRPS |
| | | FTITRDNSKNTLNLQMNSLRAED | | WIPERFSGTNSGNTATLTIS |
| | | TAVYYCARAPQWELVHEAFDIW | | RVEAGDEADYYCQVWDS |
| | | GQGTMVTVSS | | SSDHVVFGGGTKLTVL |
| Antibody | 34 | QMQLVESGGGVVQPGRSLRLSCA | 352 | SYELTQPPSVSVAPGKRAR |
| 34 | | ASGFTFRTYGMHWVRQAPGKGL | | ITCEGTYLGAKSVHWYQQ |
| | | EWVAVIWYDGSNKHYADSVKGR | | KPGQAPVLVIYDDKERPSG |
| | | FTITRDNSKNTLNLQMNSLRAED | | IPERFSASNSGNTATLTITR |
| | | TAVYYCARAPQWELVHEAFDIW | | VEAGDEADYHCQVWDDIS |
| | | GQGTMVTVSS | | DHIVFGGGTKLSVL |
| Antibody | 34 | QMQLVESGGGVVQPGRSLRLSCA | 353 | SYVLTQPPSVSVAPGQTAR |
| 35 | | ASGFTFRTYGMHWVRQAPGKGL | | ITCGGNNIGTKSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDSS |
| | | GQGTMVTVSS | | SDQSVFGPGTRVTVL |
| Antibody | 34 | QMQLVESGGGVVQPGRSLRLSCA | 354 | SYVLTQPPSVSVAPGQTAR |
| 36 | | ASGFTFRTYGMHWVRQAPGKGL | | ITCGGQSIGTKSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDSS |
| | | GQGTMVTVSS | | SDQSVFGPGTRVTVL |
| Antibody | 34 | QMQLVESGGGVVQPGRSLRLSCA | 355 | SYELTQPPSVSVAPGQAAS |
| 37 | | ASGFTFRTYGMHWVRQAPGKGL | | LTCGANSIEDKNVHWYQQ |
| | | EWVAVIWYDGSNKHYADSVKGR | | RPGQAPLLVIYDDSERPSGI |
| | | FTITRDNSKNTLNLQMNSLRAED | | PERFSGSNSGNTATLTITRV |
| | | TAVYYCARAPQWELVHEAFDIW | | AAGDEADYYCQVWDMAS |
| | | GQGTMVTVSS | | DRVVFGPGTTVSVL |
| Antibody | 34 | QMQLVESGGGVVQPGRSLRLSCA | 356 | SYELTQPPSVSVAPGQTAR |
| 38 | | ASGFTFRTYGMHWVRQAPGKGL | | ITCGGNKIGRKSVHWYQQ |
| | | EWVAVIWYDGSNKHYADSVKGR | | RPGQAPVLVVFDDSDRPS |
| | | FTITRDNSKNTLNLQMNSLRAED | | WISERFSGSNSGNTATLTIG |
| | | TAVYYCARAPQWELVHEAFDIW | | RVEAGDEADYYCQVWDS |
| | | GQGTMVTVSS | | SSDEGVFGGGTKLAVL |
| Antibody | 34 | QMQLVESGGGVVQPGRSLRLSCA | 357 | SYELTQPPSVSVAPGRTARI |
| 39 | | ASGFTFRTYGMHWVRQAPGKGL | | TCGGQNIGTKSVHWYQQK |
| | | EWVAVIWYDGSNKHYADSVKGR | | SGQAPILVVYDDSDRPSWI |
| | | FTITRDNSKNTLNLQMNSLRAED | | PERFSGSNFGTTATLTISRV |
| | | TAVYYCARAPQWELVHEAFDIW | | EAGDEADYFCQVWDSSSD |
| | | GQGTMVTVSS | | QSVFGPGTRVTVL |
| Antibody | 34 | QMQLVESGGGVVQPGRSLRLSCA | 358 | QAGLTQPPSVSVAPGQTAR |
| 40 | | ASGFTFRTYGMHWVRQAPGKGL | | ITCGGNKIGRKSVHWYQQ |
| | | EWVAVIWYDGSNKHYADSVKGR | | RPGQAPVLVVFDDSDRPS |
| | | FTITRDNSKNTLNLQMNSLRAED | | WISERFSGSNSGNTATLTIS |
| | | TAVYYCARAPQWELVHEAFDIW | | RVEAGDEADYYCQVWDS |
| | | GQGTMVTVSS | | SSDEGVFGGGTKLTVL |
| Antibody | 34 | QMQLVESGGGVVQPGRSLRLSCA | 359 | QSVLTQPPSVSVAPGQTAR |
| 41 | | ASGFTFRTYGMHWVRQAPGKGL | | ITCGGQNIGTKSVHWYQQ |
| | | EWVAVIWYDGSNKHYADSVKGR | | KSGQAPILVVYDDSDRPS |
| | | FTITRDNSKNTLNLQMNSLRAED | | WIPERFSGSNFGTTATLTIS |
| | | TAVYYCARAPQWELVHEAFDIW | | RVEAGDEADYFCQVWDSS |
| | | GQGTMVTVSS | | SDQSVFGTGTKVTVL |
| Antibody | 34 | QMQLVESGGGVVQPGRSLRLSCA | 360 | SYVLTQPPSVSVGPGKTAT |
| 42 | | ASGFTFRTYGMHWVRQAPGKGL | | ITCRGDNIETYSVHWYQRK |
| | | EWVAVIWYDGSNKHYADSVKGR | | PGQAPVLVVYDDSDRPSGI |
| | | FTITRDNSKNTLNLQMNSLRAED | | PERFSGSNSGNTATLTISRV |
| | | TAVYYCARAPQWELVHEAFDIW | | EAGDEADYYCQVWDSSSD |
| | | GQGTMVTVSS | | HVVFGGGTKLTVL |
| Antibody | 34 | QMQLVESGGGVVQPGRSLRLSCA | 361 | SYELTQPPSVSVAPGQTAR |
| 43 | | ASGFTFRTYGMHWVRQAPGKGL | | ITCGGNKIGRKSVHWYQQ |
| | | EWVAVIWYDGSNKHYADSVKGR | | RPGQAPVLVVFDDSDRPS |
| | | FTITRDNSKNTLNLQMNSLRAED | | WISERFSGSNSGNTATLTIS |
| | | TAVYYCARAPQWELVHEAFDIW | | RVDAGDEADYYCQVWDS |
| | | GQGTMVTVSS | | SSDEGVFGGGTKLTVL |
| Antibody | 34 | QMQLVESGGGVVQPGRSLRLSCA | 362 | SYELTQPPSVSVAPGQTAR |
| 44 | | ASGFTFRTYGMHWVRQAPGKGL | | ITCGGNKIGRKSVHWYQQ |
| | | EWVAVIWYDGSNKHYADSVKGR | | KPGQAPVLVVFDDSDRPS |
| | | FTITRDNSKNTLNLQMNSLRAED | | WISERFSGSNSGNTATLTIS |
| | | TAVYYCARAPQWELVHEAFDIW | | RVEAGDEADYHCQVWDT |
| | | GQGTMVTVSS | | SSDQGVFGGGTKLTVL |
| Antibody | 34 | QMQLVESGGGVVQPGRSLRLSCA | 363 | SYVLTQPPSVAVAPGQTA |
| 45 | | ASGFTFRTYGMHWVRQAPGKGL | | KITCGGDNIGSYSVHWYQ |
| | | EWVAVIWYDGSNKHYADSVKGR | | QKPGQAPVLVVYDDLKRP |
| | | FTITRDNSKNTLNLQMNSLRAED | | SWIPERFFGSNSGNTATLTI |
| | | TAVYYCARAPQWELVHEAFDIW | | SRVEAGDEAVYYCQVWD |
| | | GQGTMVTVSS | | STSGVVFGGGTTVTVL |
| Antibody | 34 | QMQLVESGGGVVQPGRSLRLSCA | 364 | SYELTQPPSVSVAPGKTAR |
| 46 | | ASGFTFRTYGMHWVRQAPGKGL | | ITCGGNNVEDYSVHWYHQ |
| | | EWVAVIWYDGSNKHYADSVKGR | | KAGQAPVVVMYDDNDRP |
| | | FTITRDNSKNTLNLQMNSLRAED | | SGVPERFSGSNSGNTATLTI |
| | | TAVYYCARAPQWELVHEAFDIW | | GRVEAGDEADYYCQVWD |
| | | GQGTMVTVSS | | SSSDHVVFGGGTKLTVL |

TABLE 5
Sequences of additional anti-TSLP antibody constructs-CDRs

| | | | | | | | | | | | | |
|---|---|---|---|---|---|---|---|---|---|---|---|---|
| | | SEQ | | SEQ | | SEQ | | SEQ | | SEQ | | SEQ |
| | | ID | | ID | | ID | | ID | | ID | | ID |
| Antibody | CDRH1 | NO: | CDRH2 | NO: | CDRH3 | NO: | CDRL1 | NO: | CDRL2 | NO: | CDRL3 | NO: |

Kabat Numbering

| | | | | | | | | | | | | |
|---|---|---|---|---|---|---|---|---|---|---|---|---|
| Antibody | TYGMH | 3 | VIWYDG | 10 | APQWEL | 13 | GGNNLG | 19 | DVSD | 25 | QVWD | 29 |
| 47 | | | SNKHYA | | VHEAFDI | | SKSVH | | RPS | | SSSDH | |
| | | | DSVKG | | | | | | | | VV | |
| Antibody | TYGMH | 3 | VIWYDG | 10 | APQWEL | 13 | GGNNLG | 19 | DDSD | 26 | QVWD | 367 |
| 48 | | | SNKHYA | | VHEAFDI | | SKSVH | | RPS | | SSSDF | |
| | | | DSVKG | | | | | | | | VV | |
| Antibody | TYGMH | 3 | VIWYDG | 10 | APQWEL | 13 | GGNNLV | 21 | DVSD | 25 | QVWD | 29 |
| 49 | | | SNKHYA | | VHEAFDI | | SKSVH | | RPS | | SSSDH | |
| | | | DSVKG | | | | | | | | VV | |
| Antibody | TYGMH | 3 | VIWYDG | 10 | APQWEL | 13 | GGNNLV | 21 | DDSD | 26 | QVWD | 367 |
| 50 | | | SNKHYA | | VHEAFDI | | SKSVH | | RPS | | SSSDF | |
| | | | DSVKG | | | | | | | | VV | |
| Antibody | TYGMH | 3 | VIWYDG | 10 | APQWEL | 13 | GGNNLG | 19 | DVSD | 25 | QVWD | 368 |
| 51 | | | SNKHYA | | VHEAFDI | | SKSVH | | RPS | | SSSTH | |
| | | | DSVKG | | | | | | | | VV | |
| Antibody | TYGMH | 3 | VIWYDG | 10 | APQWEL | 13 | GGNNLG | 19 | DVSD | 25 | QVWD | 367 |
| 52 | | | SNKHYA | | VHEAFDI | | SKSVH | | RPS | | SSSDF | |
| | | | DSVKG | | | | | | | | VV | |
| Antibody | TYGMH | 3 | VIWYDG | 10 | APQWEL | 13 | GGNNLG | 19 | DDSD | 26 | QVWD | 369 |
| 53 | | | SNKHYA | | VHEAFDI | | SKSVH | | RPS | | SSSTFV | |
| | | | DSVKG | | | | | | | | V | |
| Antibody | TYGMH | 3 | VIWYDG | 10 | APQWEL | 13 | GGNNLV | 21 | DVSD | 25 | QVWD | 368 |
| 54 | | | SNKHYA | | VHEAFDI | | SKSVH | | RPS | | SSSTH | |
| | | | DSVKG | | | | | | | | VV | |
| Antibody | TYGMH | 3 | VIWYDG | 10 | APQWEL | 13 | GGNNLV | 21 | DVSD | 25 | QVWD | 367 |
| 55 | | | SNKHYA | | VHEAFDI | | SKSVH | | RPS | | SSSDF | |
| | | | DSVKG | | | | | | | | VV | |
| Antibody | TYGMH | 3 | VIWYDG | 10 | APQWEL | 13 | GGNNLV | 21 | DDSD | 26 | QVWD | 369 |
| 56 | | | SNKHYA | | VHEAFDI | | SKSVH | | RPS | | SSSTFV | |
| | | | DSVKG | | | | | | | | V | |
| Antibody | TYGMH | 3 | VIWYDG | 10 | APQWEL | 13 | GGNNLV | 21 | DDSD | 26 | QVWD | 370 |
| 57 | | | SNKHYA | | VHEAFDI | | SKSVH | | RPS | | SSSTIV | |
| | | | DSVKG | | | | | | | | V | |
| Antibody | TYGMH | 3 | VIWYDG | 10 | APQWEL | 13 | GGNNLG | 19 | DVSD | 25 | QVWD | 369 |
| 58 | | | SNKHYA | | VHEAFDI | | SKSVH | | RPS | | SSSTFV | |
| | | | DSVKG | | | | | | | | V | |
| Antibody | TYGMH | 3 | VIWYDG | 10 | APQWEL | 13 | GGNNLV | 21 | DVSD | 25 | QVWD | 369 |
| 59 | | | SNKHYA | | VHEAFDI | | SKSVH | | RPS | | SSSTFV | |
| | | | DSVKG | | | | | | | | V | |
| Antibody | TYGMH | 3 | VIWYDG | 10 | APQWEL | 13 | GGNNLV | 21 | DVSD | 25 | QVWD | 370 |
| 60 | | | SNKHYA | | VHEAFDI | | SKSVH | | RPS | | SSSTIV | |
| | | | DSVKG | | | | | | | | V | |
| Antibody | TYGMH | 3 | VIWYDG | 10 | ALLWEL | 15 | GGNNLG | 19 | DDSD | 26 | QVWD | 29 |
| 61 | | | SNKHYA | | FHEYFDI | | SKSVH | | RPS | | SSSDH | |
| | | | DSVKG | | | | | | | | VV | |

Chothia Numbering

| | | | | | | | | | | | | |
|---|---|---|---|---|---|---|---|---|---|---|---|---|
| Antibody | GFTFRT | 6 | WYDGS | 11 | APQWEL | 13 | GGNNLG | 19 | DVSD | 25 | QVWD | 29 |
| 47 | YGMH | | N | | VHEAFDI | | SKSVH | | RPS | | SSSDH | |
| | | | | | | | | | | | VV | |
| Antibody | GFTFRT | 6 | WYDGS | 11 | APQWEL | 13 | GGNNLG | 19 | DDSD | 26 | QVWD | 367 |
| 48 | YGMH | | N | | VHEAFDI | | SKSVH | | RPS | | SSSDF | |
| | | | | | | | | | | | VV | |
| Antibody | GFTFRT | 6 | WYDGS | 11 | APQWEL | 13 | GGNNLV | 21 | DVSD | 25 | QVWD | 29 |
| 49 | YGMH | | N | | VHEAFDI | | SKSVH | | RPS | | SSSDH | |
| | | | | | | | | | | | VV | |
| Antibody | GFTFRT | 6 | WYDGS | 11 | APQWEL | 13 | GGNNLV | 21 | DDSD | 26 | QVWD | 367 |
| 50 | YGMH | | N | | VHEAFDI | | SKSVH | | RPS | | SSSDF | |
| | | | | | | | | | | | VV | |
| Antibody | GFTFRT | 6 | WYDGS | 11 | APQWEL | 13 | GGNNLG | 19 | DVSD | 25 | QVWD | 368 |
| 51 | YGMH | | N | | VHEAFDI | | SKSVH | | RPS | | SSSTH | |
| | | | | | | | | | | | VV | |
| Antibody | GFTFRT | 6 | WYDGS | 11 | APQWEL | 13 | GGNNLG | 19 | DVSD | 25 | QVWD | 367 |
| 52 | YGMH | | N | | VHEAFDI | | SKSVH | | RPS | | SSSDF | |
| | | | | | | | | | | | VV | |
| Antibody | GFTFRT | 6 | WYDGS | 11 | APQWEL | 13 | GGNNLG | 19 | DDSD | 26 | QVWD | 369 |
| 53 | YGMH | | N | | VHEAFDI | | SKSVH | | RPS | | SSSTFV | |
| | | | | | | | | | | | V | |
| Antibody | GFTFRT | 6 | WYDGS | 11 | APQWEL | 13 | GGNNLV | 21 | DVSD | 25 | QVWD | 368 |
| 54 | YGMH | | N | | VHEAFDI | | SKSVH | | RPS | | SSSTH | |
| | | | | | | | | | | | VV | |
| Antibody | GFTFRT | 6 | WYDGS | 11 | APQWEL | 13 | GGNNLV | 21 | DVSD | 25 | QVWD | 367 |
| 55 | YGMH | | N | | VHEAFDI | | SKSVH | | RPS | | SSSDF | |
| | | | | | | | | | | | VV | |
| Antibody | GFTFRT | 6 | WYDGS | 11 | APQWEL | 13 | GGNNLV | 21 | DDSD | 26 | QVWD | 369 |
| 56 | YGMH | | N | | VHEAFDI | | SKSVH | | RPS | | SSSTFV | |
| | | | | | | | | | | | V | |
| Antibody | GFTFRT | 6 | WYDGS | 11 | APQWEL | 13 | GGNNLV | 21 | DDSD | 26 | QVWD | 370 |
| 57 | YGMH | | N | | VHEAFDI | | SKSVH | | RPS | | SSSTIV | |
| | | | | | | | | | | | V | |
| Antibody | GFTFRT | 6 | WYDGS | 11 | APQWEL | 13 | GGNNLG | 19 | DVSD | 25 | QVWD | 369 |
| 58 | YGMH | | N | | VHEAFDI | | SKSVH | | RPS | | SSSTFV | |
| | | | | | | | | | | | V | |
| Antibody | GFTFRT | 6 | WYDGS | 11 | APQWEL | 13 | GGNNLV | 21 | DVSD | 25 | QVWD | 369 |
| 59 | YGMH | | N | | VHEAFDI | | SKSVH | | RPS | | SSSTFV | |
| | | | | | | | | | | | V | |
| Antibody | GFTFRT | 6 | WYDGS | 11 | APQWEL | 13 | GGNNLV | 21 | DVSD | 25 | QVWD | 370 |
| 60 | YGMH | | N | | VHEAFDI | | SKSVH | | RPS | | SSSTIV | |
| | | | | | | | | | | | V | |
| Antibody | GFTFRT | 6 | WYDGS | 11 | ALLWEL | 15 | GGNNLG | 19 | DDSD | 26 | QVWD | 29 |
| 61 | YGMH | | N | | FHEYFDI | | SKSVH | | RPS | | SSSDH | |
| | | | | | | | | | | | VV | |

IMGT Numbering

| | | | | | | | | | | | | |
|---|---|---|---|---|---|---|---|---|---|---|---|---|
| Antibody | GFTFRT | 9 | IWYDGS | 12 | ARAPQW | 16 | NLGSKS | 22 | DVS | — | QVWD | 29 |
| 47 | YG | | NK | | ELVHEAF | | | | | | SSSDH | |
| | | | | | DI | | | | | | VV | |
| Antibody | GFTFRT | 9 | IWYDGS | 12 | ARAPQW | 16 | NLGSKS | 22 | DDS | — | QVWD | 367 |
| 48 | YG | | NK | | ELVHEAF | | | | | | SSSDF | |
| | | | | | DI | | | | | | VV | |
| Antibody | GFTFRT | 9 | IWYDGS | 12 | ARAPQW | 16 | NLVSKS | 24 | DVS | — | QVWD | 29 |
| | | | | | ELVHEAF | | | | | | SSSDH | |
| 49 | YG | | NK | | DI | | | | | | VV | |
| Antibody | GFTFRT | 9 | IWYDGS | 12 | ARAPQW | 16 | NLVSKS | 24 | DDS | — | QVWD | 367 |
| | | | | | ELVHEAF | | | | | | SSSDF | |
| 50 | YG | | NK | | DI | | | | | | VV | |
| Antibody | GFTFRT | 9 | IWYDGS | 12 | ARAPQW | 16 | NLGSKS | 22 | DVS | — | QVWD | 368 |
| 51 | YG | | NK | | ELVHEAF | | | | | | SSSTH | |
| | | | | | DI | | | | | | VV | |
| Antibody | GFTFRT | 9 | IWYDGS | 12 | ARAPQW | 16 | NLGSKS | 22 | DVS | — | QVWD | 367 |
| 52 | YG | | NK | | ELVHEAF | | | | | | SSSDF | |
| | | | | | DI | | | | | | VV | |
| Antibody | GFTFRT | 9 | IWYDGS | 12 | ARAPQW | 16 | NLGSKS | 22 | DDS | — | QVWD | 369 |
| 53 | YG | | NK | | ELVHEAF | | | | | | SSSTFV | |
| | | | | | DI | | | | | | V | |
| Antibody | GFTFRT | 9 | IWYDGS | 12 | ARAPQW | 16 | NLVSKS | 24 | DVS | — | QVWD | 368 |
| 54 | YG | | NK | | ELVHEAF | | | | | | SSSTH | |
| | | | | | DI | | | | | | VV | |
| Antibody | GFTFRT | 9 | IWYDGS | 12 | ARAPQW | 16 | NLVSKS | 24 | DVS | — | QVWD | 367 |
| 55 | YG | | NK | | ELVHEAF | | | | | | SSSDF | |
| | | | | | DI | | | | | | VV | |
| Antibody | GFTFRT | 9 | IWYDGS | 12 | ARAPQW | 16 | NLVSKS | 24 | DDS | — | QVWD | 369 |
| 56 | YG | | NK | | ELVHEAF | | | | | | SSSTFV | |
| | | | | | DI | | | | | | V | |
| Antibody | GFTFRT | 9 | IWYDGS | 12 | ARAPQW | 16 | NLVSKS | 24 | DDS | — | QVWD | 370 |
| 57 | YG | | NK | | ELVHEAF | | | | | | SSSTIV | |
| | | | | | DI | | | | | | V | |
| Antibody | GFTFRT | 9 | IWYDGS | 12 | ARAPQW | 16 | NLGSKS | 22 | DVS | — | QVWD | 369 |
| 58 | YG | | NK | | ELVHEAF | | | | | | SSSTFV | |
| | | | | | DI | | | | | | V | |
| Antibody | GFTFRT | 9 | IWYDGS | 12 | ARAPQW | 16 | NLVSKS | 24 | DVS | — | QVWD | 369 |
| 59 | YG | | NK | | ELVHEAF | | | | | | SSSTFV | |
| | | | | | DI | | | | | | V | |
| Antibody | GFTFRT | 9 | IWYDGS | 12 | ARAPQW | 16 | NLVSKS | 24 | DVS | — | QVWD | 370 |
| 60 | YG | | NK | | ELVHEAF | | | | | | SSSTIV | |
| | | | | | DI | | | | | | V | |
| Antibody | GFTFRT | 9 | IWYDGS | 12 | ARALLW | 18 | NLGSKS | 22 | DDS | — | QVWD | 29 |
| 61 | YG | | NK | | ELFHEYF | | | | | | SSSDH | |
| | | | | | DI | | | | | | VV | |

TABLE 6
Sequences of additional anti-TSLP antibody constructs-VH/VL

| | SEQ | | SEQ | |
|---|---|---|---|---|
| Antibody | ID NO | VH | ID NO | VL |
| Antibody | 34 | QMQLVESGGGVVQPGRSLRLSCAASGFTFRTYGMH | 371 | SYVLTQPPSVSVAPGQTARITCGGNNLGSK |
| 47 | | WVRQAPGKGLEWVAVIWYDGSNKHYADSVKGRFT | | SVHWYQQKPGQAPVLVVYDVSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSDHVVFGGGTKLTVL |
| Antibody | 34 | QMQLVESGGGVVQPGRSLRLSCAASGFTFRTYGMH | 372 | SYVLTQPPSVSVAPGQTARITCGGNNLGSK |
| 48 | | WVRQAPGKGLEWVAVIWYDGSNKHYADSVKGRFT | | SVHWYQQKPGQAPVLVVYDDSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSDFVVFGGGTKLTVL |
| Antibody | 34 | QMQLVESGGGVVQPGRSLRLSCAASGFTFRTYGMH | 373 | SYVLTQPPSVSVAPGQTARITCGGNNLVSK |
| 49 | | WVRQAPGKGLEWVAVIWYDGSNKHYADSVKGRFT | | SVHWYQQKPGQAPVLVVYDVSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSDHVVFGGGTKLTVL |
| Antibody | 34 | QMQLVESGGGVVQPGRSLRLSCAASGFTFRTYGMH | 374 | SYVLTQPPSVSVAPGQTARITCGGNNLVSK |
| 50 | | WVRQAPGKGLEWVAVIWYDGSNKHYADSVKGRFT | | SVHWYQQKPGQAPVLVVYDDSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSDFVVFGGGTKLTVL |
| Antibody | 34 | QMQLVESGGGVVQPGRSLRLSCAASGFTFRTYGMH | 375 | SYVLTQPPSVSVAPGQTARITCGGNNLGSK |
| 51 | | WVRQAPGKGLEWVAVIWYDGSNKHYADSVKGRFT | | SVHWYQQKPGQAPVLVVYDVSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSTHVVFGGGTKLTVL |
| Antibody | 34 | QMQLVESGGGVVQPGRSLRLSCAASGFTFRTYGMH | 376 | SYVLTQPPSVSVAPGQTARITCGGNNLGSK |
| 52 | | WVRQAPGKGLEWVAVIWYDGSNKHYADSVKGRFT | | SVHWYQQKPGQAPVLVVYDVSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSDFVVFGGGTKLTVL |
| Antibody | 34 | QMQLVESGGGVVQPGRSLRLSCAASGFTFRTYGMH | 377 | SYVLTQPPSVSVAPGQTARITCGGNNLGSK |
| 53 | | WVRQAPGKGLEWVAVIWYDGSNKHYADSVKGRFT | | SVHWYQQKPGQAPVLVVYDDSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSTFVVFGGGTKLTVL |
| Antibody | 34 | QMQLVESGGGVVQPGRSLRLSCAASGFTFRTYGMH | 378 | SYVLTQPPSVSVAPGQTARITCGGNNLVSK |
| 54 | | WVRQAPGKGLEWVAVIWYDGSNKHYADSVKGRFT | | SVHWYQQKPGQAPVLVVYDVSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSTHVVFGGGTKLTVL |
| Antibody | 34 | QMQLVESGGGVVQPGRSLRLSCAASGFTFRTYGMH | 379 | SYVLTQPPSVSVAPGQTARITCGGNNLVSK |
| 55 | | WVRQAPGKGLEWVAVIWYDGSNKHYADSVKGRFT | | SVHWYQQKPGQAPVLVVYDVSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSDFVVFGGGTKLTVL |
| Antibody | 34 | QMQLVESGGGVVQPGRSLRLSCAASGFTFRTYGMH | 41 | SYVLTQPPSVSVAPGQTARITCGGNNLVSK |
| 56 | | WVRQAPGKGLEWVAVIWYDGSNKHYADSVKGRFT | | SVHWYQQKPGQAPVLVVYDDSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSTFVVFGGGTKLTVL |
| Antibody | 34 | QMQLVESGGGVVQPGRSLRLSCAASGFTFRTYGMH | 381 | SYVLTQPPSVSVAPGQTARITCGGNNLVSK |
| 57 | | WVRQAPGKGLEWVAVIWYDGSNKHYADSVKGRFT | | SVHWYQQKPGQAPVLVVYDDSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSTIVVFGGGTKLTVL |
| Antibody | 34 | QMQLVESGGGVVQPGRSLRLSCAASGFTFRTYGMH | 382 | SYVLTQPPSVSVAPGQTARITCGGNNLGSK |
| 58 | | WVRQAPGKGLEWVAVIWYDGSNKHYADSVKGRFT | | SVHWYQQKPGQAPVLVVYDVSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSTFVVFGGGTKLTVL |
| Antibody | 34 | QMQLVESGGGVVQPGRSLRLSCAASGFTFRTYGMH | 383 | SYVLTQPPSVSVAPGQTARITCGGNNLVSK |
| 59 | | WVRQAPGKGLEWVAVIWYDGSNKHYADSVKGRFT | | SVHWYQQKPGQAPVLVVYDVSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSTFVVFGGGTKLTVL |
| Antibody | 34 | QMQLVESGGGVVQPGRSLRLSCAASGFTFRTYGMH | 384 | SYVLTQPPSVSVAPGQTARITCGGNNLVSK |
| 60 | | WVRQAPGKGLEWVAVIWYDGSNKHYADSVKGRFT | | SVHWYQQKPGQAPVLVVYDVSDRPSWIP |
| | | ITRDNSKNTLNLQMNSLRAEDTAVYYCARAPQWEL | | ERFSGSNSGNTATLTISRGEAGDEADYYCQ |
| | | VHEAFDIWGQGTMVTVSS | | VWDSSSTIVVFGGGTKLTVL |
| Antibody | 35 | QMQLVESGGGVVQPGRSLRLSCAASGFTFRTYGMH | 385 | SYVLTQPPSVSVAPGQTARITCGGNNLGSK |
| 61 | | WVRQAPGKGLEWVAVIWYDGSNKHYADSVKGRFT | | SVHWYQQKPGQAPVLVVYDDSDRPSWIP |
| | | ITRDNSKNTLNLQMNSLRAEDTAVYYCARALLWEL | | ERFSGSNSGNTATLTISRGEAGDEADYYCQ |
| | | FHEYFDIWGQGTMVTVSS | | VWDSSSDHVVFGGGTKLTVL |

### VH Domains

In some embodiments, an antibody, or an antigen binding fragment thereof, provided herein comprises a VH sequence selected from any one of SEQ ID NOs: 32-36 and 386.

In some embodiments, an antibody, or an antigen binding fragment thereof, provided herein comprises a VH sequence having at least about 80%, 90%, 91%, 92%, 93%, 94%, 95%, 96%, 97%, 98%, or 99% identity to an illustrative VH sequence provided in any one of SEQ ID NOs: 32-36 and 386. In some embodiments, an antibody, or an antigen binding fragment thereof, provided herein comprises a VH sequence provided in any one of SEQ ID NOs: 32-36 and 386, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VL Domains

In some embodiments, an antibody, or an antigen binding fragment thereof, provided herein comprises a VL sequence selected from any one of SEQ ID NOs: 37-45, 328-364, 371-379, and 381-385.

In some embodiments, an antibody, or an antigen binding fragment thereof, provided herein comprises a VL sequence having at least about 80%, 90%, 91%, 92%, 93%, 94%, 95%, 96%, 97%, 98%, or 99% identity to a VL sequence provided in any one of SEQ ID NOs: 37-45, 328-364, 371-379, and 381-385. In some embodiments, an antibody, or an antigen binding fragment thereof, provided herein comprises a VL sequence provided in any one of SEQ ID NOs: 37-45, 328-364, 371-379, and 381-385, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VH-VL Combinations

In some embodiments, an antibody, or an antigen binding fragment thereof, provided herein comprises a VH sequence selected from any one of SEQ ID NOs: 32-36 and 386; and a VL sequence selected from any one of SEQ ID NOs: 37-45, 328-364, 371-379, and 381-385.

In certain aspects, any one of SEQ ID NOs: 32-36 and 386 can be combined with any one of SEQ ID NOs: 37-45, 328-364, 371-379, and 381-385.

In certain aspects, an antibody, or an antigen binding fragment thereof, provided herein comprises a VH sequence and a VL sequence of a construct provided in TABLE 4 (e.g., a VH sequence and a VL sequence from the same row of TABLE 4). In certain aspects, an antibody, or an antigen binding fragment thereof, provided herein comprises a VH sequence and a VL sequence of a construct provided in TABLE 6 (e.g., a VH sequence and a VL sequence from the same row of TABLE 6).

In certain aspects, an antibody, or an antigen binding fragment thereof, provided herein comprises a VH sequence and a VL sequence of a construct provided in PCT Application No. PCT/US2025/034623, incorporated by reference herein in its entirety. In certain aspects, an antibody, or antigen binding fragment thereof, provided herein comprises a VH sequence and a VL sequence from Table 4 or Table 11 in PCT Application No. PCT/US2025/034623 (e.g., a VH sequence and a VL sequence from the same row of Table 4 or Table 11).

In some embodiments, an antibody, or an antigen binding fragment thereof, provided herein comprises a VH sequence having at least about 80%, 90%, 91%, 92%, 93%, 94%, 95%, 96%, 97%, 98%, or 99% identity to a VH sequence provided in any one of SEQ ID NOs: 32-36 and 386; and a VL sequence having at least about 80%, 90%, 91%, 92%, 93%, 94%, 95%, 96%, 97%, 98%, or 99% identity to a VL sequence provided in any one of SEQ ID NOs: 37-45, 328-364, 371-379, and 381-385. In some embodiments, an antibody, or an antigen binding fragment thereof, provided herein comprises a VH sequence provided in any one of SEQ ID NOs: 32-36 and 386, with up to 1, 2, 3, 4, 5, 6, 7, 8, 9, 10, 11, 12, 13, 14, 15, 16, 17, 18, 19, or 20 amino acid substitutions, and a VL sequence provided in any one of SEQ ID NOs: 37-45, 328-364, 371-379, and 381-385, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In certain embodiments, the antibody, or an antigen binding fragment thereof, comprises a VH sequence set forth in SEQ ID NO: 32 and a VL sequence set forth in SEQ ID NO: 37.

In certain embodiments, the antibody, or an antigen binding fragment thereof, comprises a VH sequence set forth in SEQ ID NO: 33 and a VL sequence set forth in SEQ ID NO: 38.

In certain embodiments, the antibody, or an antigen binding fragment thereof, comprises a VH sequence set forth in SEQ ID NO: 386 and a VL sequence set forth in SEQ ID NO: 39.

In certain embodiments, the antibody, or an antigen binding fragment thereof, comprises a VH sequence set forth in SEQ ID NO: 33 and a VL sequence set forth in SEQ ID NO: 40.

In certain embodiments, the antibody, or an antigen binding fragment thereof, comprises a VH sequence set forth in SEQ ID NO: 34 and a VL sequence set forth in SEQ ID NO: 41.

In certain embodiments, the antibody, or an antigen binding fragment thereof, comprises a VH sequence set forth in SEQ ID NO: 35 and a VL sequence set forth in SEQ ID NO: 42.

In certain embodiments, the antibody, or an antigen binding fragment thereof, comprises a VH sequence set forth in SEQ ID NO: 34 and a VL sequence set forth in SEQ ID NO: 43.

In certain embodiments, the antibody, or an antigen binding fragment thereof, comprises a VH sequence set forth in SEQ ID NO: 32 and a VL sequence set forth in SEQ ID NO: 44.

In certain embodiments, the antibody, or an antigen binding fragment thereof, comprises a VH sequence set forth in SEQ ID NO: 36 and a VL sequence set forth in SEQ ID NO: 45.

In certain embodiments, the antibody, or an antigen binding fragment thereof, comprises a VH sequence set forth in SEQ ID NO: 34 and a VL sequence set forth in SEQ ID NO: 328.

In certain embodiments, the antibody, or an antigen binding fragment thereof, comprises a VH sequence set forth in SEQ ID NO: 34 and a VL sequence set forth in SEQ ID NO: 329.

In certain embodiments, the antibody, or an antigen binding fragment thereof, comprises a VH sequence set forth in SEQ ID NO: 34 and a VL sequence set forth in SEQ ID NO: 330.

In certain embodiments, the antibody, or an antigen binding fragment thereof, comprises a VH sequence set forth in SEQ ID NO: 34 and a VL sequence set forth in SEQ ID NO: 331.

In certain embodiments, the antibody, or an antigen binding fragment thereof, comprises a VH sequence set forth in SEQ ID NO: 34 and a VL sequence set forth in SEQ ID NO: 332.

In certain embodiments, the antibody, or an antigen binding fragment thereof, comprises a VH sequence set forth in SEQ ID NO: 34 and a VL sequence set forth in SEQ ID NO: 333.

In certain embodiments, the antibody, or an antigen binding fragment thereof, NO: 334.

In certain embodiments, the antibody, or an antigen binding fragment thereof, comprises a VH sequence set forth in SEQ ID NO: 34 and a VL sequence set forth in SEQ ID NO: 335.

In certain embodiments, the antibody, or an antigen binding fragment thereof, comprises a VH sequence set forth in SEQ ID NO: 34 and a VL sequence set forth in SEQ ID NO: 336.

In certain embodiments, the antibody, or an antigen binding fragment thereof, comprises a VH sequence set forth in SEQ ID NO: 34 and a VL sequence set forth in SEQ ID NO: 337.

In certain embodiments, the antibody, or an antigen binding fragment thereof, comprises a VH sequence set forth in SEQ ID NO: 34 and a VL sequence set forth in SEQ ID NO: 338.

In certain embodiments, the antibody, or an antigen binding fragment thereof, comprises a VH sequence set forth in SEQ ID NO: 34 and a VL sequence set forth in SEQ ID NO: 339.

In certain embodiments, the antibody, or an antigen binding fragment thereof, comprises a VH sequence set forth in SEQ ID NO: 34 and a VL sequence set forth in SEQ ID NO: 340.

In certain embodiments, the antibody, or an antigen binding fragment thereof, comprises a VH sequence set forth in SEQ ID NO: 34 and a VL sequence set forth in SEQ ID NO: 341.

In certain embodiments, the antibody, or an antigen binding fragment thereof, comprises a VH sequence set forth in SEQ ID NO: 34 and a VL sequence set forth in SEQ ID NO: 342.

In certain embodiments, the antibody, or an antigen binding fragment thereof, comprises a VH sequence set forth in SEQ ID NO: 34 and a VL sequence set forth in SEQ ID NO: 343.

In certain embodiments, the antibody, or an antigen binding fragment thereof, NO: 344.

In certain embodiments, the antibody, or an antigen binding fragment thereof, comprises a VH sequence set forth in SEQ ID NO: 34 and a VL sequence set forth in SEQ ID NO: 345.

In certain embodiments, the antibody, or an antigen binding fragment thereof, comprises a VH sequence set forth in SEQ ID NO: 34 and a VL sequence set forth in SEQ ID NO: 346.

In certain embodiments, the antibody, or an antigen binding fragment thereof, comprises a VH sequence set forth in SEQ ID NO: 34 and a VL sequence set forth in SEQ ID NO: 347.

In certain embodiments, the antibody, or an antigen binding fragment thereof, comprises a VH sequence set forth in SEQ ID NO: 34 and a VL sequence set forth in SEQ ID NO: 348.

In certain embodiments, the antibody, or an antigen binding fragment thereof, comprises a VH sequence set forth in SEQ ID NO: 34 and a VL sequence set forth in SEQ ID NO: 349.

In certain embodiments, the antibody, or an antigen binding fragment thereof, comprises a VH sequence set forth in SEQ ID NO: 34 and a VL sequence set forth in SEQ ID NO: 350.

In certain embodiments, the antibody, or an antigen binding fragment thereof, comprises a VH sequence set forth in SEQ ID NO: 34 and a VL sequence set forth in SEQ ID NO: 351.

In certain embodiments, the antibody, or an antigen binding fragment thereof, comprises a VH sequence set forth in SEQ ID NO: 34 and a VL sequence set forth in SEQ ID NO: 352.

In certain embodiments, the antibody, or an antigen binding fragment thereof, comprises a VH sequence set forth in SEQ ID NO: 34 and a VL sequence set forth in SEQ ID NO: 353.

In certain embodiments, the antibody, or an antigen binding fragment thereof, NO: 354.

In certain embodiments, the antibody, or an antigen binding fragment thereof, comprises a VH sequence set forth in SEQ ID NO: 34 and a VL sequence set forth in SEQ ID NO: 355.

In certain embodiments, the antibody, or an antigen binding fragment thereof, comprises a VH sequence set forth in SEQ ID NO: 34 and a VL sequence set forth in SEQ ID NO: 356.

In certain embodiments, the antibody, or an antigen binding fragment thereof, comprises a VH sequence set forth in SEQ ID NO: 34 and a VL sequence set forth in SEQ ID NO: 357.

In certain embodiments, the antibody, or an antigen binding fragment thereof, comprises a VH sequence set forth in SEQ ID NO: 34 and a VL sequence set forth in SEQ ID NO: 358.

In certain embodiments, the antibody, or an antigen binding fragment thereof, comprises a VH sequence set forth in SEQ ID NO: 34 and a VL sequence set forth in SEQ ID NO: 359.

In certain embodiments, the antibody, or an antigen binding fragment thereof, comprises a VH sequence set forth in SEQ ID NO: 34 and a VL sequence set forth in SEQ ID NO: 360.

In certain embodiments, the antibody, or an antigen binding fragment thereof, comprises a VH sequence set forth in SEQ ID NO: 34 and a VL sequence set forth in SEQ ID NO: 361.

In certain embodiments, the antibody, or an antigen binding fragment thereof, comprises a VH sequence set forth in SEQ ID NO: 34 and a VL sequence set forth in SEQ ID NO: 362.

In certain embodiments, the antibody, or an antigen binding fragment thereof, comprises a VH sequence set forth in SEQ ID NO: 34 and a VL sequence set forth in SEQ ID NO: 363.

In certain embodiments, the antibody, or an antigen binding fragment thereof, NO: 364.

In certain embodiments, the antibody, or an antigen binding fragment thereof, comprises a VH sequence set forth in SEQ ID NO: 34 and a VL sequence set forth in SEQ ID NO: 371.

In certain embodiments, the antibody, or an antigen binding fragment thereof, comprises a VH sequence set forth in SEQ ID NO: 34 and a VL sequence set forth in SEQ ID NO: 372.

In certain embodiments, the antibody, or an antigen binding fragment thereof, comprises a VH sequence set forth in SEQ ID NO: 34 and a VL sequence set forth in SEQ ID NO: 373.

In certain embodiments, the antibody, or an antigen binding fragment thereof, comprises a VH sequence set forth in SEQ ID NO: 34 and a VL sequence set forth in SEQ ID NO: 374.

In certain embodiments, the antibody, or an antigen binding fragment thereof, comprises a VH sequence set forth in SEQ ID NO: 34 and a VL sequence set forth in SEQ ID NO: 375.

In certain embodiments, the antibody, or an antigen binding fragment thereof, comprises a VH sequence set forth in SEQ ID NO: 34 and a VL sequence set forth in SEQ ID NO: 376.

In certain embodiments, the antibody, or an antigen binding fragment thereof, comprises a VH sequence set forth in SEQ ID NO: 34 and a VL sequence set forth in SEQ ID NO: 377.

In certain embodiments, the antibody, or an antigen binding fragment thereof, comprises a VH sequence set forth in SEQ ID NO: 34 and a VL sequence set forth in SEQ ID NO: 378.

In certain embodiments, the antibody, or an antigen binding fragment thereof, comprises a VH sequence set forth in SEQ ID NO: 34 and a VL sequence set forth in SEQ ID NO: 379.

In certain embodiments, the antibody, or an antigen binding fragment thereof, NO: 381.

In certain embodiments, the antibody, or an antigen binding fragment thereof, comprises a VH sequence set forth in SEQ ID NO: 34 and a VL sequence set forth in SEQ ID NO: 382.

In certain embodiments, the antibody, or an antigen binding fragment thereof, comprises a VH sequence set forth in SEQ ID NO: 34 and a VL sequence set forth in SEQ ID NO: 383.

In certain embodiments, the antibody, or an antigen binding fragment thereof, comprises a VH sequence set forth in SEQ ID NO: 34 and a VL sequence set forth in SEQ ID NO: 384.

In certain embodiments, the antibody, or an antigen binding fragment thereof, comprises a VH sequence set forth in SEQ ID NO: 35 and a VL sequence set forth in SEQ ID NO: 385.

In certain embodiments of any of the antibodies or antigen binding fragments thereof described above, the antibody further comprises a heavy chain comprising a human IgG sequence selected from a sequence set forth in any one of SEQ ID NOs: 47-270. In certain embodiments, the heavy chain comprises a human IgG sequence selected from SEQ ID NO: 61 and SEQ ID NO: 173.

Although a C-terminal lysine may be present in the corresponding coding sequence of the constant heavy chain region (e.g., in a sequence encoding any one of SEQ ID NOs: 159-270), it may be cleaved off during manufacture or after administration (resulting in, e.g., a constant heavy chain sequence of any one of SEQ ID NOs: 47-158). Accordingly, any of the antibodies or antigen binding fragments thereof described above may comprise a human IgG sequence containing a C-terminal lysine (e.g., any one of SEQ ID NOs: 159-270), a human IgG sequence lacking a C-terminal lysine (e.g., any one of SEQ ID NOs: 47-158), or a mixture thereof (e.g., a mixture of the same heavy chain constant sequence with and without a C-terminal lysine, such as a mixture of SEQ ID NO: 173 and SEQ ID NO: 61).

In certain embodiments, the antibody, or an antigen binding fragment thereof, comprises a VH sequence set forth in SEQ ID NO: 478; a VL sequence set forth in SEQ ID NO: 479; and the antibody further comprises a heavy chain comprising a human IgG sequence selected from a sequence set forth in SEQ ID NOs: 47-270. In certain embodiments, the antibody, or antigen binding fragment thereof, comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In certain embodiments of any of the antibodies described above, the antibody further comprises a constant light chain sequence comprising a sequence set forth in SEQ ID NO: 480.

In certain embodiments, the antibody, or antigen binding fragment thereof, comprises a VH sequence set forth in SEQ ID NO: 34, a VL sequence set forth in SEQ ID NO: 41, a heavy chain constant region comprising LALA/YTE substitutions, and a human lambda light chain constant region (e.g., a lambda light chain constant region of subtype 1, 2, 3, or 7, such as a lambda light chain constant region set forth in any one of SEQ ID NOs: 480-483). In certain embodiments, the antibody, or antigen binding fragment thereof, comprises a VH sequence set forth in SEQ ID NO: 34, a VL sequence set forth in SEQ ID NO: 41, a heavy chain constant region comprising a sequence set forth in SEQ ID NO: 61 or SEQ ID NO: 173, and a light chain constant region comprising a sequence set forth in SEQ ID NO: 480. In certain embodiments, the antibody, or antigen binding fragment thereof, comprises a VH sequence having at least about 80%, 90%, 91%, 92%, 93%, 94%, 95%, 96%, 97%, 98% or 99% identity to a VH sequence provided in SEQ ID NO: 34; and a VL sequence having at least about 80%, 90%, 91%, 92%, 93%, 94%, 95%, 96%, 97%, 98% or 99% identity to a VL sequence provided in SEQ ID NO: 41, a heavy chain constant region comprising LALA/YTE substitutions, and a human lambda light chain constant region (e.g., a lambda light chain constant region of subtype 1, 2, 3, or 7, such as a lambda light chain constant region set forth in any one of SEQ ID NOs: 480-483). In certain embodiments, the antibody, or antigen binding fragment thereof, comprises a VH sequence having at least about 80%, 90%, 91%, 92%, 93%, 94%, 95%, 96%, 97%, 98% or 99% identity to a VH sequence provided in SEQ ID NO: 34; and a VL sequence having at least about 80%, 90%, 91%, 92%, 93%, 94%, 95%, 96%, 97%, 98% or 99% identity to a VL sequence provided in SEQ ID NO: 41, a heavy chain constant region comprising a sequence set forth in SEQ ID NO: 61 or SEQ ID NO: 173 or a sequence having at least about 80%, 90%, 91%, 92%, 93%, 94%, 95%, 96%, 97%, 98% or 99% identity to SEQ ID NO: 61 or SEQ ID NO: 173, and a light chain constant region comprising a sequence set forth in SEQ ID NO: 480, or a sequence having at least about 80%, 90%, 91%, 92%, 93%, 94%, 95%, 96%, 97%, 98% or 99% identity to SEQ ID NO: 480.

In certain embodiments, the antibody, or antigen binding fragment thereof, comprises a VH sequence set forth in SEQ ID NO: 34, a VL sequence set forth in SEQ ID NO: 41, a heavy chain constant region comprising a sequence set forth in SEQ ID NO: 61 or SEQ ID NO: 173, and a light chain constant region comprising a sequence set forth in SEQ ID NO: 480. In certain embodiments, the antibody comprises a VH sequence set forth in SEQ ID NO: 34, a VL sequence set forth in SEQ ID NO: 41, a heavy chain constant region comprising a sequence set forth in SEQ ID NO: 173, and a light chain constant region comprising a sequence set forth in SEQ ID NO: 480. In some embodiments, the antibody comprises a heavy chain sequence set forth in SEQ ID NO: 484 or 486 and a light chain sequence set forth in SEQ ID NO: 485. In some embodiments, the antibody comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in an antibody containing the heavy chain sequence of SEQ ID NO: 486.

In certain embodiments, the antibody, or antigen binding fragment thereof, comprises a VH sequence set forth in SEQ ID NO: 34, a VL sequence set forth in SEQ ID NO: 43, a heavy chain constant region comprising LALA/YTE substitutions, and a human lambda light chain constant region (e.g., a lambda light chain constant region of subtype 1, 2, 3, or 7, such as a lambda light chain constant region set forth in any one of SEQ ID NOs: 480-483). In certain embodiments, the antibody, or antigen binding fragment thereof, comprises a VH sequence set forth in SEQ ID NO: 34, a VL sequence set forth in SEQ ID NO: 43, a heavy chain constant region comprising a sequence set forth in SEQ ID NO: 61 or SEQ ID NO: 173, and a light chain constant region comprising a sequence set forth in SEQ ID NO: 480. In certain embodiments, the antibody, or antigen binding fragment thereof, comprises a VH sequence having at least about 80%, 90%, 91%, 92%, 93%, 94%, 95%, 96%, 97%, 98% or 99% identity to a VH sequence provided in SEQ ID NO: 34; and a VL sequence having at least about 80%, 90%, 91%, 92%, 93%, 94%, 95%, 96%, 97%, 98% or 99% identity to a VL sequence provided in SEQ ID NO: 43, a heavy chain constant region comprising LALA/YTE substitutions, and a human lambda light chain constant region (e.g., a lambda light chain constant region of subtype 1, 2, 3, or 7, such as a lambda light chain constant region set forth in any one of SEQ ID NOs: 480-483). In certain embodiments, the antibody, or antigen binding fragment thereof, comprises a VH sequence having at least about 80%, 90%, 91%, 92%, 93%, 94%, 95%, 96%, 97%, 98% or 99% identity to a VH sequence provided in SEQ ID NO: 34; and a VL sequence having at least about 80%, 90%, 91%, 92%, 93%, 94%, 95%, 96%, 97%, 98% or 99% identity to a VL sequence provided in SEQ ID NO: 43, a heavy chain constant region comprising a sequence set forth in SEQ ID NO: 61 or SEQ ID NO: 173 or a sequence having at least about 80%, 90%, 91%, 92%, 93%, 94%, 95%, 96%, 97%, 98% or 99% identity to SEQ ID NO: 61 or SEQ ID NO: 173, and a light chain constant region comprising a sequence set forth in SEQ ID NO: 480 or a sequence having at least about 80%, 90%, 91%, 92%, 93%, 94%, 95%, 96%, 97%, 98% or 99% identity to SEQ ID NO: 480.

In certain embodiments, the antibody, or antigen binding fragment thereof, comprises a VH sequence set forth in SEQ ID NO: 34, a VL sequence set forth in SEQ ID NO: 43, a heavy chain constant region comprising a sequence set forth in SEQ ID NO: 61 or SEQ ID NO: 173, and a light chain constant region comprising a sequence set forth in SEQ ID NO: 480. In certain embodiments, the antibody comprises a VH sequence set forth in SEQ ID NO: 34, a VL sequence set forth in SEQ ID NO: 43, a heavy chain constant region comprising a sequence set forth in SEQ ID NO: 173, and a light chain constant region comprising a sequence set forth in SEQ ID NO: 480. In some embodiments, the antibody comprises a heavy chain sequence set forth in SEQ ID NO: 487 and a light chain sequence set forth in SEQ ID NO: 488. The heavy chain sequence of SEQ ID NO: 487 contains a C-terminal lysine, which may be cleaved off during manufacture or after administration.

### CDRs

In some embodiments, disclosed herein is an antibody, or an antigen binding fragment thereof, comprising 1, 2, 3, 4, 5, or 6 of the CDRs of TABLE 3. In some embodiments, disclosed herein is an antibody, or an antigen binding fragment thereof, comprising 6 of the Kabat CDRs of TABLE 3, 6 of the Chothia CDRs of TABLE 3, or 6 of the IMGT CDRs of TABLE 3. In some embodiments, the antibody, or an antigen binding fragment thereof, comprises 6 of the Kabat CDRs, 6 of the Chothia CDRs, or 6 of the IMGT CDRs from a single row of TABLE 3 (e.g., 6 CDRs from the same antibody).

In some embodiments, disclosed herein is an antibody, or an antigen binding fragment thereof, comprising 1, 2, 3, 4, 5, or 6 of the CDRs of TABLE 5. In some embodiments, disclosed herein is an antibody, or an antigen binding fragment thereof, comprising 6 of the Kabat CDRs of TABLE 5, 6 of the Chothia CDRs of TABLE 5, or 6 of the IMGT CDRs of TABLE 5. In some embodiments, the antibody, or an antigen binding fragment thereof, comprises 6 of the Kabat CDRs, 6 of the Chothia CDRs, or 6 of the IMGT CDRs from a single row of TABLE 5 (e.g., 6 CDRs from the same antibody).

In some embodiments, disclosed herein is an antibody, or an antigen binding fragment thereof, comprising 1, 2, 3, 4, 5, or 6 of the CDRs of an antibody provided in PCT Application No. PCT/US2025/034623, incorporated by reference herein in its entirety, such as 1, 2, 3, 4, 5, or 6 of the CDRs in Table 3 or Table 10 in PCT Application No. PCT/US2025/034623. In some embodiments, disclosed herein is an antibody, or an antigen binding fragment thereof, comprising 1, 2, 3, 4, 5, or 6 of the Kabat CDRs of an antibody, or an antigen binding fragment thereof, provided in PCT Application No. PCT/US2025/034623. In some embodiments, disclosed herein is an antibody, or an antigen binding fragment thereof, comprising 1, 2, 3, 4, 5, or 6 of the Chothia CDRs of an antibody, or an antigen binding fragment thereof, provided in PCT Application No. PCT/US2025/034623. In some embodiments, disclosed herein is an antibody, or an antigen binding fragment thereof, comprising 1, 2, 3, 4, 5, or 6 of the IMGT CDRs of an antibody provided in PCT Application No. PCT/US2025/034623. In some embodiments, the antibody, or an antigen binding fragment thereof, comprises 6 of the Kabat CDRs, 6 of the Chothia CDRs, or 6 of the IMGT CDRs from a single row of Table 3 or Table 10 in in PCT Application No. PCT/US2025/034623 (e.g., 6 CDRs from the same antibody).

In some embodiments, an antibody, or an antigen binding fragment thereof, provided herein comprises one to three CDRs of a VH domain selected from any one of SEQ ID NOs: 32-36 and 386. In some embodiments, an antibody, or an antigen binding fragment thereof, provided herein comprises two to three CDRs of a VH domain selected from any one of SEQ ID NOs: 32-36 and 386. In some embodiments, an antibody, or an antigen binding fragment thereof, provided herein comprises three CDRs of a VH domain selected from any one of SEQ ID NOs: 32-36 and 386. In some aspects, the CDRs are Exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity to a CDR-H1, CDR-H2, or CDR-H3 selected from SEQ ID NOs: 1-18. In some embodiments, the CDR-H1 is a CDR-H1 of a VH domain selected from any one of SEQ ID NOs: 32-36 and 386, with up to 1, 2, 3, 4, or 5 amino acid substitutions. In some embodiments, the CDR-H2 is a CDR-H2 of a VH domain selected from any one of SEQ ID NO: 32-36 and 386, with up to 1, 2, 3, 4, 5, 6, 7, or 8 amino acid substitutions. In some embodiments, the CDR-H3 is a CDR-H3 of a VH domain selected from any one of SEQ ID NOs: 32-36 and 386, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or an antigen binding fragment thereof, provided herein comprises one to three CDRs of a VL domain selected from any one of SEQ ID NOs: 37-45, 328-364, 371-379, and 381-385. In some embodiments, an antibody, or an antigen binding fragment thereof, provided herein comprises two to three CDRs of a VL domain selected from any one of SEQ ID NO: 37-45, 328-364, 371-379, and 381-385. In some embodiments, an antibody, or an antigen binding fragment thereof, provided herein comprises three CDRs of a VL domain selected from any one of SEQ ID NO: 37-45, 328-364, 371-379, and 381-385. In some aspects, the CDRs are Exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity to a CDR-L1, CDR-L2, or CDR-L3 selected from SEQ ID NOs: 19-31, 271-327, 367-370, 387, and amino acid sequences DVS, EDS, DDT, DDK, DDL, DDN, and DDS. In some embodiments, the CDR-L1 is a CDR-L1 of a VL domain selected from any one of SEQ ID NOs: 37-45, 328-364, 371-379, and 381-385, with up to 1, 2, 3, 4, or 5 amino acid substitutions. In some embodiments, the CDR-L2 is a CDR-L2 of a VL domain selected from any one of SEQ ID NOs: 37-45, 328-364, 371-379, and 381-385, with up to 1, 2, 3, 4, 5, 6, 7, or 8 amino acid substitutions. In some embodiments, the CDR-L3 is a CDR-L3 of a VL domain selected from any one of SEQ ID NOs: 37-45, 328-364, 371-379, and 381-385, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or an antigen binding fragment thereof, provided herein comprises one to three CDRs of a VH domain selected from any one of SEQ ID NOs: 32-36 and 386 and one to three CDRs of a VL domain selected from any one of SEQ ID NOs: 37-45, 328-364, 371-379, and 381-385. In some embodiments, an antibody, or an antigen binding fragment thereof, provided herein comprises two to three CDRs of a VH domain selected from any one of SEQ ID NOs: 32-36 and 386 and two to three CDRs of a VL domain selected from any one of SEQ ID NOs: 37-45, 328-364, 371-379, and 381-385. In some embodiments, an antibody, or an antigen binding fragment thereof, provided herein comprises three CDRs of a VH domain selected from any one of SEQ ID NOs: 32-36 and 386 and three CDRs of a VL domain selected from any one of SEQ ID NOs: 37-45, 328-364, 371-379, and 381-385. In some aspects, the CDRs are Exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, an antibody, or an antigen binding fragment thereof, provided herein comprises a CDR-H3 selected from any one of SEQ ID NOs: 13-18. In some aspects, the CDR-H3 has at least about 80%, 90%, 91%, 92%, 93%, 94%, 95%, 96%, 97%, 98%, or 99% identity to a CDR-H3 selected from any one of SEQ ID NOs: 13-18. In some embodiments, the CDR-H3 is a CDR-H3 selected from any one of SEQ ID NOs: 13-18, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or an antigen binding fragment thereof, provided herein comprises a CDR-H1 selected from any one of SEQ ID NOs: 1-9. In some aspects, the CDR-H1 has at least about 80%, 90%, 91%, 92%, 93%, 94%, 95%, 96%, 97%, 98%, or 99% identity to a CDR-H1 selected from any one of SEQ ID NOs: 1-9. In some embodiments, the CDR-H1 is a CDR-H1 selected from any one of SEQ ID NOs: 1-9, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or an antigen binding fragment thereof, provided herein comprises a CDR-H2 selected from any one of SEQ ID NOs: 10-12. In some aspects, the CDR-H2 has at least about 80%, 90%, 91%, 92%, 93%, 94%, 95%, 96%, 97%, 98%, or 99% identity to a CDR-H2 selected from any one of SEQ ID NOs: 10-12. In some embodiments, the CDR-H2 is a CDR-H2 selected from any one of SEQ ID NOs: 10-12, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or an antigen binding fragment thereof, provided herein comprises a CDR-L3 selected from any one of SEQ ID NOs: 27-31, 311-327, and 367-370. In some aspects, the CDR-L3 has at least about 80%, 90%, 91%, 92%, 93%, 94%, 95%, 96%, 97%, 98%, or 99% identity to a CDR-L3 selected from any one of SEQ ID NOs: 27-31, 311-327, and 367-370. In some embodiments, the CDR-L3 is a CDR-L3 selected from any one of SEQ ID NOs: 27-31, 311-327, and 367-370, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or an antigen binding fragment thereof, provided herein comprises a CDR-L2 selected from any one of SEQ ID NOs: 25-26, 303-310, and amino acid sequences DDS, DVS, EDS, DDT, DDK, DDL, and DDN. In some aspects, the CDR-L2 has at least about 80%, 90%, 91%, 92%, 93%, 94%, 95%, 96%, 97%, 98%, or 99% identity to a CDR-L2 selected from any one of SEQ ID NOs: 25-26, 303-310, and amino acid sequences DDS, DVS, EDS, DDT, DDK, DDL, and DDN. In some embodiments, the CDR-L2 is a CDR-L2 selected from any one of SEQ ID NOs: 25-26, 303-310, and amino acid sequences DDS, DVS, EDS, DDT, DDK, DDL, and DDN, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or an antigen binding fragment thereof, provided herein comprises a CDR-L1 selected from any one of SEQ ID NOs: 19-24, 271-302, and 387. In some aspects, the CDR-L1 has at least about 80%, 90%, 91%, 92%, 93%, 94%, 95%, 96%, 97%, 98%, or 99% identity to a CDR-L1 selected from any one of SEQ ID NOs: 19-24, 271-302, and 387. In some embodiments, the CDR-L1 is a CDR-L1 selected from any one of SEQ ID NOs: 19-24, 271-302, and 387, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or an antigen binding fragment thereof, provided herein comprises a CDR-H3 selected from any one of SEQ ID NOs: 13-18, a CDR-H2 selected from any one of SEQ ID NOs: 10-12, a CDR-H1 selected from any one of SEQ ID NOs: 1-9, a CDR-L3 selected from any one of SEQ ID NOs: 27-31, 311-327, and 367-370, a CDR-L2 selected from any one of SEQ ID NOs: 25-26, 303-310, and amino acid sequences DDS, DVS, EDS, DDT, DDK, DDL, and DDN, and a CDR-L1 selected from any one of SEQ ID NOs: 19-24, 271-302, and 387. In some embodiments, the CDR-H3 has at least about 80%, 90%, 91%, 92%, 93%, 94%, 95%, 96%, 97%, 98% or 99% identity with a CDR-H3 selected from any one of SEQ ID NOs: 13-18, the CDR-H2 has at least about 80%, 90%, 91%, 92%, 93%, 94%, 95%, 96%, 97%, 98% or 99% identity with a CDR-H2 selected from any one of SEQ ID NOs: 10-12, the CDR-H1 has at least about 80%, 90%, 91%, 92%, 93%, 94%, 95%, 96%, 97%, 98% or 99% identity with a CDR-H1 selected from any one of SEQ ID NOs: 1-9, the CDR-L3 has at least about 80%, 90%, 91%, 92%, 93%, 94%, 95%, 96%, 97%, 98% or 99% identity with a CDR-L3 selected from any one of SEQ ID NOs: 27-31, 311-327, and 367-370, the CDR-L2 has at least about 80%, 90%, 91%, 92%, 93%, 94%, 95%, 96%, 97%, 98% or 99% identity with a CDR-L2 selected from any one of SEQ ID NOs: 25-26, 303-310, and amino acid sequences DDS, DVS, EDS, DDT, DDK, DDL, DDN, DVS, and DDS, and the CDR-L1 has at least about 80%, 90%, 91%, 92%, 93%, 94%, 95%, 96%, 97%, 98% or 99% identity with a CDR-L1 selected from any one of SEQ ID NOs: 19-24, 271-302, and 387. In some embodiments, the CDR-H3 is a CDR-H3 selected from any one of SEQ ID NOs: 13-18, with up to 1, 2, 3, 4, 5, 6, 7, or 8 amino acid substitutions; the CDR-H2 is a CDR-H2 selected from any one of SEQ ID NOs: 10-12, with up to 1, 2, 3, 4, 5, 6, 7, or 8 amino acid substitutions; the CDR-H1 is a CDR-H1 selected from any one of SEQ ID NOs: 1-9, with up to 1, 2, 3, 4, or 5 amino acid substitutions; the CDR-L3 is a CDR-L3 selected from any one of SEQ ID NOs: 27-31, 311-327, and 367-370, with up to 1, 2, 3, 4, or 5 amino acid substitutions; the CDR-L2 is a CDR-L2 selected from any one of SEQ ID NOs: 25-26, 303-310, and amino acid sequences DDS, DVS, EDS, DDT, DDK, DDL, DDN, DVS, and DDS, with up to 1, 2, 3, or 4 amino acid substitutions; and the CDR-L1 is a CDR-L1 selected from any one of SEQ ID NOs: 19-24, 271-302, and 387, with up to 1, 2, 3, 4, 5, or 6 amino acid substitutions.

In some embodiments, an antibody, or an antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27. In some embodiments, the CDR-H3 has at least about 80%, 90%, 91%, 92%, 93%, 94%, 95%, 96%, 97%, 98%, or 99% identity to a CDR-H3 selected from SEQ ID NOs: 13 and 16, the CDR-H2 has at least about 80%, 90%, 91%, 92%, 93%, 94%, 95%, 96%, 97%, 98%, or 99% identity to a CDR-H2 selected from SEQ ID NOs: 10, 11, and 12, the CDR-H1 has at least about 80%, 90%, 91%, 92%, 93%, 94%, 95%, 96%, 97%, 98%, or 99% identity to a CDR-H1 selected from SEQ ID NOs: 3, 6, and 9, the CDR-L3 has at least about 80%, 90%, 91%, 92%, 93%, 94%, 95%, 96%, 97%, 98%, or 99% identity to a CDR-L3 of SEQ ID NO: 27, the CDR-L2 has at least about 80%, 90%, 91%, 92%, 93%, 94%, 95%, 96%, 97%, 98%, or 99% identity to a CDR-L2 selected from SEQ ID NO: 26 and the amino acid sequence DDS, and the CDR-L1 has at least about 80%, 90%, 91%, 92%, 93%, 94%, 95%, 96%, 97%, 98%, or 99% identity to a CDR-L1 selected from SEQ ID NOs: 21 and 24. In some embodiments, the CDR-H3 is a CDR-H3 selected from SEQ ID NOs: 13 and 16, with up to 1, 2, 3, 4, 5, 6, 7, or 8 amino acid substitutions; the CDR-H2 is a CDR-H2 selected from SEQ ID NOs: 10, 11, and 12, with up to 1, 2, 3, 4, 5, 6, 7, or 8 amino acid substitutions; the CDR-H1 is a CDR-H1 selected from SEQ ID NOs: 3, 6, and 9, with up to 1, 2, 3, 4, or 5 amino acid substitutions; the CDR-L3 is a CDR-L3 of SEQ ID NO: 27 with up to 1, 2, 3, 4, or 5 amino acid substitutions; the CDR-L2 is a CDR-L2 selected from SEQ ID NO: 26 and the amino acid sequence DDS, with up to 1, 2, 3, or 4 amino acid substitutions; and the CDR-L1 is a CDR-L1 selected from SEQ ID NOs: 21 and 24, with up to 1, 2, 3, 4, 5, or 6 amino acid substitutions.

In some embodiments, an antibody, or an antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27.

In some embodiments, an antibody, or an antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30. In some embodiments, the CDR-H3 has at least about 80%, 90%, 91%, 92%, 93%, 94%, 95%, 96%, 97%, 98%, or 99% identity to a CDR-H3 selected from SEQ ID NOs: 13 and 16, the CDR-H2 has at least about 80%, 90%, 91%, 92%, 93%, 94%, 95%, 96%, 97%, 98%, or 99% identity to a CDR-H2 selected from SEQ ID NOs: 10, 11, and 12, the CDR-H1 has at least about 80%, 90%, 91%, 92%, 93%, 94%, 95%, 96%, 97%, 98%, or 99% identity to a CDR-H1 selected from SEQ ID NOs: 3, 6, and 9, the CDR-L3 has at least about 80%, 90%, 91%, 92%, 93%, 94%, 95%, 96%, 97%, 98%, or 99% identity to a CDR-L3 of SEQ ID NO: 30, the CDR-L2 has at least about 80%, 90%, 91%, 92%, 93%, 94%, 95%, 96%, 97%, 98%, or 99% identity to a CDR-L2 selected from SEQ ID NO: 25 and the amino acid sequence DVS, and the CDR-L1 has at least about 80%, 90%, 91%, 92%, 93%, 94%, 95%, 96%, 97%, 98%, or 99% identity to a CDR-L1 selected from SEQ ID NOs: 19 and 22. In some embodiments, the CDR-H3 is a CDR-H3 selected from SEQ ID NOs: 13 and 16, with up to 1, 2, 3, 4, 5, 6, 7, or 8 amino acid substitutions; the CDR-H2 is a CDR-H2 selected from SEQ ID NOs: 10, 11, and 12, with up to 1, 2, 3, 4, 5, 6, 7, or 8 amino acid substitutions; the CDR-H1 is a CDR-H1 of SEQ ID NOs: 3, 6, and 9, with up to 1, 2, 3, 4, or 5 amino acid substitutions; the CDR-L3 is a CDR-L3 of SEQ ID NO: 30 with up to 1, 2, 3, 4, or 5 amino acid substitutions; the CDR-L2 is a CDR-L2 selected from SEQ ID NO: 25 and the amino acid sequence DVS, with up to 1, 2, 3, or 4 amino acid substitutions; and the CDR-L1 is a CDR-L1 selected from SEQ ID NOs: 19 and 22, with up to 1, 2, 3, 4, 5, or 6 amino acid substitutions.

In some embodiments, an antibody, or an antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30.

In some embodiments, an antibody, or an antigen binding fragment thereof, provided herein comprises a CDR-H1 comprising an amino acid sequence set forth in any one of SEQ ID NOs: 1, 4, and 7; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 19 and 22; a CDR-L2 comprising an amino acid sequence set forth in SEQ ID NO: 25 or the amino acid sequence DVS; and a CDR-L3 comprising an amino acid sequence set forth in SEQ ID NO: 27.

In some embodiments, an antibody, or an antigen binding fragment thereof, provided herein comprises a CDR-H1 comprising an amino acid sequence set forth in any one of SEQ ID NOs: 2, 5, and 8; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 19 and 22; a CDR-L2 comprising an amino acid sequence set forth in SEQ ID NO: 25 or the amino acid sequence DVS; and a CDR-L3 comprising an amino acid sequence set forth in SEQ ID NO: 28.

In some embodiments, an antibody, or an antigen binding fragment thereof, provided herein comprises a CDR-H1 comprising an amino acid sequence set forth in any one of SEQ ID NOs: 2, 5, and 8; a CDR-H2 comprising an amino acid sequence set forth in any one of SEQ ID NOs: 10, 11, and 12; a CDR-H3 comprising an amino acid sequence set forth in any one of SEQ ID NOs: 14 and 17; a CDR-L1 comprising an amino acid sequence set forth in any one of SEQ ID NOs: 20 and 23; a CDR-L2 comprising an amino acid sequence set forth in SEQ ID NO: 26 or the amino acid sequence DDS; and a CDR-L3 comprising an amino acid sequence set forth in SEQ ID NO: 28.

In some embodiments, an antibody, or an antigen binding fragment thereof, provided herein comprises a CDR-H1 comprising an amino acid sequence set forth in any one of SEQ ID NOs: 2, 5, and 8; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 21 and 24; a CDR-L2 comprising an amino acid sequence set forth in SEQ ID NO: 25 or the amino acid sequence DVS; and a CDR-L3 comprising an amino acid sequence set forth in SEQ ID NO: 28.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 15 and 18; a CDR-L1 comprising an amino acid sequence set forth in any one of SEQ ID NOs: 21 and 24; a CDR-L2 comprising an amino acid sequence set forth in SEQ ID NO: 25 or the amino acid sequence DVS; and a CDR-L3 comprising an amino acid sequence set forth in SEQ ID NO: 29.

In some embodiments, an antibody, or an antigen binding fragment thereof, provided herein comprises a CDR-H1 comprising an amino acid sequence set forth in any one of SEQ ID NOs: 1, 4, and 7; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 19 and 22; a CDR-L2 comprising an amino acid sequence set forth in SEQ ID NO: 26 or the amino acid sequence DDS; and a CDR-L3 comprising an amino acid sequence set forth in SEQ ID NO: 31.

In some embodiments, an antibody, or an antigen binding fragment thereof, provided herein comprises a CDR-H1 comprising an amino acid sequence set forth in any one of SEQ ID NOs: 2, 5, and 8; a CDR-H2 comprising an amino acid sequence set forth in any one of SEQ ID NOs: 10, 11, and 12; a CDR-H3 comprising an amino acid sequence set forth in any one of SEQ ID NOs: 15 and 18; a CDR-L1 comprising an amino acid sequence set forth in any one of SEQ ID NOs: 21 and 24; a CDR-L2 comprising an amino acid sequence set forth in SEQ ID NO: 26 or the amino acid sequence DDS; and a CDR-L3 comprising an amino acid sequence set forth in SEQ ID NO: 28.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 271 and 289; a CDR-L2 comprising an amino acid sequence set forth in SEQ ID NO: 303 or the amino acid sequence DVS; and a CDR-L3 comprising an amino acid sequence set forth in SEQ ID NO: 311.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 272 and 290; a CDR-L2 comprising an amino acid sequence set forth in SEQ ID NO: 304 or the amino acid sequence EDS; and a CDR-L3 comprising an amino acid sequence set forth in SEQ ID NO: 312.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 273 and 291; a CDR-L2 comprising an amino acid sequence set forth in SEQ ID NO: 26 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 273 and 291; a CDR-L2 comprising an amino acid sequence set forth in SEQ ID NO: 26 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 273 and 291; a CDR-L2 comprising an amino acid sequence set forth in SEQ ID NO: 26 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 274 and 292; a CDR-L2 comprising an amino acid sequence set forth in SEQ ID NO: 26 or the amino acid sequence DDS; and a CDR-L3 comprising an amino acid sequence set forth in SEQ ID NO: 314.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 274 and 292; a CDR-L2 comprising an amino acid sequence set forth in SEQ ID NO: 26 or the amino acid sequence DDS; and a CDR-L3 comprising an amino acid sequence set forth in SEQ ID NO: 314.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 273 and 291; a CDR-L2 comprising an amino acid sequence set forth in SEQ ID NO: 26 or the amino acid sequence DDS; and a CDR-L3 comprising an amino acid sequence set forth in SEQ ID NO: 315.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 275 and 293; a CDR-L2 comprising an amino acid sequence set forth in SEQ ID NO: 305 or the amino acid sequence DDT; and a CDR-L3 comprising an amino acid sequence set forth in SEQ ID NO: 316.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 276 and 290; a CDR-L2 comprising an amino acid sequence set forth in SEQ ID NO: 26 or the amino acid sequence DDS; and a CDR-L3 comprising an amino acid sequence set forth in SEQ ID NO: 317.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 273 and 291; a CDR-L2 comprising an amino acid sequence set forth in SEQ ID NO: 26 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 273 and 291; a CDR-L2 comprising an amino acid sequence set forth in SEQ ID NO: 306 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 274 and 292; a CDR-L2 comprising an amino acid sequence set forth in SEQ ID NO: 26 or the amino acid sequence DDS; and a CDR-L3 comprising an amino acid sequence set forth in SEQ ID NO: 314.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 277 and 291; a CDR-L2 comprising an amino acid sequence set forth in SEQ ID NO: 26 or the amino acid sequence DDS; and a CDR-L3 comprising an amino acid sequence set forth in SEQ ID NO: 318.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 278 and 294; a CDR-L2 comprising an amino acid sequence set forth in SEQ ID NO: 26 or the amino acid sequence DDS; and a CDR-L3 comprising an amino acid sequence set forth in SEQ ID NO: 318.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 273 and 291; a CDR-L2 comprising an amino acid sequence set forth in SEQ ID NO: 26 or the amino acid sequence DDS; and a CDR-L3 comprising an amino acid sequence set forth in SEQ ID NO: 319.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 279 and 295; a CDR-L2 comprising an amino acid sequence set forth in SEQ ID NO: 26 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 280 and 296; a CDR-L2 comprising an amino acid sequence set forth in SEQ ID NO: 26 or the amino acid sequence DDS; and a CDR-L3 comprising an amino acid sequence set forth in SEQ ID NO: 320.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 281 and 297; a CDR-L2 comprising an amino acid sequence set forth in SEQ ID NO: 26 or the amino acid sequence DDS; and a CDR-L3 comprising an amino acid sequence set forth in SEQ ID NO: 321.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 282 and 298; a CDR-L2 comprising an amino acid sequence set forth in SEQ ID NO: 26 or the amino acid sequence DDS; and a CDR-L3 comprising an amino acid sequence set forth in SEQ ID NO: 322.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 274 and 292; a CDR-L2 comprising an amino acid sequence set forth in SEQ ID NO: 26 or the amino acid sequence DDS; and a CDR-L3 comprising an amino acid sequence set forth in SEQ ID NO: 314.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 277 and 291; a CDR-L2 comprising an amino acid sequence set forth in SEQ ID NO: 26 or the amino acid sequence DDS; and a CDR-L3 comprising an amino acid sequence set forth in SEQ ID NO: 318.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 281 and 297; a CDR-L2 comprising an amino acid sequence set forth in SEQ ID NO: 26 or the amino acid sequence DDS; and a CDR-L3 comprising an amino acid sequence set forth in SEQ ID NO: 323.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 276 and 290; a CDR-L2 comprising an amino acid sequence set forth in SEQ ID NO: 26 or the amino acid sequence DDS; and a CDR-L3 comprising an amino acid sequence set forth in SEQ ID NO: 29.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 283, 298, and 387; a CDR-L2 comprising an amino acid sequence set forth in SEQ ID NO: 307 or the amino acid sequence DDK; and a CDR-L3 comprising an amino acid sequence set forth in SEQ ID NO: 324.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 284 and 291; a CDR-L2 comprising an amino acid sequence set forth in SEQ ID NO: 26 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 285 and 299; a CDR-L2 comprising an amino acid sequence set forth in SEQ ID NO: 26 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 286 and 300; a CDR-L2 comprising an amino acid sequence set forth in SEQ ID NO: 308 or the amino acid sequence DDS; and a CDR-L3 comprising an amino acid sequence set forth in SEQ ID NO: 325.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 274 and 292; a CDR-L2 comprising an amino acid sequence set forth in SEQ ID NO: 26 or the amino acid sequence DDS; and a CDR-L3 comprising an amino acid sequence set forth in SEQ ID NO: 314.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 273 and 291; a CDR-L2 comprising an amino acid sequence set forth in SEQ ID NO: 26 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 274 and 292; a CDR-L2 comprising an amino acid sequence set forth in SEQ ID NO: 26 or the amino acid sequence DDS; and a CDR-L3 comprising an amino acid sequence set forth in SEQ ID NO: 314.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 273 and 291; a CDR-L2 comprising an amino acid sequence set forth in SEQ ID NO: 26 or the amino acid sequence DDS; and a CDR-L3 comprising an amino acid sequence set forth in SEQ ID NO: 313.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 281 and 297; a CDR-L2 comprising an amino acid sequence set forth in SEQ ID NO: 26 or the amino acid sequence DDS; and a CDR-L3 comprising an amino acid sequence set forth in SEQ ID NO: 29.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 274 and 292; a CDR-L2 comprising an amino acid sequence set forth in SEQ ID NO: 26 or the amino acid sequence DDS; and a CDR-L3 comprising an amino acid sequence set forth in SEQ ID NO: 314.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 274 and 292; a CDR-L2 comprising an amino acid sequence set forth in SEQ ID NO: 26 or the amino acid sequence DDS; and a CDR-L3 comprising an amino acid sequence set forth in SEQ ID NO: 326.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 287 and 301; a CDR-L2 comprising an amino acid sequence set forth in SEQ ID NO: 309 or the amino acid sequence DDL; and a CDR-L3 comprising an amino acid sequence set forth in SEQ ID NO: 327.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 288 and 302; a CDR-L2 comprising an amino acid sequence set forth in SEQ ID NO: 310 or the amino acid sequence DDN; and a CDR-L3 comprising an amino acid sequence set forth in SEQ ID NO: 29.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 19 and 22; a CDR-L2 comprising an amino acid sequence set forth in SEQ ID NO: 25 or the amino acid sequence DVS; and a CDR-L3 comprising an amino acid sequence set forth in SEQ ID NO: 368.

In some embodiments, an antibody, or an antigen binding fragment thereof, provided herein comprises a CDR-H1 comprising an amino acid sequence set forth in any one of SEQ ID NOs: 3, 6, and 9; a CDR-H2 comprising an amino acid sequence set forth in any one of SEQ ID NOs: 10, 11, and 12; a CDR-H3 comprising an amino acid sequence set forth in any one of SEQ ID NOs: 13 and 16; a CDR-L1 comprising an amino acid sequence set forth in any one of SEQ ID NOs: 21 and 24; a CDR-L2 comprising an amino acid sequence set forth in SEQ ID NO: 25 or the amino acid sequence DVS; and a CDR-L3 comprising an amino acid sequence set forth in SEQ ID NO: 369.

In some embodiments, the CDR-H3 has at least about 80%, 90%, 91%, 92%, 93%, 94%, 95%, 96%, 97%, 98%, or 99% identity to a CDR-H3 of TABLE 3; the CDR-H2 has at least about 80%, 90%, 91%, 92%, 93%, 94%, 95%, 96%, 97%, 98%, or 99% identity to a CDR-H2 of TABLE 3, the CDR-H1 has at least about 80%, 90%, 91%, 92%, 93%, 94%, 95%, 96%, 97%, 98%, or 99% identity to a CDR-H1 of TABLE 3, the CDR-L3 has at least about 80%, 90%, 91%, 92%, 93%, 94%, 95%, 96%, 97%, 98%, or 99% identity to a CDR-L3 of TABLE 3, the CDR-L2 has at least about 80%, 90%, 91%, 92%, 93%, 94%, 95%, 96%, 97%, 98%, or 99% identity to a CDR-L2 of TABLE 3, and the CDR-L1 has at least about 80%, 90%, 91%, 92%, 93%, 94%, 95%, 96%, 97%, 98%, or 99% identity to a CDR-L1 of TABLE 3. In some embodiments, the CDR-H3 is a CDR-H3 of TABLE 3, with up to 1, 2, 3, 4, 5, 6, 7, or 8 amino acid substitutions; the CDR-H2 is a CDR-H2 of TABLE 3, with up to 1, 2, 3, 4, 5, 6, 7, or 8 amino acid substitutions; the CDR-H1 is a CDR-H1 of TABLE 3, with up to 1, 2, 3, 4, or 5 amino acid substitutions; the CDR-L3 is a CDR-L3 of TABLE 3 with up to 1, 2, 3, 4, or 5 amino acid substitutions; the CDR-L2 is a CDR-L2 of TABLE 3, with up to 1, 2, 3, or 4 amino acid substitutions; and the CDR-L1 is a CDR-L1 of TABLE 3, with up to 1, 2, 3, 4, 5, or 6 amino acid substitutions.

In some embodiments, the CDR-H3 has at least about 80%, 90%, 91%, 92%, 93%, 94%, 95%, 96%, 97%, 98%, or 99% identity to a CDR-H3 of TABLE 5; the CDR-H2 has at least about 80%, 90%, 91%, 92%, 93%, 94%, 95%, 96%, 97%, 98%, or 99% identity to a CDR-H2 of TABLE 5, the CDR-H1 has at least about 80%, 90%, 91%, 92%, 93%, 94%, 95%, 96%, 97%, 98%, or 99% identity to a CDR-H1 of TABLE 5, the CDR-L3 has at least about 80%, 90%, 91%, 92%, 93%, 94%, 95%, 96%, 97%, 98%, or 99% identity to a CDR-L3 of TABLE 5, the CDR-L2 has at least about 80%, 90%, 91%, 92%, 93%, 94%, 95%, 96%, 97%, 98%, or 99% identity to a CDR-L2 of TABLE 5, and the CDR-L1 has at least about 80%, 90%, 91%, 92%, 93%, 94%, 95%, 96%, 97%, 98%, or 99% identity to a CDR-L1 of TABLE 5. In some embodiments, the CDR-H3 is a CDR-H3 of TABLE 5, with up to 1, 2, 3, 4, 5, 6, 7, or 8 amino acid substitutions; the CDR-H2 is a CDR-H2 of TABLE 5, with up to 1, 2, 3, 4, 5, 6, 7, or 8 amino acid substitutions; the CDR-H1 is a CDR-H1 of TABLE 5, with up to 1, 2, 3, 4, or 5 amino acid substitutions; the CDR-L3 is a CDR-L3 of TABLE 5, with up to 1, 2, 3, 4, or 5 amino acid substitutions; the CDR-L2 is a CDR-L2 of TABLE 5, with up to 1, 2, 3, or 4 amino acid substitutions; and the CDR-L1 is a CDR-L1 of TABLE 5, with up to 1, 2, 3, 4, 5, or 6 amino acid substitutions.

In certain embodiments of any of the antibodies or antigen binding fragments thereof described above, the antibody further comprises a heavy chain comprising a human IgG sequence selected from a sequence set forth in any one of SEQ ID NOs: 47-270. In certain embodiments of any of the antibodies or antigen binding fragments thereof described above, the antibody further comprises a constant light chain sequence comprising a sequence set forth in SEQ ID NO: 480. In certain embodiments of any of the antibodies or antigen binding fragments thereof described above, the antibody further comprises a heavy chain comprising a human IgG sequence selected from SEQ ID NO: 61 or SEQ ID NO: 173 and a constant light chain sequence comprising a sequence set forth in SEQ ID NO: 480.

In some embodiments, the antibody, or an antigen binding fragment thereof, comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 21 and 24; a CDR-L2 comprising the sequence set forth in SEQ ID NO: 26 or the amino acid sequence DDS; and a CDR-L3 comprising the sequence set forth in SEQ ID NO: 27. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 61 or SEQ ID NO: 173. In certain embodiments, the antibody comprises a human lambda light chain constant region (e.g., a lambda light chain constant region of subtype 1, 2, 3, or 7, such as a lambda light chain constant region set forth in any one of SEQ ID NOs: 480-483). In some embodiments, the antibody comprises a constant light chain sequence comprising a sequence set forth in SEQ ID NO: 480.

In some embodiments, an antibody, or an antigen binding fragment thereof, provided herein comprises a CDR-H1 comprising the sequence set forth in any one of SEQ ID NOs: 3, 6, and 9; a CDR-H2 comprising the sequence set forth in any one of SEQ ID NOs: 10, 11, and 12; a CDR-H3 comprising the sequence set forth in any one of SEQ ID NOs: 13 and 16; a CDR-L1 comprising the sequence set forth in any one of SEQ ID NOs: 19 and 22; a CDR-L2 comprising the sequence set forth in SEQ ID NO: 25 or the amino acid sequence DVS; and a CDR-L3 comprising the sequence set forth in SEQ ID NO: 30. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 61 or SEQ ID NO: 173. In certain embodiments, the antibody comprises a human lambda light chain constant region (e.g., a lambda light chain constant region of subtype 1, 2, 3, or 7, such as a lambda light chain constant region set forth in any one of SEQ ID NOs: 480-483). In some embodiments, the antibody comprises a constant light chain sequence comprising a sequence set forth in SEQ ID NO: 480.

Although a C-terminal lysine may be present in the corresponding coding sequence of the constant heavy chain region (e.g., in a sequence encoding any one of SEQ ID NOs: 159-270), it may be cleaved off during manufacture or after administration (resulting in, e.g., a constant heavy chain sequence of any one of SEQ ID NOs: 40-158). Accordingly, any of the antibodies or antigen binding fragments thereof described above may comprise a human IgG sequence containing a C-terminal lysine (e.g., any one of SEQ ID NOs: 159-270), a human IgG sequence lacking a C-terminal lysine (e.g., any one of SEQ ID NOs: 40-158), or a mixture thereof (e.g., a mixture of the same heavy chain constant sequence with and without a C-terminal lysine, such as a mixture of SEQ ID NO: 173 and SEQ ID NO: 61).

In some aspects, the amino acid substitutions are conservative amino acid substitutions. In some embodiments, the antibodies described in this disclosure are referred to herein as “variants” or “clones.” In some embodiments, such variants or clones are derived from a sequence provided herein, for example, by affinity maturation, site directed mutagenesis, random mutagenesis, or any other method known in the art or described herein. In some embodiments, such variants or clones are not derived from a sequence provided herein and may, for example, be isolated de novo according to the methods provided herein for obtaining antibodies.

### Fc Region

The structures of the Fc regions of various immunoglobulins, and the glycosylation sites contained therein, are known in the art. See Schroeder and Cavacini, J. (2010) Allergy Clin. Immunol. 125:S41-52, incorporated by reference in its entirety. The Fc region may be a naturally occurring Fc region, or an Fc region modified as described in the art or elsewhere in this disclosure.

Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG1, IgG2, IgG3, IgG4, IgA1, and IgA2.

In certain embodiments, provided human IgG1 Fc regions include an SRDEL (SEQ ID NO: 507) allotype or an SREEM (SEQ ID NO: 508) allotype.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 domain can affect the binding of FcRs to the Fc. A number of amino acid modifications in the Fc region are known that can selectively alter the affinity of the Fc for different Fc-gamma receptors. In some aspects, the Fc comprises one or more modifications designed to promote selective binding of Fc-gamma receptors.

Exemplary mutations that may alter the binding of FcRs to the Fc are listed below (in EU numbering format):
S298A/E333A/K334A, S298A/E333A/K334A/K326A (Lu et al., J Immunol Methods. 2011 Feb. 28; 365(1-2): 132-41);
F243L/R292P/Y300L/V305I/P396L, F243L/R292P/Y300L/L235V/P396L (Stavenhagen et al., Cancer Res. 2007 Sep. 15; 67(18): 8882-90; Nordstrom et al., Breast Cancer Res. 2011 Nov. 30; 13(6): R123);
F243L (Stewart et al., Protein Eng Des Sel. 2011 September; 24(9): 671-8), S298A/E333A/K334A (Shields et al., J Biol Chem. 2001 Mar. 2; 276(9): 6591-604);
S239D/1332E/A330L, S239D/1332E (Lazar et al., Proc Natl Acad Sci USA. 2006 Mar. 14; 103(11): 4005-10);
S239D/S267E, S267E/L328F (Chu et al., Mol Immunol. 2008 September; 45(15): 3926-33); S239D/D265S/S298A/1332E, S239E/S298A/K326A/A327H, G237F/S298A/A330L/1332E, S239D/1332E/S298A, S239D/K326E/A330L/1332E/S298A, G236A/S239D/D270L/1332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments, an antibody, or an antigen binding fragment thereof, described herein includes modifications intended to improve its ability to mediate effector function. Such modifications that can have this effect are known in the art and include afucosylation, or engineering of the affinity of the Fc towards an activating receptor, mainly FCGR3a for ADCC, and towards C1q for CDC. The following TABLE 7 summarizes various designs reported in the literature for effector function engineering.

Methods of producing antibodies with little or no fucose on the Fc glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 2010 December; 20 (12): 1607-18). Examples of cell lines capable of producing defucosylated antibody include CHO-DG44 with stable overexpression of the bacterial oxidoreductase GDP-6-deoxy-D-lyxo-4-hexylose reductase (RMD) (see Henning von Horsten et al., Glycobiol 2010, 20:1607-1618) or Lec13 CHO cells, which are deficient in protein fucosylation (see Ripka et al., Arch. Biochem. Biophys., 1986, 249:533-545; U.S. Pat. Pub. No. 2003/0157108; and WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87:614-622; Kanda et al., Biotechnol. Bioeng., 2006, 94:680-688; and WO 2003/085107; each of which is incorporated by reference in its entirety). Another approach to obtaining antibodies with lowered levels of fucosylation can be found in U.S. Pat. No. 8,409,572, which teaches selecting cell lines for antibody production for their ability to yield lower levels of fucosylation on antibodies.

Antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

In some aspects, an antibody, or an antigen binding fragment thereof, provided herein comprises an Fc domain (e.g., an IgG1 domain) with reduced fucose content at position Asn 297 compared to a naturally occurring IgG1 domain. Such Fc domains are known to have improved ADCC. See Shields et al., J. Biol. Chem., 2002, 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In certain embodiments, an antibody, or an antigen binding fragment thereof, provided herein comprises an Fc region with one or more amino acid substitutions which improve ADCC, such as a substitution at one or more of positions 298, 333, and 334 of the Fc region. In some embodiments, an antibody, or an antigen binding fragment thereof, provided herein comprises an Fc region with one or more amino acid substitutions at positions 239, 332, and 330, as described in Lazar et al., Proc. Natl. Acad. Sci. USA, 2006, 103:4005-4010, incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, and 2004/0109865; International Pat. Pub. Nos. 2000/61739, 2001/29246, 2003/085119, 2003/084570, 2005/035586, 2005/035778; 2005/053742, and 2002/031140; Okazaki et al., J. Mol. Biol., 2004, 336:1239-1249; and Yamane-Ohnuki et al., Biotech. Bioeng., 2004, 87:614-622; each of which is incorporated by reference in its entirety.

In some embodiments, an antibody, or an antigen binding fragment thereof, provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which is incorporated by reference in its entirety.

Thus, in one embodiment, an antibody described herein can include a dimeric Fc that comprises one or more amino acid modifications as noted in TABLE 7 that may confer improved effector function. In another embodiment, the antibody can be afucosylated to improve effector function.

TABLE 7
CH2 domains and effector function engineering

| Reference | Mutations | Effect |
|---|---|---|
| Lu (2011), supra, Ferrara | Afucosylated | Increased ADCC |
| (2011), supra, Mizushima | | |
| et al. (2011) Genes Cells | | |
| 16(11): 1071-1080. | | |
| Lu (2011), supra | S298A/E333A/K334A | Increased ADCC |
| Lu (2011), supra | S298A/E333A/ | Increased ADCC |
| | K334A/K326A | |
| Stavenhagen (2007), | F243L/R292P/Y300L/ | Increased ADCC |
| supra | V305I/P396L | |
| Nordstrom (2011), supra | F243L/R292P/Y300L/ | Increased ADCC |
| | L235V/P396L | |
| Stewart (2011), supra | F243L | Increased ADCC |
| Shields (2001), supra | S298A/E333A/K334A | Increased ADCC |
| Lazar (2006), supra | S239D/I332E/A330L | Increased ADCC |
| Lazar (2006), supra | S239D/I332E | Increased ADCC |
| Bowles et al., (2006) | AME-D, not specified | Increased ADCC |
| Blood 108(8):2648-54 | mutations | |
| Heider et al., (2011) | 37.1, mutations not | Increased ADCC |
| Blood 118(15):4159-68 | disclosed | |
| Moore et al., (2010) | S267E/H268F/S324T | Increased CDC |
| MAbs 2(2):181-9 | | |

Fc modifications intended to reduce FcgR and/or complement binding and/or effector function are known in the art. Recent publications describe strategies that have been used to engineer antibodies with reduced or silenced effector activity (see Strohl, WR (2009), Curr Opin Biotech 20:685-691, and Strohl, WR and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, U.S. Patent Publication No. 2011/0212087 (Strohl), International Patent Publication No. WO 2006/105338 (Xencor), U.S. Patent Publication No. 2012/0225058 (Xencor), U.S. Patent Publication No. 2012/0251531 (Genentech), and Strop et al. ((2012) J. Mol. Biol. 420:204-219), each of which is incorporated by reference in its entirety, describe specific modifications to reduce FcgR or complement binding to the Fc.

Specific, non-limiting examples of amino acid modifications intended to reduce FcgR or complement binding to the Fc include those identified in the following TABLE 8:

TABLE 8
Modifications designed to reduce FcgR or complement binding to the Fc
Mutations
N297A
L234A/L235A
IgG2 V234A/G237A
IgG4 L235A/G237A/E318A
IgG4 S228P/L236E
IgG2/IgG4
IgG2 H268Q/V309L/A330S/A331S
C220S/C226S/C229S/P238S
C226S/C229S/E3233P/L235V/L235A
E. coli production, non glyco
L234F/L235E/P331S
Hinge mutant, e.g., C226S/P230S
D265A
L235A/G237A
L234A/L235A/G237A
L234A/L235A/P329G

In some embodiments, an antibody, or an antigen binding fragment thereof, provided herein comprises one or more alterations that are designed to improve or diminish C1q binding and/or CDC. See U.S. Pat. No. 6,194,551; WO 99/51642; and Idusogie et al., J. Immunol., 2000, 164:4178-4184; each of which is incorporated by reference in its entirety.

In certain embodiments, the heavy chain comprises a constant heavy chain sequence selected from the sequences set forth in SEQ ID NOs: 47-270. In certain embodiments, the constant heavy chain sequence, (e.g., a constant heavy chain sequence selected from SEQ ID NOs: 47-158) further comprises a C-terminal lysine (SEQ ID NOs: 159-270). Although a C-terminal lysine may be present in the corresponding coding sequence of the constant heavy chain region, it may be cleaved off during manufacture or after administration. Accordingly, sequences of heavy chain constant regions with and without the C-terminal lysine are provided herein. Consequently, a composition resulting from the manufacture of an antibody comprising a C-terminal lysine in the corresponding coding sequence of the constant heavy chain region (e.g., an antibody with a coding sequence that encodes any one of SEQ ID NOs: 159-270) may comprise antibodies having a constant heavy chain sequence containing a C-terminal lysine (e.g., selected from any one of SEQ ID NOs: 159-270), antibodies having a constant heavy chain sequence lacking a C-terminal lysine (e.g., the corresponding sequence of any one of SEQ ID NOs: 47-158), or a mixture thereof. For example, a composition comprising an antibody comprising the constant heavy chain sequence of SEQ ID NO: 173 that is administered to a subject may comprise antibodies having the constant heavy chain sequence set forth in SEQ ID NO: 173 or SEQ ID NO: 61, or a mixture thereof (e.g., a mixture of antibodies having either a constant heavy chain sequence of SEQ ID NO: 173 or a constant heavy chain sequence of SEQ ID NO: 61 and/or antibodies containing both constant heavy chain sequences (e.g., in a single antibody containing two constant heavy chain sequences)).

In certain embodiments, an antibody, or an antigen binding fragment thereof, provided herein comprises a VH sequence and a VL sequence provided in TABLE 4 (e.g., a VH sequence and a VL sequence from the same row of TABLE 4), together with a heavy chain constant region selected from a sequence set forth in SEQ ID NOs: 47-270. In certain embodiments, an antibody, or an antigen binding fragment thereof, provided herein comprises a VH sequence and a VL sequence provided in TABLE 4, together with a heavy chain constant region and a light chain constant region selected from the sequences set forth in SEQ ID NOs: 46 and 480-483.

In certain embodiments, an antibody, or an antigen binding fragment thereof, provided herein comprises a VH sequence and a VL sequence provided in TABLE 6 (e.g., a VH sequence and a VL sequence from the same row of TABLE 6), together with a heavy chain constant region selected from a sequence set forth in SEQ ID NOs: 47-270. In certain embodiments, an antibody, or an antigen binding fragment thereof, provided herein comprises a VH sequence and a VL sequence provided in TABLE 6, together with a heavy chain constant region and a light chain constant region selected from the sequences set forth in SEQ ID NOs: 46 and 480-483.

In certain embodiments, the antibody, or antigen binding fragment thereof, comprises a VH sequence set forth in SEQ ID NO: 34 and a VL sequence set forth in SEQ ID NO: 41; and the constant heavy chain comprises a human IgG sequence selected from a sequence set forth in SEQ ID NOs: 47-270 (e.g., SEQ ID NO: 61 or SEQ ID NO: 173). In certain embodiments, the antibody, or an antigen binding fragment thereof, comprises a VH sequence set forth in SEQ ID NO: 34 and a VL sequence set forth in SEQ ID NO: 43; and the constant heavy chain comprises a human IgG sequence selected from a sequence set forth in SEQ ID NOs: 47-270 (e.g., SEQ ID NO: 61 or SEQ ID NO: 173). In certain embodiments, the antibody comprises a human lambda light chain constant region sequence (e.g., any one of SEQ ID NOs: 480-483).

In certain embodiments, the antibody comprises a heavy chain comprising an amino acid sequence set forth in SEQ ID NO: 484 and/or SEQ ID NO: 486. In certain embodiments, the antibody comprises a light chain comprising an amino acid sequence set forth in SEQ ID NO: 485. In certain embodiments, the antibody comprises a heavy chain comprising an amino acid sequence set forth in SEQ ID NO: 484 and a light chain comprising an amino acid sequence set forth in SEQ ID NO: 485. In certain embodiments, the antibody comprises a heavy chain comprising an amino acid sequence set forth in SEQ ID NO: 486 and a light chain comprising an amino acid sequence set forth in SEQ ID NO: 485.

In certain embodiments, the antibody comprises a heavy chain comprising an amino acid sequence set forth in SEQ ID NO: 487. In certain embodiments, the antibody comprises a light chain comprising an amino acid sequence set forth in SEQ ID NO: 488. In certain embodiments, the antibody comprises a heavy chain comprising an amino acid sequence set forth in SEQ ID NO: 487 and a light chain comprising an amino acid sequence set forth in SEQ ID NO: 488.

In certain embodiments, the antibody comprises a heavy chain comprising an amino acid sequence set forth in SEQ ID NO: 489. In certain embodiments, the antibody comprises a light chain comprising an amino acid sequence set forth in SEQ ID NO: 490. In certain embodiments, the antibody comprises a heavy chain comprising an amino acid sequence set forth in SEQ ID NO: 489 and a light chain comprising an amino acid sequence set forth in SEQ ID NO: 490.

In certain embodiments, the antibody comprises a heavy chain comprising an amino acid sequence set forth in SEQ ID NO: 491. In certain embodiments, the antibody comprises a light chain comprising an amino acid sequence set forth in SEQ ID NO: 492. In certain embodiments, the antibody comprises a heavy chain comprising an amino acid sequence set forth in SEQ ID NO: 491 and a light chain comprising an amino acid sequence set forth in SEQ ID NO: 492.

In certain embodiments, the antibody comprises a heavy chain comprising an amino acid sequence set forth in SEQ ID NO: 493. In certain embodiments, the antibody comprises a light chain comprising an amino acid sequence set forth in SEQ ID NO: 494. In certain embodiments, the antibody comprises a heavy chain comprising an amino acid sequence set forth in SEQ ID NO: 493 and a light chain comprising an amino acid sequence set forth in SEQ ID NO: 494.

In certain embodiments, the antibody comprises a heavy chain comprising an amino acid sequence set forth in SEQ ID NO: 495. In certain embodiments, the antibody comprises a light chain comprising an amino acid sequence set forth in SEQ ID NO: 496. In certain embodiments, the antibody comprises a heavy chain comprising an amino acid sequence set forth in SEQ ID NO: 495 and a light chain comprising an amino acid sequence set forth in SEQ ID NO: 496.

In certain embodiments, the antibody comprises a heavy chain comprising an amino acid sequence set forth in SEQ ID NO: 497. In certain embodiments, the antibody comprises a light chain comprising an amino acid sequence set forth in SEQ ID NO: 498. In certain embodiments, the antibody comprises a heavy chain comprising an amino acid sequence set forth in SEQ ID NO: 497 and a light chain comprising an amino acid sequence set forth in SEQ ID NO: 498.

In certain embodiments, the antibody comprises a heavy chain comprising an amino acid sequence set forth in SEQ ID NO: 499. In certain embodiments, the antibody comprises a light chain comprising an amino acid sequence set forth in SEQ ID NO: 500. In certain embodiments, the antibody comprises a heavy chain comprising an amino acid sequence set forth in SEQ ID NO: 499 and a light chain comprising an amino acid sequence set forth in SEQ ID NO: 500.

In certain embodiments, the antibody comprises a heavy chain comprising an amino acid sequence set forth in SEQ ID NO: 501. In certain embodiments, the antibody comprises a light chain comprising an amino acid sequence set forth in SEQ ID NO: 502. In certain embodiments, the antibody comprises a heavy chain comprising an amino acid sequence set forth in SEQ ID NO: 501 and a light chain comprising an amino acid sequence set forth in SEQ ID NO: 502.

In certain embodiments, the antibody comprises a heavy chain comprising an amino acid sequence set forth in SEQ ID NO: 503. In certain embodiments, the antibody comprises a light chain comprising an amino acid sequence set forth in SEQ ID NO: 504. In certain embodiments, the antibody comprises a heavy chain comprising an amino acid sequence set forth in SEQ ID NO: 503 and a light chain comprising an amino acid sequence set forth in SEQ ID NO: 504.

In certain embodiments, the antibody comprises a heavy chain comprising an amino acid sequence set forth in SEQ ID NO: 505. In certain embodiments, the antibody comprises a light chain comprising an amino acid sequence set forth in SEQ ID NO: 506. In certain embodiments, the antibody comprises a heavy chain comprising an amino acid sequence set forth in SEQ ID NO: 505 and a light chain comprising an amino acid sequence set forth in SEQ ID NO: 506.

In certain embodiments, the Fc region comprises one or more amino acid substitutions, wherein the one or more amino acid substitutions result in an increase in one or more of antibody half-life, ADCC activity, ADCP activity, or CDC activity compared with the Fc region without the one or more amino acid substitutions. In certain embodiments, the one or more amino acid substitutions results in increased antibody half-life at pH 6.0 compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to tezepelumab. In certain embodiments, the antibody has an increased half-life that is about 2.5-fold, 2.4-fold, 2.3-fold, 2.2-fold, 2.1-fold, 2.0-fold, 1.9-fold, or 1.8-fold longer compared to tezepelumab. In certain embodiments, the one or more amino acid substitutions results in increased antibody half-life in a subject (e.g., in a human subject) compared to an antibody comprising a wild-type Fc region.

In certain embodiments, an antibody described herein comprises a heavy chain constant domain having a means for increasing the half-life of the antibody.

In some embodiments, in a non-human primate, the half-life of an anti-TSLP antibody described herein is at least 14, 15, 16, 17, 18, or 19 days when the anti-TSLP antibody is administered intravenously.

In some embodiments, in a non-human primate, the half-life of an anti-TSLP antibody described herein is at least 17, 18, 19, 20, 21, 22, or 23 days when the anti-TSLP antibody is administered subcutaneously.

In certain embodiments, the Fc region comprises one or more amino acid substitutions, wherein the one or more substitutions result in a decrease in one or more of ADCC activity, ADCP activity, or CDC activity compared with the Fc without the one or more substitutions.

In certain embodiments, the one or more amino acid substitutions is selected from the group consisting of S228P (SP), T250Q, M252Y, S254T, T256E, T256D, H285D, T307A, T307Q, T307R, T307W, L309D, Q411H, Q311V, A378V, E380A, M428L, N434A, N434S, N297A, D265A, L234A, L235A, and N434W (or, e.g., L239A, L240A, M257Y, S259T, and T261E using direct numbering). In certain embodiments, the one or more amino acid substitutions comprises a specific combination of amino acid substitutions selected from the group consisting of M428L/N434S (LS), M252Y/S254T/T256E (YTE) or M257Y/S259T/T261E (YTE) using direct numbering, T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA) or L239A/L240A (LALA) using direct numbering, M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

Although the EU numbering system is typically used to identify the positions of the various Fc mutations described herein, direct numbering can also be used. For example, in certain embodiments, an antibody described herein comprises an Fc region with YTE mutations at positions 257, 259, and 261, respectively. In certain embodiments, an antibody described herein comprises an Fc region with LALA mutations at positions 239 and 240, respectively. In certain embodiments, an antibody described herein comprises an Fc region with YTE mutations at positions 257, 259, and 261, respectively, and with LALA mutations at positions 239 and 240, respectively. In certain embodiments, an antibody described herein comprises the VH and VL of Antibody 5 and an Fc region comprising YTE mutations at positions 257, 259, and 261 (M257Y/S259T/T261E), respectively, and with LALA mutations at positions 239 and 240 (L239A/L240A), respectively.

In certain embodiments the human Fc region comprises a human IgG1 Fc region with LALA mutations. In certain embodiments, the human Fc region comprises a human IgG1 Fc region with YTE mutations. In certain embodiments, the human Fc region comprises a human IgG1 Fc region with LALA and YTE mutations. In certain embodiments, when direct numbering is used, “YTE” and “LALA” mutations can be located at different amino acid position numbers. For example, a human Fc region can comprise a human IgG1 Fc region with LALA mutations at L239A/L240A and/or YTE mutations at M257Y/S259T/T261E.

In certain embodiments, the Fc region binds an Fcγ Receptor selected from the group consisting of: FcγRI, FcγRIIa, FcγRIIb, FcγRIIc, FcγRIIIa, and FcγRIIIb. In certain embodiments, the Fc region binds an Fcγ Receptor with higher affinity at pH 6.0 compared to an antibody comprising a wild-type Fc region.

### Binding

The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule (i.e., an TSLP) and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the affinity of an anti-TSLP antibody, or an antigen binding fragment thereof, for a non-target molecule is less than about 50% of the affinity for TSLP. In some embodiments, the affinity of an anti-TSLP antibody, or an antigen binding fragment thereof, for a non-target molecule is less than about 40% of the affinity for TSLP. In some embodiments, the affinity of an anti-TSLP antibody, or an antigen binding fragment thereof, for a non-target molecule is less than about 30% of the affinity for TSLP. In some embodiments, the affinity of an anti-TSLP antibody, or an antigen binding fragment thereof, for a non-target molecule is less than about 20% of the affinity for TSLP. In some embodiments, the affinity of an anti-TSLP antibody, or an antigen binding fragment thereof, for a non-target molecule is less than about 10% of the affinity for TSLP. In some embodiments, the affinity of an anti-TSLP antibody, or an antigen binding fragment thereof, for a non-target molecule is less than about 1% of the affinity for TSLP. In some embodiments, the affinity of an anti-TSLP antibody, or an antigen binding fragment thereof, for a non-target molecule is less than about 0.1% of the affinity for TSLP.

When used herein in the context of two or more antibodies, the term “competes with” or “cross-competes with” indicates that the two or more antibodies compete for binding to an antigen (e.g., TSLP). In one exemplary assay, TSLP is coated on a surface and contacted with a first anti-TSLP antibody, after which a second anti-TSLP antibody is added. In another exemplary assay, a first anti-TSLP antibody is coated on a surface and contacted with TSLP, and then a second anti-TSLP antibody is added. If the presence of the first anti-TSLP antibody reduces binding of the second anti-TSLP antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, at least 95%, or at least 99% as measured in a competitive binding assay. A skilled artisan can select the concentrations of the antibodies used in the competition assays based on the affinities of the antibodies for TSLP and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

A test antibody competes with a reference antibody if an excess of a test antibody (e.g., at least 2×, 5×, 10×, 20×, or 100×) inhibits or blocks binding of the reference antibody by, e.g., at least 50%, 60%, 70%, 75%, 80%, 85%, 90%, 95%, or 99% as measured in a competitive binding assay. Antibodies identified by competition assay (competing antibody) include antibodies binding to the same epitope as the reference antibody and antibodies binding to an adjacent epitope sufficiently proximal to the epitope bound by the reference antibody for steric hindrance to occur. For example, a second, competing antibody can be identified that competes for binding to TSLP with a first antibody described herein. In certain instances, the second antibody can block or inhibit binding of the first antibody by, e.g., at least 50%, 60%, 70%, 75%, 80%, 85%, 90%, 95%, or 99% as measured in a competitive binding assay. In certain instances, the second antibody can displace the first antibody by greater than 50%, 60%, 70%, 75%, 80%, 85%, 90%, 95%, or 99%.

In certain embodiments, the antibody, or an antigen binding fragment thereof, binds a TSLP sequence set forth in SEQ ID NO: 365 or 366.

Provided herein is an antibody or an antigen-binding fragment thereof that binds an epitope of TSLP within amino acids 15-31 of SEQ ID NO: 365 (YLSTISKDLITYMSGTK; SEQ ID NO: 402). In certain embodiments, the epitope e.g., an epitope of TSLP within amino acids 15-31 of SEQ ID NO: 365 is measured by cross-linking mass spectrometry.

Also provided herein is an antibody or an antigen-binding fragment thereof that binds an epitope of TSLP within amino acids 20-26 of SEQ ID NO: 365 (SKDLITY; SEQ ID NO: 408) and amino acids 66-70 of SEQ ID NO: 365 (AKEMF; SEQ ID NO: 401) but not amino acids 120-128 of SEQ ID NO: 365 (WRRFNRPLL; SEQ ID NO: 404).

In certain embodiments, the antibody, or an antigen binding fragment thereof, binds to a TSLP sequence set forth in SEQ ID NO: 365 or 366 with a K_{D} of less than or equal to about 1, 2, 3, 4, 5, 6, 7, 8, or 9×10⁻⁹ M, as measured by surface plasmon resonance (SPR). In certain embodiments, the antibody, or an antigen binding fragment thereof, binds to a TSLP sequence set forth in SEQ ID NO: 365 or 366 with a K_{D} of less than or equal to about 1×10⁻¹⁰ M, as measured by SPR. In certain embodiments, the antibody, or an antigen binding fragment thereof, binds to human TSLP with a K_{D} of less than or equal to about 1×10⁻⁹M, as measured by SPR.

In some embodiments, an antibody, or an antigen binding fragment thereof, provided herein binds TSLP with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁸ M, as measured by ELISA or any other suitable method known in the art. In some embodiments, an antibody, or an antigen binding fragment thereof, provided herein binds TSLP with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁹ M, as measured by ELISA or any other suitable method known in the art.

In some embodiments, the K_{D} of the antibody, or an antigen binding fragment thereof, provided herein for the binding of TSLP is between about 0.001-0.01, 0.01-0.1, 0.01-0.05, 0.05-0.1, 0.1-0.5, 0.5-1, 0.25-0.75, 0.25-0.5, 0.5-0.75, 0.75-1, 0.75-2, 1.1-1.2, 1.2-1.3, 1.3-1.4, 1.4-1.5, 1.5-1.6, 1.6-1.7, 1.7-1.8, 1.8-1.9, 1.9-2, 1-2, 1-5, 2-7, 3-8, 3-5, 4-6, 5-7, 6-8, 7-9, 7-10, or 5-10×10⁻⁸ M, as measured by ELISA or any other suitable method known in the art. In some embodiments, an antibody, or an antigen binding fragment thereof, provided herein binds TSLP with a K_{D} of less than or equal to about 1×10⁻⁸ M, or less than or equal to about 1×10⁻⁹ M as measured by ELISA or any other suitable method known in the art.

In some embodiments, the antibody, or an antigen binding fragment thereof, provided herein binds TSLP with a K_{D} of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁸ M, or less, as measured by ELISA or any other suitable method known in the art. In some embodiments, the antibody, or an antigen binding fragment thereof, provided herein binds TSLP with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

In some embodiments, the antibody, or an antigen binding fragment thereof, provided herein binds FcRn with an affinity at pH 7.4 compared to pH 6.0 at a ratio (pH 7.4/pH 6.0) of about 10,000, 1,000, 500, 100, 50, 20, 10, 9, 8, 7, 6, 5, 4.5, 4, 3.5, 3, 2.5, 2, 1.95, 1.9, 1.85, 1.8, 1.75, 1.7, 1.65, 1.6, 1.55, 1.50, 1.45, 1.4, 1.3, 1.2, 1.1, or 1.05, as measured by ELISA or any other suitable method known in the art. In some embodiments, the antibody, or an antigen binding fragment thereof, provided herein binds FcRn with an affinity at pH 6.0 compared to pH 7.4 at a ratio (pH 6.0/pH 7.4) of about 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

### Function

“Effector functions” refer to those biological activities mediated by the Fc region of an antibody, which activities may vary depending on the antibody isotype. Examples of antibody effector functions include receptor ligand blocking, agonism, or antagonism, C1q binding to activate complement dependent cytotoxicity (CDC), Fc receptor binding to activate antibody-dependent cellular cytotoxicity (ADCC), and antibody dependent cellular phagocytosis (ADCP). In some embodiments, the anti-TSLP antibody described herein includes modifications to improve its ability to mediate effector function. Such modifications are known in the art and include afucosylation, or engineering of the affinity of the Fc towards an activating receptor, mainly FCGR3a for antibody-dependent cellular cytotoxicity (ADCC), and towards C1q for complement-dependent cytotoxicity (CDC).

In some aspects, the anti-TSLP antibody provided herein comprises a Fc domain (e.g., IgG1) with reduced fucose content at position Asn 297 (EU numbering) compared to a naturally occurring Fc domain. Such Fc domains are known to have improved ADCC. In some aspects, such antibodies do not comprise any fucose at position Asn 297.

In some embodiments, the anti-TSLP antibody described herein comprises an Fc region with one or more amino acid substitutions which improve ADCC, such as a substitution at one or more of positions 298, 333, and 334 of the Fc region. In some embodiments, the anti-TSLP antibody provided herein comprises an Fc region with one or more amino acid substitutions at positions 239, 332, and 330.

### Pharmaceutical Compositions

The present application provides compositions comprising the antibodies or antigen binding fragments thereof described herein, including pharmaceutical compositions comprising any one or more of the antibodies or antigen binding fragments thereof described herein with one or more pharmaceutically acceptable excipients. In some embodiments the composition is sterile. The pharmaceutical compositions generally comprise an effective amount of an antibody, or an antigen binding fragment thereof. In some embodiments, the pharmaceutical compositions further comprise a hyaluronidase or a variant thereof.

These compositions can comprise, in addition to one or more of the antibodies or antigen binding fragments thereof disclosed herein, a pharmaceutically acceptable excipient, carrier, buffer, stabilizer, or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, and intraperitoneal routes.

Pharmaceutical compositions for oral administration can be in tablet, capsule, powder, or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose, or other saccharide solution or glycols such as ethylene glycol, propylene glycol, or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, and Lactated Ringer's Injection. Preservatives, stabilizers, buffers, antioxidants, and/or other additives can be included, as required.

For the anti-TSLP antibody, or an antigen binding fragment thereof, that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on a number of factors, e.g., the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration, and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Methods

### Methods of Preparation

Antibodies or antigen binding fragments thereof described herein can be produced using recombinant methods and compositions, e.g., as described in U.S. Pat. No. 4,816,567. In one embodiment, an isolated nucleic acid encoding an antibody, or an antigen binding fragment thereof, described herein is provided. Such a nucleic acid may encode an amino acid sequence comprising the VL and/or an amino acid sequence comprising the VH of the antibody (e.g., the light and/or heavy chains of the antibody) or an amino acid sequence comprising the VHH of a single domain antibody. In a further embodiment, one or more vectors (e.g., expression vectors) comprising such a nucleic acid are provided. In one embodiment, the nucleic acid is provided in a multicistronic vector. In a further embodiment, a host cell comprising such a nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g. a Chinese Hamster Ovary (CHO) cell, human embryonic kidney (HEK) cell, or lymphoid cell (e.g., Y0, NS0, Sp20 cell). In one embodiment, a method of making an antibody is provided, wherein the method comprises culturing a host cell comprising nucleic acid encoding the antibody, as provided above, under conditions suitable for expression of the antibody, and optionally recovering the antibody from the host cell (or host cell culture medium).

For recombinant production of the antibody, a nucleic acid encoding an antibody, e.g., as described above, is isolated and inserted into one or more vectors for further cloning and/or expression in a host cell. Such a nucleic acid may be readily isolated and sequenced using conventional procedures (e.g., by using oligonucleotide probes that are capable of binding specifically to genes encoding the heavy and light chains of the antibody).

When an antibody or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the antibody or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, a “substantially purified” antibody produced by the methods described herein has a purity level of at least about 30%, at least about 35%, at least about 40%, at least about 45%, at least about 50%, at least about 55%, at least about 60%, at least about 65%, at least about 70%, specifically, a purity level of at least about 75%, at least about 80%, or at least about 85%, and more specifically, a purity level of at least about 90%, a purity level of at least about 95%, or a purity level of at least about 99% or greater as determined by appropriate methods such as SDS/PAGE analysis, RP-HPLC, SEC, and capillary electrophoresis.

Suitable host cells for cloning or expression of antibody-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp2O, CV-1, VERO-76, HeLa, HepG2, Per.C6, or BHK.

For example, an antibody may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the antibodies described herein are produced in stable mammalian cells, by a method comprising: transfecting at least one stable mammalian cell with a nucleic acid encoding the antibody, in a predetermined ratio, and expressing the nucleic acid in the at least one mammalian cell. In some embodiments, the predetermined ratio of the nucleic acid is determined in transient transfection experiments to determine the relative ratio of input nucleic acids that results in the highest percentage of the antibody in the expressed product.

In some embodiments, the method of producing an antibody in stable mammalian cells as described herein results in an expression product of the at least one stable mammalian cell comprising a larger percentage of the desired glycosylated antibody as compared to the monomeric heavy or light chain polypeptides, or other antibodies.

In some embodiments, the method of producing a glycosylated antibody in stable mammalian cells described herein comprises identifying and purifying the desired glycosylated antibody. In some embodiments, the said identification is by one or both of liquid chromatography and mass spectrometry.

If required, the antibodies can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antibodies. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed or immobilized anti-flag antibody if a flag-tag is used. For general guidance in suitable purification techniques, see, e.g. incorporated entirely by reference Protein Purification: Principles and Practice, 3rd Ed., Scopes, Springer-Verlag, NY, 1994, incorporated entirely by reference. The degree of purification necessary will vary depending on the use of the antibodies. In some instances, no purification is necessary.

In certain embodiments, the antibodies are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antibodies described herein can be chemically synthesized using techniques known in the art (e.g., see Creighton, 1983, Proteins: Structures and Molecular Principles, W. H. Freeman & Co., N.Y; and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Nonclassical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

### Methods of Use

In an aspect, the present application provides methods of contacting TSLP with an anti-TSLP antibody, or an antigen binding fragment thereof, such as a human or humanized antibody. This can result in inhibition of TSLP binding to TSLPR.

In an aspect, the present application provides methods of using the isolated anti-TSLP antibodies, or antigen binding fragments thereof, described herein for treatment of a disorder or disease in a subject. In certain aspects, described herein are methods of treating a disorder or disease in a human patient comprising co-administering to the patient any one of the antibodies, or antigen binding fragments thereof, that binds TSLP described herein and a hyaluronidase or variant thereof.

In an aspect, the present application provides methods of using the isolated anti-TSLP antibodies or antigen binding fragments thereof described herein for treatment of a disorder or disease in a subject. In certain aspects, described herein is a method for treating a subject in need thereof with an anti-TSLP antibody, or an antigen binding fragment thereof, the method comprising administering to the subject (e.g., a mammalian subject) a therapeutically effective amount of an anti-TSLP antibody, or an antigen binding fragment thereof, or pharmaceutical composition comprising an anti-TSLP antibody, or an antigen binding fragment thereof, described herein. In certain embodiments, the present application provides methods of treating a disorder or disease associated with elevated levels of TSLP in a subject.

In certain aspects, described herein are methods for treating a pathology associated with TSLP activity, the method comprising administering to a mammalian subject a therapeutically effective amount of an isolated anti-TSLP antibody, or an antigen binding fragment thereof, or a pharmaceutical composition comprising an isolated anti-TSLP antibody, or an antigen binding fragment thereof, described herein.

In certain aspects, the antibodies and antibody fragments disclosed herein are useful for treating diseases and disorders which are improved, inhibited, or ameliorated by reducing TSLP activity. These disorders include those characterized by abnormal or excess expression of TSLP or by an abnormal host response to TSLP production. TSLP related disorders which are treated by the antibodies or antibody fragments of the disclosure include an inflammatory disorder or disease. In certain embodiments, the anti-TSLP antibody, or an antigen binding fragment thereof, is used in the treatment of atopic dermatitis. In certain embodiments, the treatment reduces disease severity in a subject and wherein disease severity is assessed by an atopic dermatitis disease severity outcome measure. In certain embodiments, the anti-TSLP antibody, or an antigen binding fragment thereof, is used in the treatment of asthma. In certain embodiments, the anti-TSLP antibody, or an antigen binding fragment thereof, is used in the treatment of idiopathic pulmonary fibrosis. In certain embodiments, the anti-TSLP antibody, or an antigen binding fragment thereof, is used in the treatment of alopecia areata. In certain embodiments, the anti-TSLP antibody, or an antigen binding fragment thereof, is used in the treatment of chronic sinusitis with nasal polyps. In certain embodiments, the anti-TSLP antibody, or an antigen binding fragment thereof, is used in the treatment of Chronic Rhinosinusitis without Nasal Polyps (CRSsNP). In certain embodiments, the anti-TSLP antibody, or an antigen binding fragment thereof, is used in the treatment of eosinophilic esophagitis (EoE). In certain embodiments, the anti-TSLP antibody, or an antigen binding fragment thereof, is used in the treatment of an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE). In certain embodiments, the anti-TSLP antibody, or an antigen binding fragment thereof, is used in the treatment of Churg-Strauss syndrome/Eosinophilic granulomatosis with polyangiitis (EGPA). In certain embodiments, the anti-TSLP antibody, or an antigen binding fragment thereof, is used in the treatment of Prurigo Nodularis (PN). In certain embodiments, the anti-TSLP antibody, or an antigen binding fragment thereof, is used in the treatment of Chronic Spontaneous Urticaria (CSU). In certain embodiments, the anti-TSLP antibody, or an antigen binding fragment thereof, is used in the treatment of Chronic Pruritis of Unknown Origin (CPUO). In certain embodiments, the anti-TSLP antibody, or an antigen binding fragment thereof, is used in the treatment of Bullous Pemphigoid (BP). In certain embodiments, the anti-TSLP antibody, or an antigen binding fragment thereof, is used in the treatment of Cold Inducible Urticaria (ColdU). In certain embodiments, the anti-TSLP antibody, or an antigen binding fragment thereof, is used in the treatment of Allergic Fungal Rhinosinusitis (AFRS). In certain embodiments, the anti-TSLP antibody, or an antigen binding fragment thereof, is used in the treatment of Allergic Bronchopulmonary Aspergillosis (ABPA). In certain embodiments, the anti-TSLP antibody, or an antigen binding fragment thereof, is used in the treatment of Chronic Obstructive Pulmonary Disease (COPD). In certain embodiments, the anti-TSLP antibody, or an antigen binding fragment thereof, is used in the treatment of an inflammatory bowel disease, such as Crohn's disease or ulcerative colitis. In certain embodiments, the anti-TSLP antibody, or an antigen binding fragment thereof, is used in the treatment of psoriasis. In certain embodiments, the anti-TSLP antibody, or an antigen binding fragment thereof, is used in the treatment of lupus. In certain embodiments, the anti-TSLP antibody, or an antigen binding fragment thereof, is used in the treatment of rheumatoid arthritis. In certain embodiments, the anti-TSLP antibody, or an antigen binding fragment thereof, is used in the treatment of hidradenitis suppurativa. In certain embodiments, the anti-TSLP antibody, or an antigen binding fragment thereof, is used in the treatment of celiac disease. In certain embodiments, the anti-TSLP antibody, or an antigen binding fragment thereof, is used in the treatment of systemic sclerosis.

In certain aspects, described herein is a method for treating an inflammatory disorder or disease in a mammalian subject in need thereof, the method comprising administering to the mammalian subject a therapeutically effective amount of an anti-TSLP antibody, or an antigen binding fragment thereof, described herein or a pharmaceutical composition described herein. In certain embodiments of the methods described herein, the inflammatory disorder or disease is atopic dermatitis. In certain embodiments, the inflammatory disorder or disease is asthma. In certain embodiments, the inflammatory disorder or disease is idiopathic pulmonary fibrosis. In certain embodiments, the inflammatory disorder or disease is alopecia areata. In certain embodiments, the inflammatory disorder or disease is chronic sinusitis with nasal polyps. In certain embodiments, the inflammatory disorder or disease is Chronic Rhinosinusitis without Nasal Polyps (CRSsNP). In certain embodiments, the inflammatory disorder or disease is eosinophilic esophagitis (EoE). In certain embodiments, the inflammatory disorder or disease is an Eosinophilic gastrointestinal disorder or disease (EGID) selected from the group consisting of Eosinophilic Gastritis (EoG), Eosinophilic enteritis (EoN), Eosinophilic colitis (EoC), and Eosinophilic Gastroenteritis (EGE). In certain embodiments, the inflammatory disorder or disease is Churg-Strauss syndrome/Eosinophilic granulomatosis with polyangiitis (EGPA). In certain embodiments, the inflammatory disorder or disease is Prurigo Nodularis (PN). In certain embodiments, the inflammatory disorder or disease is Chronic Spontaneous Urticaria (CSU). In certain embodiments, the inflammatory disorder or disease is Chronic Pruritis of Unknown Origin (CPUO). In certain embodiments, the inflammatory disorder or disease is Bullous Pemphigoid (BP). In certain embodiments, the inflammatory disorder or disease is Cold Inducible Urticaria (ColdU). In certain embodiments, the inflammatory disorder or disease is Allergic Fungal Rhinosinusitis (AFRS). In certain embodiments, the inflammatory disorder or disease is Allergic Bronchopulmonary Aspergillosis (ABPA). In certain embodiments, the inflammatory disorder or disease is Chronic Obstructive Pulmonary Disease (COPD). In certain embodiments, the inflammatory disorder or disease is inflammatory bowel disease, such as Crohn's disease or ulcerative colitis. In certain embodiments, the inflammatory disorder or disease is psoriasis. In certain embodiments, the inflammatory disorder or disease is lupus. In certain embodiments, the inflammatory disorder or disease is rheumatoid arthritis. In certain embodiments, the inflammatory disorder or disease is celiac disease. In certain embodiments, the inflammatory disorder or disease is hidradenitis suppurativa. In certain embodiments, the inflammatory disorder or disease is systemic sclerosis.

In certain aspects, described herein are methods for treating an inflammatory disorder or disease in a mammalian subject in need thereof, the method comprising administering to the mammalian subject a therapeutically effective amount of the antibody, or an antigen binding fragment thereof, or a pharmaceutical composition described herein. In certain embodiments, the inflammatory disorder or disease is atopic dermatitis. In certain embodiments, the inflammatory disorder or disease is asthma. In certain embodiments, the inflammatory disorder or disease is nasal polyps.

In certain aspects, described herein are methods for treating a pathology associated with elevated levels of TSLP and/or TSLPR in a mammalian subject in need thereof, the method comprising administering to the mammalian subject a therapeutically effective amount of an antibody, or an antigen binding fragment thereof, or a pharmaceutical composition described herein.

In certain aspects, described herein are methods of reducing biological activity of TSLP and/or TSLPR in a mammalian subject in need thereof, the method comprising administering to the mammalian subject a therapeutically effective amount of an antibody, or an antigen binding fragment thereof, or a pharmaceutical composition described herein.

In certain aspects, described herein are methods of preventing an inflammatory disorder or disease in a mammalian subject in need thereof, the method comprising administering to the mammalian subject a therapeutically effective amount of an antibody, or an antigen binding fragment thereof, or a pharmaceutical composition described herein.

In certain aspects, described herein are methods for reducing a level of cytokine and/or a chemokine in a mammalian subject in need thereof, the method comprising administering to the mammalian subject a therapeutically effective amount of an anti-TSLP antibody, or an antigen binding fragment thereof, or a pharmaceutical composition described herein. In certain embodiments, the chemokine is a type 2 chemokine. In certain embodiments, the type 2 chemokine is TARC or MDC.

In certain aspects, described herein are methods for reducing a level of a type 2 chemokine in a mammalian subject in need thereof, the method comprising administering to the mammalian subject a therapeutically effective amount of an anti-TSLP antibody, or an antigen binding fragment thereof, or a pharmaceutical composition described herein. In certain embodiments, the method reduces the level of the type 2 chemokine by at least about 50%, 60%, 70%, 80%, 85%, 90%, or 95%.

In certain aspects, described herein are methods of reducing a level of Thymus and Activation Regulated Chemokine (TARC)/CCL17 in a mammalian subject in need thereof, the method comprising administering to the mammalian subject a therapeutically effective amount of an antibody, or an antigen binding fragment thereof, or a pharmaceutical composition described herein. In certain embodiments, the method reduces the level of TARC/CCL17 by at least about 50%, 60%, 70%, 80%, 85%, 90%, or 95%.

In certain aspects, described herein are methods of reducing a level of macrophage-derived chemokine (MDC (CCL22)) in a mammalian subject in need thereof, the method comprising administering to the mammalian subject a therapeutically effective amount of an antibody, or an antigen binding fragment thereof, or a pharmaceutical composition described herein. In certain embodiments, the method reduces the level of MDC by at least about 50%, 60%, 70%, 80%, 85%, 90%, or 95%.

In certain aspects, described herein are methods for treating asthma in a patient, wherein the method comprises administering an anti-TSLP antibody, or an antigen binding fragment thereof.

Any of the above-described methods may further include administration of a hyaluronidase or a variant thereof (e.g., rHuPH20). In some embodiments, the hyaluronidase or variant thereof is included in the same composition as the anti-TSLP antibody, or antigen binding fragment thereof. In other embodiments, the hyaluronidase or variant thereof is administered separately from the anti-TSLP antibody, or antigen binding fragment thereof. The hyaluronidase or variant thereof may be administered before or after the anti-TSLP antibody, or antigen binding fragment thereof, or may be administered at about the same time as the anti-TSLP antibody or antigen binding fragment thereof (e.g., in a separate composition or via co-formulation).

### Methods of Administration

In some embodiments, the methods provided herein are useful for the treatment of a disease or disorder in an individual. In an embodiment, the individual is a human and the antibody is an anti-TSLP antibody, or an antigen binding fragment thereof, described herein. Described herein, in certain embodiments, are methods of treating an inflammatory disorder or disease in a patient in need thereof, the method comprising subcutaneously or intravenously administering to the patient an effective amount of an anti-TSLP antibody, or an antigen binding fragment thereof, comprising a modified Fc region. Further described herein, in certain embodiments, are methods of treating an inflammatory disorder or disease in a patient in need thereof, the method comprising subcutaneously or intravenously administering to the patient an effective amount of an anti-TSLP antibody, or an antigen binding fragment thereof, wherein the anti-TSLP antibody or the antigen binding fragment thereof specifically binds to an epitope of TSLP and comprises a Fc domain comprising amino acid modifications M252Y, S254T, and T256E (YTE) or M257Y/S259T/T261E (YTE) using direct numbering and/or M428L and N434S (LS) or M433L/N439S (LS) using direct numbering. The Fc domain may further comprise amino acid modifications L234A/L235A (LALA) or L239A/L240A (LALA) using direct numbering (e.g., LALA/YTE or LALA/LS).

In some embodiments, an antibody, or an antigen binding fragment thereof, is administered intravenously, intramuscularly, subcutaneously, topically, orally, transdermally, intraperitoneally, intraorbitally, by implantation, by inhalation, intrathecally, intraventricularly, or intranasally. An effective amount of an anti-TSLP antibody, or an antigen binding fragment thereof, may be administered for the treatment of a disease or disorder. In some embodiments, administration of the anti-TSLP antibody, or an antigen binding fragment thereof, is intravenous or subcutaneous. In some embodiments, administration of the anti-TSLP antibody, or an antigen binding fragment thereof, is intravenous. In some embodiments, administration of the anti-TSLP antibody, or an antigen binding fragment thereof, is subcutaneous. The appropriate dosage of the anti-TSLP antibody, or an antigen binding fragment thereof, may be determined based on the type of disease or disorder to be treated, the type of the anti-TSLP antibody, the severity and course of the disease or disorder, the clinical condition of the individual, the individual's clinical history and response to the treatment, and the discretion of the attending physician.

In certain embodiments, the anti-TSLP antibody, or an antigen binding fragment thereof, is administered in an amount of about 50 mg to about 1500 mg, for example, in an amount of about 150 mg, about 300 mg, about 600 mg, or about 1200 mg. In certain embodiments, the anti-TSLP antibody, or an antigen binding fragment thereof, is administered weekly, every two weeks, every three weeks, every four weeks, every six weeks, or every two months.

### Compositions, Combinations, and Methods Involving Co-Administration

In certain aspects, described herein are compositions comprising any of the antibodies, or antigen binding fragments thereof, that bind thymic stromal lymphopoietin (TSLP) described herein and a hyaluronidase or variant thereof.

In certain aspects, described herein are a combination of any one of the antibodies, or antigen binding fragments thereof, that bind TSLP described herein and a hyaluronidase or variant thereof.

In certain aspects, described herein are methods of treating an inflammatory disorder or disease in a human patient comprising co-administering to the patient any one of the antibodies, or antigen binding fragments thereof, that bind TSLP described herein and a hyaluronidase or variant thereof.

In certain embodiments, the antibody, or antigen binding fragment thereof, is a humanized, human, or chimeric antibody. In certain embodiments, the antibody, or antigen binding fragment thereof, is a monoclonal antibody. In certain embodiments, the antibody, or antigen binding fragment thereof, comprises a heavy chain human constant region of a class selected from IgG, IgA, IgD, IgE, and IgM. In certain embodiments, the antibody, or antigen binding fragment thereof, comprises a human Fc region comprising a human heavy chain constant region of the class IgG and a subclass selected from IgG1, IgG2, IgG3, and IgG4. In certain embodiments, the human Fc region comprises a human IgG1 Fc region.

In certain embodiments the human Fc region comprises a human IgG1 Fc region with LALA mutations. In certain embodiments the human Fc region comprises a human IgG1 Fc region with YTE mutations. In certain embodiments the human Fc region comprises a human IgG1 Fc region with LALA and YTE mutations. In certain embodiments, when direct numbering is used these “YTE” and “LALA” mutations can be located at different amino acid position numbers. For example, in one embodiment, the human Fc region comprises a human IgG1 Fc region with LALA mutations at L239A/L240A and/or YTE mutations at M257Y/S259T/T261E.

In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In certain embodiments, the antibody, or antigen binding fragment thereof, that binds TSLP comprises a VH sequence set forth in SEQ ID NO: 34 and a VL sequence set forth in SEQ ID NO: 41. In certain embodiments, the antibody, or antigen binding fragment thereof, that binds TSLP comprises a human Fc region comprising a human IgG sequence selected from a sequence set forth in any one of SEQ ID NOs: 47-270. In certain embodiments, the antibody, or antigen binding fragment thereof, that binds TSLP comprises a heavy chain constant region sequence set forth in SEQ ID NO: 61 or 173 and a light chain constant region sequence set forth in SEQ ID NO: 480. In certain embodiments, the antibody that binds TSLP comprises a heavy chain sequence set forth in SEQ ID NO: 484 and/or 486 and a light chain sequence set forth in SEQ ID NO: 485. In certain embodiments, the antibody that binds TSLP comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486.

In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29. In certain embodiments, the antibody, or antigen binding fragment thereof, that binds TSLP comprises a VH sequence set forth in SEQ ID NO: 478 and a VL sequence set forth in SEQ ID NO: 479. In certain embodiments, the antibody, or antigen binding fragment thereof, that binds TSLP comprises a human Fc region comprising a human IgG sequence selected from a sequence set forth in any one of SEQ ID NOs: 47-270. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In certain embodiments, the antibody that binds TSLP comprises a heavy chain sequence set forth in SEQ ID NO: 476 and a light chain sequence set forth in SEQ ID NO: 477.

In certain embodiments, the antibody, or antigen binding fragment thereof, binds an TSLP sequence set forth in SEQ ID NO: 365 or 366. In certain embodiments, the Fc region of the antibody, or antigen binding fragment thereof, binds to Neonatal Fc receptor (FcRn). In certain embodiments, the Fc region of the antibody binds an FcRn with higher affinity at pH 6.0 compared to an antibody comprising a wild-type Fc region.

Any suitable hyaluronidase or variant thereof can be used in the compositions, combinations, and methods described herein. In certain embodiments, the hyaluronidase is a soluble hyaluronidase. In certain embodiments, the hyaluronidase is a recombinant human hyaluronidase. In certain embodiments, the recombinant human hyaluronidase or variant thereof comprises the amino acid sequence set forth in any one of SEQ ID NOs: 409-418 and 420-475, or a mixture thereof (e.g., the recombinant human hyaluronidase may comprise one or more of SEQ ID NOs: 409-418 and 420-475). In certain embodiments, the recombinant human hyaluronidase is rHuPH20 (i.e., a composition comprising one or more of SEQ ID NOs: 411-416). In certain embodiments, the rHuPH20 formulation is ENHANZE®.

In one embodiment, the recombinant human hyaluronidase includes a sequence of amino acids in any one of SEQ ID NOs: 409-418 and 420-475, or has at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence of amino acids included in SEQ ID NO:409-418 and 420-475 and retains hyaluronidase activity. In one embodiment, the recombinant human hyaluronidase comprises a sequence having at least 95%, 96%, 97%, 98%, or 99% to the amino acid sequence of SEQ ID NO: 409. In one embodiment, the recombinant human hyaluronidase comprises a sequence having at least 95%, 96%, 97%, 98%, or 99% to the amino acid sequence of SEQ ID NO: 411. In another embodiment, the recombinant human hyaluronidase comprises the amino acid sequence set forth in SEQ ID NO:409. In another embodiment, the recombinant human hyaluronidase consists of the amino acid sequence set forth in SEQ ID NO:409. In another embodiment, the recombinant human hyaluronidase comprises the amino acid sequence set forth in SEQ ID NO:410. In another embodiment, the recombinant human hyaluronidase consists of the amino acid sequence set forth in SEQ ID NO:410. In another embodiment, the recombinant human hyaluronidase comprises the amino acid sequence set forth in SEQ ID NO: 411. In another embodiment, the recombinant human hyaluronidase consists of the amino acid sequence set forth in SEQ ID NO:411. In another embodiment, the recombinant human hyaluronidase comprises the amino acid sequence set forth in SEQ ID NO:412. In another embodiment, the recombinant human hyaluronidase consists of the amino acid sequence set forth in SEQ ID NO:412. In another embodiment, the recombinant human hyaluronidase comprises the amino acid sequence set forth in SEQ ID NO:413. In another embodiment, the recombinant human hyaluronidase consists of the amino acid sequence set forth in SEQ ID NO:413. In another embodiment, the recombinant human hyaluronidase comprises the amino acid sequence set forth in SEQ ID NO:414. In another embodiment, the recombinant human hyaluronidase consists of the amino acid sequence set forth in SEQ ID NO:414. In another embodiment, the recombinant human hyaluronidase comprises the amino acid sequence set forth in SEQ ID NO: 415. In another embodiment, the recombinant human hyaluronidase consists of the amino acid sequence set forth in SEQ ID NO:415. In another embodiment, the recombinant human hyaluronidase comprises the amino acid sequence set forth in SEQ ID NO:416. In another embodiment, the recombinant human hyaluronidase consists of the amino acid sequence set forth in SEQ ID NO:416. In another embodiment, the recombinant human hyaluronidase comprises the amino acid sequence set forth in SEQ ID NO:417. In another embodiment, the recombinant human hyaluronidase consists of the amino acid sequence set forth in SEQ ID NO:417. In another embodiment, the recombinant human hyaluronidase comprises the amino acid sequence set forth in SEQ ID NO:418. In another embodiment, the recombinant human hyaluronidase consists of the amino acid sequence set forth in SEQ ID NO:418. In another embodiment, the recombinant human hyaluronidase comprises the amino acid sequence set forth in SEQ ID NO: 420. In another embodiment, the recombinant human hyaluronidase consists of the amino acid sequence set forth in SEQ ID NO:420. In another embodiment, the recombinant human hyaluronidase comprises the amino acid sequence set forth in SEQ ID NO:421. In another embodiment, the recombinant human hyaluronidase consists of the amino acid sequence set forth in SEQ ID NO:421. In another embodiment, the recombinant human hyaluronidase comprises the amino acid sequence set forth in SEQ ID NO:422. In another embodiment, the recombinant human hyaluronidase consists of the amino acid sequence set forth in SEQ ID NO:422. In another embodiment, the recombinant human hyaluronidase comprises the amino acid sequence set forth in SEQ ID NO:423. In another embodiment, the recombinant human hyaluronidase consists of the amino acid sequence set forth in SEQ ID NO:423. In another embodiment, the recombinant human hyaluronidase comprises the amino acid sequence set forth in SEQ ID NO: 424. In another embodiment, the recombinant human hyaluronidase consists of the amino acid sequence set forth in SEQ ID NO:424. In another embodiment, the recombinant human hyaluronidase comprises the amino acid sequence set forth in SEQ ID NO:425. In another embodiment, the recombinant human hyaluronidase consists of the amino acid sequence set forth in SEQ ID NO:425. In another embodiment, the recombinant human hyaluronidase comprises the amino acid sequence set forth in SEQ ID NO:426. In another embodiment, the recombinant human hyaluronidase consists of the amino acid sequence set forth in SEQ ID NO:426. In another embodiment, the recombinant human hyaluronidase comprises the amino acid sequence set forth in SEQ ID NO:427. In another embodiment, the recombinant human hyaluronidase consists of the amino acid sequence set forth in SEQ ID NO:427. In another embodiment, the recombinant human hyaluronidase comprises the amino acid sequence set forth in SEQ ID NO: 428. In another embodiment, the recombinant human hyaluronidase consists of the amino acid sequence set forth in SEQ ID NO:428. In another embodiment, the recombinant human hyaluronidase comprises the amino acid sequence set forth in SEQ ID NO:429. In another embodiment, the recombinant human hyaluronidase consists of the amino acid sequence set forth in SEQ ID NO:429. In another embodiment, the recombinant human hyaluronidase comprises the amino acid sequence set forth in SEQ ID NO:430. In another embodiment, the recombinant human hyaluronidase consists of the amino acid sequence set forth in SEQ ID NO:430. In another embodiment, the recombinant human hyaluronidase comprises the amino acid sequence set forth in SEQ ID NO:431. In another embodiment, the recombinant human hyaluronidase consists of the amino acid sequence set forth in SEQ ID NO:431. In another embodiment, the recombinant human hyaluronidase comprises the amino acid sequence set forth in SEQ ID NO: 432. In another embodiment, the recombinant human hyaluronidase consists of the amino acid sequence set forth in SEQ ID NO:432. In another embodiment, the recombinant human hyaluronidase comprises the amino acid sequence set forth in SEQ ID NO:433. In another embodiment, the recombinant human hyaluronidase consists of the amino acid sequence set forth in SEQ ID NO:433. In another embodiment, the recombinant human hyaluronidase comprises the amino acid sequence set forth in SEQ ID NO:434. In another embodiment, the recombinant human hyaluronidase consists of the amino acid sequence set forth in SEQ ID NO:434. In another embodiment, the recombinant human hyaluronidase comprises the amino acid sequence set forth in SEQ ID NO:435. In another embodiment, the recombinant human hyaluronidase consists of the amino acid sequence set forth in SEQ ID NO:435. In another embodiment, the recombinant human hyaluronidase comprises the amino acid sequence set forth in SEQ ID NO:436. In another embodiment, the recombinant human hyaluronidase consists of the amino acid sequence set forth in SEQ ID NO:436. In another embodiment, the recombinant human hyaluronidase comprises the amino acid sequence set forth in SEQ ID NO:437. In another embodiment, the recombinant human hyaluronidase consists of the amino acid sequence set forth in SEQ ID NO:437. In another embodiment, the recombinant human hyaluronidase comprises the amino acid sequence set forth in SEQ ID NO:438. In another embodiment, the recombinant human hyaluronidase consists of the amino acid sequence set forth in SEQ ID NO: 438. In another embodiment, the recombinant human hyaluronidase comprises the amino acid sequence set forth in SEQ ID NO:439. In another embodiment, the recombinant human hyaluronidase consists of the amino acid sequence set forth in SEQ ID NO:439. In another embodiment, the recombinant human hyaluronidase comprises the amino acid sequence set forth in SEQ ID NO:440. In another embodiment, the recombinant human hyaluronidase consists of the amino acid sequence set forth in SEQ ID NO:440. In another embodiment, the recombinant human hyaluronidase comprises the amino acid sequence set forth in SEQ ID NO:441. In another embodiment, the recombinant human hyaluronidase consists of the amino acid sequence set forth in SEQ ID NO:441. In another embodiment, the recombinant human hyaluronidase comprises the amino acid sequence set forth in SEQ ID NO:442. In another embodiment, the recombinant human hyaluronidase consists of the amino acid sequence set forth in SEQ ID NO: 442. In another embodiment, the recombinant human hyaluronidase comprises the amino acid sequence set forth in SEQ ID NO:443. In another embodiment, the recombinant human hyaluronidase consists of the amino acid sequence set forth in SEQ ID NO:443. In another embodiment, the recombinant human hyaluronidase comprises the amino acid sequence set forth in SEQ ID NO:444. In another embodiment, the recombinant human hyaluronidase consists of the amino acid sequence set forth in SEQ ID NO:444. In another embodiment, the recombinant human hyaluronidase comprises the amino acid sequence set forth in SEQ ID NO:445. In another embodiment, the recombinant human hyaluronidase consists of the amino acid sequence set forth in SEQ ID NO:445. In another embodiment, the recombinant human hyaluronidase comprises the amino acid sequence set forth in SEQ ID NO:446. In another embodiment, the recombinant human hyaluronidase consists of the amino acid sequence set forth in SEQ ID NO: 446. In another embodiment, the recombinant human hyaluronidase comprises the amino acid sequence set forth in SEQ ID NO:447. In another embodiment, the recombinant human hyaluronidase consists of the amino acid sequence set forth in SEQ ID NO:447. In another embodiment, the recombinant human hyaluronidase comprises the amino acid sequence set forth in SEQ ID NO:448. In another embodiment, the recombinant human hyaluronidase consists of the amino acid sequence set forth in SEQ ID NO:448. In another embodiment, the recombinant human hyaluronidase comprises the amino acid sequence set forth in SEQ ID NO:449. In another embodiment, the recombinant human hyaluronidase consists of the amino acid sequence set forth in SEQ ID NO:449. In another embodiment, the recombinant human hyaluronidase comprises the amino acid sequence set forth in SEQ ID NO:450. In another embodiment, the recombinant human hyaluronidase consists of the amino acid sequence set forth in SEQ ID NO: 450. In another embodiment, the recombinant human hyaluronidase comprises the amino acid sequence set forth in SEQ ID NO:451. In another embodiment, the recombinant human hyaluronidase consists of the amino acid sequence set forth in SEQ ID NO:451. In another embodiment, the recombinant human hyaluronidase comprises the amino acid sequence set forth in SEQ ID NO:452. In another embodiment, the recombinant human hyaluronidase consists of the amino acid sequence set forth in SEQ ID NO:452. In another embodiment, the recombinant human hyaluronidase comprises the amino acid sequence set forth in SEQ ID NO:453. In another embodiment, the recombinant human hyaluronidase consists of the amino acid sequence set forth in SEQ ID NO:453. In another embodiment, the recombinant human hyaluronidase comprises the amino acid sequence set forth in SEQ ID NO:454. In another embodiment, the recombinant human hyaluronidase consists of the amino acid sequence set forth in SEQ ID NO: 454. In another embodiment, the recombinant human hyaluronidase comprises the amino acid sequence set forth in SEQ ID NO:455. In another embodiment, the recombinant human hyaluronidase consists of the amino acid sequence set forth in SEQ ID NO:455. In another embodiment, the recombinant human hyaluronidase comprises the amino acid sequence set forth in SEQ ID NO:456. In another embodiment, the recombinant human hyaluronidase consists of the amino acid sequence set forth in SEQ ID NO:456. In another embodiment, the recombinant human hyaluronidase comprises the amino acid sequence set forth in SEQ ID NO:457. In another embodiment, the recombinant human hyaluronidase consists of the amino acid sequence set forth in SEQ ID NO:457. In another embodiment, the recombinant human hyaluronidase comprises the amino acid sequence set forth in SEQ ID NO:458. In another embodiment, the recombinant human hyaluronidase consists of the amino acid sequence set forth in SEQ ID NO: 458. In another embodiment, the recombinant human hyaluronidase comprises the amino acid sequence set forth in SEQ ID NO:459. In another embodiment, the recombinant human hyaluronidase consists of the amino acid sequence set forth in SEQ ID NO:459. In another embodiment, the recombinant human hyaluronidase comprises the amino acid sequence set forth in SEQ ID NO:460. In another embodiment, the recombinant human hyaluronidase consists of the amino acid sequence set forth in SEQ ID NO:460. In another embodiment, the recombinant human hyaluronidase comprises the amino acid sequence set forth in SEQ ID NO:461. In another embodiment, the recombinant human hyaluronidase consists of the amino acid sequence set forth in SEQ ID NO:461. In another embodiment, the recombinant human hyaluronidase comprises the amino acid sequence set forth in SEQ ID NO:462. In another embodiment, the recombinant human hyaluronidase consists of the amino acid sequence set forth in SEQ ID NO: 462. In another embodiment, the recombinant human hyaluronidase comprises the amino acid sequence set forth in SEQ ID NO:463. In another embodiment, the recombinant human hyaluronidase consists of the amino acid sequence set forth in SEQ ID NO:463. In another embodiment, the recombinant human hyaluronidase comprises the amino acid sequence set forth in SEQ ID NO:464. In another embodiment, the recombinant human hyaluronidase consists of the amino acid sequence set forth in SEQ ID NO:464. In another embodiment, the recombinant human hyaluronidase comprises the amino acid sequence set forth in SEQ ID NO:465. In another embodiment, the recombinant human hyaluronidase consists of the amino acid sequence set forth in SEQ ID NO:465. In another embodiment, the recombinant human hyaluronidase comprises the amino acid sequence set forth in SEQ ID NO:466. In another embodiment, the recombinant human hyaluronidase consists of the amino acid sequence set forth in SEQ ID NO: 466. In another embodiment, the recombinant human hyaluronidase comprises the amino acid sequence set forth in SEQ ID NO:467. In another embodiment, the recombinant human hyaluronidase consists of the amino acid sequence set forth in SEQ ID NO:467. In another embodiment, the recombinant human hyaluronidase comprises the amino acid sequence set forth in SEQ ID NO:468. In another embodiment, the recombinant human hyaluronidase consists of the amino acid sequence set forth in SEQ ID NO:468. In another embodiment, the recombinant human hyaluronidase comprises the amino acid sequence set forth in SEQ ID NO:469. In another embodiment, the recombinant human hyaluronidase consists of the amino acid sequence set forth in SEQ ID NO:469. In another embodiment, the recombinant human hyaluronidase comprises the amino acid sequence set forth in SEQ ID NO:470. In another embodiment, the recombinant human hyaluronidase consists of the amino acid sequence set forth in SEQ ID NO: 470. In another embodiment, the recombinant human hyaluronidase comprises the amino acid sequence set forth in SEQ ID NO:471. In another embodiment, the recombinant human hyaluronidase consists of the amino acid sequence set forth in SEQ ID NO:471. In another embodiment, the recombinant human hyaluronidase comprises the amino acid sequence set forth in SEQ ID NO:472. In another embodiment, the recombinant human hyaluronidase consists of the amino acid sequence set forth in SEQ ID NO:472. In another embodiment, the recombinant human hyaluronidase comprises the amino acid sequence set forth in SEQ ID NO:473. In another embodiment, the recombinant human hyaluronidase consists of the amino acid sequence set forth in SEQ ID NO:473. In another embodiment, the recombinant human hyaluronidase comprises the amino acid sequence set forth in SEQ ID NO:474. In another embodiment, the recombinant human hyaluronidase consists of the amino acid sequence set forth in SEQ ID NO: 474. In another embodiment, the recombinant human hyaluronidase comprises the amino acid sequence set forth in SEQ ID NO:475. In another embodiment, the recombinant human hyaluronidase consists of the amino acid sequence set forth in SEQ ID NO:475.

In certain embodiments, the hyaluronidase or variant thereof and the anti-TSLP antibody, or antigen binding fragment thereof, are administered in separate formulations. In certain embodiments, the hyaluronidase or variant thereof and the anti-TSLP antibody, or antigen binding fragment thereof, are administered simultaneously in separate formulations. In certain embodiments, the hyaluronidase or variant thereof is administered to the patient prior to administration of the anti-TSLP antibody, or antigen binding fragment thereof. In certain embodiments, the hyaluronidase or variant thereof is administered to the patient after administration of the anti-TSLP antibody, or antigen binding fragment thereof. In certain embodiments, the hyaluronidase or variant thereof and the anti-TSLP antibody, or antigen binding fragment thereof, are mixed and administered in a single formulation. In certain embodiments, the hyaluronidase or variant thereof and the anti-TSLP antibody, or antigen binding fragment thereof, are administered by subcutaneous injection. In certain embodiments, the hyaluronidase or variant thereof and the anti-TSLP antibody, or antigen binding fragment thereof, are administered by intravenous injection.

In certain embodiments, the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

In certain embodiments, methods of treating an inflammatory disorder or disease in a human patient are provided, wherein the method comprises co-administering to the patient any one of the antibodies, or antigen binding fragments thereof, that bind TSLP described herein and a hyaluronidase or variant thereof. In certain embodiments, the inflammatory disorder or disease is atopic dermatitis. In certain embodiments, the treatment reduces disease severity in the patient and wherein disease severity is assessed by an atopic dermatitis disease severity outcome measure. In certain embodiments, the inflammatory disorder or disease is selected from the group consisting of asthma, idiopathic pulmonary fibrosis, alopecia areata, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), inflammatory bowel disease, such as Crohn's disease or ulcerative colitis, lupus, rheumatoid arthritis (RA), psoriasis, hidradenitis suppurativa, celiac disease, and systemic sclerosis. In certain embodiments, the inflammatory disorder or disease is an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE).

In certain aspects, described herein are compositions comprising an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and a hyaluronidase or variant thereof. In other aspects, described herein are compositions comprising an antibody, or antigen binding fragment thereof, that binds TSLP, wherein the antibody, or antigen binding fragment thereof, NO: 41, and a hyaluronidase or variant thereof. In certain embodiments, the antibody, or antigen binding fragment thereof, that binds TSLP comprises a heavy chain constant region set forth in any one of SEQ ID NOs: 47-270. In certain embodiments, the antibody, or antigen binding fragment thereof, that binds TSLP comprises a heavy chain constant region set forth in SEQ ID NO: 61 or 173 and a light chain constant region set forth in SEQ ID NO: 480. In other aspects, described herein are compositions comprising an antibody that binds TSLP, wherein the antibody comprises a heavy chain sequence set forth in SEQ ID NO: 484 and/or 486 and a light chain sequence set forth in SEQ ID NO: 485, and a hyaluronidase or variant thereof. In certain embodiments, the antibody that binds TSLP comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486.

In certain aspects, described herein are compositions comprising an antibody, or antigen binding fragment thereof, that binds TSLP and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29. In other aspects, described herein are compositions comprising an antibody, or antigen binding fragment thereof, that binds TSLP and a hyaluronidase or variant thereof, wherein the antibody, or antigen binding fragment thereof, comprises a VH sequence set forth in SEQ ID NO: 478 and a VL sequence set forth in SEQ ID NO: 479. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In other aspects, described herein are compositions comprising an antibody that binds TSLP and a hyaluronidase or variant thereof, wherein the antibody comprises a heavy chain sequence set forth in SEQ ID NO: 476 and a light chain sequence set forth in SEQ ID NO: 477.

In certain aspects, described herein are a combination of an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and a hyaluronidase or variant thereof. In other aspects, described herein are a combination of an antibody, or antigen binding fragment thereof, that binds TSLP, wherein the antibody, or antigen binding fragment thereof, comprises a VH sequence set forth in SEQ ID NO: 34 and a VL sequence set forth in SEQ ID NO: 41, and a hyaluronidase or variant thereof. In certain embodiments, the antibody, or antigen binding fragment thereof, that binds TSLP comprises a heavy chain constant region set forth in any one of SEQ ID NOs: 47-270. In certain embodiments, the antibody, or antigen binding fragment thereof, that binds TSLP comprises a heavy chain constant region set forth in SEQ ID NO: 61 or 173 and a light chain constant region set forth in SEQ ID NO: 480. In other aspects, described herein are a combination of an antibody that binds TSLP, wherein the antibody comprises a heavy chain sequence set forth in SEQ ID NO: 484 and/or 486 and a light chain sequence set forth in SEQ ID NO: 485, and a hyaluronidase or variant thereof. In certain embodiments, the antibody that binds TSLP comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486.

In certain aspects, described herein are a combination of an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29, and a hyaluronidase or variant thereof. In other aspects, described herein are a combination of an antibody, or antigen binding fragment thereof, that binds TSLP, wherein the antibody, or antigen binding fragment thereof, comprises a VH sequence set forth in SEQ ID NO: 478 and a VL sequence set forth in SEQ ID NO: 479, and a hyaluronidase or variant thereof. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In other aspects, described herein are a combination of an antibody that binds TSLP, wherein the antibody comprises a heavy chain sequence set forth in SEQ ID NO: 476 and a light chain sequence set forth in SEQ ID NO: 477, and a hyaluronidase or variant thereof.

In certain aspects, described herein are methods of treating an inflammatory disorder or disease in a human patient comprising co-administering to the patient an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27, and a hyaluronidase or variant thereof. In other aspects, described herein are methods of treating an inflammatory disorder or disease in a human patient comprising co-administering to the patient an antibody, or antigen binding fragment thereof, that binds TSLP, wherein the antibody, or antigen binding fragment thereof, comprises a VH sequence set forth in SEQ ID NO: 34 and a VL sequence set forth in SEQ ID NO: 41, and a hyaluronidase or variant thereof. In certain embodiments, the antibody, or antigen binding fragment thereof, comprises a heavy chain comprising a constant heavy chain sequence set forth in SEQ ID NOs: 47-270. In certain embodiments, the antibody, or antigen binding fragment thereof, comprises a heavy chain comprising a constant heavy chain sequence set forth in SEQ ID NO: 61 or 173. In certain embodiments, the antibody, or antigen binding fragment thereof, comprises a light chain comprising a constant light chain sequence set forth in SEQ ID NO: 480. In certain embodiments, the antibody that binds TSLP comprises a heavy chain sequence set forth in SEQ ID NO: 484 and/or 486 and a light chain sequence set forth in SEQ ID NO: 485. In certain embodiments, the antibody that binds TSLP comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486.

In certain aspects, described herein are methods of treating an inflammatory disorder or disease in a human patient comprising co-administering to the patient an antibody, or antigen binding fragment thereof, that binds TSLP, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29, and a hyaluronidase or variant thereof. In other aspects, described herein are methods of treating an inflammatory disorder or disease in a human patient comprising co-administering to the patient an antibody, or antigen binding fragment thereof, that binds TSLP, wherein the antibody, or antigen binding fragment thereof, comprises a VH sequence set forth in SEQ ID NO: 478 and a VL sequence set forth in SEQ ID NO: 479, and a hyaluronidase or variant thereof. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In other aspects, described herein are methods of treating an inflammatory disorder or disease in a human patient comprising co-administering to the patient an antibody that binds TSLP, wherein the antibody comprises a heavy chain sequence set forth in SEQ ID NO: 476 and a light chain sequence set forth in SEQ ID NO: 477, and a hyaluronidase or variant thereof.

### Kits and Articles of Manufacture

The present application provides kits comprising any one or more of the antibody compositions described herein and instructions for use. In some embodiments, the kits further contain a component selected from any of secondary antibodies, reagents for immunohistochemistry analysis, a pharmaceutically acceptable excipient, a package insert, and an instruction manual and any combination thereof. In some embodiments, the kits further contain a hyaluronidase or variant thereof. In one specific embodiment, the kit comprises a pharmaceutical composition comprising any one or more of the antibody compositions described herein, with one or more pharmaceutically acceptable excipients. In some embodiments, the pharmaceutical composition further comprises a hyaluronidase or variant thereof.

The present application also provides articles of manufacture comprising any one of the antibody compositions or kits described herein. Examples of an article of manufacture include vials (including sealed vials).

In certain aspects, described herein are kits for treating an inflammatory disorder or disease in a human patient, the kit comprising: (a) a dose of any one of the antibodies, or antigen binding fragments thereof, that binds TSLP described herein (e.g., an antibody, or an antigen binding fragment thereof, that binds TSLP in unit dosage form); (b) a dose of a hyaluronidase or variant thereof (e.g., a hyaluronidase or variant thereof in unit dosage form); and (c) instructions. In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In certain embodiments, the antibody, or antigen binding fragment thereof, comprises a VH sequence set forth in SEQ ID NO: 34 and a VL sequence set forth in SEQ ID NO: 41. In certain embodiments, the antibody, or antigen binding fragment thereof, comprises a heavy chain constant region set forth in SEQ ID NO: 61 or 173 and a light chain constant region set forth in SEQ ID NO: 480. In certain embodiments, the antibody comprises a heavy chain sequence set forth in SEQ ID NO: 484 and/or 486 and a light chain sequence set forth in SEQ ID NO: 485. In certain embodiments, the antibody comprises a heavy chain sequence set forth in SEQ ID NO: 484 and a light chain sequence set forth in SEQ ID NO: 485. The heavy chain sequence of SEQ ID NO: 484 contains a C-terminal lysine, which may be cleaved off during manufacture or after administration, resulting in the heavy chain sequence of SEQ ID NO: 486. In other embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29. In other embodiments, the antibody, or antigen binding fragment thereof, comprises a VH sequence set forth in SEQ ID NO: 478 and a VL sequence set forth in SEQ ID NO: 479. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In other embodiments, the antibody, or antigen binding fragment thereof, comprises a heavy chain sequence set forth in SEQ ID NO: 476 and a light chain sequence set forth in SEQ ID NO: 477.

In certain embodiments, the hyaluronidase is a soluble hyaluronidase. In certain embodiments, the hyaluronidase is a recombinant human hyaluronidase or variant thereof. In certain embodiments, the hyaluronidase is a recombinant human hyaluronidase. In certain embodiments, the recombinant human hyaluronidase comprises the amino acid sequence set forth in any one of SEQ ID NOs: 409-418 and 420-475, or mixture thereof. In certain embodiments, the recombinant human hyaluronidase is rHuPH20 (i.e., a composition comprising one or more of SEQ ID NOs: 411-416). In certain embodiments, the rHuPH20 formulation is ENHANZE®.

In certain embodiments, the kit is for use in treating an inflammatory disorder or disease. In certain embodiments, the inflammatory disorder or disease is atopic dermatitis. In certain embodiments, the inflammatory disorder or disease is asthma, idiopathic pulmonary fibrosis, alopecia areata, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), inflammatory bowel disease, such as Crohn's disease or ulcerative colitis, lupus, rheumatoid arthritis (RA), psoriasis, hidradenitis suppurativa, celiac disease, or systemic sclerosis. In certain embodiments, the inflammatory disorder or disease is an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE).

### EXAMPLES

Below is an example of specific embodiments for carrying out the present invention. The example is offered for illustrative purposes only, and is not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Clinical Study

A clinical study is conducted to assess the safety, tolerability, pharmacokinetics, pharmacodynamics, and immunogenicity of an isolated antibody, or antigen binding fragment thereof, that binds TSLP administered in combination with hyaluronidase or a variant thereof.

In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27. In certain embodiments, the antibody, or antigen binding fragment thereof, that binds TSLP comprises a VH sequence set forth in SEQ ID NO: 34 and a VL sequence set forth in SEQ ID NO: 41. In certain embodiments, the antibody, or antigen binding fragment thereof, that binds TSLP comprises a heavy chain constant region set forth in SEQ ID NO: 61 or 173 and a light chain constant region set forth in SEQ ID NO: 480. A C-terminal lysine is present in SEQ ID NO: 173, which may be cleaved off during manufacture or after administration, resulting in the sequence of SEQ ID NO: 61. Accordingly, a composition comprising an antibody comprising the constant heavy chain of SEQ ID NO: 173 that is administered to a subject may comprise antibodies having the constant heavy chain sequence set forth in SEQ ID NO: 173 or SEQ ID NO: 61, or a mixture thereof (e.g., a composition comprising anti-TSLP antibodies may contain a mixture of antibodies having either a constant heavy chain sequence of SEQ ID NO: 173 or a constant heavy chain sequence of SEQ ID NO: 61 and/or antibodies containing both constant heavy chain sequences (e.g., in a single antibody containing two constant heavy chain sequences)). In certain embodiments, the antibody, or antigen binding fragment thereof, that binds TSLP comprises a heavy chain sequence set forth in SEQ ID NO: 484 and/or 486 and a light chain sequence set forth in SEQ ID NO: 485. In one embodiment, the antibody that binds TSLP comprises the heavy chain sequence set forth in SEQ ID NO: 484 and the light chain sequence set forth in SEQ ID NO: 485. The C-terminal lysine in SEQ ID NO: 484 may not be present if cleavage occurs during manufacture or after administration (which would result in the heavy chain sequence of SEQ ID NO: 486).

In certain embodiments, the antibody, or antigen binding fragment thereof, that binds TSLP comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29. In certain embodiments, the antibody, or antigen binding fragment thereof, that binds TSLP comprises a VH sequence set forth in SEQ ID NO: 478 and a VL sequence set forth in SEQ ID NO: 479. In certain embodiments, the antibody, or antigen binding fragment thereof, that binds TSLP comprises a human IgG sequence selected from a sequence set forth in any one of SEQ ID NOs: 47-50, 52-162, and 164-270. In certain embodiments, the antibody does not comprise a human IgG2 constant heavy chain sequence (e.g., SEQ ID NO: 51 or SEQ ID NO: 163). In certain embodiments, the antibody that binds TSLP comprises a heavy chain sequence set forth in SEQ ID NO: 476 and a light chain sequence set forth in SEQ ID NO: 477.

In one embodiment, the hyaluronidase is a recombinant human hyaluronidase, such as rHuPH20 (i.e., a composition comprising one or more of SEQ ID NOs: 411-416). In certain embodiments, the rHuPH20 formulation is ENHANZER.

In certain embodiments, the hyaluronidase or variant thereof and the anti-TSLP antibody, or antigen binding fragment thereof, are administered in separate formulations. In certain embodiments, the hyaluronidase or variant thereof and the anti-TSLP antibody, or antigen binding fragment thereof, are administered simultaneously in separate formulations. In certain embodiments, the hyaluronidase or variant thereof is administered to the patient prior to administration of the anti-TSLP antibody, or antigen binding fragment thereof. In certain embodiments, the hyaluronidase or variant thereof is administered to the patient after administration of the anti-TSLP antibody, or antigen binding fragment thereof. In certain embodiments, the hyaluronidase or variant thereof and the anti-TSLP antibody, or antigen binding fragment thereof, are mixed and administered in a single formulation. In certain embodiments, the hyaluronidase or variant thereof and the anti-TSLP antibody, or antigen binding fragment thereof, are administered by subcutaneous injection. In certain embodiments, the hyaluronidase or variant thereof and the anti-TSLP antibody, or antigen binding fragment thereof, are administered by intravenous injection.

In certain embodiments, the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

Exemplary embodiments of the invention are described in the enumerated paragraphs below.
E1. A composition comprising an isolated antibody, or antigen binding fragment thereof, that binds thymic stromal lymphopoietin (TSLP) and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27.
E2. The composition of E1, wherein the antibody, or antigen binding fragment thereof, comprises a VH sequence set forth in SEQ ID NO:34 and a VL sequence set forth in SEQ ID NO: 41.
E3. A composition comprising an isolated antibody, or antigen binding fragment thereof, that binds TSLP and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29.
E4. The composition of E3, wherein the antibody, or antigen binding fragment thereof, comprises a VH sequence set forth in SEQ ID NO:478 and a VL sequence set forth in SEQ ID NO:479.
E5. The composition of any one of E1-E4, wherein the antibody, or antigen binding fragment thereof, is a humanized, human, or chimeric antibody.
E6. The composition of E5, wherein the antibody, or antigen binding fragment thereof, is a humanized antibody.
E7. The composition of any one of E1-E6, wherein the antibody, or antigen binding fragment thereof, comprises a heavy chain human constant region of a class selected from IgG, IgA, IgD, IgE, and IgM.
E8. The composition of E7, wherein the antibody, or antigen binding fragment thereof, comprises a heavy chain human constant region of the class IgG and a subclass selected from IgG1, IgG2, IgG3, and IgG4.
E9 The composition of any one of E1-E8, wherein the antibody, or antigen binding fragment thereof, comprises a human IgG1 Fc region.
E10. The composition of any one of E1-E9, wherein the antibody, or antigen binding fragment thereof, comprises a human Fc region comprising a human IgG sequence selected from a sequence set forth in any one of SEQ ID NOs: 47-270.
E11. The composition of any one of E1-E10, wherein the antibody, or antigen binding fragment thereof, comprises a heavy chain comprising a constant heavy chain region having a means for extending the half-life of the antibody.
E12. The composition of any one of E1-E11, wherein the antibody, or antigen binding fragment thereof, comprises a light chain comprising a constant light chain sequence set forth in SEQ ID NO: 480.
E13. The composition of any one of E1-E12, wherein the antibody, or antigen binding fragment thereof, comprises an Fc region comprising one or more amino acid substitutions, wherein the one or more amino acid substitutions result in an increase in one or more of antibody half-life, ADCC activity, ADCP activity, or CDC activity compared with the Fc region without the one or more amino acid substitutions.
E14. The composition of any one of E1-E12, wherein the antibody, or antigen binding fragment thereof, comprises an Fc region comprising one or more amino acid substitutions, wherein the one or more amino acid substitutions result in a decrease in one or more of, ADCC activity, ADCP activity or CDC activity compared to an antibody comprising a wild-type Fc region.
E15. The composition of E13 or E14, wherein the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions comprises a plurality of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.
E16. The composition of E15, wherein the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.
E17. The composition of any one of E1-E16, wherein the antibody, or antigen binding fragment thereof, comprises a human IgG1 Fc region with LALA and YTE mutations.
E18. The composition of any one of E1-E17, wherein the antibody, or antigen binding fragment thereof, comprises a human IgG1 Fc region with LALA mutations at positions L239A/L240A by direct numbering and YTE mutations at positions M257Y/S259T/T261E by direct numbering.
E19. The composition of any one of E1-E18, wherein the antibody, or antigen binding fragment thereof, comprises a heavy chain comprising a constant heavy chain sequence set forth in SEQ ID NO: 173 and/or SEQ ID NO: 61.
E20. The composition of E19, wherein the antibody, or antigen binding fragment thereof, comprises a heavy chain comprising a constant heavy chain sequence set forth in SEQ ID NO: 173.
E21. The composition of E19, wherein the antibody, or antigen binding fragment thereof, comprises a heavy chain comprising a constant heavy chain sequence set forth in SEQ ID NO: 61.
E22. The composition of any one of E1, E2, and E5-E21, wherein the antibody, or antigen binding fragment thereof, comprises a VH domain comprising the amino acid sequence set forth in SEQ ID NO: 34, a VL domain comprising the amino acid sequence set forth in SEQ ID NO: 41, and a human IgG1 Fc region comprising LALA and YTE substitutions.
E23. The composition of any one of E1, E2, and E5-E22, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173 and/or a constant heavy chain sequence set forth in SEQ ID NO: 61, and a constant light chain sequence set forth in SEQ ID NO: 480.
E24. The composition of E23, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a constant light chain sequence set forth in SEQ ID NO: 480.
E25. The composition of E24, wherein the antibody, or antigen binding fragment thereof, comprises a heavy chain sequence set forth in SEQ ID NO: 484 and a light chain sequence set forth in SEQ ID NO: 485.
E26. The composition of E23, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61, and a constant light chain sequence set forth in SEQ ID NO: 480.
E27. The composition of E26, wherein the antibody, or antigen binding fragment thereof, comprises a heavy chain sequence set forth in SEQ ID NO: 486 and a light chain sequence set forth in SEQ ID NO: 485.
E28. The composition of E3 or E4, wherein the antibody, or antigen binding fragment thereof, comprises a heavy chain sequence set forth in SEQ ID NO: 476 and a light chain sequence set forth in SEQ ID NO: 477.
E29. The composition of any one of E1-E28, wherein the Fc region of the antibody, or antigen binding fragment thereof, binds to Neonatal Fc receptor (FcRn).
E30. The composition of E28, wherein the Fc region of the antibody, or antigen binding fragment thereof, binds an FcRn with higher affinity at pH 6.0 compared to an antibody comprising a wild-type Fc region.
E31. The composition of any one of E1-E30, wherein the antibody, or antigen binding fragment thereof, is a monoclonal antibody.
E32. The composition of any one of E1-E31, wherein the antibody, or antigen binding fragment thereof, binds a TSLP sequence set forth in SEQ ID NO: 365 or 366.
E33. The composition of any one of E1-E32, wherein the hyaluronidase is a recombinant human hyaluronidase.
E34. The composition of E33, wherein the recombinant human hyaluronidase comprises an amino acid sequence set forth in any one of SEQ ID NOs: 409-418 and 420-475 (e.g., SEQ ID NO: 411), or a mixture thereof.
E35. The composition of E33, wherein the recombinant human hyaluronidase is a soluble hyaluronidase provided as a composition designated rHuPH20.
E36. The composition of E35, wherein the rHuPH20 formulation is ENHANZE®.
E37. The composition of any one of E1-E36, wherein the composition is formulated for administration by subcutaneous injection.
E38. The composition of any one of E1-E36, wherein the composition is formulated for administration by intravenous injection.
E39. A combination comprising an isolated antibody, or antigen binding fragment thereof, that binds thymic stromal lymphopoietin (TSLP) and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27.
E40. The combination of E39, wherein the antibody, or antigen binding fragment thereof, comprises a VH sequence set forth in SEQ ID NO:34 and a VL sequence set forth in SEQ ID NO: 41.
E41. A combination comprising an isolated antibody, or antigen binding fragment thereof, that binds TSLP and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29.
E42. The combination of E41, wherein the antibody, or antigen binding fragment thereof, comprises a VH sequence set forth in SEQ ID NO:478 and a VL sequence set forth in SEQ ID NO:479.
E43. The combination of any one of E39-E42, wherein the antibody, or antigen binding fragment thereof, is a humanized, human, or chimeric antibody.
E44. The combination of E43, wherein the antibody, or antigen binding fragment thereof, is a humanized antibody.
E45. The combination of any one of E39-E44, wherein the antibody, or antigen binding fragment thereof, comprises a heavy chain human constant region of a class selected from IgG, IgA, IgD, IgE, and IgM.
E46. The combination of E45, wherein the antibody, or antigen binding fragment thereof, comprises a heavy chain human constant region of the class IgG and a subclass selected from IgG1, IgG2, IgG3, and IgG4.
E47. The combination of any one of E39-E46, wherein the antibody, or antigen binding fragment thereof, comprises a human IgG1 Fc region.
E48. The combination of any one of E39-E47, wherein the antibody, or antigen binding fragment thereof, comprises a human Fc region comprising a human IgG sequence selected from a sequence set forth in any one of SEQ ID NOs: 47-270.
E49. The combination of any one of E39-E48, wherein the antibody, or antigen binding fragment thereof, comprises a heavy chain comprising a constant heavy chain region having a means for extending the half-life of the antibody.
E50. The combination of any one of E39-E49, wherein the antibody, or antigen binding fragment thereof, comprises a light chain comprising a constant light chain sequence set forth in SEQ ID NO: 480.
E51. The combination of any one of E39-E50, wherein the antibody, or antigen binding fragment thereof, comprises an Fc region comprising one or more amino acid substitutions, wherein the one or more amino acid substitutions result in an increase in one or more of antibody half-life, ADCC activity, ADCP activity, or CDC activity compared with the Fc region without the one or more amino acid substitutions.
E52. The combination of any one of E39-E50, wherein the antibody, or antigen binding fragment thereof, comprises an Fc region comprising one or more amino acid substitutions, wherein the one or more amino acid substitutions result in a decrease in one or more of, ADCC activity, ADCP activity or CDC activity compared to an antibody comprising a wild-type Fc region.
E53. The combination of E51 or E52, wherein the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions comprises a plurality of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.
E54. The combination of E53, wherein the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.
E55. The combination of any one of E39-E54, wherein the antibody, or antigen binding fragment thereof, comprises a human IgG1 Fc region with LALA and YTE mutations.
E56. The combination of any one of E39-E55, wherein the antibody, or antigen binding fragment thereof, comprises a human IgG1 Fc region with LALA mutations at positions L239A/L240A by direct numbering and YTE mutations at positions M257Y/S259T/T261E by direct numbering.
E57. The combination of any one of E39-E56, wherein the antibody, or antigen binding fragment thereof, comprises a heavy chain comprising a constant heavy chain sequence set forth in SEQ ID NO: 173 and/or SEQ ID NO: 61.
E58. The combination of E57, wherein the antibody, or antigen binding fragment thereof, comprises a heavy chain comprising a constant heavy chain sequence set forth in SEQ ID NO: 173.
E59. The combination of E57, wherein the antibody, or antigen binding fragment thereof, comprises a heavy chain comprising a constant heavy chain sequence set forth in SEQ ID NO: 61.
E60. The combination of any one of E39, E40, and E43-E59, wherein the antibody, or antigen binding fragment thereof, comprises a VH domain comprising the amino acid sequence set forth in SEQ ID NO: 34, a VL domain comprising the amino acid sequence set forth in SEQ ID NO: 41, and a human IgG1 Fc region comprising LALA and YTE substitutions.
E61. The combination of any one of E39, E40, and E43-E60, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173 and/or a constant heavy chain sequence set forth in SEQ ID NO: 61, and a constant light chain sequence set forth in SEQ ID NO: 480.
E62. The combination of E61, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a constant light chain sequence set forth in SEQ ID NO: 480.
E63. The combination of E62, wherein the antibody, or antigen binding fragment thereof, comprises a heavy chain sequence set forth in SEQ ID NO: 484 and a light chain sequence set forth in SEQ ID NO: 485.
E64. The combination of E61, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61, and a constant light chain sequence set forth in SEQ ID NO: 480.
E65. The combination of E64, wherein the antibody, or antigen binding fragment thereof, comprises a heavy chain sequence set forth in SEQ ID NO: 486 and a light chain sequence set forth in SEQ ID NO: 485.
E66. The combination of E41 or E42, wherein the antibody, or antigen binding fragment thereof, comprises a heavy chain sequence set forth in SEQ ID NO: 476 and a light chain sequence set forth in SEQ ID NO: 477.
E67. The combination of any one of E39-E66, wherein the Fc region of the antibody, or antigen binding fragment thereof, binds to Neonatal Fc receptor (FcRn).
E68. The combination of E67, wherein the Fc region of the antibody, or antigen binding fragment thereof, binds an FcRn with higher affinity at pH 6.0 compared to an antibody comprising a wild-type Fc region.
E69. The combination of any one of E39-E68, wherein the antibody, or antigen binding fragment thereof, is a monoclonal antibody.
E70. The combination of any one of E39-E69, wherein the antibody, or antigen binding fragment thereof, binds a TSLP sequence set forth in SEQ ID NO: 365 or 366.
E71. The combination of any one of E39-E70, wherein the hyaluronidase is a recombinant human hyaluronidase.
E72. The combination of E71, wherein the recombinant human hyaluronidase comprises an amino acid sequence set forth in any one of SEQ ID NOs: 409-418 and 420-475 (e.g., SEQ ID NO: 411), or a mixture thereof.
E73. The combination of E71, wherein the recombinant human hyaluronidase is a soluble hyaluronidase provided as a composition designated rHuPH20.
E74. The combination of E73, wherein the rHuPH20 formulation is ENHANZE®.
E75. The combination of any one of E39-E74, wherein the hyaluronidase and the antibody, or antigen binding fragment thereof, are administered in separate formulations.
E76. The combination of E75, wherein the hyaluronidase and the antibody, or antigen binding fragment thereof, are administered simultaneously in separate formulations.
E77. The combination of E75, wherein the hyaluronidase is administered to the patient prior to administration of the antibody, or antigen binding fragment thereof.
E78. The combination of E75, wherein the hyaluronidase is administered to the patient after administration of the antibody, or antigen binding fragment thereof.
E79. The combination of any one of E39-E74, wherein the hyaluronidase and the antibody, or antigen binding fragment thereof, are mixed and administered in a single formulation.
E80. The combination of any one of E39-E79, wherein the hyaluronidase and the antibody, or antigen binding fragment thereof, are administered by subcutaneous injection.
E81. The combination of any one of E39-E79, wherein the hyaluronidase and the antibody, or antigen binding fragment thereof, are administered by intravenous injection.
E82. The combination of any one of E39-E81, wherein the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.
E83. The composition of any one of E1-E38 or the combination of any one of E39-E82, for use in the treatment of an inflammatory disorder or a disease.
E84. The composition or combination for use according to E83, wherein the inflammatory disorder or disease is atopic dermatitis; asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); idiopathic pulmonary fibrosis; alopecia areata; psoriasis; celiac disease; hidradenitis suppurativa; or systemic sclerosis.
E85. A method of treating an inflammatory disorder or disease in a human patient in need thereof comprising administering to the patient an isolated antibody, or antigen binding fragment thereof, that binds thymic stromal lymphopoietin (TSLP) and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27.
E86. The method of E85, wherein the antibody, or antigen binding fragment thereof, comprises a VH sequence set forth in SEQ ID NO:34 and a VL sequence set forth in SEQ ID NO: 41.
E87. A method of treating an inflammatory disorder or disease in a human patient in need thereof comprising administering to the patient an isolated antibody, or antigen binding fragment thereof, that binds TSLP and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 19; a CDR-L2 comprising the sequence set forth in SEQ ID NO: 26; and a CDR-L3 comprising the sequence set forth in SEQ ID NO: 29.
E88. The method of E87, wherein the antibody, or antigen binding fragment thereof, comprises a VH sequence set forth in SEQ ID NO:478 and a VL sequence set forth in SEQ ID NO:479.
E89. The method of any one of E85-E88, wherein the antibody, or antigen binding fragment thereof, is a humanized, human, or chimeric antibody.
E90. The method of E89, wherein the antibody, or antigen binding fragment thereof, is a humanized antibody.
E91. The method of any one of E85-E90, wherein the antibody, or antigen binding fragment thereof, comprises a heavy chain human constant region of a class selected from IgG, IgA, IgD, IgE, and IgM.
E92. The method of E91, wherein the antibody, or antigen binding fragment thereof, comprises a heavy chain human constant region of the class IgG and a subclass selected from IgG1, IgG2, IgG3, and IgG4.
E93. The method of any one of E85-E92, wherein the antibody, or antigen binding fragment thereof, comprises a human IgG1 Fc region.
E94. The method of any one of E85-E93, wherein the antibody, or antigen binding fragment thereof, comprises a human Fc region comprising a human IgG sequence selected from a sequence set forth in any one of SEQ ID NOs: 47-270.
E95. The method of any one of E85-E94, wherein the antibody, or antigen binding fragment thereof, comprises a heavy chain comprising a constant heavy chain region having a means for extending the half-life of the antibody.
E96. The method of any one of E85-E95, wherein the antibody, or antigen binding fragment thereof, comprises a light chain comprising a constant light chain sequence set forth in SEQ ID NO: 480.
E97. The method of any one of E85-E96, wherein the antibody, or antigen binding fragment thereof, comprises an Fc region comprising one or more amino acid substitutions, wherein the one or more amino acid substitutions result in an increase in one or more of antibody half-life, ADCC activity, ADCP activity, or CDC activity compared with the Fc region without the one or more amino acid substitutions.
E98. The method of any one of E85-E96, wherein the antibody, or antigen binding fragment thereof, comprises an Fc region comprising one or more amino acid substitutions, wherein the one or more amino acid substitutions result in a decrease in one or more of, ADCC activity, ADCP activity or CDC activity compared to an antibody comprising a wild-type Fc region.
E99. The method of E97 or E98, wherein the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions comprises a plurality of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.
E100. The method of E99, wherein the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.
E101. The method of any one of E85-E100, wherein the antibody, or antigen binding fragment thereof, comprises a human IgG1 Fc region with LALA and YTE mutations.
E102. The method of any one of E85-E101, wherein the antibody, or antigen binding fragment thereof, comprises a human IgG1 Fc region with LALA mutations at positions L239A/L240A by direct numbering and YTE mutations at positions M257Y/S259T/T261E by direct numbering.
E103. The method of any one of E85-E102, wherein the antibody, or antigen binding fragment thereof, comprises a heavy chain comprising a constant heavy chain sequence set forth in SEQ ID NO: 173 and/or SEQ ID NO: 61.
E104. The method of E103, wherein the antibody, or antigen binding fragment thereof, comprises a heavy chain comprising a constant heavy chain sequence set forth in SEQ ID NO: 173.
E105. The method of E103, wherein the antibody, or antigen binding fragment thereof, comprises a heavy chain comprising a constant heavy chain sequence set forth in SEQ ID NO: 61.
E106. The method of any one of E85, E86, and E89-E105, wherein the antibody, or antigen binding fragment thereof, comprises a VH domain comprising the amino acid sequence set forth in SEQ ID NO: 34, a VL domain comprising the amino acid sequence set forth in SEQ ID NO: 41, and a human IgG1 Fc region comprising LALA and YTE substitutions.
E107. The method of any one of E85, E86, and E89-E106, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173 and/or a constant heavy chain sequence set forth in SEQ ID NO: 61, and a constant light chain sequence set forth in SEQ ID NO: 480.
E108. The method of E107, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a constant light chain sequence set forth in SEQ ID NO: 480.
E109. The method of E108, wherein the antibody, or antigen binding fragment thereof, comprises a heavy chain sequence set forth in SEQ ID NO: 484 and a light chain sequence set forth in SEQ ID NO: 485.
E110. The method of E107, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 61, and a constant light chain sequence set forth in SEQ ID NO: 480.
E111. The method of E110, wherein the antibody, or antigen binding fragment thereof, comprises a heavy chain sequence set forth in SEQ ID NO: 486 and a light chain sequence set forth in SEQ ID NO: 485.
E112. The method of E87 or E88, wherein the antibody, or antigen binding fragment thereof, comprises a heavy chain sequence set forth in SEQ ID NO: 476 and a light chain sequence set forth in SEQ ID NO: 477.
E113. The method of any one of E85-E112, wherein the Fc region of the antibody, or antigen binding fragment thereof, binds to Neonatal Fc receptor (FcRn).
E114. The method of E113, wherein the Fc region of the antibody, or antigen binding fragment thereof, binds an FcRn with higher affinity at pH 6.0 compared to an antibody comprising a wild-type Fc region.
E115. The method of any one of E85-E114, wherein the antibody, or antigen binding fragment thereof, is a monoclonal antibody.
E116. The method of any one of E85-E115, wherein the antibody, or antigen binding fragment thereof, binds a TSLP sequence set forth in SEQ ID NO: 365 or 366.
E117. The method of any one of E85-E116, wherein the hyaluronidase is a recombinant human hyaluronidase.
E118. The method of E117, wherein the recombinant human hyaluronidase comprises an amino acid sequence set forth in any one of SEQ ID NOs: 409-418 and 420-475 (e.g., SEQ ID NO: 411), or a mixture thereof.
E119. The method of E117, wherein the recombinant human hyaluronidase is a soluble hyaluronidase provided as a composition designated rHuPH20.
E120. The method of E119, wherein the rHuPH20 formulation is ENHANZE®.
E121. The method of any one of E85-E120, wherein the hyaluronidase and the antibody, or antigen binding fragment thereof, are administered in separate formulations.
E122. The method of E121, wherein the hyaluronidase and the antibody, or antigen binding fragment thereof, are administered simultaneously in separate formulations.
E123. The method of E121, wherein the hyaluronidase is administered to the patient prior to administration of the antibody, or antigen binding fragment thereof.
E124. The method of E121, wherein the hyaluronidase is administered to the patient after administration of the antibody, or antigen binding fragment thereof.
E125. The method of any one of E85-E120, wherein the hyaluronidase and the antibody, or antigen binding fragment thereof, are mixed and administered in a single formulation.
E126. The method of any one of E85-E125, wherein the hyaluronidase and the antibody, or antigen binding fragment thereof, are administered by subcutaneous injection.
E127. The method of any one of E85-E125, wherein the hyaluronidase and the antibody, or antigen binding fragment thereof, are administered by intravenous injection.
E128. The method of any one of E85-E127, wherein the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.
E130. The method of any one of E85-E128, wherein the inflammatory disorder or disease is atopic dermatitis; asthma; chronic sinusitis with nasal polyps; Chronic Rhinosinusitis without Nasal Polyps (CRSsNP); eosinophilic esophagitis (EoE); an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE); Churg-Strauss syndrome/Eosinophilic granulomatosis with polyangiitis (EGPA); Prurigo Nodularis (PN); Chronic Spontaneous Urticaria (CSU); Chronic Pruritis of Unknown Origin (CPUO); Bullous Pemphigoid (BP); Cold Inducible Urticaria (ColdU); Allergic Fungal Rhinosinusitis (AFRS); Allergic Bronchopulmonary Aspergillosis (ABPA); Chronic Obstructive Pulmonary Disease (COPD); an inflammatory bowel disease, such as Crohn's disease or ulcerative colitis; lupus; rheumatoid arthritis (RA); idiopathic pulmonary fibrosis; alopecia areata; psoriasis; celiac disease; hidradenitis suppurativa; or systemic sclerosis.
E131. A kit for comprising:
   (a) an isolated antibody, or antigen binding fragment thereof, that binds TSLP in unit dosage form, wherein the antibody, or antigen binding fragment thereof, comprises a VH sequence set forth in SEQ ID NO: 34 and a VL sequence set forth in SEQ ID NO: 41;
   (b) a hyaluronidase or variant thereof in unit dosage form; and
   (c) instructions.
E132. A kit for comprising:
   (a) an isolated antibody, or antigen binding fragment thereof, that binds TSLP in unit dosage form, wherein the antibody, or antigen binding fragment thereof, comprises a VH sequence set forth in SEQ ID NO: 478 and a VL sequence set forth in SEQ ID NO: 479;
   (b) a hyaluronidase or variant thereof in unit dosage form; and
   (c) instructions.
E133. The kit of E131 or E132, wherein the hyaluronidase is a recombinant human hyaluronidase.
E134. The kit of E133, wherein the recombinant human hyaluronidase comprises the amino acid sequence set forth in any one of SEQ ID NOs: 409-418 and 420-475.
E135. The kit of E133, wherein the recombinant human hyaluronidase is a soluble hyaluronidase provided as a composition designated rHuPH20.
E136. The kit of E135, wherein the rHuPH20 formulation is ENHANZE®.
E137. The kit of any one of E131-E136 for use in treating an inflammatory disorder or disease.
E138. The kit of E137, wherein the inflammatory disorder or disease is atopic dermatitis.
E139. The kit of E137, wherein the inflammatory disorder or disease is selected from the group consisting of asthma, idiopathic pulmonary fibrosis, alopecia areata, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Crohn's disease, lupus, rheumatoid arthritis (RA), psoriasis, ulcerative colitis, hidradenitis suppurativa, celiac disease, and systemic sclerosis.
E140. The kit of any one of E131-E139, wherein the antibody, or antigen binding fragment thereof, comprises a human Fc region comprising a human IgG sequence selected from a sequence set forth in any one of SEQ ID NOs: 47-270.
E141. The kit of any one of E131-E140, wherein the antibody, or antigen binding fragment thereof, comprises a light chain comprising a constant light chain sequence set forth in SEQ ID NO: 480.
E142. The kit of any one of E131-E141, wherein the antibody, or antigen binding fragment thereof, comprises a human IgG1 Fc region with LALA and YTE mutations.
E143. The kit of any one of E131-E142, wherein the antibody, or antigen binding fragment thereof, comprises a human IgG1 Fc with LALA mutations at positions L239A/L240A by direct numbering and YTE mutations at positions M257Y/S259T/T261E by direct numbering.
E144. The kit of any one of E131-E143, wherein the antibody, or antigen binding fragment thereof, comprises a heavy chain comprising a constant heavy chain sequence set forth in SEQ ID NO: 173 and/or SEQ ID NO: 61.
E145. The kit of E144, wherein the antibody, or antigen binding fragment thereof, comprises a VH domain comprising the amino acid sequence set forth in SEQ ID NO: 34, a VL domain comprising the amino acid sequence set forth in SEQ ID NO: 41, a constant heavy chain sequence set forth in SEQ ID NO: 173, and a constant light chain sequence set forth in SEQ ID NO: 480.
E146. The kit of E145, wherein the antibody, or antigen binding fragment thereof, comprises a heavy chain sequence set forth in SEQ ID NO: 484 and a light chain sequence set forth in SEQ ID NO: 485.
E156. The kit of any one of E132-E141, wherein the antibody, or antigen binding fragment thereof, comprises a heavy chain sequence set forth in SEQ ID NO: 476 and a light chain sequence set forth in SEQ ID NO: 477.

INFORMAL SEQUENCE LISTING

| | | SEQ ID |
|---|---|---|
| Description | Sequence | NO |
| HCDR1 | TYTMH | 1 |
| HCDR1 | TYPMH | 2 |
| HCDR1 | TYGMH | 3 |
| HCDR1 | GFTFRTYTMH | 4 |
| HCDR1 | GFTFRTYPMH | 5 |
| HCDR1 | GFTFRTYGMH | 6 |
| HCDR1 | GFTFRTYT | 7 |
| HCDR1 | GFTFRTYP | 8 |
| HCDR1 | GFTFRTYG | 9 |
| HCDR2 | VIWYDGSNKHYADSVKG | 10 |
| HCDR2 | WYDGSN | 11 |
| HCDR3 | IWYDGSNK | 12 |
| HCDR3 | APQWELVHEAFDI | 13 |
| HCDR3 | ALIWELFHEYFDI | 14 |
| HCDR3 | ALLWELFHEYFDI | 15 |
| HCDR3 | ARAPQWELVHEAFDI | 16 |
| HCDR3 | ARALIWELFHEYFDI | 17 |
| HCDR3 | ARALLWELFHEYFDI | 18 |
| LCDR1 | GGNNLGSKSVH | 19 |
| LCDR1 | GGNNLISKSVH | 20 |
| LCDR1 | GGNNLVSKSVH | 21 |
| LCDR1 | NLGSKS | 22 |
| LCDR1 | NLISKS | 23 |
| LCDR1 | NLVSKS | 24 |
| LCDR2 | DVSDRPS | 25 |
| LCDR2 | DDSDRPS | 26 |
| LCDR3 | QVWDSSSTFVV | 27 |
| LCDR3 | QVWDSSSTYVV | 28 |
| LCDR3 | QVWDSSSDHVV | 29 |
| LCDR3 | QVWDSSSTIVV | 30 |
| LCDR3 | QVWDKSSTYVV | 31 |
| VH | QMQLVESGGGVVQPGRSLRLSCAASGFTFRTYTMHWVRQA | 32 |
| | PGKGLEWVAVIWYDGSNKHYADSVKGRFTITRDNSKNTLN | |
| | LQMNSLRAEDTAVYYCARAPQWELVHEAFDIWGQGTMVT | |
| | VSS | |
| VH | QMQLVESGGGVVQPGRSLRLSCAASGFTFRTYGMHWVRQ | 34 |
| | APGKGLEWVAVIWYDGSNKHYADSVKGRFTITRDNSKNTL | |
| | NLQMNSLRAEDTAVYYCARAPQWELVHEAFDIWGQGTMV | |
| | TVSS | |
| VH | QMQLVESGGGVVQPGRSLRLSCAASGFTFRTYPMHWVRQA | 33 |
| | PGKGLEWVAVIWYDGSNKHYADSVKGRFTITRDNSKNTLN | |
| | LQMNSLRAEDTAVYYCARAPQWELVHEAFDIWGQGTMVT | |
| | VSS | |
| VH | QMQLVESGGGVVQPGRSLRLSCAASGFTFRTYPMHWVRQA | 386 |
| | PGKGLEWVAVIWYDGSNKHYADSVKGRFTITRDNSKNTLN | |
| | LQMNSLRAEDTAVYYCARALIWELFHEYFDIWGQGTMVTV | |
| | SS | |
| VH | QMQLVESGGGVVQPGRSLRLSCAASGFTFRTYGMHWVRQ | 35 |
| | APGKGLEWVAVIWYDGSNKHYADSVKGRFTITRDNSKNTL | |
| | NLQMNSLRAEDTAVYYCARALLWELFHEYFDIWGQGTMV | |
| | TVSS | |
| VH | QMQLVESGGGVVQPGRSLRLSCAASGFTFRTYPMHWVRQA | 36 |
| | PGKGLEWVAVIWYDGSNKHYADSVKGRFTITRDNSKNTLN | |
| | LQMNSLRAEDTAVYYCARALLWELFHEYFDIWGQGTMVT | |
| | VSS | |
| VL | SYVLTQPPSVSVAPGQTARITCGGNNLGSKSVHWYQQKPG | 37 |
| | QAPVLVVYDVSDRPSWIPERFSGSNSGNTATLTISRGEAGDE | |
| | ADYYCQVWDSSSTFVVFGGGTKLTVL | |
| VL | SYVLTQPPSVSVAPGQTARITCGGNNLGSKSVHWYQQKPG | 38 |
| | QAPVLVVYDVSDRPSWIPERFSGSNSGNTATLTISRGEAGDE | |
| | ADYYCQVWDSSSTYVVFGGGTKLTVL | |
| VL | SYVLTQPPSVSVAPGQTARITCGGNNLISKSVHWYQQKPGQ | 39 |
| | APVLVVYDDSDRPSWIPERFSGSNSGNTATLTISRGEAGDEA | |
| | DYYCQVWDSSSTYVVFGGGTKLTVL | |
| VL | SYVLTQPPSVSVAPGQTARITCGGNNLVSKSVHWYQQKPG | 40 |
| | QAPVLVVYDVSDRPSWIPERFSGSNSGNTATLTISRGEAGDE | |
| | ADYYCQVWDSSSTYVVFGGGTKLTVL | |
| VL | SYVLTQPPSVSVAPGQTARITCGGNNLVSKSVHWYQQKPG | 41 |
| | QAPVLVVYDDSDRPSWIPERFSGSNSGNTATLTISRGEAGDE | |
| | ADYYCQVWDSSSTFVVFGGGTKLTVL | |
| VL | SYVLTQPPSVSVAPGQTARITCGGNNLVSKSVHWYQQKPG | 42 |
| | QAPVLVVYDVSDRPSWIPERFSGSNSGNTATLTISRGEAGDE | |
| | ADYYCQVWDSSSDHVVFGGGTKLTVL | |
| VL | SYVLTQPPSVSVAPGQTARITCGGNNLGSKSVHWYQQKPG | 43 |
| | QAPVLVVYDVSDRPSWIPERFSGSNSGNTATLTISRGEAGDE | |
| | ADYYCQVWDSSSTIVVFGGGTKLTVL | |
| VL | SYVLTQPPSVSVAPGQTARITCGGNNLGSKSVHWYQQKPG | 44 |
| | QAPVLVVYDDSDRPSWIPERFSGSNSGNTATLTISRGEAGDE | |
| | ADYYCQVWDKSSTYVVFGGGTKLTVL | |
| VL | SYVLTQPPSVSVAPGQTARITCGGNNLVSKSVHWYQQKPG | 45 |
| | QAPVLVVYDDSDRPSWIPERFSGSNSGNTATLTISRGEAGDE | |
| | ADYYCQVWDSSSTYVVFGGGTKLTVL | |
| Human kappa LC | RTVAAPSVFIFPPSDEQLKSGTASVVCLLNNFYPREAKVQW | 46 |
| | KVDNALQSGNSQESVTEQDSKDSTYSLSSTLTLSKADYEKH | |
| | KVYACEVTHQGLSSPVTKSFNRGEC | |
| Heavy Chain | ASTKGPSVFPLAPSSKSTSGGTAALGCLVKDYFPEPVTVSW | 47 |
| Constant Region | NSGALTSGVHTFPAVLQSSGLYSLSSVVTVPSSSLGTQTYIC | |
| hIgG1* | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPCSRSTSESTAALGCLVKDYFPEPVTVSWN | 48 |
| Constant Region | SGALTSGVHTFPAVLQSSGLYSLSSVVTVPSSSLGTKTYTCN | |
| hIgG4 | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 49 |
| Constant Region | SGALTSGVHTFPAVLQSSGLYSLSSVVTVPSSSLGTKTYTCN | |
| IgG4-SP | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 50 |
| Constant Region | SGALTSGVHTFPAVLQSSGLYSLSSVVTVPSSSLGTKTYTCN | |
| IgG4-SPLE | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 51 |
| Constant Region | SGALTSGVHTFPAVLQSSGLYSLSSVVTVPSSNFGTQTYTCN | |
| IgG2 | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSW | 52 |
| Constant Region | NSGALTSGVHTFPAVLQSSGLYSLSSVVTVPSSSLGTQTYIC | |
| hIgG1-N297A | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 53 |
| Constant Region | NSGALTSGVHTFPAVLQSSGLYSLSSVVTVPSSSLGTQTYIC | |
| hIgG1-D265A | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 54 |
| Constant Region | NSGALTSGVHTFPAVLQSSGLYSLSSVVTVPSSSLGTQTYIC | |
| hIgG1-LALA | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 55 |
| Constant Region | NSGALTSGVHTFPAVLQSSGLYSLSSVVTVPSSSLGTQTYIC | |
| hIgG1-LAGA | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 56 |
| Constant Region | NSGALTSGVHTFPAVLQSSGLYSLSSVVTVPSSSLGTQTYIC | |
| hIgG1-LALAGA | NVNHKPSNTKVDKKVEPKSCDKTHTCPPCPAPEAAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 57 |
| Constant Region | NSGALTSGVHTFPAVLQSSGLYSLSSVVTVPSSSLGTQTYIC | |
| hIgG1-LALAPG | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 58 |
| Constant Region | NSGALTSGVHTFPAVLQSSGLYSLSSVVTVPSSSLGTQTYIC | |
| hIgG1-YTE | NVNHKPSNTKVDKKVEPKSCDKTHTCPPCPAPELLGGPSVF | |
| | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 59 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/YTE | LFPPKPKDTLYITREPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 60 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/YTE | LFPPKPKDTLYITREPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 61 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 62 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 63 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 64 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 65 |
| Constant Region | NSGALTSGVHTFPAVLQSSGLYSLSSVVTVPSSSLGTQTYIC | |
| hIgG1-LS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 66 |
| Constant Region | NSGALTSGVHTFPAVLQSSGLYSLSSVVTVPSSSLGTQTYIC | |
| hIgG1-N297A/LS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 67 |
| Constant Region | NSGALTSGVHTFPAVLQSSGLYSLSSVVTVPSSSLGTQTYIC | |
| hIgG1-D265A/LS | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 68 |
| Constant Region | NSGALTSGVHTFPAVLQSSGLYSLSSVVTVPSSSLGTQTYIC | |
| hIgG1-LALA/LS | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 69 |
| Constant Region | NSGALTSGVHTFPAVLQSSGLYSLSSVVTVPSSSLGTQTYIC | |
| hIgG1-LAGA/LS | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 70 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/LS | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 71 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/LS | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| Heavy Chain | ASTKGPSVFPLAPSSKSTSGGTAALGCLVKDYFPEPVTVSW | 72 |
| Constant Region | NSGALTSGVHTFPAVLQSSGLYSLSSVVTVPSSSLGTQTYIC | |
| hIgG1-DHS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 73 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DHS | LFPPKPKDTLMISRTPEVTCVVVDVSHEDPEVKFNWYVDGV | |
| | EVHNAKTKPREEQYASTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 74 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DHS | LFPPKPKDTLMISRTPEVTCVVVAVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 75 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 76 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 77 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPSSKSTSGGTAALGCLVKDYFPEPVTVSW | 78 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | G | |
| Heavy Chain | ASTKGPSVFPLAPCSRSTSESTAALGCLVKDYFPEPVTVSWN | 79 |
| Constant Region | SGALTSGVHTFPAVLQSSGLYSLSSVVTVPSSSLGTKTYTCN | |
| hIgG4-YTE | VDHKPSNTKVDKRVESKYGPPCPSCPAPEFLGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 80 |
| Constant Region | SGALTSGVHTFPAVLQSSGLYSLSSVVTVPSSSLGTKTYTCN | |
| hIgG4-SP/YTE | VDHKPSNTKVDKRVESKYGPPCPPCPAPEFLGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 81 |
| Constant Region | SGALTSGVHTFPAVLQSSGLYSLSSVVTVPSSSLGTKTYTCN | |
| hIgG4-SPLE/YTE | VDHKPSNTKVDKRVESKYGPPCPPCPAPEELGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPCSRSTSESTAALGCLVKDYFPEPVTVSWN | 82 |
| Constant Region | SGALTSGVHTFPAVLQSSGLYSLSSVVTVPSSSLGTKTYTCN | |
| hIgG4-LS | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLG | |
| Heavy Chain | ASTKGPSVFPLAPCSRSTSESTAALGCLVKDYFPEPVTVSWN | 83 |
| Constant Region | SGALTSGVHTFPAVLQSSGLYSLSSVVTVPSSSLGTKTYTCN | |
| hIgG4-SP/LS | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLG | |
| Heavy Chain | ASTKGPSVFPLAPCSRSTSESTAALGCLVKDYFPEPVTVSWN | 84 |
| Constant Region | SGALTSGVHTFPAVLQSSGLYSLSSVVTVPSSSLGTKTYTCN | |
| hIgG4-SPLE/LS | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLG | |
| Heavy Chain | ASTKGPSVFPLAPCSRSTSESTAALGCLVKDYFPEPVTVSWN | 85 |
| Constant Region | SGALTSGVHTFPAVLQSSGLYSLSSVVTVPSSSLGTKTYTCN | |
| hIgG4-DHS | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLG | |
| Heavy Chain | ASTKGPSVFPLAPCSRSTSESTAALGCLVKDYFPEPVTVSWN | 86 |
| Constant Region | SGALTSGVHTFPAVLQSSGLYSLSSVVTVPSSSLGTKTYTCN | |
| hIgG4-SP/DHS | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLG | |
| Heavy Chain | ASTKGPSVFPLAPCSRSTSESTAALGCLVKDYFPEPVTVSWN | 87 |
| Constant Region | SGALTSGVHTFPAVLQSSGLYSLSSVVTVPSSSLGTKTYTCN | |
| hIgG4-SPLE/DHS | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLG | |
| Heavy Chain | ASTKGPSVFPLAPCSRSTSESTAALGCLVKDYFPEPVTVSWN | 88 |
| Constant Region | SGALTSGVHTFPAVLQSSGLYSLSSVVTVPSSNFGTQTYTCN | |
| hIgG2-YTE | VDHKPSNTKVDKTVERKCCVECPPCPAPPVAGPSVFLFPPKP | |
| | KDTLYITREPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHNHYTQKSLSLSPG | |
| Heavy Chain | ASTKGPSVFPLAPCSRSTSESTAALGCLVKDYFPEPVTVSWN | 89 |
| Constant Region | SGALTSGVHTFPAVLQSSGLYSLSSVVTVPSSNFGTQTYTCN | |
| hIgG2-LS | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVLHEALHSHYTQKSLSLSPG | |
| Heavy Chain | ASTKGPSVFPLAPCSRSTSESTAALGCLVKDYFPEPVTVSWN | 90 |
| Constant Region | SGALTSGVHTFPAVLQSSGLYSLSSVVTVPSSNFGTQTYTCN | |
| hIgG2-DHS | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVDHHDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHSHYTQKSLSLSPG | |
| Heavy Chain | ASTKGPSVFPLAPCSRSTSESTAALGCLVKDYFPEPVTVSWN | 91 |
| Constant Region | SGALTSGVHTFPAVLQSSGLYSLSSVVTVPSSSLGTKTYTCN | |
| IgG4-SP | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLG | |
| Heavy Chain | ASTKGPSVFPLAPSSKSTSGGTAALGCLVKDYFPEPVTVSW | 92 |
| Constant Region | NSGALTSGVHTFPAVLQSSGLYSLSSVVTVPSSSLGTQTYIC | |
| hIgG1-LA | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 93 |
| Constant Region | NSGALTSGVHTFPAVLQSSGLYSLSSVVTVPSSSLGTQTYIC | |
| hIgG1-N297A/LA | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 94 |
| Constant Region | NSGALTSGVHTFPAVLQSSGLYSLSSVVTVPSSSLGTQTYIC | |
| hIgG1-D265A/LA | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 95 |
| Constant Region | NSGALTSGVHTFPAVLQSSGLYSLSSVVTVPSSSLGTQTYIC | |
| hIgG1-LALA/LA | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 96 |
| Constant Region | NSGALTSGVHTFPAVLQSSGLYSLSSVVTVPSSSLGTQTYIC | |
| hIgG1-LAGA/LA | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 97 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/LA | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 98 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/LA | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| Heavy Chain | ASTKGPSVFPLAPSSKSTSGGTAALGCLVKDYFPEPVTVSW | 99 |
| Constant Region | NSGALTSGVHTFPAVLQSSGLYSLSSVVTVPSSSLGTQTYIC | |
| hIgG1-N434A | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 100 |
| Constant Region | NSGALTSGVHTFPAVLQSSGLYSLSSVVTVPSSSLGTQTYIC | |
| hIgG1-N297A/ | NVNHKPSNTKVDKKVEPKSCDKTHTCPPCPAPELLGGPSVF | |
| N434A | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 101 |
| Constant Region | NSGALTSGVHTFPAVLQSSGLYSLSSVVTVPSSSLGTQTYIC | |
| hIgG1-D265A/ | NVNHKPSNTKVDKKVEPKSCDKTHTCPPCPAPELLGGPSVF | |
| N434A | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 102 |
| Constant Region | NSGALTSGVHTFPAVLQSSGLYSLSSVVTVPSSSLGTQTYIC | |
| hIgG1-LALA/ | NVNHKPSNTKVDKKVEPKSCDKTHTCPPCPAPEAAGGPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 103 |
| Constant Region | NSGALTSGVHTFPAVLQSSGLYSLSSVVTVPSSSLGTQTYIC | |
| hIgG1-LAGA/ | NVNHKPSNTKVDKKVEPKSCDKTHTCPPCPAPELAGAPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 104 |
| Constant Region | NSGALTSGVHTFPAVLQSSGLYSLSSVVTVPSSSLGTQTYIC | |
| hIgG1-LALAGA/ | NVNHKPSNTKVDKKVEPKSCDKTHTCPPCPAPEAAGAPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 105 |
| Constant Region | NSGALTSGVHTFPAVLQSSGLYSLSSVVTVPSSSLGTQTYIC | |
| hIgG1-LALAPG/ | NVNHKPSNTKVDKKVEPKSCDKTHTCPPCPAPEAAGGPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | G | |
| Heavy Chain | ASTKGPSVFPLAPSSKSTSGGTAALGCLVKDYFPEPVTVSW | 106 |
| Constant Region | NSGALTSGVHTFPAVLQSSGLYSLSSVVTVPSSSLGTQTYIC | |
| hIgG1-N434W | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 107 |
| Constant Region | NSGALTSGVHTFPAVLQSSGLYSLSSVVTVPSSSLGTQTYIC | |
| hIgG1-N297A/ | NVNHKPSNTKVDKKVEPKSCDKTHTCPPCPAPELLGGPSVF | |
| N434W | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 108 |
| Constant Region | NSGALTSGVHTFPAVLQSSGLYSLSSVVTVPSSSLGTQTYIC | |
| hIgG1-D265A/ | NVNHKPSNTKVDKKVEPKSCDKTHTCPPCPAPELLGGPSVF | |
| N434W | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 109 |
| Constant Region | NSGALTSGVHTFPAVLQSSGLYSLSSVVTVPSSSLGTQTYIC | |
| hIgG1-LALA/ | NVNHKPSNTKVDKKVEPKSCDKTHTCPPCPAPEAAGGPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 110 |
| Constant Region | NSGALTSGVHTFPAVLQSSGLYSLSSVVTVPSSSLGTQTYIC | |
| hIgG1-LAGA/ | NVNHKPSNTKVDKKVEPKSCDKTHTCPPCPAPELAGAPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 111 |
| Constant Region | NSGALTSGVHTFPAVLQSSGLYSLSSVVTVPSSSLGTQTYIC | |
| hIgG1-LALAGA/ | NVNHKPSNTKVDKKVEPKSCDKTHTCPPCPAPEAAGAPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 112 |
| Constant Region | NSGALTSGVHTFPAVLQSSGLYSLSSVVTVPSSSLGTQTYIC | |
| hIgG1-LALAPG/ | NVNHKPSNTKVDKKVEPKSCDKTHTCPPCPAPEAAGGPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | G | |
| Heavy Chain | ASTKGPSVFPLAPSSKSTSGGTAALGCLVKDYFPEPVTVSW | 113 |
| Constant Region | NSGALTSGVHTFPAVLQSSGLYSLSSVVTVPSSSLGTQTYIC | |
| hIgG1-DQ | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 114 |
| Constant Region | NSGALTSGVHTFPAVLQSSGLYSLSSVVTVPSSSLGTQTYIC | |
| hIgG1-N297A/DQ | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYASTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 115 |
| Constant Region | NSGALTSGVHTFPAVLQSSGLYSLSSVVTVPSSSLGTQTYIC | |
| hIgG1-D265A/DQ | NVNHKPSNTKVDKKVEPKSCDKTHTCPPCPAPELLGGPSVF | |
| | LFPPKPKDTLMISRDPEVTCVVVAVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 116 |
| Constant Region | NSGALTSGVHTFPAVLQSSGLYSLSSVVTVPSSSLGTQTYIC | |
| hIgG1-LALA/DQ | NVNHKPSNTKVDKKVEPKSCDKTHTCPPCPAPEAAGGPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 117 |
| Constant Region | NSGALTSGVHTFPAVLQSSGLYSLSSVVTVPSSSLGTQTYIC | |
| hIgG1-LAGA/DQ | NVNHKPSNTKVDKKVEPKSCDKTHTCPPCPAPELAGAPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 118 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DQ | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 119 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DQ | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 120 |
| Constant Region | NSGALTSGVHTFPAVLQSSGLYSLSSVVTVPSSSLGTQTYIC | |
| hIgG1-DW | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 121 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DW | LFPPKPKDTLMISRDPEVTCVVVDVSHEDPEVKFNWYVDGV | |
| | EVHNAKTKPREEQYASTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 122 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DW | LFPPKPKDTLMISRDPEVTCVVVAVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 123 |
| Constant Region | NSGALTSGVHTFPAVLQSSGLYSLSSVVTVPSSSLGTQTYIC | |
| hIgG1-LALA/DW | NVNHKPSNTKVDKKVEPKSCDKTHTCPPCPAPEAAGGPSVF | |
| | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 124 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 125 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 126 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 127 |
| Constant Region | NSGALTSGVHTFPAVLQSSGLYSLSSVVTVPSSSLGTQTYIC | |
| hIgG1-YD | NVNHKPSNTKVDKKVEPKSCDKTHTCPPCPAPELLGGPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 128 |
| Constant Region | NSGALTSGVHTFPAVLQSSGLYSLSSVVTVPSSSLGTQTYIC | |
| hIgG1-N297A/YD | NVNHKPSNTKVDKKVEPKSCDKTHTCPPCPAPELLGGPSVF | |
| | LFPPKPKDTLYISRDPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 129 |
| Constant Region | NSGALTSGVHTFPAVLQSSGLYSLSSVVTVPSSSLGTQTYIC | |
| hIgG1-D265A/YD | NVNHKPSNTKVDKKVEPKSCDKTHTCPPCPAPELLGGPSVF | |
| | LFPPKPKDTLYISRDPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 130 |
| Constant Region | NSGALTSGVHTFPAVLQSSGLYSLSSVVTVPSSSLGTQTYIC | |
| hIgG1-LALA/YD | NVNHKPSNTKVDKKVEPKSCDKTHTCPPCPAPEAAGGPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 131 |
| Constant Region | NSGALTSGVHTFPAVLQSSGLYSLSSVVTVPSSSLGTQTYIC | |
| hIgG1-LAGA/YD | NVNHKPSNTKVDKKVEPKSCDKTHTCPPCPAPELAGAPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 132 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/YD | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 133 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/YD | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 134 |
| Constant Region | NSGALTSGVHTFPAVLQSSGLYSLSSVVTVPSSSLGTQTYIC | |
| hIgG1-QVV | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 135 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/QVV | LFPPKPKDTLMISRTPEVTCVVVDVSHEDPEVKFNWYVDGV | |
| | EVHNAKTKPREEQYASTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 136 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/QVV | LFPPKPKDTLMISRTPEVTCVVVAVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 137 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 138 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 139 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 140 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALGAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 141 |
| Constant Region | NSGALTSGVHTFPAVLQSSGLYSLSSVVTVPSSSLGTQTYIC | |
| hIgG1-DDRVV | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 142 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DDRVV | LFPPKPKDTLMISRDPEVTCVVVDVSHEDPEVKFNWYVDGV | |
| | EVDNAKTKPREEQYASTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 143 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DDRVV | LFPPKPKDTLMISRDPEVTCVVVAVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 144 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 145 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 146 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 147 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALGAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | G | |
| Heavy Chain | ASTKGPSVFPLAPSSKSTSGGTAALGCLVKDYFPEPVTVSW | 148 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| Q311R/M428L | LFPPKPKDTLMISRTPEVTCVVVDVSHEDPEVKFNWYVDGV | |
| | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPCSRSTSESTAALGCLVKDYFPEPVTVSWN | 149 |
| Constant Region | SGALTSGVHTFPAVLQSSGLYSLSSVVTVPSSSLGTKTYTCN | |
| hIgG4- | VDHKPSNTKVDKRVESKYGPPCPSCPAPEFLGGPSVFLFPPK | |
| Q311R/M428L | PKDTLMISRTPEVTCVVVDVSQEDPEVQFNWYVDGVEVHN | |
| | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLG | |
| Heavy Chain | ASTKGPSVFPLAPCSRSTSESTAALGCLVKDYFPEPVTVSWN | 150 |
| Constant Region | SGALTSGVHTFPAVLQSSGLYSLSSVVTVPSSSLGTKTYTCN | |
| IgG4- | VDHKPSNTKVDKRVESKYGPPCPPCPAPEFLGGPSVFLFPPK | |
| SP/Q311R/M428L | PKDTLMISRTPEVTCVVVDVSQEDPEVQFNWYVDGVEVHN | |
| | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLG | |
| Heavy Chain | ASTKGPSVFPLAPCSRSTSESTAALGCLVKDYFPEPVTVSWN | 151 |
| Constant Region | SGALTSGVHTFPAVLQSSGLYSLSSVVTVPSSSLGTKTYTCN | |
| IgG4- | VDHKPSNTKVDKRVESKYGPPCPPCPAPEELGGPSVFLFPPK | |
| SPLE/Q311R/M4 | PKDTLMISRTPEVTCVVVDVSQEDPEVQFNWYVDGVEVHN | |
| 28L | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLG | |
| Heavy Chain | ASTKGPSVFPLAPCSRSTSESTAALGCLVKDYFPEPVTVSWN | 152 |
| Constant Region | SGALTSGVHTFPAVLQSSGLYSLSSVVTVPSSNFGTQTYTCN | |
| IgG2- | VDHKPSNTKVDKTVERKCCVECPPCPAPPVAGPSVFLFPPKP | |
| Q311R/M428L | KDTLMISRTPEVTCVVVDVSHEDPEVQFNWYVDGVEVHNA | |
| | KTKPREEQFNSTFRVVSVLTVVHRDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVLHEALHNHYTQKSLSLSPG | |
| Heavy Chain | ASTKGPSVFPLAPSSKSTSGGTAALGCLVKDYFPEPVTVSW | 153 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/Q311R/M | LFPPKPKDTLMISRTPEVTCVVVDVSHEDPEVKFNWYVDGV | |
| 428L | EVHNAKTKPREEQYASTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 154 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/Q311R/ | LFPPKPKDTLMISRTPEVTCVVVAVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 155 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 156 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 157 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 158 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| Heavy Chain | ASTKGPSVFPLAPSSKSTSGGTAALGCLVKDYFPEPVTVSW | 159 |
| Constant Region | NSGALTSGVHTFPAVLQSSGLYSLSSVVTVPSSSLGTQTYIC | |
| hIgG1* | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPCSRSTSESTAALGCLVKDYFPEPVTVSWN | 160 |
| Constant Region | SGALTSGVHTFPAVLQSSGLYSLSSVVTVPSSSLGTKTYTCN | |
| hIgG4 | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 161 |
| Constant Region | SGALTSGVHTFPAVLQSSGLYSLSSVVTVPSSSLGTKTYTCN | |
| IgG4-SP | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 162 |
| Constant Region | SGALTSGVHTFPAVLQSSGLYSLSSVVTVPSSSLGTKTYTCN | |
| IgG4-SPLE | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 163 |
| Constant Region | SGALTSGVHTFPAVLQSSGLYSLSSVVTVPSSNFGTQTYTCN | |
| IgG2 | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSW | 164 |
| Constant Region | NSGALTSGVHTFPAVLQSSGLYSLSSVVTVPSSSLGTQTYIC | |
| hIgG1-N297A | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 165 |
| Constant Region | NSGALTSGVHTFPAVLQSSGLYSLSSVVTVPSSSLGTQTYIC | |
| hIgG1-D265A | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 166 |
| Constant Region | NSGALTSGVHTFPAVLQSSGLYSLSSVVTVPSSSLGTQTYIC | |
| hIgG1-LALA | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 167 |
| Constant Region | NSGALTSGVHTFPAVLQSSGLYSLSSVVTVPSSSLGTQTYIC | |
| hIgG1-LAGA | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 168 |
| Constant Region | NSGALTSGVHTFPAVLQSSGLYSLSSVVTVPSSSLGTQTYIC | |
| hIgG1-LALAGA | NVNHKPSNTKVDKKVEPKSCDKTHTCPPCPAPEAAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 169 |
| Constant Region | NSGALTSGVHTFPAVLQSSGLYSLSSVVTVPSSSLGTQTYIC | |
| hIgG1-LALAPG | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 170 |
| Constant Region | NSGALTSGVHTFPAVLQSSGLYSLSSVVTVPSSSLGTQTYIC | |
| hIgG1-YTE | NVNHKPSNTKVDKKVEPKSCDKTHTCPPCPAPELLGGPSVF | |
| | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 171 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/YTE | LFPPKPKDTLYITREPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 172 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/YTE | LFPPKPKDTLYITREPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 173 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 174 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 175 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 176 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/YTE | LFPPKPKDTLYITREPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 177 |
| Constant Region | NSGALTSGVHTFPAVLQSSGLYSLSSVVTVPSSSLGTQTYIC | |
| hIgG1-LS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 178 |
| Constant Region | NSGALTSGVHTFPAVLQSSGLYSLSSVVTVPSSSLGTQTYIC | |
| hIgG1-N297A/LS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 179 |
| Constant Region | NSGALTSGVHTFPAVLQSSGLYSLSSVVTVPSSSLGTQTYIC | |
| hIgG1-D265A/LS | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 180 |
| Constant Region | NSGALTSGVHTFPAVLQSSGLYSLSSVVTVPSSSLGTQTYIC | |
| hIgG1-LALA/LS | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 181 |
| Constant Region | NSGALTSGVHTFPAVLQSSGLYSLSSVVTVPSSSLGTQTYIC | |
| hIgG1-LAGA/LS | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 182 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/LS | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 183 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/LS | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHSHYTQKSLSLSPG | |
| | K | |
| Heavy Chain | ASTKGPSVFPLAPSSKSTSGGTAALGCLVKDYFPEPVTVSW | 184 |
| Constant Region | NSGALTSGVHTFPAVLQSSGLYSLSSVVTVPSSSLGTQTYIC | |
| hIgG1-DHS | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 185 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DHS | LFPPKPKDTLMISRTPEVTCVVVDVSHEDPEVKFNWYVDGV | |
| | EVHNAKTKPREEQYASTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 186 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DHS | LFPPKPKDTLMISRTPEVTCVVVAVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 187 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 188 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 189 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPSSKSTSGGTAALGCLVKDYFPEPVTVSW | 190 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DHS | LFPPKPKDTLMISRTPEVTCVVVDVSHEDPEVKFNWYVDGV | |
| | EVHNAKTKPREEQYNSTYRVVSVLTVDHHDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHSHYTQKSLSLSP | |
| | GK | |
| Heavy Chain | ASTKGPSVFPLAPCSRSTSESTAALGCLVKDYFPEPVTVSWN | 191 |
| Constant Region | SGALTSGVHTFPAVLQSSGLYSLSSVVTVPSSSLGTKTYTCN | |
| hIgG4-YTE | VDHKPSNTKVDKRVESKYGPPCPSCPAPEFLGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 192 |
| Constant Region | SGALTSGVHTFPAVLQSSGLYSLSSVVTVPSSSLGTKTYTCN | |
| hIgG4-SP/YTE | VDHKPSNTKVDKRVESKYGPPCPPCPAPEFLGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 193 |
| Constant Region | SGALTSGVHTFPAVLQSSGLYSLSSVVTVPSSSLGTKTYTCN | |
| hIgG4-SPLE/YTE | VDHKPSNTKVDKRVESKYGPPCPPCPAPEELGGPSVFLFPPK | |
| | PKDTLYITREPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPCSRSTSESTAALGCLVKDYFPEPVTVSWN | 194 |
| Constant Region | SGALTSGVHTFPAVLQSSGLYSLSSVVTVPSSSLGTKTYTCN | |
| hIgG4-LS | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLGK | |
| Heavy Chain | ASTKGPSVFPLAPCSRSTSESTAALGCLVKDYFPEPVTVSWN | 195 |
| Constant Region | SGALTSGVHTFPAVLQSSGLYSLSSVVTVPSSSLGTKTYTCN | |
| hIgG4-SP/LS | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLGK | |
| Heavy Chain | ASTKGPSVFPLAPCSRSTSESTAALGCLVKDYFPEPVTVSWN | 196 |
| Constant Region | SGALTSGVHTFPAVLQSSGLYSLSSVVTVPSSSLGTKTYTCN | |
| hIgG4-SPLE/LS | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHSHYTQKSLSLSLGK | |
| Heavy Chain | ASTKGPSVFPLAPCSRSTSESTAALGCLVKDYFPEPVTVSWN | 197 |
| Constant Region | SGALTSGVHTFPAVLQSSGLYSLSSVVTVPSSSLGTKTYTCN | |
| hIgG4-DHS | VDHKPSNTKVDKRVESKYGPPCPSCPAPEFLGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLGK | |
| Heavy Chain | ASTKGPSVFPLAPCSRSTSESTAALGCLVKDYFPEPVTVSWN | 198 |
| Constant Region | SGALTSGVHTFPAVLQSSGLYSLSSVVTVPSSSLGTKTYTCN | |
| hIgG4-SP/DHS | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLGK | |
| Heavy Chain | ASTKGPSVFPLAPCSRSTSESTAALGCLVKDYFPEPVTVSWN | 199 |
| Constant Region | SGALTSGVHTFPAVLQSSGLYSLSSVVTVPSSSLGTKTYTCN | |
| hIgG4-SPLE/DHS | VDHKPSNTKVDKRVESKYGPPCPPCPAPEELGGPSVFLFPPK | |
| | PKDTLMISRTPEVTCVVVDVSQEDPEVQFNWYVDGVEVHN | |
| | AKTKPREEQFNSTYRVVSVLTVDHHDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHSHYTQKSLSLSLGK | |
| Heavy Chain | ASTKGPSVFPLAPCSRSTSESTAALGCLVKDYFPEPVTVSWN | 200 |
| Constant Region | SGALTSGVHTFPAVLQSSGLYSLSSVVTVPSSNFGTQTYTCN | |
| hIgG2-YTE | VDHKPSNTKVDKTVERKCCVECPPCPAPPVAGPSVFLFPPKP | |
| | KDTLYITREPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHNHYTQKSLSLSPGK | |
| Heavy Chain | ASTKGPSVFPLAPCSRSTSESTAALGCLVKDYFPEPVTVSWN | 201 |
| Constant Region | SGALTSGVHTFPAVLQSSGLYSLSSVVTVPSSNFGTQTYTCN | |
| hIgG2-LS | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVLHEALHSHYTQKSLSLSPGK | |
| Heavy Chain | ASTKGPSVFPLAPCSRSTSESTAALGCLVKDYFPEPVTVSWN | 202 |
| Constant Region | SGALTSGVHTFPAVLQSSGLYSLSSVVTVPSSNFGTQTYTCN | |
| hIgG2-DHS | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVDHHDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVMHEALHSHYTQKSLSLSPGK | |
| Heavy Chain | ASTKGPSVFPLAPCSRSTSESTAALGCLVKDYFPEPVTVSWN | 203 |
| Constant Region | SGALTSGVHTFPAVLQSSGLYSLSSVVTVPSSSLGTKTYTCN | |
| IgG4-SP | VDHKPSNTKVDKRVESKYGPPCPPCPAPEFLGGPSVFLFPPK | |
| | PKDTLMISRTPEVTCVVVDVSQEDPEVQFNWYVDGVEVHN | |
| | AKTKPREEQFNSTYRVVSVLTVLHQDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVMHEALHNHYTQKSLSLSLGK | |
| Heavy Chain | ASTKGPSVFPLAPSSKSTSGGTAALGCLVKDYFPEPVTVSW | 204 |
| Constant Region | NSGALTSGVHTFPAVLQSSGLYSLSSVVTVPSSSLGTQTYIC | |
| hIgG1-LA | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 205 |
| Constant Region | NSGALTSGVHTFPAVLQSSGLYSLSSVVTVPSSSLGTQTYIC | |
| hIgG1-N297A/LA | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 206 |
| Constant Region | NSGALTSGVHTFPAVLQSSGLYSLSSVVTVPSSSLGTQTYIC | |
| hIgG1-D265A/LA | NVNHKPSNTKVDKKVEPKSCDKTHTCPPCPAPELLGGPSVF | |
| | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 207 |
| Constant Region | NSGALTSGVHTFPAVLQSSGLYSLSSVVTVPSSSLGTQTYIC | |
| hIgG1-LALA/LA | NVNHKPSNTKVDKKVEPKSCDKTHTCPPCPAPEAAGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 208 |
| Constant Region | NSGALTSGVHTFPAVLQSSGLYSLSSVVTVPSSSLGTQTYIC | |
| hIgG1-LAGA/LA | NVNHKPSNTKVDKKVEPKSCDKTHTCPPCPAPELAGAPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 209 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/LA | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 210 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/LA | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHAHYTQKSLSLSPG | |
| | K | |
| Heavy Chain | ASTKGPSVFPLAPSSKSTSGGTAALGCLVKDYFPEPVTVSW | 211 |
| Constant Region | NSGALTSGVHTFPAVLQSSGLYSLSSVVTVPSSSLGTQTYIC | |
| hIgG1-N434A | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 212 |
| Constant Region | NSGALTSGVHTFPAVLQSSGLYSLSSVVTVPSSSLGTQTYIC | |
| hIgG1-N297A/ | NVNHKPSNTKVDKKVEPKSCDKTHTCPPCPAPELLGGPSVF | |
| N434A | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 213 |
| Constant Region | NSGALTSGVHTFPAVLQSSGLYSLSSVVTVPSSSLGTQTYIC | |
| hIgG1-D265A/ | NVNHKPSNTKVDKKVEPKSCDKTHTCPPCPAPELLGGPSVF | |
| N434A | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 214 |
| Constant Region | NSGALTSGVHTFPAVLQSSGLYSLSSVVTVPSSSLGTQTYIC | |
| hIgG1-LALA/ | NVNHKPSNTKVDKKVEPKSCDKTHTCPPCPAPEAAGGPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 215 |
| Constant Region | NSGALTSGVHTFPAVLQSSGLYSLSSVVTVPSSSLGTQTYIC | |
| hIgG1-LAGA/ | NVNHKPSNTKVDKKVEPKSCDKTHTCPPCPAPELAGAPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 216 |
| Constant Region | NSGALTSGVHTFPAVLQSSGLYSLSSVVTVPSSSLGTQTYIC | |
| hIgG1-LALAGA/ | NVNHKPSNTKVDKKVEPKSCDKTHTCPPCPAPEAAGAPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 217 |
| Constant Region | NSGALTSGVHTFPAVLQSSGLYSLSSVVTVPSSSLGTQTYIC | |
| hIgG1-LALAPG/ | NVNHKPSNTKVDKKVEPKSCDKTHTCPPCPAPEAAGGPSVF | |
| N434A | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHAHYTQKSLSLSP | |
| | GK | |
| Heavy Chain | ASTKGPSVFPLAPSSKSTSGGTAALGCLVKDYFPEPVTVSW | 218 |
| Constant Region | NSGALTSGVHTFPAVLQSSGLYSLSSVVTVPSSSLGTQTYIC | |
| hIgG1-N434W | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 219 |
| Constant Region | NSGALTSGVHTFPAVLQSSGLYSLSSVVTVPSSSLGTQTYIC | |
| hIgG1-N297A/ | NVNHKPSNTKVDKKVEPKSCDKTHTCPPCPAPELLGGPSVF | |
| N434W | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 220 |
| Constant Region | NSGALTSGVHTFPAVLQSSGLYSLSSVVTVPSSSLGTQTYIC | |
| hIgG1-D265A/ | NVNHKPSNTKVDKKVEPKSCDKTHTCPPCPAPELLGGPSVF | |
| N434W | LFPPKPKDTLMISRTPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 221 |
| Constant Region | NSGALTSGVHTFPAVLQSSGLYSLSSVVTVPSSSLGTQTYIC | |
| hIgG1-LALA/ | NVNHKPSNTKVDKKVEPKSCDKTHTCPPCPAPEAAGGPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 222 |
| Constant Region | NSGALTSGVHTFPAVLQSSGLYSLSSVVTVPSSSLGTQTYIC | |
| hIgG1-LAGA/ | NVNHKPSNTKVDKKVEPKSCDKTHTCPPCPAPELAGAPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 223 |
| Constant Region | NSGALTSGVHTFPAVLQSSGLYSLSSVVTVPSSSLGTQTYIC | |
| hIgG1-LALAGA/ | NVNHKPSNTKVDKKVEPKSCDKTHTCPPCPAPEAAGAPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 224 |
| Constant Region | NSGALTSGVHTFPAVLQSSGLYSLSSVVTVPSSSLGTQTYIC | |
| hIgG1-LALAPG/ | NVNHKPSNTKVDKKVEPKSCDKTHTCPPCPAPEAAGGPSVF | |
| N434W | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHWHYTQKSLSLSP | |
| | GK | |
| Heavy Chain | ASTKGPSVFPLAPSSKSTSGGTAALGCLVKDYFPEPVTVSW | 225 |
| Constant Region | NSGALTSGVHTFPAVLQSSGLYSLSSVVTVPSSSLGTQTYIC | |
| hIgG1-DQ | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 226 |
| Constant Region | NSGALTSGVHTFPAVLQSSGLYSLSSVVTVPSSSLGTQTYIC | |
| hIgG1-N297A/DQ | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYASTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 227 |
| Constant Region | NSGALTSGVHTFPAVLQSSGLYSLSSVVTVPSSSLGTQTYIC | |
| hIgG1-D265A/DQ | NVNHKPSNTKVDKKVEPKSCDKTHTCPPCPAPELLGGPSVF | |
| | LFPPKPKDTLMISRDPEVTCVVVAVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 228 |
| Constant Region | NSGALTSGVHTFPAVLQSSGLYSLSSVVTVPSSSLGTQTYIC | |
| hIgG1-LALA/DQ | NVNHKPSNTKVDKKVEPKSCDKTHTCPPCPAPEAAGGPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 229 |
| Constant Region | NSGALTSGVHTFPAVLQSSGLYSLSSVVTVPSSSLGTQTYIC | |
| hIgG1-LAGA/DQ | NVNHKPSNTKVDKKVEPKSCDKTHTCPPCPAPELAGAPSVF | |
| | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 230 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DQ | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 231 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DQ | LFPPKPKDTLMISRDPEVTCVVVDVSHEDPEVKFNWYVDGV | |
| | EVHNAKTKPREEQYNSTYRVVSVLQVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 232 |
| Constant Region | NSGALTSGVHTFPAVLQSSGLYSLSSVVTVPSSSLGTQTYIC | |
| hIgG1-DW | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 233 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DW | LFPPKPKDTLMISRDPEVTCVVVDVSHEDPEVKFNWYVDGV | |
| | EVHNAKTKPREEQYASTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 234 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DW | LFPPKPKDTLMISRDPEVTCVVVAVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 235 |
| Constant Region | NSGALTSGVHTFPAVLQSSGLYSLSSVVTVPSSSLGTQTYIC | |
| hIgG1-LALA/DW | NVNHKPSNTKVDKKVEPKSCDKTHTCPPCPAPEAAGGPSVF | |
| | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 236 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 237 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 238 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DW | LFPPKPKDTLMISRDPEVTCVVVDVSHEDPEVKFNWYVDGV | |
| | EVHNAKTKPREEQYNSTYRVVSVLWVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 239 |
| Constant Region | NSGALTSGVHTFPAVLQSSGLYSLSSVVTVPSSSLGTQTYIC | |
| hIgG1-YD | NVNHKPSNTKVDKKVEPKSCDKTHTCPPCPAPELLGGPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 240 |
| Constant Region | NSGALTSGVHTFPAVLQSSGLYSLSSVVTVPSSSLGTQTYIC | |
| hIgG1-N297A/YD | NVNHKPSNTKVDKKVEPKSCDKTHTCPPCPAPELLGGPSVF | |
| | LFPPKPKDTLYISRDPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 241 |
| Constant Region | NSGALTSGVHTFPAVLQSSGLYSLSSVVTVPSSSLGTQTYIC | |
| hIgG1-D265A/YD | NVNHKPSNTKVDKKVEPKSCDKTHTCPPCPAPELLGGPSVF | |
| | LFPPKPKDTLYISRDPEVTCVVVAVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 242 |
| Constant Region | NSGALTSGVHTFPAVLQSSGLYSLSSVVTVPSSSLGTQTYIC | |
| hIgG1-LALA/YD | NVNHKPSNTKVDKKVEPKSCDKTHTCPPCPAPEAAGGPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 243 |
| Constant Region | NSGALTSGVHTFPAVLQSSGLYSLSSVVTVPSSSLGTQTYIC | |
| hIgG1-LAGA/YD | NVNHKPSNTKVDKKVEPKSCDKTHTCPPCPAPELAGAPSVF | |
| | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 244 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/YD | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 245 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/YD | LFPPKPKDTLYISRDPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 246 |
| Constant Region | NSGALTSGVHTFPAVLQSSGLYSLSSVVTVPSSSLGTQTYIC | |
| hIgG1-QVV | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 247 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/QVV | LFPPKPKDTLMISRTPEVTCVVVDVSHEDPEVKFNWYVDGV | |
| | EVHNAKTKPREEQYASTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 248 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/QVV | LFPPKPKDTLMISRTPEVTCVVVAVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 249 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 250 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 251 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 252 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/QVV | LFPPKPKDTLMISRTPEVTCVVVDVSHEDPEVKFNWYVDGV | |
| | EVHNAKTKPREEQYNSTYRVVSVLQVLHVDWLNGKEYKC | |
| | KVSNKALGAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 253 |
| Constant Region | NSGALTSGVHTFPAVLQSSGLYSLSSVVTVPSSSLGTQTYIC | |
| hIgG1-DDRVV | NVNHKPSNTKVDKKVEPKSCDKTHTCPPCPAPELLGGPSVF | |
| | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 254 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/DDRVV | LFPPKPKDTLMISRDPEVTCVVVDVSHEDPEVKFNWYVDGV | |
| | EVDNAKTKPREEQYASTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 255 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/DDRVV | LFPPKPKDTLMISRDPEVTCVVVAVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 256 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 257 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 258 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 259 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/DDRVV | LFPPKPKDTLMISRDPEVTCVVVDVSHEDPEVKFNWYVDGV | |
| | EVDNAKTKPREEQYNSTYRVVSVLRVLHVDWLNGKEYKC | |
| | KVSNKALGAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSP | |
| | GK | |
| Heavy Chain | ASTKGPSVFPLAPSSKSTSGGTAALGCLVKDYFPEPVTVSW | 260 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| Q311R/M428L | LFPPKPKDTLMISRTPEVTCVVVDVSHEDPEVKFNWYVDGV | |
| | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPCSRSTSESTAALGCLVKDYFPEPVTVSWN | 261 |
| Constant Region | SGALTSGVHTFPAVLQSSGLYSLSSVVTVPSSSLGTKTYTCN | |
| hIgG4- | VDHKPSNTKVDKRVESKYGPPCPSCPAPEFLGGPSVFLFPPK | |
| Q311R/M428L | PKDTLMISRTPEVTCVVVDVSQEDPEVQFNWYVDGVEVHN | |
| | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLGK | |
| Heavy Chain | ASTKGPSVFPLAPCSRSTSESTAALGCLVKDYFPEPVTVSWN | 262 |
| Constant Region | SGALTSGVHTFPAVLQSSGLYSLSSVVTVPSSSLGTKTYTCN | |
| IgG4- | VDHKPSNTKVDKRVESKYGPPCPPCPAPEFLGGPSVFLFPPK | |
| SP/Q311R/M428L | PKDTLMISRTPEVTCVVVDVSQEDPEVQFNWYVDGVEVHN | |
| | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLGK | |
| Heavy Chain | ASTKGPSVFPLAPCSRSTSESTAALGCLVKDYFPEPVTVSWN | 263 |
| Constant Region | SGALTSGVHTFPAVLQSSGLYSLSSVVTVPSSSLGTKTYTCN | |
| IgG4- | VDHKPSNTKVDKRVESKYGPPCPPCPAPEELGGPSVFLFPPK | |
| SPLE/Q311R/ | PKDTLMISRTPEVTCVVVDVSQEDPEVQFNWYVDGVEVHN | |
| M428L | AKTKPREEQFNSTYRVVSVLTVLHRDWLNGKEYKCKVSNK | |
| | GLPSSIEKTISKAKGQPREPQVYTLPPSQEEMTKNQVSLTCL | |
| | VKGFYPSDIAVEWESNGQPENNYKTTPPVLDSDGSFFLYSRL | |
| | TVDKSRWQEGNVFSCSVLHEALHNHYTQKSLSLSLGK | |
| Heavy Chain | ASTKGPSVFPLAPCSRSTSESTAALGCLVKDYFPEPVTVSWN | 264 |
| Constant Region | SGALTSGVHTFPAVLQSSGLYSLSSVVTVPSSNFGTQTYTCN | |
| IgG2- | VDHKPSNTKVDKTVERKCCVECPPCPAPPVAGPSVFLFPPKP | |
| Q311R/M428L | KDTLMISRTPEVTCVVVDVSHEDPEVQFNWYVDGVEVHNA | |
| | KTKPREEQFNSTFRVVSVLTVVHRDWLNGKEYKCKVSNKG | |
| | LPAPIEKTISKTKGQPREPQVYTLPPSREEMTKNQVSLTCLV | |
| | KGFYPSDIAVEWESNGQPENNYKTTPPMLDSDGSFFLYSKL | |
| | TVDKSRWQQGNVFSCSVLHEALHNHYTQKSLSLSPGK | |
| Heavy Chain | ASTKGPSVFPLAPSSKSTSGGTAALGCLVKDYFPEPVTVSW | 265 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| N297A/Q311R/M | LFPPKPKDTLMISRTPEVTCVVVDVSHEDPEVKFNWYVDGV | |
| 428L | EVHNAKTKPREEQYASTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 266 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELLGGPSVF | |
| D265A/Q311R/ | LFPPKPKDTLMISRTPEVTCVVVAVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 267 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALA/Q311R/M4 | LFPPKPKDTLMISRTPEVTCVVVDVSHEDPEVKFNWYVDGV | |
| 28L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 268 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPELAGAPSVF | |
| LAGA/Q311R/M | LFPPKPKDTLMISRTPEVTCVVVDVSHEDPEVKFNWYVDGV | |
| 428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 269 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGAPSVF | |
| LALAGA/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALPAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| Heavy Chain | ASTKGPSVFPLAPSSKSTSGGTAALGCLVKDYFPEPVTVSW | 270 |
| Constant Region | NSGALTSGVHTFPAVLQSSGLYSLSSVVTVPSSSLGTQTYIC | |
| hIgG1- | NVNHKPSNTKVDKKVEPKSCDKTHTCPPCPAPEAAGGPSVF | |
| LALAPG/Q311R/ | LFPPKPKDTLMISRTPEVTCVVVDVSHEDPEVKFNWYVDGV | |
| M428L | EVHNAKTKPREEQYNSTYRVVSVLTVLHRDWLNGKEYKC | |
| | KVSNKALGAPIEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVLHEALHNHYTQKSLSLSPG | |
| | K | |
| LCDR1 | RTSQNIRYYLN | 271 |
| LCDR1 | GGDNIGSKSVH | 272 |
| LCDR1 | GGQNIGTKSVH | 273 |
| LCDR1 | GGNKIGRKSVH | 274 |
| LCDR1 | GGNNIGGKSVH | 275 |
| LCDR1 | GGNNIGSKSVH | 276 |
| LCDR1 | GGHNIGTKSVH | 277 |
| LCDR1 | GGDSIGSKYVH | 278 |
| LCDR1 | GGQNIGTRSVH | 279 |
| LCDR1 | GGNNIGSETVH | 280 |
| LCDR1 | RGDNIETYSVH | 281 |
| LCDR1 | GGNNIGRKSAH | 282 |
| LCDR1 | EGTYLGAKSVH | 283 |
| LCDR1 | GGNNIGTKSVH | 284 |
| LCDR1 | GGQSIGTKSVH | 285 |
| LCDR1 | GANSIEDKNVH | 286 |
| LCDR1 | GGDNIGSYSVH | 287 |
| LCDR1 | GGNNVEDYSVH | 288 |
| LCDR1 | QNIRYY | 289 |
| LCDR1 | NIGSKS | 290 |
| LCDR1 | NIGTKS | 291 |
| LCDR1 | KIGRKS | 292 |
| LCDR1 | NIGGKS | 293 |
| LCDR1 | SIGSKY | 294 |
| LCDR1 | NIGTRS | 295 |
| LCDR1 | NIGSET | 296 |
| LCDR1 | NIETYS | 297 |
| LCDR1 | NIGRKS | 298 |
| LCDR1 | SIGTKS | 299 |
| LCDR1 | SIEDKN | 300 |
| LCDR1 | NIGSYS | 301 |
| LCDR1 | NVEDYS | 302 |
| LCDR2 | DVSSLQS | 303 |
| LCDR2 | EDSDRPS | 304 |
| LCDR2 | DDTDRPS | 305 |
| LCDR2 | DDSDRPP | 306 |
| LCDR2 | DDKERPS | 307 |
| LCDR2 | DDSERPS | 308 |
| LCDR2 | DDLKRPS | 309 |
| LCDR2 | DDNDRPS | 310 |
| LCDR3 | QQGYSPPLT | 311 |
| LCDR3 | QVWDSDSDHVV | 312 |
| LCDR3 | QVWDSSSDQSV | 313 |
| LCDR3 | QVWDSSSDEGV | 314 |
| LCDR3 | QVWDGSSDQSV | 315 |
| LCDR3 | QVWDTTTDHVV | 316 |
| LCDR3 | QVWDSSSDPVV | 317 |
| LCDR3 | QVWDRSSDQSV | 318 |
| LCDR3 | QVWDSGSGQTV | 319 |
| LCDR3 | HVWDSSSDHVM | 320 |
| LCDR3 | QVWDSDSDHLE | 321 |
| LCDR3 | QVWDSYSNHVV | 322 |
| LCDR3 | QVWDSGGDHVV | 323 |
| LCDR3 | QVWDDISDHIV | 324 |
| LCDR3 | QVWDMASDRVV | 325 |
| LCDR3 | QVWDTSSDQGV | 326 |
| LCDR3 | QVWDSTSGVVF | 327 |
| VL | DIQLTQSPSSLSASVGDRVAITCRTSQNIRYYLNWYQRKPGK | 328 |
| | APRLLIYDVSSLQSGVPSRFSGRGSGTDFTLTISSLQAEDFAT | |
| | YYCQQGYSPPLTFGGGTKVEIK | |
| VL | NFMLTQPPSVSVAPGKTAKMTCGGDNIGSKSVHWYQQMPG | 329 |
| | QAPVLVIYEDSDRPSGTPERFSGSNSGHTATLTISRVEAGDE | |
| | ADYYCQVWDSDSDHVVFGGGTTLTVL | |
| VL | QSVLTQPPSVSVAPGQTARITCGGQNIGTKSVHWYQQKSGQ | 330 |
| | APILVVYDDSDRPSWIPERFSGSNFGTTATLTISRVEAGDEA | |
| | DYFCQVWDSSSDQSVFGPGTRVTVL | |
| VL | SYELTQPPSVSVAPGQTARITCGGQNIGTKSVHWYQQKSGQ | 331 |
| | APILVVYDDSDRPSWIPERFSGSNFGTTATLTISRVEAGDEA | |
| | DYFCQVWDSSSDQSVFGPGTRVTVL | |
| VL | SYVLTQPPSVSVAPGQTARITCGGQNIGTKSVHWYQQKSGQ | 332 |
| | APILVVYDDSDRPSWIPERFSGSNFGTTATLTISRVEAGDEA | |
| | DYFCQVWDSSSDQSVFGPGTRVTVL | |
| VL | SYVLTQPPSVSVAPGQTARITCGGNKIGRKSVHWYQQRPGQ | 333 |
| | APVLVVFDDSDRPSWISERFSGSNSGNTATLTISRVEAGDEA | |
| | DYYCQVWDSSSDEGVFGGGTKLTVL | |
| VL | SYELTQPPSVSVAPGQAARITCGGNKIGRKSVHWYQQRPGQ | 334 |
| | APVLVVFDDSDRPSWISERFSGSNSGNTATLTISRVEAGDEA | |
| | DYYCQVWDSSSDEGVFGGGTKLTVL | |
| VL | QSVLTQPPSVSVAPGQTARITCGGQNIGTKSVHWYQQKSGQ | 335 |
| | APILVVYDDSDRPSWIPERFSGSNFGTTATLTISRVEAGDEA | |
| | DYFCQVWDGSSDQSVFGPGTRVTVL | |
| VL | SYELTQPPSVSVAPGQTASITCGGNNIGGKSVHWYQLKPGQ | 336 |
| | APVLVVYDDTDRPSWIPERFSGSNSANTATLTISGVEAGDEA | |
| | DYQCQVWDTTTDHVVFGGGTKLTVL | |
| VL | SYVLTQPPSVSVAPGQTARITCGGNNIGSKSVHWYQQKPGQ | 337 |
| | APLLVVYDDSDRPSWIPERFSGSNSGNAATLTVSRVEAGDE | |
| | ADYYCQVWDSSSDPVVFGGGTKLTVL | |
| VL | FYVLTQPPSVSVAPGQTARITCGGQNIGTKSVHWYQQKSGQ | 338 |
| | APILVVYDDSDRPSWIPERFSGSNFGTTATLTISRVEAGDEA | |
| | DYFCQVWDSSSDQSVFGPGTRVTVL | |
| VL | QSVLTQPPSVSVAPGQTARITCGGQNIGTKSVHWYQQKSGQ | 339 |
| | APILVVYDDSDRPPWIPERFSGSNFGTTATLTISRVEAGDEA | |
| | DYFCQVWDSSSDQSVFGPGTRVTVL | |
| VL | QAVLTQPPSVSVAPGQTARITCGGNKIGRKSVHWYQQRPGQ | 340 |
| | APVLVVFDDSDRPSWISERFSGSNSGNTATLTISRVEAGDEA | |
| | DYYCQVWDSSSDEGVFGGGTKLTVL | |
| VL | SYELTQPPSVSVAPGQTARITCGGHNIGTKSVHWYQQKSGQ | 341 |
| | APILVVYDDSDRPSWIPERFSGSNFGTTATLTISRVEAGDEA | |
| | DYFCQVWDRSSDQSVFGPGTRVTVL | |
| VL | SYVLTQPPSVSVAPGQTARVTCGGDSIGSKYVHWYQQKPG | 342 |
| | QAPVLVVYDDSDRPSWIPERFSGSNFGTTATLTISRVEAGDE | |
| | ADYFCQVWDRSSDQSVFGPGTRVTVL | |
| VL | SYELTQPPSVSVAPGQTARITCGGQNIGTKSVHWYQQKSGQ | 343 |
| | APILVVFDDSDRPSWVTERFSGSNFGTTATLTINGVEAGDEA | |
| | DYFCQVWDSGSGQTVFGPGTRVTVS | |
| VL | SYELTQPPSVSVAPGQTARITCGGQNIGTRSVHWYQQKSGQ | 344 |
| | APILVVYDDSDRPSWIPERFSGSNFGTTATLTISRVEAGDEA | |
| | DYFCQVWDSSSDQSVFGPGTRVTVL | |
| VL | SYELTQPPSVSVAPGQTARITCGGNNIGSETVHWYRQKAGQ | 345 |
| | APVLVVYDDSDRPSGIPERFSGSNSGNTAALTISRVEVGDEA | |
| | DYYCHVWDSSSDHVMFGGGTKLTVL | |
| VL | SYELTQPPSVSVGPGKTATITCRGDNIETYSVHWYQQKPGQ | 346 |
| | APVLVVYDDSDRPSGIPERFSGSNSGNTATLTINRVGAGDEA | |
| | DYYCQVWDSDSDHLEFGGGTKLTVL | |
| VL | SYELTQPPSVSVAPGQTARITCGGNNIGRKSAHWYQQRPGQ | 347 |
| | APVLVVYDDSDRPSWIPERFSGSNSGNTATLTISRVEAGDEA | |
| | DYYCQVWDSYSNHVVFGGGTKLTVL | |
| VL | LPVLTQPPSVSVAPGQTARITCGGNKIGRKSVHWYQQRPGQ | 348 |
| | APVLVVFDDSDRPSWISERFSGSNSGNTATLTISRVEAGDEA | |
| | DYYCQVWDSSSDEGVFGGGTKLTVL | |
| VL | SYVLTQPPSVSVAPGQTARITCGGHNIGTKSVHWYQQKSGQ | 349 |
| | APILAVYDDSDRPSWIPERFSGSNFGTTATLTISRVEAGDEA | |
| | DYFCQVWDRSSDQSVFGPGTRVTVL | |
| VL | SYELTQPPSVSVGPGKTATITCRGDNIETYSVHWYQQKPGQ | 350 |
| | APVLVVYDDSDRPSGIPERFSGSNSGNTATLTINRVEARDEA | |
| | DYFCQVWDSGGDHVVFGGGTKLTVL | |
| VL | SYVLTQPPSVSVAPGQTARITCGGNNIGSKSVHWYQQKPGQ | 351 |
| | APVLVVYDDSDRPSWIPERFSGTNSGNTATLTISRVEAGDEA | |
| | DYYCQVWDSSSDHVVFGGGTKLTVL | |
| VL | SYELTQPPSVSVAPGKRARITCEGTYLGAKSVHWYQQKPGQ | 352 |
| | APVLVIYDDKERPSGIPERFSASNSGNTATLTITRVEAGDEA | |
| | DYHCQVWDDISDHIVFGGGTKLSVL | |
| VL | SYVLTQPPSVSVAPGQTARITCGGNNIGTKSVHWYQQKSGQ | 353 |
| | APILVVYDDSDRPSWIPERFSGSNFGTTATLTISRVEAGDEA | |
| | DYFCQVWDSSSDQSVFGPGTRVTVL | |
| VL | SYVLTQPPSVSVAPGQTARITCGGQSIGTKSVHWYQQKSGQ | 354 |
| | APILVVYDDSDRPSWIPERFSGSNFGTTATLTISRVEAGDEA | |
| | DYFCQVWDSSSDQSVFGPGTRVTVL | |
| VL | SYELTQPPSVSVAPGQAASLTCGANSIEDKNVHWYQQRPGQ | 355 |
| | APLLVIYDDSERPSGIPERFSGSNSGNTATLTITRVAAGDEAD | |
| | YYCQVWDMASDRVVFGPGTTVSVL | |
| VL | SYELTQPPSVSVAPGQTARITCGGNKIGRKSVHWYQQRPGQ | 356 |
| | APVLVVFDDSDRPSWISERFSGSNSGNTATLTIGRVEAGDEA | |
| | DYYCQVWDSSSDEGVFGGGTKLAVL | |
| VL | SYELTQPPSVSVAPGRTARITCGGQNIGTKSVHWYQQKSGQ | 357 |
| | APILVVYDDSDRPSWIPERFSGSNFGTTATLTISRVEAGDEA | |
| | DYFCQVWDSSSDQSVFGPGTRVTVL | |
| VL | QAGLTQPPSVSVAPGQTARITCGGNKIGRKSVHWYQQRPGQ | 358 |
| | APVLVVFDDSDRPSWISERFSGSNSGNTATLTISRVEAGDEA | |
| | DYYCQVWDSSSDEGVFGGGTKLTVL | |
| VL | QSVLTQPPSVSVAPGQTARITCGGQNIGTKSVHWYQQKSGQ | 359 |
| | APILVVYDDSDRPSWIPERFSGSNFGTTATLTISRVEAGDEA | |
| | DYFCQVWDSSSDQSVFGTGTKVTVL | |
| VL | SYVLTQPPSVSVGPGKTATITCRGDNIETYSVHWYQRKPGQ | 360 |
| | APVLVVYDDSDRPSGIPERFSGSNSGNTATLTISRVEAGDEA | |
| | DYYCQVWDSSSDHVVFGGGTKLTVL | |
| VL | SYELTQPPSVSVAPGQTARITCGGNKIGRKSVHWYQQRPGQ | 361 |
| | APVLVVFDDSDRPSWISERFSGSNSGNTATLTISRVDAGDEA | |
| | DYYCQVWDSSSDEGVFGGGTKLTVL | |
| VL | SYELTQPPSVSVAPGQTARITCGGNKIGRKSVHWYQQKPGQ | 362 |
| | APVLVVFDDSDRPSWISERFSGSNSGNTATLTISRVEAGDEA | |
| | DYHCQVWDTSSDQGVFGGGTKLTVL | |
| VL | SYVLTQPPSVAVAPGQTAKITCGGDNIGSYSVHWYQQKPGQ | 363 |
| | APVLVVYDDLKRPSWIPERFFGSNSGNTATLTISRVEAGDEA | |
| | VYYCQVWDSTSGVVFGGGTTVTVL | |
| VL | SYELTQPPSVSVAPGKTARITCGGNNVEDYSVHWYHQKAG | 364 |
| | QAPVVVMYDDNDRPSGVPERFSGSNSGNTATLTIGRVEAGD | |
| | EADYYCQVWDSSSDHVVFGGGTKLTVL | |
| TSLP (human) | MFPFALLYVLSVSFRKIFILQLVGLVLTYDFTNCDFEKIKAA | 365 |
| UniProt Accession | YLSTISKDLITYMSGTKSTEFNNTVSCSNRPHCLTEIQSLTFN | |
| No. Q969D9 | PTAGCASLAKEMFAMKTKAALAIWCPGYSETQINATQAMK | |
| | KRRKRKVTTNKCLEQVSQLQGLWRRFNRPLLKQQ | |
| TSLP | MKSLGQSKKEEVSFRKFFIFQLVGLVLTYDFTNCDFQKIEAD | 366 |
| (Cynomolgus | YLRTISKDLITYMSGTKSTDFNNTVSCSNRPHCLTEIQSLTFN | |
| monkey) | PTPRCASLAKEMFARKTKATLALWCPGYSETQINATQAMK | |
| UniProt Accession | KRRKRKVTTNKCLEQVSQLLGLWRRFIRTLLKKQ | |
| No. G7P819 | | |
| CDR-L3 | QVWDSSSDFVV | 367 |
| CDR-L3 | QVWDSSSTHVV | 368 |
| CDR-L3 | QVWDSSSTFVV | 369 |
| CDR-L3 | QVWDSSSTIVV | 370 |
| Antibody 47 VL | SYVLTQPPSVSVAPGQTARITCGGNNLGSKSVHWYQQKPG | 371 |
| | QAPVLVVYDVSDRPSWIPERFSGSNSGNTATLTISRGEAGDE | |
| | ADYYCQVWDSSSDHVVFGGGTKLTVL | |
| Antibody 48 VL | SYVLTQPPSVSVAPGQTARITCGGNNLGSKSVHWYQQKPG | 372 |
| | QAPVLVVYDDSDRPSWIPERFSGSNSGNTATLTISRGEAGDE | |
| | ADYYCQVWDSSSDFVVFGGGTKLTVL | |
| Antibody 49 VL | SYVLTQPPSVSVAPGQTARITCGGNNLVSKSVHWYQQKPG | 373 |
| | QAPVLVVYDVSDRPSWIPERFSGSNSGNTATLTISRGEAGDE | |
| | ADYYCQVWDSSSDHVVFGGGTKLTVL | |
| Antibody 50 VL | SYVLTQPPSVSVAPGQTARITCGGNNLVSKSVHWYQQKPG | 374 |
| | QAPVLVVYDDSDRPSWIPERFSGSNSGNTATLTISRGEAGDE | |
| | ADYYCQVWDSSSDFVVFGGGTKLTVL | |
| Antibody 51 VL | SYVLTQPPSVSVAPGQTARITCGGNNLGSKSVHWYQQKPG | 375 |
| | QAPVLVVYDVSDRPSWIPERFSGSNSGNTATLTISRGEAGDE | |
| | ADYYCQVWDSSSTHVVFGGGTKLTVL | |
| Antibody 52 VL | SYVLTQPPSVSVAPGQTARITCGGNNLGSKSVHWYQQKPG | 376 |
| | QAPVLVVYDVSDRPSWIPERFSGSNSGNTATLTISRGEAGDE | |
| | ADYYCQVWDSSSDFVVFGGGTKLTVL | |
| Antibody 53 VL | SYVLTQPPSVSVAPGQTARITCGGNNLGSKSVHWYQQKPG | 377 |
| | QAPVLVVYDDSDRPSWIPERFSGSNSGNTATLTISRGEAGDE | |
| | ADYYCQVWDSSSTFVVFGGGTKLTVL | |
| Antibody 54 VL | SYVLTQPPSVSVAPGQTARITCGGNNLVSKSVHWYQQKPG | 378 |
| | QAPVLVVYDVSDRPSWIPERFSGSNSGNTATLTISRGEAGDE | |
| | ADYYCQVWDSSSTHVVFGGGTKLTVL | |
| Antibody 55 VL | SYVLTQPPSVSVAPGQTARITCGGNNLVSKSVHWYQQKPG | 379 |
| | QAPVLVVYDVSDRPSWIPERFSGSNSGNTATLTISRGEAGDE | |
| | ADYYCQVWDSSSDFVVFGGGTKLTVL | |
| Antibody 57 VL | SYVLTQPPSVSVAPGQTARITCGGNNLVSKSVHWYQQKPG | 381 |
| | QAPVLVVYDDSDRPSWIPERFSGSNSGNTATLTISRGEAGDE | |
| | ADYYCQVWDSSSTIVVFGGGTKLTVL | |
| Antibody 58 VL | SYVLTQPPSVSVAPGQTARITCGGNNLGSKSVHWYQQKPG | 382 |
| | QAPVLVVYDVSDRPSWIPERFSGSNSGNTATLTISRGEAGDE | |
| | ADYYCQVWDSSSTFVVFGGGTKLTVL | |
| Antibody 59 VL | SYVLTQPPSVSVAPGQTARITCGGNNLVSKSVHWYQQKPG | 383 |
| | QAPVLVVYDVSDRPSWIPERFSGSNSGNTATLTISRGEAGDE | |
| | ADYYCQVWDSSSTFVVFGGGTKLTVL | |
| Antibody 60 VL | SYVLTQPPSVSVAPGQTARITCGGNNLVSKSVHWYQQKPG | 384 |
| | QAPVLVVYDVSDRPSWIPERFSGSNSGNTATLTISRGEAGDE | |
| | ADYYCQVWDSSSTIVVFGGGTKLTVL | |
| Antibody 61 VL | SYVLTQPPSVSVAPGQTARITCGGNNLGSKSVHWYQQKPG | 385 |
| | QAPVLVVYDDSDRPSWIPERFSGSNSGNTATLTISRGEAGDE | |
| | ADYYCQVWDSSSDHVVFGGGTKLTVL | |
| CDR-L1 | YLGAKS | 387 |
| lambda-1 light | QSALTQPPSASGSLGQSVTISCTGTSSDVGGYNYVSWYQQH | 388 |
| chain | AGKAPKVIIYEVNKRPSGVPDRFSGSKSGNTASLTVSGLQAE | |
| | DEADYYCSSYEGSDNFVFGTGTKVTVLGQPKANPTVTLFPP | |
| | SSEELQANKATLVCLISDFYPGAVTVAWKADGSPVKAGVET | |
| | TKPSKQSNNKYAASSYLSLTPEQWKSHRSYSCQVTHEGSTV | |
| | EKTVAPTECS | |
| TSLP residues 66- | AKEMF | 401 |
| 70 | | |
| TSLP residues 15- | YLSTISKDLITYMSGTK | 402 |
| 31 | | |
| TSLP residues 64- | SLAKEMFAMK | 403 |
| 73 | | |
| TSLP residues | WRRFNRPLL | 404 |
| 120-128 | | |
| TSLP residues 19- | ISKDLITY | 405 |
| 26 | | |
| TSLP residues 67- | KEMFAMK | 406 |
| 73 | | |
| TSLP residues | RRFNRPLLK | 407 |
| 121-129 | | |
| TSLP residues 20- | SKDLITY | 408 |
| 26 | | |

*SEQ ID NOs: 47 and 159 are hIgG1 sequences having a SRDEL (SEQ ID NO: 507) allotype.
The disclosure also encompasses SREEM (SEQ ID NO: 508) allotype variants of any of
the SRDEL (SEQ ID NO: 507) allotype sequences set forth in the table above, and either
a SRDEL (SEQ ID NO: 507) allotype heavy chain constant region sequence or a SREEM
(SEQ ID NO: 508) allotype heavy chain constant region sequence may be included in an
anti-TSLP antibody described herein.
SEQ ID NO: 409
MGVLKFKHIFFRSFVKSSGVSQIVFTFLLIPCCLTLNFRAPPVIPNVPFLWAWNAPSEFCLGKFD
EPLDMSLESFIGSPRINATGQGVTIFYVDRLGYYPYIDSITGVTVNGGIPQKISLQDHLDKAKKD
ITFYMPVDNLGMAVIDWEEWRPTWARNWKPKDVYKNRSIELVQQQNVQLSLTEATEKAKQEFEKA
GKDFLVETIKLGKLLRPNHLWGYYLFPDCYNHHYKKPGYNGSCENVEIKRNDDLSWLWNESTALY
PSIYLNTQQSPVAATLYVRNRVREAIRVSKIPDAKSPLPVFAYTRIVFTDQVLKELSQDELVYTE
GETVALGASGIVIWGTLSIMRSMKSCLLLDNYMETILNPYIINVTLAAKMCSQVLCQEQGVCIRK
NWNSSDYLHLNPDNFAIQLEKGGKFTVRGKPTLEDLEQFSEKFYCSCYSTLSCKEKADVKDTDAV
DVCIADGVCIDAFLKPPMETEEPQIFYNASPSTLSATMFIVSILFLIISSVASL
SEQ ID NO: 410
LNFRAPPVIPNVPFLWAWNAPSEFCLGKFDEPLDMSLFSFIGSPRINATGQGVTIFYVDRLGYYP
YIDSITGVTVNGGIPQKISLQDHLDKAKKDITFYMPVDNLGMAVIDWEEWRPTWARNWKPKDVYK
NRSIELVQQQNVQLSLTEATEKAKQEFEKAGKDELVETIKLGKLLRPNHLWGYYLFPDCYNHHYK
KPGYNGSCENVEIKRNDDLSWLWNESTALYPSIYLNTQQSPVAATLYVRNRVREAIRVSKIPDAK
SPLPVFAYTRIVFTDQVLKFLSQDELVYTFGETVALGASGIVIWGTLSIMRSMKSCLLLDNYMET
ILNPYIINVTLAAKMCSQVLCQEQGVCIRKNWNSSDYLHLNPDNFAIQLEKGGKFTVRGKPTLED
LEQFSEKFYCSCYSTLSCKEKADVKDTDAVDVCIADGVCIDAFLKPPMETEEPQIFYNASPSTLS
ATMFIVSILFLIISSVASL
SEQ ID NO: 411
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKELSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQIFY
SEQ ID NO: 412
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCFNVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQIF
SEQ ID NO: 413
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQI
SEQ ID NO: 414
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQ
SEQ ID NO: 415
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVETDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEP
SEQ ID NO: 416
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEE
SEQ ID NO: 417
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDELVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDA
SEQ ID NO: 418
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCENVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQIFYN
SEQ ID NO: 419
atgggagtgc taaaattcaa gcacatcttt ttcagaagct ttgttaaatc aagtggagta tcccagatag ttttcacctt
ccttctgatt
ccatgttgct tgactctgaa tttcagagca cctcctgtta ttccaaatgt gcctttcctc tgggcctgga atgccccaag
tgaattttgt
cttggaaaat ttgatgagcc actagatatg agcctcttct ctttcatagg aagcccccga ataaacgcca ccgggcaagg
tgttacaata
ttttatgttg atagacttgg ctactatcct tacatagatt caatcacagg agtaactgtg aatggaggaa tcccccagaa
gatttcctta
caagaccatc tggacaaagc taagaaagac attacatttt atatgccagt agacaatttg ggaatggctg ttattgactg
ggaagaatgg
agacccactt gggcaagaaa ctggaaacct aaagatgttt acaagaatag gtctattgaa ttggttcagc aacaaaatgt
acaacttagt
ctcacagagg ccactgagaa agcaaaacaa gaatttgaaa aggcagggaa ggatttcctg gtagagacta taaaattggg
aaaattactt
cggccaaatc acttgtgggg ttattatctt tttccggatt gttacaacca tcactataag aaacccggtt acaatggaag
ttgcttcaat
gtagaaataa aaagaaatga tgatctcagc tggttgtgga atgaaagcac tgctctttac ccatccattt atttgaacac
tcagcagtct
cctgtagctg ctacactcta tgtgcgcaat cgagttcggg aagccatcag agtttccaaa atacctgatg caaaaagtcc
acttccggtt
tttgcatata cccgcatagt ttttactgat caagttttga aattcctttc tcaagatgaa cttgtgtata catttggcga
aactgttgct
ctgggtgctt ctggaattgt aatatgggga accctcagta taatgcgaag tatgaaatct tgcttgctcc tagacaatta
catggagact
atactgaatc cttacataat caacgtcaca ctagcagcca aaatgtgtag ccaagtgctt tgccaggagc aaggagtgtg
tataaggaaa
aactggaatt caagtgacta tcttcacctc aacccagata attttgctat tcaacttgag aaaggtggaa agttcacagt
acgtggaaaa
ccgacacttg aagacctgga gcaattttct gaaaaatttt attgcagctg ttatagcacc ttgagttgta aggagaaagc
tgatgtaaaa
gacactgatg ctgttgatgt gtgtattgct gatggtgtct gtatagatgc ttttctaaaa cctcccatgg agacagaaga
acctcaaatt
ttctac
SEQ ID NO: 420
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Gly Pro Tyr Ile Ile Asn Val Thr Leu Ala Ala Lys Met
Cys Ser Gln Val Leu Cys Gln Glu Gln Gly Val Cys Ile Arg Lys Asn Trp Asn Ser Ser Asp
Tyr Leu His Leu Asn Pro Asp Asn Phe Ala Ile Gln Leu Glu Lys Gly Gly Lys Phe Thr Val
Arg Gly Lys Pro Thr Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala Val Asp Val Cys Ile
Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 421
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Phe Ile Leu Asn Val Thr Ser Gly Ala Leu
Leu Cys Ser Gln Ala Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 422
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Gly Pro Phe Ile Leu Asn Val Thr Ser Gly Ala Leu
Leu Cys Ser Gln Ala Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 423
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Val Ser Trp Glu Asn Thr Arg Thr Lys Glu Ser Cys Gln Ala
Ile Lys Glu Tyr Met Asp Thr Thr Leu Gly Pro Tyr Ile Ile Asn Val Thr Leu Ala Ala Lys Met
Cys Ser Gln Val Leu Cys Gln Glu Gln Gly Val Cys Ile Arg Lys Asn Trp Asn Ser Ser Asp
Tyr Leu His Leu Asn Pro Asp Asn Phe Ala Ile Gln Leu Glu Lys Gly Gly Lys Phe Thr Val
Arg Gly Lys Pro Thr Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala Val Asp Val Cys Ile
Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 424
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys AlaGly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 425
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Val Ser Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 426
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 427
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 428
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 429
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Phe Ile Leu Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 430
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Phe Ile Leu Asn Val Thr Ser Gly Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 431
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Leu Ser Ile Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr Asn Ala Ser Pro Ser Thr Leu Ser
SEQ ID NO: 432
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile
SEQ ID NO: 433
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe
SEQ ID NO: 434
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu
Gly Met Ala Val Ile Asp Trp Glu Glu Trp Arg Pro Thr Trp Ala Arg Asn Trp Lys Pro Lys
Asp Val Tyr Lys Asn Arg Ser Ile Glu Leu Val Gln Gln Gln Asn Val Gln Leu Ser Leu Thr
Glu Ala Thr Glu Lys Ala Lys Gln Glu Phe Glu Lys Ala Gly Lys Asp Phe Leu Val Glu
Thr Ile Lys Leu Gly Lys Leu Leu Arg Pro Asn His Leu Trp Gly Tyr Tyr Leu Phe Pro Asp
Cys Tyr Asn His His Tyr Lys Lys Pro Gly Tyr Asn Gly Ser Cys Phe Asn Val Glu Ile Lys
Arg Asn Asp Asp Leu Ser Trp Leu Trp Asn Glu Ser Thr Ala Leu Tyr Pro Ser Ile Tyr Leu
Asn Thr Gln Gln Ser Pro Val Ala Ala Thr Leu Tyr Val Arg Asn Arg Val Arg Glu Ala Ile
Arg Val Ser Lys Ile Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr Thr Phe Gly Glu
Thr Val Ala Leu Gly Ala Ser Gly Ile Val Ile Trp Gly Thr Leu Ser Ile Thr Arg Thr Lys Glu
Ser Cys Gln Ala Ile Lys Glu Tyr Met Asp Thr Thr Leu Asn Pro Tyr Ile Ile Asn Val Thr
Leu Ala Ala Lys Met Cys Ser Gln Val Leu Cys Gln Glu Gln Gly Val Cys Ile Arg Lys Asn
Trp Asn Ser Ser Asp Tyr Leu His Leu Asn Pro Asp Asn Phe Ala Ile Gln Leu Glu Lys Gly
Gly Lys Phe Thr Val Arg Gly Lys Pro Thr Leu Glu Asp Leu Glu Gln Phe Ser Glu Lys
Phe Tyr Cys Ser Cys Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu Lys Pro
SEQ ID NO: 435
Phe Arg Gly Pro Leu Leu Pro Asn Arg Pro Phe Leu Trp Ala Trp Asn Ala Pro Ser Glu Phe
Cys Leu Gly Lys Phe Asp Glu Pro Leu Asp Met Ser Leu Phe Ser Phe Ile Gly Ser Pro Arg
Ile Asn Ala Thr Gly Gln Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile Ser Leu Gln Asp His Leu
Asp Lys Ala Lys Lys Asp Ile Thr Phe Tyr Met Pro Val Asp Asn Leu Gly Met Ala Val Ile
Asp Trp Glu Glu Trp Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr Glu Ala Thr Glu Lys
Ala Lys Gln Glu Phe Glu Lys Ala Gly Lys Asp Phe Leu Val Glu Thr Ile Lys Leu Gly Lys
Leu Leu Arg Pro Asn His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg Asn Asp Asp Leu Ser
Trp Leu Trp Asn Glu Ser Thr Ala Leu Tyr Pro Ser Ile Tyr Leu Asn Thr Gln Gln Ser Pro
Val Ala Ala Thr Leu Tyr Val Arg Asn Arg Val Arg Glu Ala Ile Arg Val Ser Lys Ile Pro
Asp Ala Lys Ser Pro Leu Pro Val Phe Ala Tyr Thr Arg Ile Val Phe Thr Asp Gln Val Leu
Lys Phe Leu Ser Gln Asp Glu Leu Val Tyr Thr Phe Gly Glu Thr Val Ala Leu Gly Ala Ser
Gly Ile Val Ile Trp Gly Thr Leu Ser Ile Thr Arg Thr Lys Glu Ser Cys Gln Ala Ile Lys Glu
Tyr Met Asp Thr Thr Leu Asn Pro Tyr Ile Ile Asn Val Thr Leu Ala Ala Lys Met Cys Ser
Gln Val Leu Cys Gln Glu Gln Gly Val Cys Ile Arg Lys Asn Trp Asn Ser Ser Asp Tyr Leu
His Leu Asn Pro Asp Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys Ser Cys Tyr Ser Thr
Leu Ser Cys Lys Glu Lys Ala Asp Val Lys Asp Thr Asp Ala Val Asp Val Cys Ile Ala Asp
Gly Val Cys Ile Asp Ala Phe Leu Lys Pro Pro Met Glu Thr Glu Glu Pro Gln Ile Phe Tyr
Asn Ala Ser Pro Ser Thr Leu Ser
SEQ ID NO: 436
Phe Arg Gly Pro Leu Leu Pro Asn Arg Pro Phe Thr Thr Val Trp Asn Ala Pro Ser Glu Phe
Cys Leu Gly Lys Phe Asp Glu Pro Leu Asp Met Ser Leu Phe Ser Phe Ile Gly Ser Pro Arg
Ile Asn Ala Thr Gly Gln Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile Ser Leu Gln Asp His Leu
Asp Lys Ala Lys Lys Asp Ile Thr Phe Tyr Met Pro Val Asp Asn Leu Gly Met Ala Val Ile
Asp Trp Glu Glu Trp Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr Glu Ala Thr Glu Lys
Ala Lys Gln Glu Phe Glu Lys Ala Gly Lys Asp Phe Leu Val Glu Thr Ile Lys Leu Gly Lys
Leu Leu Arg Pro Asn His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg Asn Asp Asp Leu Ser
Trp Leu Trp Asn Glu Ser Thr Ala Leu Tyr Pro Ser Ile Tyr Leu Asn Thr Gln Gln Ser Pro
Val Ala Ala Thr Leu Tyr Val Arg Asn Arg Val Arg Glu Ala Ile Arg Val Ser Lys Ile Pro
Asp Ala Lys Ser Pro Leu Pro Val Phe Ala Tyr Thr Arg Ile Val Phe Thr Asp Gln Val Leu
Lys Phe Leu Ser Gln Asp Glu Leu Val Tyr Thr Phe Gly Glu Thr Val Ala Leu Gly Ala Ser
Gly Ile Val Ile Trp Gly Thr Leu Ser Ile Thr Arg Thr Lys Glu Ser Cys Gln Ala Ile Lys Glu
Tyr Met Asp Thr Thr Leu Asn Pro Tyr Ile Ile Asn Val Thr Leu Ala Ala Lys Met Cys Ser
Gln Val Leu Cys Gln Glu Gln Gly Val Cys Ile Arg Lys Asn Trp Asn Ser Ser Asp Tyr Leu
His Leu Asn Pro Asp Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys Ser Cys Tyr Ser Thr
Leu Ser Cys Lys Glu Lys Ala Asp Val Lys Asp Thr Asp Ala Val Asp Val Cys Ile Ala Asp
Gly Val Cys Ile Asp Ala Phe Leu Lys Pro Pro Met Glu Thr Glu Glu Pro Gln Ile Phe Tyr
Asn Ala Ser Pro Ser Thr Leu Ser
SEQ ID NO: 437
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys
SEQ ID NO: 438
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 439
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Ser Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 440
Ala Pro Pro Val Ile Pro Asn Val Pro Phe Leu Trp Ala Trp Asn Ala Pro Ser Glu Phe Cys
Leu Gly Lys Phe Asp Glu Pro Leu Asp Met Ser Leu Phe Ser Phe Ile Gly Ser Pro Arg Ile
Asn Ala Thr Gly Gln Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile Ser Leu Gln Asp His Leu
Asp Lys Ala Lys Lys Asp Ile Thr Phe Tyr Met Pro Val Asp Asn Leu Gly Met Ala Val Ile
Asp Trp Glu Glu Trp Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr Glu Ala Thr Glu Lys
Ala Lys Gln Glu Phe Glu Lys Ala Gly Lys Asp Phe Leu Val Glu Thr Ile Lys Leu Gly Lys
Leu Leu Arg Pro Asn His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg Asn Asp Asp Leu Ser
Trp Leu Trp Asn Glu Ser Thr Ala Leu Tyr Pro Ser Ile Tyr Leu Asn Thr Gln Gln Ser Pro
Val Ala Ala Thr Leu Tyr Val Arg Asn Arg Val Arg Glu Ala Ile Arg Val Ser Lys Ile Pro
Asp Ala Lys Ser Pro Leu Pro Val Phe Ala Tyr Thr Arg Ile Val Phe Thr Asp Gln Val Leu
Lys Phe Leu Ser Gln Asp Glu Leu Val Tyr Thr Phe Gly Glu Thr Val Ala Leu Gly Ala Ser
Gly Ile Val Ile Trp Gly Thr Leu Ser Ile Thr Arg Thr Lys Glu Ser Cys Gln Ala Ile Lys Glu
Tyr Met Asp Thr Thr Leu Asn Pro Tyr Ile Ile Asn Val Thr Leu Ala Ala Lys Met Cys Ser
Gln Val Leu Cys Gln Glu Gln Gly Val Cys Ile Arg Lys Asn Trp Asn Ser Ser Asp Tyr Leu
His Leu Asn Pro Asp Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys Ser Cys Tyr Ser Thr
Leu Ser Cys Lys Glu Lys Ala Asp Val Lys Asp Thr Asp Ala Val Asp Val Cys Ile Ala Asp
Gly Val Cys Ile Asp Ala Phe Leu Lys Pro Pro Met Glu Thr Glu Glu Pro Gln Ile Phe Tyr
Asn Ala Ser Pro Ser Thr Leu Ser
SEQ ID NO: 441
Pro Val Ile Pro Asn Val Pro Phe Leu Trp Ala Trp Asn Ala Pro Ser Glu Phe Cys Leu Gly
Lys Phe Asp Glu Pro Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr Pro Tyr Ile Asp Ser Ile
Thr Gly Val Thr Val Asn Gly Gly Ile Pro Gln Lys Ile Ser Leu Gln Asp His Leu Asp Lys
Ala Lys Lys Asp Ile Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val Tyr Lys Asn Arg Ser
Ile Glu Leu Val Gln Gln Gln Asn Val Gln Leu Ser Leu Thr Glu Ala Thr Glu Lys Ala Lys
Gln Glu Phe Glu Lys Ala Gly Lys Asp Phe Leu Val Glu Thr Ile Lys Leu Gly Lys Leu Leu
Arg Pro Asn His Leu Trp Gly Tyr Tyr Leu Phe Pro Asp Cys Tyr Asn His His Tyr Lys Lys
Pro Gly Tyr Asn Gly Ser Cys Phe Asn Val Glu Ile Lys Arg Asn Asp Asp Leu Ser Trp Leu
Trp Asn Glu Ser Thr Ala Leu Tyr Pro Ser Ile Tyr Leu Asn Thr Gln Gln Ser Pro Val Ala Ala
Thr Leu Tyr Val Arg Asn Arg Val Arg Glu Ala Ile Arg Val Ser Lys Ile Pro Asp Ala Lys
Ser Pro Leu Pro Val Phe Ala Tyr Thr Arg Ile Val Phe Thr Asp Gln Val Leu Lys Phe Leu
Ser Gln Asp Glu Leu Val Tyr Thr Phe Gly Glu Thr Val Ala Leu Gly Ala Ser Gly Ile Val Ile
Trp Gly Thr Leu Ser Ile Thr Arg Thr Lys Glu Ser Cys Gln Ala Ile Lys Glu Tyr Met Asp
Thr Thr Leu Asn Pro Tyr Ile Ile Asn Val Thr Leu Ala Ala Lys Met Cys Ser Gln Val Leu
Cys Gln Glu Gln Gly Val Cys Ile Arg Lys Asn Trp Asn Ser Ser Asp Tyr Leu His Leu Asn
Pro Asp Asn Phe Ala Ile Gln Leu Glu Lys Gly Gly Lys Phe Thr Val Arg Gly Lys Pro Thr
Leu Glu Asp Leu Glu Gln Phe Ser Glu Lys Phe Tyr Cys Ser Cys Tyr Ser Thr Leu Ser Cys
Lys Glu Lys Ala Asp Val Lys Asp Thr Asp Ala Val Asp Val Cys Ile Ala Asp Gly Val Cys
Ile Asp Ala Phe Leu Lys Pro Pro Met Glu Thr Glu Glu Pro Gln Ile Phe Tyr Asn Ala Ser Pro
Ser Thr Leu Ser
SEQ ID NO: 442
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala
SEQ ID NO: 443
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys
SEQ ID NO: 444
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Pro Thr Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala Val Asp Val Cys Ile
Ala Asp
SEQ ID NO: 445
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
SEQ ID NO: 446
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu
Gly Met Ala Val Ile Asp Trp Glu Glu Trp Arg Pro Thr Trp Ala Arg Asn Trp Lys Pro Lys
Asp Val Tyr Lys Asn Arg Ser Ile Glu Leu Val Gln Gln Gln Asn Val Gln Leu Ser Leu Thr
Glu Ala Thr Glu Lys Ala Lys Gln Glu Phe Glu Lys Ala Gly Lys Asp Phe Leu Val Glu
Thr Ile Lys Leu Gly Lys Leu Leu Arg Pro Asn His Leu Trp Gly Tyr Tyr Leu Phe Pro Asp
Cys Tyr Asn His His Tyr Lys Lys Pro Gly Tyr Asn Gly Ser Cys Phe Asn Val Glu Ile Lys
Arg Asn Asp Asp Leu Ser Trp Leu Trp Asn Glu Ser Thr Ala Leu Tyr Pro Ser Ile Tyr Leu
Asn Thr Gln Gln Ser Pro Val Ala Ala Thr Leu Tyr Val Arg Asn Arg Val Arg Glu Ala Ile
Arg Val Ser Lys Ile Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr Thr Phe Gly Glu
Thr Val Ala Leu Gly Ala Ser Gly Ile Val Ile Trp Gly Thr Trp Glu Asn Thr Arg Thr Lys
Glu Ser Cys Gln Ala Ile Lys Glu Tyr Met Asp Thr Thr Leu Asn Pro Tyr Ile Ile Asn Val
Thr Leu Ala Ala Lys Met Cys Ser Gln Val Leu Cys Gln Glu Gln Gly Val Cys Ile Arg Lys
Asn Trp Asn Ser Ser Asp Tyr Leu His Leu Asn Pro Asp Asn Phe Ala Ile Gln Leu Glu Lys
Gly Gly Lys Phe Thr Val Arg Gly Lys Pro Thr Leu Glu Asp Leu Glu Gln Phe Ser Glu
Lys Phe Tyr Cys Ser Cys Tyr Ser Thr Leu Ser Cys Lys Glu Lys Ala Asp Val Lys Asp Thr
Asp Ala Val
SEQ ID NO: 447
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys
SEQ ID NO: 448
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro
SEQ ID NO: 449
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met
SEQ ID NO: 450
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu
SEQ ID NO: 451
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr
SEQ ID NO: 452
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu
SEQ ID NO: 453
Asn Phe Arg Ala Pro Pro Val Ile Pro Asn Val Pro Phe Leu Trp Ala Trp Asn Ala Pro Ser
Glu Phe Cys Leu Gly Lys Phe Asp Glu Pro Leu Asp Met Ser Leu Phe Ser Phe Ile Gly Ser
Pro Arg Ile Asn Ala Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro Gln Lys Ile Ser Leu Gln
Asp His Leu Asp Lys Ala Lys Lys Asp Ile Thr Phe Tyr Met Pro Val Asp Asn Leu Gly Met
Ala Val Ile Asp Trp Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu Ser Leu Thr Glu Ala
Thr Glu Lys Ala Lys Gln Glu Phe Glu Lys Ala Gly Lys Asp Phe Leu Val Glu Thr Ile Lys
Leu Gly Lys Leu Leu Arg Pro Asn His Leu Trp Gly Tyr Tyr Leu Phe Pro Asp Cys Tyr Asn
His His Tyr Lys Lys Pro Gly Tyr Asn Gly Ser Cys Phe Asn Val Glu Ile Lys Arg Asn Asp
Asp Leu Ser Trp Leu Trp Asn Glu Ser Thr Ala Leu Tyr Pro Ser Ile Tyr Leu Asn Thr Gln
Gln Ser Pro Val Ala Ala Thr Leu Tyr Val Arg Asn Arg Val Arg Glu Ala Ile Arg Val Ser
Lys Ile Pro Asp Ala Lys Ser Pro Leu Pro Val Phe Ala Tyr Thr Arg Ile Val Phe Thr Asp Gln
Val Leu Lys Phe Leu Ser Gln Asp Glu Leu Val Tyr Thr Phe Gly Glu Thr Val Ala Leu Gly
Ala Ser Gly Ile Val Ile Trp Gly Thr Leu Ser Ile Thr Arg Thr Lys Glu Ser Cys Gln Ala Ile
Lys Glu Tyr Met Asp Thr Thr Leu Asn Pro Tyr Ile Ile Asn Val Thr Leu Ala Ala Lys Met
Cys Ser Gln Val Leu Cys Gln Glu Gln Gly Val Cys Ile Arg Lys Asn Trp Asn Ser Ser Asp
Tyr Leu His Leu Asn Pro Asp Asn Phe Ala Ile Gln Leu Glu Lys Gly Gly Lys Phe Thr Val
Arg Gly Lys Pro Thr Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala Val Asp Val Cys Ile
Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 454
Arg Ala Pro Pro Val Ile Pro Asn Val Pro Phe Leu Trp Ala Trp Asn Ala Pro Ser Glu Phe
Cys Leu Gly Lys Phe Asp Glu Pro Leu Asp Met Ser Leu Phe Ser Phe Ile Gly Ser Pro Arg
Ile Asn Ala Thr Gly Gln Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile Ser Leu Gln Asp His Leu
Asp Lys Ala Lys Lys Asp Ile Thr Phe Tyr Met Pro Val Asp Asn Leu Gly Met Ala Val Ile
Asp Trp Glu Glu Trp Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr Glu Ala Thr Glu Lys
Ala Lys Gln Glu Phe Glu Lys Ala Gly Lys Asp Phe Leu Val Glu Thr Ile Lys Leu Gly Lys
Leu Leu Arg Pro Asn His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg Asn Asp Asp Leu Ser
Trp Leu Trp Asn Glu Ser Thr Ala Leu Tyr Pro Ser Ile Tyr Leu Asn Thr Gln Gln Ser Pro
Val Ala Ala Thr Leu Tyr Val Arg Asn Arg Val Arg Glu Ala Ile Arg Val Ser Lys Ile Pro
Asp Ala Lys Ser Pro Leu Pro Val Phe Ala Tyr Thr Arg Ile Val Phe Thr Asp Gln Val Leu
Lys Phe Leu Ser Gln Asp Glu Leu Val Tyr Thr Phe Gly Glu Thr Val Ala Leu Gly Ala Ser
Gly Ile Val Ile Trp Gly Thr Leu Ser Ile Thr Arg Thr Lys Glu Ser Cys Gln Ala Ile Lys Glu
Tyr Met Asp Thr Thr Leu Asn Pro Tyr Ile Ile Asn Val Thr Leu Ala Ala Lys Met Cys Ser
Gln Val Leu Cys Gln Glu Gln Gly Val Cys Ile Arg Lys Asn Trp Asn Ser Ser Asp Tyr Leu
His Leu Asn Pro Asp Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys Ser Cys Tyr Ser Thr
Leu Ser Cys Lys Glu Lys Ala Asp Val Lys Asp Thr Asp Ala Val Asp Val Cys Ile Ala Asp
Gly Val Cys Ile Asp Ala Phe Leu Lys Pro Pro Met Glu Thr Glu Glu Pro Gln Ile Phe Tyr
Asn Ala Ser Pro Ser Thr Leu Ser
SEQ ID NO: 455
Pro Pro Val Ile Pro Asn Val Pro Phe Leu Trp Ala Trp Asn Ala Pro Ser Glu Phe Cys Leu
Gly Lys Phe Asp Glu Pro Leu Asp Met Ser Leu Phe Ser Phe Ile Gly Ser Pro Arg Ile Asn
Ala Thr Gly Gln Gly Val Thr Ile Phe Tyr Val Asp Arg Leu Gly Tyr Tyr Pro Tyr Ile Asp Ser
Ile Thr Gly Val Thr Val Asn Gly Gly Ile Pro Gln Lys Ile Ser Leu Gln Asp His Leu Asp Lys
Ala Lys Lys Asp Ile Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val Tyr Lys Asn Arg Ser
Ile Glu Leu Val Gln Gln Gln Asn Val Gln Leu Ser Leu Thr Glu Ala Thr Glu Lys Ala Lys
Gln Glu Phe Glu Lys Ala Gly Lys Asp Phe Leu Val Glu Thr Ile Lys Leu Gly Lys Leu Leu
Arg Pro Asn His Leu Trp Gly Tyr Tyr Leu Phe Pro Asp Cys Tyr Asn His His Tyr Lys Lys
Pro Gly Tyr Asn Gly Ser Cys Phe Asn Val Glu Ile Lys Arg Asn Asp Asp Leu Ser Trp Leu
Trp Asn Glu Ser Thr Ala Leu Tyr Pro Ser Ile Tyr Leu Asn Thr Gln Gln Ser Pro Val Ala Ala
Thr Leu Tyr Val Arg Asn Arg Val Arg Glu Ala Ile Arg Val Ser Lys Ile Pro Asp Ala Lys
Ser Pro Leu Pro Val Phe Ala Tyr Thr Arg Ile Val Phe Thr Asp Gln Val Leu Lys Phe Leu
Ser Gln Asp Glu Leu Val Tyr Thr Phe Gly Glu Thr Val Ala Leu Gly Ala Ser Gly Ile Val Ile
Trp Gly Thr Leu Ser Ile Thr Arg Thr Lys Glu Ser Cys Gln Ala Ile Lys Glu Tyr Met Asp
Thr Thr Leu Asn Pro Tyr Ile Ile Asn Val Thr Leu Ala Ala Lys Met Cys Ser Gln Val Leu
Cys Gln Glu Gln Gly Val Cys Ile Arg Lys Asn Trp Asn Ser Ser Asp Tyr Leu His Leu Asn
Pro Asp Asn Phe Ala Ile Gln Leu Glu Lys Gly Gly Lys Phe Thr Val Arg Gly Lys Pro Thr
Leu Glu Asp Leu Glu Gln Phe Ser Glu Lys Phe Tyr Cys Ser Cys Tyr Ser Thr Leu Ser Cys
Lys Glu Lys Ala Asp Val Lys Asp Thr Asp Ala Val Asp Val Cys Ile Ala Asp Gly Val Cys
Ile Asp Ala Phe Leu Lys Pro Pro Met Glu Thr Glu Glu Pro Gln Ile Phe Tyr Asn Ala Ser Pro
Ser Thr Leu Ser
SEQ ID NO: 456
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile
SEQ ID NO: 457
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp
SEQ ID NO: 458
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala
SEQ ID NO: 459
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 460
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro
SEQ ID NO: 461
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile
SEQ ID NO: 462
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr
SEQ ID NO: 463
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr Asn Ala
SEQ ID NO: 464
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr Asn Ala Ser Pro
SEQ ID NO: 465
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr Asn Ala Ser Pro Ser Thr
SEQ ID NO: 466
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Gly Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 467
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Ala Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 468
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Cys Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 469
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Asp Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 470
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Leu Ser Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 471
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Leu Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 472
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ala Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 473
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Gly Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 474
Phe Arg Gly Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 475
Phe Arg Gly Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 476
QMQLVESGGGVVQPGRSLRLSCAASGFTFRTYGMHWVRQAPGKGLEWVAVIW
YDGSNKHYADSVKGRFTITRDNSKNTLNLQMNSLRAEDTAVYYCARAPQWELV
HEAFDIWGQGTMVTVSSASTKGPSVFPLAPCSRSTSESTAALGCLVKDYFPEPVTV
SWNSGALTSGVHTFPAVLQSSGLYSLSSVVTVPSSNFGTQTYTCNVDHKPSNTKV
DKTVERKCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYPSDIAVE
WESNGQPENNYKTTPPMLDSDGSFFLYSKLTVDKSRWQQGNVFSCSVMHEALH
NHYTQKSLSLSPGK
SEQ ID NO: 477
SYVLTQPPSVSVAPGQTARITCGGNNLGSKSVHWYQQKPGQAPVLVVYDDSDRP
SWIPERFSGSNSGNTATLTISRGEAGDEADYYCQVWDSSSDHVVFGGGTKLTVLG
QPKAAPSVTLFPPSSEELQANKATLVCLISDFYPGAVTVAWKADSSPVKAGVETT
TPSKQSNNKYAASSYLSLTPEQWKSHRSYSCQVTHEGSTVEKTVAPTECS
SEQ ID NO: 478
QMQLVESGGGVVQPGRSLRLSCAASGFTFRTYGMHWVRQAPGKGLEWVAVIW
YDGSNKHYADSVKGRFTITRDNSKNTLNLQMNSLRAEDTAVYYCARAPQWELV
HEAFDIWGQGTMVTVSS
SEQ ID NO: 479
SYVLTQPPSVSVAPGQTARITCGGNNLGSKSVHWYQQKPGQAPVLVVYDDSDRP
SWIPERFSGSNSGNTATLTISRGEAGDEADYYCQVWDSSSDHVVFGGGTKLTVL

| | | |
|---|---|---|
| Human lambda-2 | GQPKAAPSVTLFPPSSEELQANKATLVCLISDFYPGAVTVA | 480 |
| light chain | WKADSSPVKAGVETTTPSKQSNNKYAASSYLSLTPEQWKS | |
| constant region | HRSYSCQVTHEGSTVEKTVAPTECS | |
| (lambda light | | |
| chain constant | | |
| region, subtype 2) | | |
| UniProt P0DOY2 | | |
| Human lambda-1 | GQPKANPTVTLFPPSSEELQANKATLVCLISDFYPGAVTVA | 481 |
| light chain | WKADGSPVKAGVETTKPSKQSNNKYAASSYLSLTPEQWKS | |
| constant region | HRSYSCQVTHEGSTVEKTVAPTECS | |
| (lambda light | | |
| chain constant | | |
| region, subtype 1) | | |
| UniProt P0CG04 | | |
| Human lambda-3 | GQPKAAPSVTLFPPSSEELQANKATLVCLISDFYPGAVTVA | 482 |
| light chain | WKADSSPVKAGVETTTPSKQSNNKYAASSYLSLTPEQWKS | |
| constant region | HKSYSCQVTHEGSTVEKTVAPTECS | |
| (lambda light | | |
| chain constant | | |
| region, subtype 3) | | |
| UniProt P0DOY3 | | |
| Human lambda-7 | GQPKAAPSVTLFPPSSEELQANKATLVCLVSDFNPGAVTVA | 483 |
| light chain | WKADGSPVKVGVETTKPSKQSNNKYAASSYLSLTPEQWKS | |
| constant region | HRSYSCRVTHEGSTVEKTVAPAECS | |
| (lambda light | | |
| chain constant | | |
| region, subtype 7) | | |
| UniProt A0M8Q6 | | |
| Antibody 5 Heavy | QMQLVESGGGVVQPGRSLRLSCAASGFTFRTYGMHWVRQ | 484 |
| Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 5 Light | SYVLTQPPSVSVAPGQTARITCGGNNLVSKSVHWYQQKPG | 485 |
| Chain | QAPVLVVYDDSDRPSWIPERFSGSNSGNTATLTISRGEAGDE | |
| | ADYYCQVWDSSSTFVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| Antibody 5 Heavy | QMQLVESGGGVVQPGRSLRLSCAASGFTFRTYGMHWVRQ | 486 |
| Chain without C- | APGKGLEWVAVIWYDGSNKHYADSVKGRFTITRDNSKNTL | |
| terminal lysine | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPG | |
| Antibody 7 Heavy | QMQLVESGGGVVQPGRSLRLSCAASGFTFRTYGMHWVRQ | 487 |
| Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 7 Light | SYVLTQPPSVSVAPGQTARITCGGNNLGSKSVHWYQQKPG | 488 |
| Chain | QAPVLVVYDVSDRPSWIPERFSGSNSGNTATLTISRGEAGDE | |
| | ADYYCQVWDSSSTIVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| Antibody 23 | QMQLVESGGGVVQPGRSLRLSCAASGFTFRTYGMHWVRQ | 489 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 23 Light | SYELTQPPSVSVAPGQTARITCGGHNIGTKSVHWYQQKSGQ | 490 |
| Chain | APILVVYDDSDRPSWIPERFSGSNFGTTATLTISRVEAGDEA | |
| | DYFCQVWDRSSDQSVFGPGTRVTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 29 | QMQLVESGGGVVQPGRSLRLSCAASGFTFRTYGMHWVRQ | 491 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 29 Light | SYELTQPPSVSVAPGQTARITCGGNNIGRKSAHWYQQRPGQ | 492 |
| Chain | APVLVVYDDSDRPSWIPERFSGSNSGNTATLTISRVEAGDEA | |
| | DYYCQVWDSYSNHVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| Antibody 31 | QMQLVESGGGVVQPGRSLRLSCAASGFTFRTYGMHWVRQ | 493 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 31 Light | SYVLTQPPSVSVAPGQTARITCGGHNIGTKSVHWYQQKSGQ | 494 |
| Chain | APILAVYDDSDRPSWIPERFSGSNFGTTATLTISRVEAGDEA | |
| | DYFCQVWDRSSDQSVFGPGTRVTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 36 | QMQLVESGGGVVQPGRSLRLSCAASGFTFRTYGMHWVRQ | 495 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 36 Light | SYVLTQPPSVSVAPGQTARITCGGQSIGTKSVHWYQQKSGQ | 496 |
| Chain | APILVVYDDSDRPSWIPERFSGSNFGTTATLTISRVEAGDEA | |
| | DYFCQVWDSSSDQSVFGPGTRVTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 38 | QMQLVESGGGVVQPGRSLRLSCAASGFTFRTYGMHWVRQ | 497 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 38 Light | SYELTQPPSVSVAPGQTARITCGGNKIGRKSVHWYQQRPGQ | 498 |
| Chain | APVLVVFDDSDRPSWISERFSGSNSGNTATLTIGRVEAGDEA | |
| | DYYCQVWDSSSDEGVFGGGTKLAVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 39 | QMQLVESGGGVVQPGRSLRLSCAASGFTFRTYGMHWVRQ | 499 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 39 Light | SYELTQPPSVSVAPGRTARITCGGQNIGTKSVHWYQQKSGQ | 500 |
| Chain | APILVVYDDSDRPSWIPERFSGSNFGTTATLTISRVEAGDEA | |
| | DYFCQVWDSSSDQSVFGPGTRVTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 44 | QMQLVESGGGVVQPGRSLRLSCAASGFTFRTYGMHWVRQ | 501 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 44 Light | SYELTQPPSVSVAPGQTARITCGGNKIGRKSVHWYQQKPGQ | 502 |
| Chain | APVLVVFDDSDRPSWISERFSGSNSGNTATLTISRVEAGDEA | |
| | DYHCQVWDTSSDQGVFGGGTKLTVLGQPKAAPSVTLFPPSS | |
| | EELQANKATLVCLISDFYPGAVTVAWKADSSPVKAGVETTT | |
| | PSKQSNNKYAASSYLSLTPEQWKSHRSYSCQVTHEGSTVEK | |
| | TVAPTECS | |
| Antibody 51 | QMQLVESGGGVVQPGRSLRLSCAASGFTFRTYGMHWVRQ | 503 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 51 Light | SYVLTQPPSVSVAPGQTARITCGGNNLGSKSVHWYQQKPG | 504 |
| Chain | QAPVLVVYDVSDRPSWIPERFSGSNSGNTATLTISRGEAGDE | |
| | ADYYCQVWDSSSTHVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| Antibody 59 | QMQLVESGGGVVQPGRSLRLSCAASGFTFRTYGMHWVRQ | 505 |
| Heavy Chain | APGKGLEWVAVIWYDGSNKHYADSVKGRFTITRDNSKNTL | |
| | NLQMNSLRAEDTAVYYCARAPQWELVHEAFDIWGQGTMV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGGP | |
| | SVFLFPPKPKDTLYITREPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSRDELTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SLSPGK | |
| Antibody 59 Light | SYVLTQPPSVSVAPGQTARITCGGNNLVSKSVHWYQQKPG | 506 |
| Chain | QAPVLVVYDVSDRPSWIPERFSGSNSGNTATLTISRGEAGDE | |
| | ADYYCQVWDSSSTFVVFGGGTKLTVLGQPKAAPSVTLFPPS | |
| | SEELQANKATLVCLISDFYPGAVTVAWKADSSPVKAGVETT | |
| | TPSKQSNNKYAASSYLSLTPEQWKSHRSYSCQVTHEGSTVE | |
| | KTVAPTECS | |
| SRDEL allotype | SRDEL | 507 |
| SREEM allotype | SREEM | 508 |

1. A composition comprising an isolated antibody, or antigen binding fragment thereof, that binds thymic stromal lymphopoietin (TSLP) and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27.

2. (canceled)

3. The composition of claim 1, wherein the antibody, or antigen binding fragment thereof, comprises a VH sequence set forth in SEQ ID NO:34 and a VL sequence set forth in SEQ ID NO:41.

4. (canceled)

5. A combination comprising an isolated antibody, or antigen binding fragment thereof, that binds TSLP and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27.

6. (canceled)

7. The combination of claim 5, wherein the antibody, or antigen binding fragment thereof, comprises a VH sequence set forth in SEQ ID NO:34 and a VL sequence set forth in SEQ ID NO:41.

8. (canceled)

9. A method of treating an inflammatory disorder or disease in a human patient comprising administering to the patient an isolated antibody, or antigen binding fragment thereof, that binds TSLP and a hyaluronidase or variant thereof, wherein the antibody, or antigen binding fragment thereof, comprises a CDR-H1 comprising the sequence set forth in SEQ ID NO: 3; a CDR-H2 comprising the sequence set forth in SEQ ID NO: 10; a CDR-H3 comprising the sequence set forth in SEQ ID NO: 13; a CDR-L1 comprising the sequence set forth in SEQ ID NO: 21; a CDR-L2 comprising the sequence set forth in SEQ ID NO: 26; and a CDR-L3 comprising the sequence set forth in SEQ ID NO: 27.

10. (canceled)

11. The method of claim 9, wherein the antibody, or antigen binding fragment thereof, comprises a VH sequence set forth in SEQ ID NO:34 and a VL sequence set forth in SEQ ID NO:41.

12. (canceled)

13. The composition of claim 1, wherein the antibody, or antigen binding fragment thereof, is a humanized, human, or chimeric antibody.

14.-16. (canceled)

17. The composition of claim 1, wherein the antibody, or antigen binding fragment thereof, comprises a human IgG1 Fc region.

18. The composition of claim 1, wherein the antibody, or antigen binding fragment thereof, comprises a constant heavy chain sequence set forth in any one of SEQ ID NOs: 47-270.

19. The composition of claim 1, wherein the antibody, or antigen binding fragment thereof, comprises a constant heavy chain sequence set forth in SEQ ID NO: 173 or SEQ ID NO: 61.

20. The composition of claim 1, wherein the antibody, or antigen binding fragment thereof, comprises a constant light chain sequence set forth in SEQ ID NO: 480.

21. The composition of claim 1, wherein the antibody, or antigen binding fragment thereof, comprises a constant heavy chain sequence set forth in SEQ ID NO: 173 or SEQ ID NO: 61 and a constant light chain sequence set forth in SEQ ID NO: 480.

22. The composition of claim 1, wherein;
(a) the antibody, or antigen binding fragment thereof, is a monoclonal antibody; or
(b) the antibody, or antigen binding fragment thereof, binds a TSLP sequence set forth in SEQ ID NO: 365 or 366.

23. (canceled)

24. The composition of claim 1, wherein the antibody, or antigen binding fragment thereof, comprises a human IgG1 Fc region with LALA and YTE mutations.

25. The composition of claim 1, wherein the antibody, or antigen binding fragment thereof, comprises a human IgG1 Fc region with LALA mutations at positions 239 and 240 (L239A/L240A) and YTE mutations at positions 257, 259, and 261 (M257Y/S259T/T261E) by direct numbering.

26. The composition of claim 1, wherein the antibody, or antigen binding fragment thereof, comprises a heavy chain comprising a constant heavy chain region having a means for extending the half-life of the antibody.

27.-28. (canceled)

29. The composition of claim 1, wherein the hyaluronidase or variant thereof is a recombinant human hyaluronidase.

30. The composition of claim 29, wherein the recombinant human hyaluronidase or variant thereof comprises an amino acid sequence set forth in any one of SEQ ID NOs: 409-418 and 420-475.

31. The composition of claim 29, wherein the recombinant human hyaluronidase is rHuPH20.

32. The composition claim 31, wherein the rHuPH20 formulation is ENHANZE®.

33.-37. (canceled)

38. The composition of claim 1, wherein the hyaluronidase or variant thereof and antibody, or antigen binding fragment thereof, are formulated for administration administered by subcutaneous or intravenous injection.

39.-44. (canceled)

45. A kit for treating an inflammatory disorder or disease in a human patient, the kit comprising:
(a) a dose of an isolated antibody, or antigen binding fragment thereof, that binds TSLP, wherein the antibody, or antigen binding fragment thereof, comprises a VH sequence set forth in SEQ ID NO: 34 and a VL sequence set forth in SEQ ID NO: 41;
(b) a dose of a hyaluronidase or variant thereof; and
(c) instructions.

46.-55. (canceled)
